SEQUENCE LISTING (741 sequences)

SEQ ID NO: 1 - protein, 39 aa, Heloderma horridum
HSDGTFTSDLSKQMEEEAVRLFIEWLKNGGPSSGAPPPS

SEQ ID NO: 2 - protein, 39 aa, Heloderma suspectum
HGEGTFTSDLSKQMEEEAVRLFIEWLKNGGPSSGAPPPS

SEQ ID NO: 3 - protein, 39 aa, Artificial Sequence
  Synthetic polypeptide
HGEGTFTSDLSKQLEEEAVRLFIEWLKNGGPSSGAPPPS

SEQ ID NO: 4 - protein, 37 aa, Artificial Sequence
  Synthetic polypeptide
HGEGTFTSDLSKQLEEEAVRLFIEWLKQGGPSKEIIS

SEQ ID NO: 5 - protein, 31 aa, Homo sapiens
HAEGTFTSDVSSYLEGQAAKEFIAWLVKGRG

SEQ ID NO: 6 - protein, 37 aa, Rattus norvegicus
KCNTATCATQRLANFLVRSSNNLGPVLPPTNVGSNTY

SEQ ID NO: 7 - protein, 37 aa, Homo sapiens [sequence omitted]

SEQ ID NO: 8 - protein, 37 aa, Artificial Sequence
  Synthetic polypeptide
KCNTATCATQRLANFLVHSSNNFGPILPPTNVGSNTY

SEQ ID NO: 9 - protein, 32 aa, Homo sapiens
CGNLSTCMLGTYTQDFNKFHTFPQTAIGVGAP

SEQ ID NO: 10 - protein, 32 aa, Oncorhynchus gorbuscha
CSNLSTCVLGKLSQELHKLQTYPRTNTGSGTP

SEQ ID NO: 11 - protein, 32 aa, Artificial Sequence
  Synthetic polypeptide
KCNTATCVLGRLSQELHRLQTYPRTNTGSNTY

SEQ ID NO: 12 - protein, 28 aa, Artificial Sequence
  Synthetic polypeptide
HGEGTFTSDLSKQMEEEAVRLFIEWLKN

SEQ ID NO: 13-22 - skipped (10 entries)

SEQ ID NO: 23 - protein, 46 aa, Artificial Sequence
  Synthetic polypeptide
LAEAKVLANRELDKYGVSDYYKNLINNAKTVEGVKALIDEILAALP

SEQ ID NO: 24 - protein, 46 aa, Artificial Sequence
  Synthetic polypeptide
LAEAKVLANRELDKYGVSDFYKSYINRAKTVEGVHTLIGHILAALP

SEQ ID NO: 25 - protein, 46 aa, Artificial Sequence
  Synthetic polypeptide
LAEAKVLANRELDKYGVSDFYKRLINKAKTVEGVNALTHHILAALP

SEQ ID NO: 26 - protein, 46 aa, Artificial Sequence
  Synthetic polypeptide
LAEAKVLANRELDKYGVSDYYKNLINRARTVEGVHALIDHILAALP

SEQ ID NO: 27 - protein, 46 aa, Artificial Sequence
  Synthetic polypeptide
LAEAKVLANRELDKYGVSDYYKNIINRAKTVEGVRALKLHILAALP

SEQ ID NO: 28 - protein, 46 aa, Artificial Sequence
  Synthetic polypeptide
LAEAKVLANRELDKYGVSDFYKNLINRAKTVEGVSSLKGHILAALP

SEQ ID NO: 29 - protein, 46 aa, Artificial Sequence
  Synthetic polypeptide
LAEAKVLANRELDKYGVSDYYKNLINKAKTVEGVEALTLHILAALP

SEQ ID NO: 30 - protein, 46 aa, Artificial Sequence
  Synthetic polypeptide
LAEAKVLANRELDKYGVSDFYKNLINRAKTVEGVDALIAHILAALP

SEQ ID NO: 31 - protein, 46 aa, Artificial Sequence
  Synthetic polypeptide
LAEAKVLANRELDKYGVSDFYKSLINRAKTVEGVDALTSHILAALP

SEQ ID NO: 32 - protein, 46 aa, Artificial Sequence
  Synthetic polypeptide
LAEAKVLANRELDKYGVSDFYKNLINRAKTVEGVNSLTSHILAALP

SEQ ID NO: 33 - protein, 46 aa, Artificial Sequence
  Synthetic polypeptide
LAEAKVLANRELDKYGVSDFYKNVINKAKTVEGVEALIADILAALP

SEQ ID NO: 34 - protein, 46 aa, Artificial Sequence
  Synthetic polypeptide
LAEAKVLANRELDKYGVSDYYKNLINKAKTVEGVQALIAHILAALP

SEQ ID NO: 35 - protein, 46 aa, Artificial Sequence
  Synthetic polypeptide
LAEAKVLANRELDKYGVSDFYKRLINKAKTVEGVEALKLHILAALP

SEQ ID NO: 36 - protein, 46 aa, Artificial Sequence; identical to SEQ ID NO: 27
  Synthetic polypeptide

SEQ ID NO: 37-39 - skipped (3 entries)

SEQ ID NO: 40 - protein, 93 aa, Artificial Sequence
  Synthetic polypeptide
HGEGTFTSDLSKQLEEEAVRLFIEWLKNGGPSSGAPPPSTGGGGSASLAEAKVLANRELDKYGVSDFYKRLINKAKTVEGVEALKLHILAALP

SEQ ID NO: 41 - protein, 105 aa, Artificial Sequence
  Synthetic polypeptide
HGEGTFTSDLSKQLEEEAVRLFIEWLKNGGPSSGAPPPSTGGGGSGGGSGGGSGGGSASLAEAKVLANRELDKYGVSDFYKRLINKAKTVEGVEALKLHILAALP

SEQ ID NO: 42 - protein, 91 aa, Artificial Sequence
  Synthetic polypeptide
HGEGTFTSDLSKQLEEEAVRLFIEWLKQGGPSKEIISTGGGGGASLAEAKVLANRELDKYGVSDFYKRLINKAKTVEGVEALKLHILAALP

SEQ ID NO: 43 - protein, 103 aa, Artificial Sequence
  Synthetic polypeptide
HGEGTFTSDLSKQLEEEAVRLFIEWLKQGGPSKEIISTGGGGSGGGSGGGSGGGSASLAEAKVLANRELDKYGVSDFYKRLINKAKTVEGVEALKLHILAALP

SEQ ID NO: 44-48 - skipped (5 entries)

SEQ ID NO: 49 - protein, 6 aa, Artificial Sequence
  Synthetic polypeptide
HHHHHH

SEQ ID NO: 50 - protein, 111 aa, Artificial Sequence
  Synthetic polypeptide
MAHHHHHHVGTGSNENLYFQHGEGTFTSDLSKQLEEEAVRLFIEWLKQGGPSKEIISTGGGGSASLAEAKVLANRELDKYGVSDFYKRLINKAKTVEGVEALKLHILAALP

SEQ ID NO: 51 - protein, 82 aa, Artificial Sequence
  Synthetic polypeptide
HGEGTFTSDLSKQMEEEAVRLFIEWLKNTGGGGSASLAEAKVLANRELDKYGVSDFYKRLINKAKTVEGVEALKLHILAALP

SEQ ID NO: 52 - protein, 82 aa, Artificial Sequence
  Synthetic polypeptide
HGEGTFTSDLSKQLEEEAVRLFIEWLKNTGGGGSASLAEAKVLANRELDKYGVSDFYKRLINKAKTVEGVEALKLHILAALP

SEQ ID NO: 53 - protein, 94 aa, Artificial Sequence
  Synthetic polypeptide
HGEGTFTSDLSKQMEEEAVRLFIEWLKNTGGGGSGGGSGGGSGGGSASLAEAKVLANRELDKYGVSDFYKRLINKAKTVEGVEALKLHILAALP

SEQ ID NO: 54 - protein, 94 aa, Artificial Sequence
  Synthetic polypeptide
HGEGTFTSDLSKQLEEEAVRLFIEWLKNTGGGGSGGGSGGGSGGGSASLAEAKVLANRELDKYGVSDFYKRLINKAKTVEGVEALKLHILAALP

SEQ ID NO: 55 - protein, 93 aa, Artificial Sequence
  Synthetic polypeptide
HGEGTFTSDLSKQMEEEAVRLFIEWLKNGGPSSGAPPPSTGGGGSASLAEAKVLANRELDKYGVSDFYKRLINKAKTVEGVEALKLHILAALP

SEQ ID NO: 56 - protein, 105 aa, Artificial Sequence
  Synthetic polypeptide
HGEGTFTSDLSKQMEEEAVRLFIEWLKNGGPSSGAPPPSTGGGGSGGGSGGGSGGGSASLAEAKVLANRELDKYGVSDFYKRLINKAKTVEGVEALKLHILAALP

SEQ ID NO: 57 - protein, 91 aa, Artificial Sequence
  Synthetic polypeptide
HGEGTFTSDLSKQMEEEAVRLFIEWLKQGGPSKEIISTGGGGSASLAEAKVLANRELDKYGVSDFYKRLINKAKTVEGVEALKLHILAALP

SEQ ID NO: 58 - protein, 103 aa, Artificial Sequence
  Synthetic polypeptide
HGEGTFTSDLSKQMEEEAVRLFIEWLKQGGPSKEIISTGGGGSGGGSGGGSGGGSASLAEAKVLANRELDKYGVSDFYKRLINKAKTVEGVEALKLHILAALP

SEQ ID NO: 59 - protein, 98 aa, Artificial Sequence
  Synthetic polypeptide
HGEGTFTSDLSKQMEEEAVRLFIEWLKNGGPSSGAPPSKKKKKKTGGGGSASLAEAKVLANRELDKYGVSDFYKRLINKAKTVEGVEALKLHILAALP

SEQ ID NO: 60 - protein, 109 aa, Artificial Sequence
  Synthetic polypeptide
HGEGTFTSDLSKQMEEEAVRLFIEWLKNGGPSSGAPPSKKKKKKTGGGGSGGGSGGGGGGSASLAEAKVLANRELDKYGVSDFYKRLINKAKTVEGVEALKLHILAALP

SEQ ID NO: 61 - protein, 88 aa, Artificial Sequence
  Synthetic polypeptide
HGEGTFTSDLSKQLEEEAVRLFIEWLKNGGPSSGAPPPSGGGLAEAKVLANRELDKYGVSDFYKRLINKAKTVEGVEALKLHILAALP

SEQ ID NO: 62 - protein, 94 aa, Artificial Sequence
  Synthetic polypeptide
HGEGTFTSDLSKQMEEEAVRLFIEWLKNTGGGGSGGGSGGGSGGGSASLAEAKVLANRELDKYGVSDYYKNIINRAKTVEGVRALKLHILAALP

SEQ ID NO: 63 - protein, 93 aa, Artificial Sequence
  Synthetic polypeptide
HGEGTFTSDLSKQLEEEAVRLFIEWLKNGGPSSGAPPPSTGGGGSASLAEAKVLANRELDKYGVSDYYKNIINRAKTVEGVRALKLHILAALP

SEQ ID NO: 64 - protein, 105 aa, Artificial Sequence
  Synthetic polypeptide
HGEGTFTSDLSKQLEEEAVRLFIEWLKNGGPSSGAPPPSTGGGGSGGGSGGGSGGGSASLAEAKVLANRELDKYGVSDYYKNIINRAKTVEGVRALKLHILAALP

SEQ ID NO: 65 - protein, 91 aa, Artificial Sequence
  Synthetic polypeptide
HGEGTFTSDLSKQLEEEAVRLFIEWLKQGGPSKEIISTGGGGSASLAEAKVLANRELDKYGVSDYYKNIINRAKTVEGVRALKLHILAALP

SEQ ID NO: 66 - protein, 103 aa, Artificial Sequence
  Synthetic polypeptide
HGEGTFTSDLSKQLEEEAVRLFIEWLKQGGPSKEIISTGGGGSGGGSGGGSGGGSASLAEAKVLANRELDKYGVSDYYKNIINRAKTVEGVRALKLHILAALP

SEQ ID NO: 67 - protein, 82 aa, Artificial Sequence
  Synthetic polypeptide
HGEGTFTSDLSKQMEEEAVRLFIEWLKNTGGGGSASLAEAKVLANRELDKYGVSDYYKNIINRAKTVEGVRALKLHILAALP

SEQ ID NO: 68 - protein, 82 aa, Artificial Sequence
  Synthetic polypeptide
HGEGTFTSDLSKQLEEEAVRLFIEWLKNTGGGGSASLAEAKVLANRELDKYGVSDYYKNIINRAKTVEGVRALKLHILAALP

SEQ ID NO: 69 - skipped

SEQ ID NO: 70 - protein, 94 aa, Artificial Sequence
  Synthetic polypeptide
HGEGTFTSDLSKQLEEEAVRLFIEWLKNTGGGGSGGGSGGGSGGGSASLAEAKVLANRELDKYGVSDYYKNIINRAKTVEGVRALKLHILAALP

SEQ ID NO: 71 - protein, 93 aa, Artificial Sequence
  Synthetic polypeptide
HGEGTFTSDLSKQMEEEAVRLFIEWLKNGGPSSGAPPPSTGGGGSASLAEAKVLANRELDKYGVSDYYKNIINRAKTVEGVRALKLHILAALP

SEQ ID NO: 72 - protein, 105 aa, Artificial Sequence
  Synthetic polypeptide
HGEGTFTSDLSKQMEEEAVRLFIEWLKNGGPSSGAPPPSTGGGGSGGGSGGGSGGGSASLAEAKVLANRELDKYGVSDYYKNIINRAKTVEGVRALKLHILAALP

SEQ ID NO: 73 - protein, 91 aa, Artificial Sequence
  Synthetic polypeptide
HGEGTFTSDLSKQMEEEAVRLFIEWLKQGGPSKEIISTGGGGSASLAEAKVLANRELDKYGVSDYYKNIINRAKTVEGVRALKLHILAALP

SEQ ID NO: 74 - protein, 103 aa, Artificial Sequence
  Synthetic polypeptide
HGEGTFTSDLSKQMEEEAVRLFIEWLKQGGPSKEIISTGGGGSGGGSGGGSGGGSASLAEAKVLANRELDKYGVSDYYKNIINRAKTVEGVRALKLHILAALP

SEQ ID NO: 75 - protein, 98 aa, Artificial Sequence
  Synthetic polypeptide
HGEGTFTSDLSKQMEEEAVRLFIEWLKNGGPSSGAPPSKKKKKKTGGGGSASLAEAKVLANRELDKYGVSDYYKNIINRAKTVEGVRALKLHILAALP

SEQ ID NO: 76 - protein, 110 aa, Artificial Sequence
  Synthetic polypeptide
HGEGTFTSDLSKQMEEEAVRLFIEWLKNGGPSSGAPPSKKKKKKTGGGGSGGGSGGGSGGGSASLAEAKVLANRELDKYGVSDYYKNIINRAKTVEGVRALKLHILAALP

SEQ ID NO: 77 - protein, 88 aa, Artificial Sequence
  Synthetic polypeptide
HGEGTFTSDLSKQLEEEAVRLFIEWLKNGGPSSGAPPPSGGGLAEAKVLANRELDKYGVSDYYKNIINRAKTVEGVRALKLHILAALP

SEQ ID NO: 78 - protein, 88 aa, Artificial Sequence; identical to SEQ ID NO: 61
  Synthetic polypeptide

SEQ ID NO: 79 - protein, 86 aa, Artificial Sequence
  Synthetic polypeptide
HGEGTFTSDLSKQLEEEAVRLFIEWLKQGGPSKEIISGGGLAEAKVLANRELDKYGVSDFYKRLINKAKTVEGVEALKLHILAALP

SEQ ID NO: 80 - protein, 77 aa, Artificial Sequence
  Synthetic polypeptide
HGEGTFTSDLSKQMEEEAVRLFIEWLKNGGGLAEAKVLANRELDKYGVSDFYKRLINKAKTVEGVEALKLHILAALP

SEQ ID NO: 81 - protein, 77 aa, Artificial Sequence
  Synthetic polypeptide
HGEGTFTSDLSKQLEEEAVRLFIEWLKNGGGLAEAKVLANRELDKYGVSDFYKRLINKAKTVEGVEALKLHILAALP

SEQ ID NO: 82 - protein, 88 aa, Artificial Sequence
  Synthetic polypeptide
HGEGTFTSDLSKQMEEEAVRLFIEWLKNGGPSSGAPPPSGGGLAEAKVLANRELDKYGVSDFYKRLINKAKTVEGVEALKLHILAALP

SEQ ID NO: 83 - protein, 86 aa, Artificial Sequence
  Synthetic polypeptide
HGEGTFTSDLSKQMEEEAVRLFIEWLKQGGPSKEIISGGGLAEAKVLANRELDKYGVSDFYKRLINKAKTVEGVEALKLHILAALP

SEQ ID NO: 84 - protein, 93 aa, Artificial Sequence
  Synthetic polypeptide
HGEGTFTSDLSKQMEEEAVRLFIEWLKNGGPSSGAPPSKKKKKKGGGLAEAKVLANRELDKYGVSDFYKRLINKAKTVEGVEALKLHILAALP

SEQ ID NO: 85 - protein, 88 aa, Artificial Sequence; identical to SEQ ID NO: 61
  Synthetic polypeptide

SEQ ID NO: 86 - protein, 77 aa, Artificial Sequence
  Synthetic polypeptide
HGEGTFTSDLSKQMEEEAVRLFIEWLKNGGGLAEAKVLANRELDKYGVSDYYKNIINRAKTVEGVRALKLHILAALP

SEQ ID NO: 87 - protein, 88 aa, Artificial Sequence; identical to SEQ ID NO: 77
  Synthetic polypeptide

SEQ ID NO: 88 - protein, 86 aa, Artificial Sequence
  Synthetic polypeptide
HGEGTFTSDLSKQLEEEAVRLFIEWLKQGGPSKEIISGGGLAEAKVLANRELDKYGVSDYYKNIINRAKTVEGVRALKLHILAALP

SEQ ID NO: 89 - protein, 77 aa, Artificial Sequence
  Synthetic polypeptide
HGEGTFTSDLSKQLEEEAVRLFIEWLKNGGGLAEAKVLANRELDKYGVSDYYKNIINRAKTVEGVRALKLHILAALP

SEQ ID NO: 90 - protein, 77 aa, Artificial Sequence; identical to SEQ ID NO: 89
  Synthetic polypeptide

SEQ ID NO: 91 - protein, 88 aa, Artificial Sequence
  Synthetic polypeptide
HGEGTFTSDLSKQMEEEAVRLFIEWLKNGGPSSGAPPPSGGGLAEAKVLANRELDKYGVSDYYKNIINRAKTVEGVRALKLHILAALP

SEQ ID NO: 92 - protein, 86 aa, Artificial Sequence
  Synthetic polypeptide
HGEGTFTSDLSKQMEEEAVRLFIEWLKQGGPSKEIISGGGLAEAKVLANRELDKYGVSDYYKNIINRAKTVEGVRALKLHILAALP

SEQ ID NO: 93 - protein, 93 aa, Artificial Sequence
  Synthetic polypeptide
HGEGTFTSDLSKQMEEEAVRLFIEWLKNGGPSSGAPPSKKKKKKGGGLAEAKVLANRELDKYGVSDYYKNIINRAKTVEGVRALKLHILAALP

SEQ ID NO: 94 - protein, 88 aa, Artificial Sequence
  Synthetic polypeptide
HGEGTFTSDLSKQLEEEAVRLFIEWLKNGGPSSGAPPPSGGSLAEAKVLANRELDKYGVSDYYKNIINRAKTVEGVRALKLHILAALP

SEQ ID NO: 95 - protein, 88 aa, Artificial Sequence
  Synthetic polypeptide
HGEGTFTSDLSKQLEEEAVRLFIEWLKNGGPSSGAPPPSGGSLAEAKVLANRELDKYGVSDFYKRLINKAKTVEGVEALKLHILAALP

SEQ ID NO: 96 - protein, 86 aa, Artificial Sequence
  Synthetic polypeptide
HGEGTFTSDLSKQLEEEAVRLFIEWLKQGGPSKEIISGGSLAEAKVLANRELDKYGVSDFYKRLINKAKTVEGVEALKLHILAALP

SEQ ID NO: 97 - protein, 77 aa, Artificial Sequence
  Synthetic polypeptide
HGEGTFTSDLSKQMEEEAVRLFIEWLKNGGSLAEAKVLANRELDKYGVSDFYKRLINKAKTVEGVEALKLHILAALP

SEQ ID NO: 98 - protein, 77 aa, Artificial Sequence
  Synthetic polypeptide
HGEGTFTSDLSKQLEEEAVRLFIEWLKNGGSLAEAKVLANRELDKYGVSDFYKRLINKAKTVEGVEALKLHILAALP

SEQ ID NO: 99 - protein, 88 aa, Artificial Sequence
  Synthetic polypeptide
HGEGTFTSDLSKQMEEEAVRLFIEWLKNGGPSSGAPPPSGGSLAEAKVLANRELDKYGVSDFYKRLINKAKTVEGVEALKLHILAALP

SEQ ID NO: 100 - protein, 86 aa, Artificial Sequence
  Synthetic polypeptide
HGEGTFTSDLSKQMEEEAVRLFIEWLKQGGPSKEIISGGSLAEAKVLANRELDKYGVSDFYKRLINKAKTVEGVEALKLHILAALP

SEQ ID NO: 101 - protein, 93 aa, Artificial Sequence
  Synthetic polypeptide
HGEGTFTSDLSKQMEEEAVRLFIEWLKNGGPSSGAPPSKKKKKKGGSLAEAKVLANRELDKYGVSDFYKRLINKAKTVEGVEALKLHILAALP

SEQ ID NO: 102 - protein, 77 aa, Artificial Sequence
  Synthetic polypeptide
HGEGTFTSDLSKQMEEEAVRLFIEWLKNGGSLAEAKVLANRELDKYGVSDYYKNIINRAKTVEGVRALKLHILAALP

SEQ ID NO: 103 - protein, 88 aa, Artificial Sequence; identical to SEQ ID NO: 94
  Synthetic polypeptide

SEQ ID NO: 104 - protein, 86 aa, Artificial Sequence
  Synthetic polypeptide
HGEGTFTSDLSKQLEEEAVRLFIEWLKQGGPSKEIISGGSLAEAKVLANRELDKYGVSDYYKNIINRAKTVEGVRALKLHILAALP

SEQ ID NO: 105 - protein, 77 aa, Artificial Sequence
  Synthetic polypeptide
HGEGTFTSDLSKQLEEEAVRLFIEWLKNGGSLAEAKVLANRELDKYGVSDYYKNIINRAKTVEGVRALKLHILAALP

SEQ ID NO: 106 - protein, 77 aa, Artificial Sequence; identical to SEQ ID NO: 105
  Synthetic polypeptide

SEQ ID NO: 107 - protein, 88 aa, Artificial Sequence
  Synthetic polypeptide
HGEGTFTSDLSKQMEEEAVRLFIEWLKNGGPSSGAPPPSGGSLAEAKVLANRELDKYGVSDYYKNIINRAKTVEGVRALKLHILAALP

SEQ ID NO: 108 - protein, 86 aa, Artificial Sequence
  Synthetic polypeptide
HGEGTFTSDLSKQMEEEAVRLFIEWLKQGGPSKEIISGGSLAEAKVLANRELDKYGVSDYYKNIINRAKTVEGVRALKLHILAALP

SEQ ID NO: 109 - protein, 93 aa, Artificial Sequence
  Synthetic polypeptide
HGEGTFTSDLSKQMEEEAVRLFIEWLKNGGPSSGAPPSKKKKKKGGSLAEAKVLANRELDKYGVSDYYKNIINRAKTVEGVRALKLHILAALP

SEQ ID NO: 110 - skipped

SEQ ID NO: 111 - protein, 37 aa, Artificial Sequence
  Synthetic polypeptide
HGEGTFTSDLSKQMEEEAVRLFIEWLKQGGPSKEIIS

SEQ ID NO: 112 - protein, 38 aa, Artificial Sequence
  Synthetic polypeptide
HGEGTFTSDLSKQLEEEAVRLFIEWLKNGGPSSGAPPS

SEQ ID NO: 113 - protein, 38 aa, Artificial Sequence
  Synthetic polypeptide
HGEGTFTSDLSKQMEEEAVRLFIEWLKNGGPSSGAPPS

SEQ ID NO: 114 - protein, 43 aa, Artificial Sequence
  Synthetic polypeptide
HGEGTFTSDLSKQLEEEAVRLFIEWLKQGGPSKEIISKKKKKK

SEQ ID NO: 115 - protein, 45 aa, Artificial Sequence
  Synthetic polypeptide
HGEGTFTSDLSKQMEEEAVRLFIEWLKNGGPSSGAPPPSKKKKKK

SEQ ID NO: 116 - protein, 43 aa, Artificial Sequence
  Synthetic polypeptide
HGEGTFTSDLSKQMEEEAVRLFIEWLKQGGPSKEIISKKKKKK

SEQ ID NO: 117 - protein, 44 aa, Artificial Sequence
  Synthetic polypeptide
HGEGTFTSDLSKQLEEEAVRLFIEWLKNGGPSSGAPPSKKKKKK

SEQ ID NO: 118 - protein, 44 aa, Artificial Sequence
  Synthetic polypeptide
HGEGTFTSDLSKQMEEEAVRLFIEWLKNGGPSSGAPPSKKKKKK

SEQ ID NO: 119 - protein, 26 aa, Artificial Sequence
  Synthetic polypeptide
  VARIANT (5)..(5): Xaa is Tyr or Phe
  VARIANT (8)..(8): Xaa is Asn, Arg or Ser
  VARIANT (9)..(9): Xaa is Val, Ile, Leu, MEt, Phe or Tyr
  VARIANT (11)..(11): Xaa is Asn, Ser, Glu or Asp
  VARIANT (12)..(12): Xaa is Arg, Lys or Asn
  VARIANT (14)..(14): Xaa is Lys or Arg
  VARIANT (20)..(20): Xaa is Asp, Asn, Qln, Glu, His, Ser, Arg or Lys
  VARIANT (23)..(23): Xaa is Lys, Ile or Thr
  VARIANT (24)..(24): Xaa is Ala, Ser, Thr, Gly, His, Leu or Asp
  VARIANT (25)..(25): Xaa is His, Glu or Asp
GVSDXYKXXIXXAXTVEGVXALXXXI

SEQ ID NO: 120 - protein, 26 aa, Artificial Sequence
  Synthetic polypeptide
GVSDYYKNLINNAKTVEGVKALIDEI

SEQ ID NO: 121 - protein, 46 aa, Artificial Sequence
  Synthetic polypeptide
  VARIANT (6)..(6): Xaa is Val or Glu
  VARIANT (7)..(7): Xaa is Leu, Glu or Asp
  VARIANT (9)..(9): Xaa is Asn, Leu or Ile
  VARIANT (10)..(10): Xaa is Arg or Lys
  VARIANT (13)..(13): Xaa is Asp or Lys
  VARIANT (20)..(20): Xaa is Tyr or Phe
  VARIANT (23)..(23): Xaa is Asn, Arg or Ser
  VARIANT (24)..(24): Xaa is Val, Ile, Leu, Met, Phe or Tyr
  VARIANT (26)..(26): Xaa is Asn, Ser, Glu or Asp
  VARIANT (27)..(27): Xaa is Arg, Lys or Asn
  VARIANT (29)..(29): Xaa is Lys or Arg
  VARIANT (35)..(35): Xaa is Asp, Asn, Qln, Glu, Hi,s Ser, Arg or Lys
  VARIANT (38)..(38): Xaa is Lys, Ile or Thr
  VARIANT (39)..(39): Xaa is Ala, Ser, Thr, Gly, His, Leu or Asp
  VARIANT (40)..(40): Xaa is His, Glu or Asp
LAEAKXXAXXELXKYGVSDXYKXXIXXAXTVEGVXALXXXILAALP

SEQ ID NO: 122 - protein, 46 aa, Artificial Sequence
  Synthetic polypeptide
LAEAKEDAIKELDKYGVSDYYKRLISKAKTVEGVKALISEILAALP

SEQ ID NO: 123 - protein, 46 aa, Artificial Sequence
  Synthetic polypeptide
LAEAKEDAIKELDKYGVSDYYKNLINKAKTVEGVEALTLHILAALP

SEQ ID NO: 124 - protein, 46 aa, Artificial Sequence
  Synthetic polypeptide
LAEAKEDAIKELDKYGVSDYYKNLINKAKTVEGVEALISEILAALP

SEQ ID NO: 125 - protein, 4 aa, Artificial Sequence
  Synthetic polypeptide
GGGG

SEQ ID NO: 126 - protein, 5 aa, Artificial Sequence
  Synthetic polypeptide
GGGGG

SEQ ID NO: 127 - protein, 7 aa, Artificial Sequence
  Synthetic polypeptide
  MISC_FEATURE (2)..(2): Xaa is Gly or absent
  MISC_FEATURE (3)..(3): Xaa is Gly or absent
  MISC_FEATURE (4)..(4): Xaa is Gly or absent
  MISC_FEATURE (5)..(5): Xaa is Gly or absent
  MISC_FEATURE (6)..(6): Xaa is Gly or absent
GXXXXXE

SEQ ID NO: 128 - protein, 7 aa, Artificial Sequence
  Synthetic polypeptide
  MISC_FEATURE (2)..(2): Xaa is Gly or absent
  MISC_FEATURE (3)..(3): Xaa is Gly or absent
  MISC_FEATURE (4)..(4): Xaa is Gly or absent
  MISC_FEATURE (5)..(5): Xaa is Gly or absent
  MISC_FEATURE (6)..(6): Xaa is Gly or absent
GXXXXXK

SEQ ID NO: 129 - protein, 7 aa, Artificial Sequence
  Synthetic polypeptide
  MISC_FEATURE (2)..(2): Xaa is Gly or absent
  MISC_FEATURE (3)..(3): Xaa is Gly or absent
  MISC_FEATURE (4)..(4): Xaa is Gly or absent
  MISC_FEATURE (5)..(5): Xaa is Gly or absent
  MISC_FEATURE (6)..(6): Xaa is Gly or absent
GXXXXXD

SEQ ID NO: 130 - protein, 7 aa, Artificial Sequence
  Synthetic polypeptide
  MISC_FEATURE (2)..(2): Xaa is Gly or absent
  MISC_FEATURE (3)..(3): Xaa is Gly or absent
  MISC_FEATURE (4)..(4): Xaa is Gly or absent
  MISC_FEATURE (5)..(5): Xaa is Gly or absent
  MISC_FEATURE (6)..(6): Xaa is Gly or absent
GXXXXXR

SEQ ID NO: 131 - protein, 8 aa, Artificial Sequence
  Synthetic polypeptide
GGGKGGGG

SEQ ID NO: 132 - protein, 8 aa, Artificial Sequence
  Synthetic polypeptide
GGGNGSGG

SEQ ID NO: 133 - protein, 8 aa, Artificial Sequence
  Synthetic polypeptide
GGGCGGGG

SEQ ID NO: 134 - protein, 5 aa, Artificial Sequence
  Synthetic polypeptide
GPNGG

SEQ ID NO: 135 - protein, 12 aa, Artificial Sequence
  Synthetic polypeptide
  MISC_FEATURE (3)..(4): Residues may be absent
  MISC_FEATURE (5)..(6): Residues may be absent
  MISC_FEATURE (7)..(8): Residues may be absent
  MISC_FEATURE (9)..(10): Residues may be absent
  MISC_FEATURE (11)..(12): Residues may be absent
GSGSGSGSGSGS

SEQ ID NO: 136 - protein, 18 aa, Artificial Sequence
  Synthetic polypeptide
  MISC_FEATURE (4)..(6): Residues may be absent
  MISC_FEATURE (7)..(9): Residues may be absent
  MISC_FEATURE (10)..(12): Residues may be absent
  MISC_FEATURE (13)..(15): Residues may be absent
  MISC_FEATURE (16)..(18): Residues may be absent
GGSGGSGGSGGSGGSGGS

SEQ ID NO: 137 - protein, 24 aa, Artificial Sequence
  Synthetic polypeptide
  MISC_FEATURE (5)..(8): Residues may be absent
  MISC_FEATURE (9)..(12): Residues may be absent
  MISC_FEATURE (13)..(16): Residues may be absent
  MISC_FEATURE (17)..(20): Residues may be absent
  MISC_FEATURE (21)..(24): Residues may be absent
GGGSGGGSGGGSGGGSGGGSGGGS

SEQ ID NO: 138 - protein, 30 aa, Artificial Sequence
  Synthetic polypeptide
  MISC_FEATURE (6)..(10): Residues may be absent
  MISC_FEATURE (11)..(15): Residues may be absent
  MISC_FEATURE (16)..(20): Residues may be absent
  MISC_FEATURE (21)..(25): Residues may be absent
  MISC_FEATURE (26)..(30): Residues may be absent
GGGGSGGGGSGGGGSGGGGSGGGGSGGGGS

SEQ ID NO: 139 - protein, 20 aa, Artificial Sequence
  Synthetic polypeptide
  MISC_FEATURE (3)..(4): Resdiues may be absent
  MISC_FEATURE (5)..(6): Resdiues may be absent
  MISC_FEATURE (7)..(8): Resdiues may be absent
  MISC_FEATURE (9)..(10): Resdiues may be absent
  MISC_FEATURE (11)..(12): Resdiues may be absent
  MISC_FEATURE (13)..(14): Resdiues may be absent
  MISC_FEATURE (15)..(16): Resdiues may be absent
  MISC_FEATURE (17)..(18): Resdiues may be absent
  MISC_FEATURE (19)..(20): Resdiues may be absent
GEGEGEGEGEGEGEGEGEGE

SEQ ID NO: 140 - protein, 30 aa, Artificial Sequence
  Synthetic polypeptide
  MISC_FEATURE (4)..(6): Residues may be absent
  MISC_FEATURE (7)..(9): Residues may be absent
  MISC_FEATURE (10)..(12): Residues may be absent
  MISC_FEATURE (13)..(15): Residues may be absent
  MISC_FEATURE (16)..(18): Residues may be absent
  MISC_FEATURE (19)..(21): Residues may be absent
  MISC_FEATURE (22)..(24): Residues may be absent
  MISC_FEATURE (25)..(27): Residues may be absent
  MISC_FEATURE (28)..(30): Residues may be absent
GGEGGEGGEGGEGGEGGEGGEGGEGGEGGE

SEQ ID NO: 141 - protein, 40 aa, Artificial Sequence
  Synthetic polypeptide
  MISC_FEATURE (5)..(8): Residues may be absent
  MISC_FEATURE (9)..(12): Residues may be absent
  MISC_FEATURE (13)..(16): Residues may be absent
  MISC_FEATURE (17)..(20): Residues may be absent
  MISC_FEATURE (21)..(24): Residues may be absent
  MISC_FEATURE (25)..(28): Residues may be absent
  MISC_FEATURE (29)..(32): Residues may be absent
  MISC_FEATURE (33)..(36): Residues may be absent
  MISC_FEATURE (37)..(40): Residues may be absent
GGGEGGGEGGGEGGGEGGGEGGGEGGGEGGGEGGGEGGGE

SEQ ID NO: 142 - protein, 50 aa, Artificial Sequence
  Synthetic polypeptide
  MISC_FEATURE (6)..(10): Residues may be absent
  MISC_FEATURE (11)..(15): Residues may be absent
  MISC_FEATURE (16)..(20): Residues may be absent
  MISC_FEATURE (21)..(25): Residues may be absent
  MISC_FEATURE (26)..(30): Residues may be absent
  MISC_FEATURE (31)..(35): Residues may be absent
  MISC_FEATURE (36)..(40): Residues may be absent
  MISC_FEATURE (41)..(45): Residues may be absent
  MISC_FEATURE (46)..(50): Residues may be absent
GGGGEGGGGEGGGGEGGGGEGGGGEGGGGEGGGGEGGGGEGGGGEGGGGE

SEQ ID NO: 143 - protein, 20 aa, Artificial Sequence
  Synthetic polypeptide
  MISC_FEATURE (3)..(4): Residues may be absent
  MISC_FEATURE (5)..(6): Residues may be absent
  MISC_FEATURE (7)..(8): Residues may be absent
  MISC_FEATURE (9)..(10): Residues may be absent
  MISC_FEATURE (11)..(12): Residues may be absent
  MISC_FEATURE (13)..(14): Residues may be absent
  MISC_FEATURE (15)..(16): Residues may be absent
  MISC_FEATURE (17)..(18): Residues may be absent
  MISC_FEATURE (19)..(20): Residues may be absent
GDGDGDGDGDGDGDGDGDGD

SEQ ID NO: 144 - protein, 30 aa, Artificial Sequence
  Synthetic polypeptide
  MISC_FEATURE (2)..(3): Residues may be absent
  MISC_FEATURE (4)..(6): Residues may be absent
  MISC_FEATURE (7)..(9): Residues may be absent
  MISC_FEATURE (10)..(12): Residues may be absent
  MISC_FEATURE (16)..(18): Residues may be absent
  MISC_FEATURE (19)..(21): Residues may be absent
  MISC_FEATURE (22)..(24): Residues may be absent
  MISC_FEATURE (25)..(27): Residues may be absent
  MISC_FEATURE (28)..(30): Residues may be absent
GGDGGDGGDGGDGGDGGDGGDGGDGGDGGD

SEQ ID NO: 145 - protein, 40 aa, Artificial Sequence
  Synthetic polypeptide
  MISC_FEATURE (5)..(8): Residues may be absent
  MISC_FEATURE (9)..(12): Residues may be absent
  MISC_FEATURE (13)..(16): Residues may be absent
  MISC_FEATURE (17)..(20): Residues may be absent
  MISC_FEATURE (21)..(24): Residues may be absent
  MISC_FEATURE (25)..(28): Residues may be absent
  MISC_FEATURE (29)..(32): Residues may be absent
  MISC_FEATURE (33)..(36): Residues may be absent
  MISC_FEATURE (37)..(40): Residues may be absent
GGGDGGGDGGGDGGGDGGGDGGGDGGGDGGGDGGGDGGGD

SEQ ID NO: 146 - protein, 50 aa, Artificial Sequence
  Synthetic polypeptide
  MISC_FEATURE (6)..(10): Residues may be absent
  MISC_FEATURE (11)..(15): Residues may be absent
  MISC_FEATURE (16)..(20): Residues may be absent
  MISC_FEATURE (21)..(25): Residues may be absent
  MISC_FEATURE (26)..(30): Residues may be absent
  MISC_FEATURE (31)..(35): Residues may be absent
  MISC_FEATURE (36)..(40): Residues may be absent
  MISC_FEATURE (41)..(45): Residues may be absent
  MISC_FEATURE (46)..(50): Residues may be absent
GGGGDGGGGDGGGGDGGGGDGGGGDGGGGDGGGGDGGGGDGGGGDGGGGD

SEQ ID NO: 147 - protein, 20 aa, Artificial Sequence
  Synthetic polypeptide
  MISC_FEATURE (3)..(4): Residues may be absent
  MISC_FEATURE (5)..(6): Residues may be absent
  MISC_FEATURE (7)..(8): Residues may be absent
  MISC_FEATURE (9)..(10): Residues may be absent
  MISC_FEATURE (11)..(12): Residues may be absent
  MISC_FEATURE (13)..(14): Residues may be absent
  MISC_FEATURE (15)..(16): Residues may be absent
  MISC_FEATURE (17)..(18): Residues may be absent
  MISC_FEATURE (19)..(20): Residues may be absent
GKGKGKGKGKGKGKGKGKGK

SEQ ID NO: 148 - protein, 30 aa, Artificial Sequence
  Synthetic polypeptide
  MISC_FEATURE (4)..(6): Residues may be absent
  MISC_FEATURE (7)..(9): Residues may be absent
  MISC_FEATURE (10)..(12): Residues may be absent
  MISC_FEATURE (13)..(15): Residues may be absent
  MISC_FEATURE (16)..(18): Residues may be absent
  MISC_FEATURE (19)..(21): Residues may be absent
  MISC_FEATURE (22)..(24): Residues may be absent
  MISC_FEATURE (25)..(27): Residues may be absent
  MISC_FEATURE (28)..(30): Residues may be absent
GGKGGKGGKGGKGGKGGKGGKGGKGGKGGK

SEQ ID NO: 149 - protein, 40 aa, Artificial Sequence
  Synthetic polypeptide
  MISC_FEATURE (5)..(8): Residues may be absent
  MISC_FEATURE (9)..(12): Residues may be absent
  MISC_FEATURE (13)..(16): Residues may be absent
  MISC_FEATURE (17)..(20): Residues may be absent
  MISC_FEATURE (21)..(24): Residues may be absent
  MISC_FEATURE (25)..(28): Residues may be absent
  MISC_FEATURE (29)..(32): Residues may be absent
  MISC_FEATURE (33)..(36): Residues may be absent
  MISC_FEATURE (37)..(40): Residues may be absent
GGGKGGGKGGGKGGGKGGGKGGGKGGGKGGGKGGGKGGGK

SEQ ID NO: 150 - protein, 50 aa, Artificial Sequence
  Synthetic polypeptide
  MISC_FEATURE (6)..(10): Residues may be absent
  MISC_FEATURE (11)..(15): Residues may be absent
  MISC_FEATURE (16)..(20): Residues may be absent
  MISC_FEATURE (21)..(25): Residues may be absent
  MISC_FEATURE (26)..(30): Residues may be absent
  MISC_FEATURE (31)..(35): Residues may be absent
  MISC_FEATURE (36)..(40): Residues may be absent
  MISC_FEATURE (41)..(45): Residues may be absent
  MISC_FEATURE (46)..(50): Residues may be absent
GGGGKGGGGKGGGGKGGGGKGGGGKGGGGKGGGGKGGGGKGGGGKGGGGK

SEQ ID NO: 151 - protein, 20 aa, Artificial Sequence
  Synthetic polypeptide
  MISC_FEATURE (3)..(4): Residues may be absent
  MISC_FEATURE (5)..(6): Residues may be absent
  MISC_FEATURE (7)..(8): Residues may be absent
  MISC_FEATURE (9)..(10): Residues may be absent
  MISC_FEATURE (11)..(12): Residues may be absent
  MISC_FEATURE (13)..(14): Residues may be absent
  MISC_FEATURE (15)..(16): Residues may be absent
  MISC_FEATURE (17)..(18): Residues may be absent
  MISC_FEATURE (19)..(20): Residues may be absent
GRGRGRGRGRGRGRGRGRGR

SEQ ID NO: 152 - protein, 30 aa, Artificial Sequence
  Synthetic polypeptide
  MISC_FEATURE (4)..(6): Residues may be absent
  MISC_FEATURE (7)..(9): Residues may be absent
  MISC_FEATURE (10)..(12): Residues may be absent
  MISC_FEATURE (13)..(15): Residues may be absent
  MISC_FEATURE (16)..(18): Residues may be absent
  MISC_FEATURE (19)..(21): Residues may be absent
  MISC_FEATURE (22)..(24): Residues may be absent
  MISC_FEATURE (25)..(27): Residues may be absent
  MISC_FEATURE (28)..(30): Residues may be absent
GGRGGRGGRGGRGGRGGRGGRGGRGGRGGR

SEQ ID NO: 153 - protein, 40 aa, Artificial Sequence
  Synthetic polypeptide
  MISC_FEATURE (5)..(8): Residues may be absent
  MISC_FEATURE (9)..(12): Residues may be absent
  MISC_FEATURE (13)..(16): Residues may be absent
  MISC_FEATURE (17)..(20): Residues may be absent
  MISC_FEATURE (21)..(24): Residues may be absent
  MISC_FEATURE (25)..(28): Residues may be absent
  MISC_FEATURE (29)..(32): Residues may be absent
  MISC_FEATURE (33)..(36): Residues may be absent
  MISC_FEATURE (37)..(40): Residues may be absent
GGGRGGGRGGGRGGGRGGGRGGGRGGGRGGGRGGGRGGGR

SEQ ID NO: 154 - protein, 50 aa, Artificial Sequence
  Synthetic polypeptide
  MISC_FEATURE (6)..(10): Residues may be absent
  MISC_FEATURE (11)..(15): Residues may be absent
  MISC_FEATURE (16)..(20): Residues may be absent
  MISC_FEATURE (21)..(25): Residues may be absent
  MISC_FEATURE (26)..(30): Residues may be absent
  MISC_FEATURE (31)..(35): Residues may be absent
  MISC_FEATURE (36)..(40): Residues may be absent
  MISC_FEATURE (41)..(45): Residues may be absent
  MISC_FEATURE (46)..(50): Residues may be absent
GGGGRGGGGRGGGGRGGGGRGGGGRGGGGRGGGGRGGGGRGGGGRGGGGR

SEQ ID NO: 155 - protein, 50 aa, Artificial Sequence
  Synthetic polypeptide
  MISC_FEATURE (6)..(10): Residues may be absent
  MISC_FEATURE (11)..(15): Residues may be absent
  MISC_FEATURE (16)..(20): Residues may be absent
  MISC_FEATURE (21)..(25): Residues may be absent
  MISC_FEATURE (26)..(30): Residues may be absent
  MISC_FEATURE (31)..(35): Residues may be absent
  MISC_FEATURE (36)..(40): Residues may be absent
  MISC_FEATURE (41)..(45): Residues may be absent
  MISC_FEATURE (46)..(50): Residues may be absent
EAAAKEAAAKEAAAKEAAAKEAAAKEAAAKEAAAKEAAAKEAAAKEAAAK

SEQ ID NO: 156 - protein, 114 aa, Artificial Sequence
  Synthetic polypeptide
MAHHHHHHVGTGSNENLYFQHGEGTFTSDLSKQMEEEAVRLFIEWLKNTGGGGSGGGSGGGSGGGSASLAEAKVLANRELDKYGVSDFYKRLINKAKTVEGVEALKLHILAALP

SEQ ID NO: 157 - protein, 102 aa, Artificial Sequence
  Synthetic polypeptide
MAHHHHHHVGTGSNENLYFQHGEGTFTSDLSKQMEEEAVRLFIEWLKNTGGGGSASLAEAKVLANRELDKYGVSDFYKRLINKAKTVEGVEALKLHILAALP

SEQ ID NO: 158 - protein, 114 aa, Artificial Sequence
  Synthetic polypeptide
MAHHHHHHVGTGSNENLYFQHGEGTFTSDLSKQMEEEAVRLFIEWLKNTGGGGSGGGSGGGSGGGSASLAEAKVLANRELDKYGVSDYYKNIINRAKTVEGVRALKLHILAALP

SEQ ID NO: 159 - protein, 102 aa, Artificial Sequence
  Synthetic polypeptide
MAHHHHHHVGTGSNENLYFQHGEGTFTSDLSKQMEEEAVRLFIEWLKNTGGGGSASLAEAKVLANRELDKYGVSDYYKNIINRAKTVEGVRALKLHILAALP

SEQ ID NO: 160 - protein, 263 aa, Artificial Sequence
  Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQLEEEAVRLFIEWLKNGGPSSGAPPPSTGGGGSGGGSGGGSGGGSASLAEAKVLANRELDKYGVSDFYKRLINKAKTVEGVEALKLHILAAL

SEQ ID NO: 161 - protein, 253 aa, Artificial Sequence
  Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQMEEEAVRLFIEWLKNTGGGGSGGGSGGGSGGGSASLAEAKVLANRELDKYGVSDYYKNIINRAKTVEGVRALKLHILAALP

SEQ ID NO: 162 - protein, 241 aa, Artificial Sequence
  Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQMEEEAVRLFIEWLKNTGGGGSASLAEAKVLANRELDKYGVSDYYKNIINRAKTVEGVRALKLHILAALP

SEQ ID NO: 163 - protein, 91 aa, Artificial Sequence
  Synthetic polypeptide
HGEGTFTSDLSKQLEEEAVRLFIEWLKQGGPSKEIISTGGGGSASLAEAKVLANRELDKYGVSDFYKRLINKAKTVEGVEALKLHILAALP

SEQ ID NO: 164 - protein, 74 aa, Artificial Sequence
  Synthetic polypeptide
HAEGTFTSDVSSYLEGQAAKEFIAWLVKLAEAKVLANRELDKYGVSDFYKRLINKAKTVEGVEALKLHILAALP

SEQ ID NO: 165 - protein, 73 aa, Artificial Sequence
  Synthetic polypeptide
HGEGTFTSDLSKQMEEEAVRLFIEWLKLAEAKVLANRELDKYGVSDFYKRLINKAKTVEGVEALKLHILAALP

SEQ ID NO: 166 - protein, 79 aa, Artificial Sequence
  Synthetic polypeptide
HGEGTFTSDLSKQMEEEAVRLFIEWLKNGGPSSGAPPPSTGGGGSASYGVSDFYKRLINKAKTVEGVEALKLHILAALP

SEQ ID NO: 167 - protein, 72 aa, Artificial Sequence
  Synthetic polypeptide
HGEGTFTSDLSKQMEEEAVRLFIEWLKNGGPSSGAPPPSGYGVSDFYKRLINKAKTVEGVEALKLHILAALP

SEQ ID NO: 168 - protein, 89 aa, Artificial Sequence
  Synthetic polypeptide
HGEGTFTSDLSKQMEEEAVRLFIEWLKNGGPSSGAPPPSGGSLKNAKEDAIAELKKAGITSDFYFNAVNKAKTVEEVNALKNEILKALP

SEQ ID NO: 169 - protein, 88 aa, Artificial Sequence
  Synthetic polypeptide
HGEGTFTSDLSKQMEEEAVRLFIEWLKNGGPSSGAPPPSGGSLSEAKEMAIRELDANGVSDFYKDKIDDAKTVEGVVALKDLILNSLP

SEQ ID NO: 170 - protein, 88 aa, Artificial Sequence
  Synthetic polypeptide
HGEGTFTSDLSKQMEEEAVRLFIEWLKNGGPSSGAPPPSGGSLAKAKADAIEILKKYGIGDYYIKLINNGKTAEGVTALKDEILASLP

SEQ ID NO: 171 - protein, 61 aa, Artificial Sequence
  Synthetic polypeptide
  misc_feature (2)..(2): Xaa can be any naturally occurring amino acid
HXQGTFTSDYSKYLDEQAAKEFIAWLMNTYGVSDFYKRLINKAKTVEGVEALKLHILAALP

SEQ ID NO: 172 - protein, 61 aa, Artificial Sequence
  Synthetic polypeptide
HSQGTFTSDYSKYLDEQAAKEFIAWLMNTYGVSDFYKRLINKAKTVEGVEALKLHILAALP

SEQ ID NO: 173 - protein, 65 aa, Artificial Sequence
  Synthetic polypeptide
HSQGTFTSDYSKYLDEQAAKEFIAWLMNTGGGSYGVSDFYKRLINKAKTVEGVEALKLHILAALP

SEQ ID NO: 174-175 - skipped (2 entries)

SEQ ID NO: 176 - protein, 47 aa, Artificial Sequence
  Synthetic polypeptide
SLAEAKVLANRELDKYGVSDFYKRLINKAKTVEGVEALKLHILAALP

SEQ ID NO: 177 - protein, 46 aa, Artificial Sequence
  Synthetic polypeptide
SLAEAKVLANRELDKYGVSDFYKRLINKAKTVEGVEALKLHILAAL

SEQ ID NO: 178 - protein, 48 aa, Artificial Sequence
  Synthetic polypeptide
GSLAEAKVLANRELDKYGVSDFYKRLINKAKTVEGVEALKLHILAALP

SEQ ID NO: 179 - protein, 48 aa, Artificial Sequence
  Synthetic polypeptide
ASLAEAKVLANRELDKYGVSDFYKRLINKAKTVEGVEALKLHILAALP

SEQ ID NO: 180 - protein, 47 aa, Artificial Sequence
  Synthetic polypeptide
GSLAEAKVLANRELDKYGVSDFYKRLINKAKTVEGVEALKLHILAAL

SEQ ID NO: 181 - protein, 47 aa, Artificial Sequence
  Synthetic polypeptide
ASLAEAKVLANRELDKYGVSDFYKRLINKAKTVEGVEALKLHILAAL

SEQ ID NO: 182 - protein, 15 aa, Artificial Sequence
  Synthetic polypeptide
  VARIANT (6)..(6): Xaa is Val or Glu
  VARIANT (7)..(7): Xaa is Leu, Glu or Asp
  VARIANT (9)..(9): Xaa is Asn, Leu or Ile
  VARIANT (10)..(10): Xaa is Arg or Lys
  VARIANT (13)..(13): Xaa is Asp or Lys
LAEAKXXAXXELXKY

SEQ ID NO: 183 - protein, 5 aa, Artificial Sequence
  Synthetic polypeptide
LAALP

SEQ ID NO: 184 - protein, 50 aa, Artificial Sequence
  Synthetic polypeptide
KKKKKKHGEGTFTSDLSKQMEEEAVRLFIEWLKNGGPSSGAPPSKKKKKK

SEQ ID NO: 185 - protein, 38 aa, Artificial Sequence
  Synthetic polypeptide
HGEGTFTSDLSKQMEEEAVRLFIEWLWLKNGGPSSGAS

SEQ ID NO: 186 - protein, 42 aa, Artificial Sequence
  Synthetic polypeptide
KKKKKKHGEGTFTSDLSKQMEEEAVRLFIEWLKNGGPSSGAS

SEQ ID NO: 187 - protein, 42 aa, Artificial Sequence
  Synthetic polypeptide
NEEEEEHGEGTFTSDLSKQMEEEAVRLFIEWLKNGGPSSGAS

SEQ ID NO: 188 - protein, 42 aa, Artificial Sequence
  Synthetic polypeptide
HGEGTFTSDLSKQMEEEAVRLFIEWLKNGGPSSGASKKKKKK

SEQ ID NO: 189 - protein, 48 aa, Artificial Sequence
  Synthetic polypeptide
KKKKKKHGEGTFTSDLSKQMEEEAVRLFIEWLKNGGPSSGASKKKKKK

SEQ ID NO: 190 - protein, 48 aa, Artificial Sequence
  Synthetic polypeptide
DEEEEEHGEGTFTSDLSKQMEEEAVRLFIEWLKNGGPSSGASKKKKKK

SEQ ID NO: 191 - protein, 6 aa, Artificial Sequence
  Synthetic polypeptide
KKKKKK

SEQ ID NO: 192 - protein, 4 aa, Artificial Sequence
  Synthetic polypeptide
GGGS

SEQ ID NO: 193 - protein, 7 aa, Artificial Sequence
  Synthetic polypeptide
TGGGGAS

SEQ ID NO: 194 - protein, 8 aa, Artificial Sequence
  Synthetic polypeptide
TGGGGGAS

SEQ ID NO: 195 - protein, 8 aa, Artificial Sequence
  Synthetic polypeptide
TGGGGSAS

SEQ ID NO: 196 - protein, 4 aa, Artificial Sequence
  Synthetic polypeptide
GGGG

SEQ ID NO: 197 - protein, 5 aa, Artificial Sequence
  Synthetic polypeptide
GGGGG

SEQ ID NO: 198 - protein, 6 aa, Artificial Sequence
  Synthetic polypeptide
TGGGGS

SEQ ID NO: 199 - protein, 10 aa, Artificial Sequence
  Synthetic polypeptide
TGGGGSGGGS

SEQ ID NO: 200 - protein, 14 aa, Artificial Sequence
  Synthetic polypeptide
TGGGGSGGGSGGGS

SEQ ID NO: 201 - protein, 18 aa, Artificial Sequence
  Synthetic polypeptide
TGGGGSGGGSGGGSGGGS

SEQ ID NO: 202 - protein, 22 aa, Artificial Sequence
  Synthetic polypeptide
TGGGGSGGGSGGGSGGGSGGGS

SEQ ID NO: 203 - protein, 6 aa, Artificial Sequence
  Synthetic polypeptide
GGGSAS

SEQ ID NO: 204 - protein, 10 aa, Artificial Sequence
  Synthetic polypeptide
GGGSGGGSAS

SEQ ID NO: 205 - protein, 14 aa, Artificial Sequence
  Synthetic polypeptide
GGGSGGGSGGGSAS

SEQ ID NO: 206 - protein, 18 aa, Artificial Sequence
  Synthetic polypeptide
GGGSGGGSGGGSGGGSAS

SEQ ID NO: 207 - protein, 22 aa, Artificial Sequence
  Synthetic polypeptide
GGGSGGGSGGGSGGGSGGGSAS

SEQ ID NO: 208 - protein, 8 aa, Artificial Sequence
  Synthetic polypeptide
TGGGGSAS

SEQ ID NO: 209 - protein, 12 aa, Artificial Sequence
  Synthetic polypeptide
TGGGGSGGGSAS

SEQ ID NO: 210 - protein, 16 aa, Artificial Sequence
  Synthetic polypeptide
TGGGGSGGGSGGGSAS

SEQ ID NO: 211 - protein, 20 aa, Artificial Sequence
  Synthetic polypeptide
TGGGGSGGGSGGGSGGGSAS

SEQ ID NO: 212 - protein, 24 aa, Artificial Sequence
  Synthetic polypeptide
TGGGGSGGGSGGGSGGGSGGGSAS

SEQ ID NO: 213-299 - skipped (87 entries)

SEQ ID NO: 300 - protein, 46 aa, Artificial Sequence
  Synthetic polypeptide
  VARIANT (3)..(3): Xaa is Glu, Ser, Gln or Cys
  VARIANT (6)..(6): Xaa is Glu, Ser or Cys
  VARIANT (7)..(7): Xaa is Ala or Ser
  VARIANT (10)..(10): Xaa is Ala, Ser or Arg
  VARIANT (14)..(14): Xaa is Ala, Ser, Cys or Lys
  VARIANT (26)..(26): Xaa is Asp or Glu
  VARIANT (39)..(39): Xaa is Asp or Glu
  VARIANT (40)..(40): Xaa is Ala or Glu
  VARIANT (43)..(43): Xaa is Ala or Lys
  VARIANT (44)..(44): Xaa is Ala, Ser or Glu
  MISC_FEATURE (45)..(45): Residue may be absent
  MISC_FEATURE (46)..(46): Residue may be absent
LAXAKXXANXELDXYGVSDFYKRLIXKAKTVEGVEALKXXILXXLP

SEQ ID NO: 301 - protein, 46 aa, Artificial Sequence
  Synthetic polypeptide
LASAKEAANAELDAYGVSDFYKRLIDKAKTVEGVEALKDAILAALP

SEQ ID NO: 302 - protein, 46 aa, Artificial Sequence
  Synthetic polypeptide
LASAKEAANSELDAYGVSDFYKRLIDKAKTVEGVEALKDAILAALP

SEQ ID NO: 303 - protein, 46 aa, Artificial Sequence
  Synthetic polypeptide
LASAKESANSELDAYGVSDFYKRLIDKAKTVEGVEALKDAILAALP

SEQ ID NO: 304 - protein, 46 aa, Artificial Sequence
  Synthetic polypeptide
LASAKESANAELDAYGVSDFYKRLIDKAKTVEGVEALKDAILAALP

SEQ ID NO: 305 - protein, 46 aa, Artificial Sequence
  Synthetic polypeptide
LASAKSAANAELDAYGVSDFYKRLIDKAKTVEGVEALKDAILAALP

SEQ ID NO: 306 - protein, 46 aa, Artificial Sequence
  Synthetic polypeptide
LASAKSAANSELDAYGVSDFYKRLIDKAKTVEGVEALKDAILAALP

SEQ ID NO: 307 - protein, 46 aa, Artificial Sequence
  Synthetic polypeptide
LASAKEAANAELDSYGVSDFYKRLIDKAKTVEGVEALKDAILAALP

SEQ ID NO: 308 - protein, 46 aa, Artificial Sequence
  Synthetic polypeptide
LASAKEAANSELDSYGVSDFYKRLIDKAKTVEGVEALKDAILAALP

SEQ ID NO: 309 - protein, 46 aa, Artificial Sequence
  Synthetic polypeptide
LASAKESANSELDSYGVSDFYKRLIDKAKTVEGVEALKDAILAALP

SEQ ID NO: 310 - protein, 46 aa, Artificial Sequence
  Synthetic polypeptide
LASAKESANAELDSYGVSDFYKRLIDKAKTVEGVEALKDAILAALP

SEQ ID NO: 311 - protein, 46 aa, Artificial Sequence
  Synthetic polypeptide
LASAKSAANAELDSYGVSDFYKRLIDKAKTVEGVEALKDAILAALP

SEQ ID NO: 312 - protein, 46 aa, Artificial Sequence
  Synthetic polypeptide
LASAKSAANSELDSYGVSDFYKRLIDKAKTVEGVEALKDAILAALP

SEQ ID NO: 313 - protein, 46 aa, Artificial Sequence
  Synthetic polypeptide
LAEAKEAANAELDSYGVSDFYKRLIDKAKTVEGVEALKDAILAALP

SEQ ID NO: 314 - protein, 46 aa, Artificial Sequence
  Synthetic polypeptide
LAEAKEAANSELDSYGVSDFYKRLIDKAKTVEGVEALKDAILAALP

SEQ ID NO: 315 - protein, 46 aa, Artificial Sequence
  Synthetic polypeptide
LAEAKESANSELDSYGVSDFYKRLIDKAKTVEGVEALKDAILAALP

SEQ ID NO: 316 - protein, 46 aa, Artificial Sequence
  Synthetic polypeptide
LAEAKESANAELDSYGVSDFYKRLIDKAKTVEGVEALKDAILAALP

SEQ ID NO: 317 - protein, 46 aa, Artificial Sequence
  Synthetic polypeptide
LAEAKSAANAELDSYGVSDFYKRLIDKAKTVEGVEALKDAILAALP

SEQ ID NO: 318 - protein, 46 aa, Artificial Sequence
  Synthetic polypeptide
LAEAKSAANSELDSYGVSDFYKRLIDKAKTVEGVEALKDAILAALP

SEQ ID NO: 319 - protein, 46 aa, Artificial Sequence
  Synthetic polypeptide
LAEAKEAANAELDAYGVSDFYKRLIDKAKTVEGVEALKDAILAALP

SEQ ID NO: 320 - protein, 46 aa, Artificial Sequence
  Synthetic polypeptide
LAEAKEAANSELDAYGVSDFYKRLIDKAKTVEGVEALKDAILAALP

SEQ ID NO: 321 - protein, 46 aa, Artificial Sequence
  Synthetic polypeptide
LAEAKESANSELDAYGVSDFYKRLIDKAKTVEGVEALKDAILAALP

SEQ ID NO: 322 - protein, 46 aa, Artificial Sequence
  Synthetic polypeptide
LAEAKESANAELDAYGVSDFYKRLIDKAKTVEGVEALKDAILAALP

SEQ ID NO: 323 - protein, 46 aa, Artificial Sequence
  Synthetic polypeptide
LAEAKSAANAELDAYGVSDFYKRLIDKAKTVEGVEALKDAILAALP

SEQ ID NO: 324 - protein, 46 aa, Artificial Sequence
  Synthetic polypeptide
LAEAKSAANSELDAYGVSDFYKRLIDKAKTVEGVEALKDAILAALP

SEQ ID NO: 325 - protein, 46 aa, Artificial Sequence
  Synthetic polypeptide
LAQAKEAANAELDAYGVSDFYKRLIDKAKTVEGVEALKDAILAALP

SEQ ID NO: 326 - protein, 46 aa, Artificial Sequence
  Synthetic polypeptide
LAQAKEAANSELDAYGVSDFYKRLIDKAKTVEGVEALKDAILAALP

SEQ ID NO: 327 - protein, 46 aa, Artificial Sequence
  Synthetic polypeptide
LAQAKESANSELDAYGVSDFYKRLIDKAKTVEGVEALKDAILAALP

SEQ ID NO: 328 - protein, 46 aa, Artificial Sequence
  Synthetic polypeptide
LAQAKESANAELDAYGVSDFYKRLIDKAKTVEGVEALKDAILAALP

SEQ ID NO: 329 - protein, 46 aa, Artificial Sequence
  Synthetic polypeptide
LAQAKSAANAELDAYGVSDFYKRLIDKAKTVEGVEALKDAILAALP

SEQ ID NO: 330 - protein, 46 aa, Artificial Sequence
  Synthetic polypeptide
LAQAKSAANSELDAYGVSDFYKRLIDKAKTVEGVEALKDAILAALP

SEQ ID NO: 331 - protein, 46 aa, Artificial Sequence
  Synthetic polypeptide
LAQAKEAANAELDSYGVSDFYKRLIDKAKTVEGVEALKDAILAALP

SEQ ID NO: 332 - protein, 46 aa, Artificial Sequence
  Synthetic polypeptide
LAQAKEAANSELDSYGVSDFYKRLIDKAKTVEGVEALKDAILAALP

SEQ ID NO: 333 - protein, 46 aa, Artificial Sequence
  Synthetic polypeptide
LAQAKESANSELDSYGVSDFYKRLIDKAKTVEGVEALKDAILAALP

SEQ ID NO: 334 - protein, 46 aa, Artificial Sequence
  Synthetic polypeptide
LAQAKESANAELDSYGVSDFYKRLIDKAKTVEGVEALKDAILAALP

SEQ ID NO: 335 - protein, 46 aa, Artificial Sequence
  Synthetic polypeptide
LAQAKSAANAELDSYGVSDFYKRLIDKAKTVEGVEALKDAILAALP

SEQ ID NO: 336 - protein, 46 aa, Artificial Sequence
  Synthetic polypeptide
LAQAKSAANSELDSYGVSDFYKRLIDKAKTVEGVEALKDAILAALP

SEQ ID NO: 337 - protein, 46 aa, Artificial Sequence
  Synthetic polypeptide
LASAKEAANAELDCYGVSDFYKRLIDKAKTVEGVEALKDAILAALP

SEQ ID NO: 338 - protein, 46 aa, Artificial Sequence
  Synthetic polypeptide
LASAKEAANSELDCYGVSDFYKRLIDKAKTVEGVEALKDAILAALP

SEQ ID NO: 339 - protein, 46 aa, Artificial Sequence
  Synthetic polypeptide
LASAKESANSELDCYGVSDFYKRLIDKAKTVEGVEALKDAILAALP

SEQ ID NO: 340 - protein, 46 aa, Artificial Sequence
  Synthetic polypeptide
LASAKESANAELDCYGVSDFYKRLIDKAKTVEGVEALKDAILAALP

SEQ ID NO: 341 - protein, 46 aa, Artificial Sequence
  Synthetic polypeptide
LASAKSAANAELDCYGVSDFYKRLIDKAKTVEGVEALKDAILAALP

SEQ ID NO: 342 - protein, 46 aa, Artificial Sequence
  Synthetic polypeptide
LASAKSAANSELDCYGVSDFYKRLIDKAKTVEGVEALKDAILAALP

SEQ ID NO: 343 - protein, 46 aa, Artificial Sequence
  Synthetic polypeptide
LASAKEAANAELDKYGVSDFYKRLIDKAKTVEGVEALKDAILAALP

SEQ ID NO: 344 - protein, 46 aa, Artificial Sequence
  Synthetic polypeptide
LASAKEAANSELDKYGVSDFYKRLIDKAKTVEGVEALKDAILAALP

SEQ ID NO: 345 - protein, 46 aa, Artificial Sequence
  Synthetic polypeptide
LASAKESANSELDKYGVSDFYKRLIDKAKTVEGVEALKDAILAALP

SEQ ID NO: 346 - protein, 46 aa, Artificial Sequence
  Synthetic polypeptide
LASAKESANAELDKYGVSDFYKRLIDKAKTVEGVEALKDAILAALP

SEQ ID NO: 347 - protein, 46 aa, Artificial Sequence
  Synthetic polypeptide
LASAKSAANAELDKYGVSDFYKRLIDKAKTVEGVEALKDAILAALP

SEQ ID NO: 348 - protein, 46 aa, Artificial Sequence
  Synthetic polypeptide
LASAKSAANSELDKYGVSDFYKRLIDKAKTVEGVEALKDAILAALP

SEQ ID NO: 349 - protein, 46 aa, Artificial Sequence
  Synthetic polypeptide
LAEAKEAANAELDCYGVSDFYKRLIDKAKTVEGVEALKDAILAALP

SEQ ID NO: 350 - protein, 46 aa, Artificial Sequence
  Synthetic polypeptide
LAEAKEAANSELDCYGVSDFYKRLIDKAKTVEGVEALKDAILAALP

SEQ ID NO: 351 - protein, 46 aa, Artificial Sequence
  Synthetic polypeptide
LAEAKESANSELDCYGVSDFYKRLIDKAKTVEGVEALKDAILAALP

SEQ ID NO: 352 - protein, 46 aa, Artificial Sequence
  Synthetic polypeptide
LAEAKESANAELDCYGVSDFYKRLIDKAKTVEGVEALKDAILAALP

SEQ ID NO: 353 - protein, 46 aa, Artificial Sequence
  Synthetic polypeptide
LAEAKSAANAELDCYGVSDFYKRLIDKAKTVEGVEALKDAILAALP

SEQ ID NO: 354 - protein, 46 aa, Artificial Sequence
  Synthetic polypeptide
LAEAKSAANSELDCYGVSDFYKRLIDKAKTVEGVEALKDAILAALP

SEQ ID NO: 355 - protein, 46 aa, Artificial Sequence
  Synthetic polypeptide
LAEAKEAANAELDKYGVSDFYKRLIDKAKTVEGVEALKDAILAALP

SEQ ID NO: 356 - protein, 46 aa, Artificial Sequence
  Synthetic polypeptide
LAEAKEAANSELDKYGVSDFYKRLIDKAKTVEGVEALKDAILAALP

SEQ ID NO: 357 - protein, 46 aa, Artificial Sequence
  Synthetic polypeptide
LAEAKESANSELDKYGVSDFYKRLIDKAKTVEGVEALKDAILAALP

SEQ ID NO: 358 - protein, 46 aa, Artificial Sequence
  Synthetic polypeptide
LAEAKESANAELDKYGVSDFYKRLIDKAKTVEGVEALKDAILAALP

SEQ ID NO: 359 - protein, 46 aa, Artificial Sequence
  Synthetic polypeptide
LAEAKSAANAELDKYGVSDFYKRLIDKAKTVEGVEALKDAILAALP

SEQ ID NO: 360 - protein, 46 aa, Artificial Sequence
  Synthetic polypeptide
LAEAKSAANSELDKYGVSDFYKRLIDKAKTVEGVEALKDAILAALP

SEQ ID NO: 361 - protein, 46 aa, Artificial Sequence
  Synthetic polypeptide
LAQAKEAANAELDCYGVSDFYKRLIDKAKTVEGVEALKDAILAALP

SEQ ID NO: 362 - protein, 46 aa, Artificial Sequence
  Synthetic polypeptide
LAQAKEAANSELDCYGVSDFYKRLIDKAKTVEGVEALKDAILAALP

SEQ ID NO: 363 - protein, 46 aa, Artificial Sequence
  Synthetic polypeptide
LAQAKESANSELDCYGVSDFYKRLIDKAKTVEGVEALKDAILAALP

SEQ ID NO: 364 - protein, 46 aa, Artificial Sequence
  Synthetic polypeptide
LAQAKESANAELDCYGVSDFYKRLIDKAKTVEGVEALKDAILAALP

SEQ ID NO: 365 - protein, 46 aa, Artificial Sequence
  Synthetic polypeptide
LAQAKSAANAELDCYGVSDFYKRLIDKAKTVEGVEALKDAILAALP

SEQ ID NO: 366 - protein, 46 aa, Artificial Sequence
  Synthetic polypeptide
LAQAKSAANSELDCYGVSDFYKRLIDKAKTVEGVEALKDAILAALP

SEQ ID NO: 367 - protein, 46 aa, Artificial Sequence
  Synthetic polypeptide
LAQAKEAANAELDKYGVSDFYKRLIDKAKTVEGVEALKDAILAALP

SEQ ID NO: 368 - protein, 46 aa, Artificial Sequence
  Synthetic polypeptide
LAQAKEAANSELDKYGVSDFYKRLIDKAKTVEGVEALKDAILAALP

SEQ ID NO: 369 - protein, 46 aa, Artificial Sequence
  Synthetic polypeptide
LAQAKESANSELDKYGVSDFYKRLIDKAKTVEGVEALKDAILAALP

SEQ ID NO: 370 - protein, 46 aa, Artificial Sequence
  Synthetic polypeptide
LAQAKESANAELDKYGVSDFYKRLIDKAKTVEGVEALKDAILAALP

SEQ ID NO: 371 - protein, 46 aa, Artificial Sequence
  Synthetic polypeptide
LAQAKSAANAELDKYGVSDFYKRLIDKAKTVEGVEALKDAILAALP

SEQ ID NO: 372 - protein, 46 aa, Artificial Sequence
  Synthetic polypeptide
LAQAKSAANSELDKYGVSDFYKRLIDKAKTVEGVEALKDAILAALP

SEQ ID NO: 373 - protein, 46 aa, Artificial Sequence
  Synthetic polypeptide
LACAKEAANAELDCYGVSDFYKRLIDKAKTVEGVEALKDAILAALP

SEQ ID NO: 374 - protein, 46 aa, Artificial Sequence
  Synthetic polypeptide
LACAKEAANSELDCYGVSDFYKRLIDKAKTVEGVEALKDAILAALP

SEQ ID NO: 375 - protein, 46 aa, Artificial Sequence
  Synthetic polypeptide
LACAKESANSELDCYGVSDFYKRLIDKAKTVEGVEALKDAILAALP

SEQ ID NO: 376 - protein, 46 aa, Artificial Sequence
  Synthetic polypeptide
LACAKESANAELDCYGVSDFYKRLIDKAKTVEGVEALKDAILAALP

SEQ ID NO: 377 - protein, 46 aa, Artificial Sequence
  Synthetic polypeptide
LACAKSAANAELDCYGVSDFYKRLIDKAKTVEGVEALKDAILAALP

SEQ ID NO: 378 - protein, 46 aa, Artificial Sequence
  Synthetic polypeptide
LACAKSAANSELDCYGVSDFYKRLIDKAKTVEGVEALKDAILAALP

SEQ ID NO: 379 - protein, 46 aa, Artificial Sequence
  Synthetic polypeptide
LACAKEAANAELDKYGVSDFYKRLIDKAKTVEGVEALKDAILAALP

SEQ ID NO: 380 - protein, 46 aa, Artificial Sequence
  Synthetic polypeptide
LACAKEAANSELDKYGVSDFYKRLIDKAKTVEGVEALKDAILAALP

SEQ ID NO: 381 - protein, 46 aa, Artificial Sequence
  Synthetic polypeptide
LACAKESANSELDKYGVSDFYKRLIDKAKTVEGVEALKDAILAALP

SEQ ID NO: 382 - protein, 46 aa, Artificial Sequence
  Synthetic polypeptide
LACAKESANAELDKYGVSDFYKRLIDKAKTVEGVEALKDAILAALP

SEQ ID NO: 383 - protein, 46 aa, Artificial Sequence
  Synthetic polypeptide
LACAKSAANAELDKYGVSDFYKRLIDKAKTVEGVEALKDAILAALP

SEQ ID NO: 384 - protein, 46 aa, Artificial Sequence
  Synthetic polypeptide
LACAKSAANSELDKYGVSDFYKRLIDKAKTVEGVEALKDAILAALP

SEQ ID NO: 385 - protein, 46 aa, Artificial Sequence
  Synthetic polypeptide
LACAKEAANAELDSYGVSDFYKRLIDKAKTVEGVEALKDAILAALP

SEQ ID NO: 386 - protein, 46 aa, Artificial Sequence
  Synthetic polypeptide
LACAKEAANSELDSYGVSDFYKRLIDKAKTVEGVEALKDAILAALP

SEQ ID NO: 387 - protein, 46 aa, Artificial Sequence
  Synthetic polypeptide
LACAKESANSELDSYGVSDFYKRLIDKAKTVEGVEALKDAILAALP

SEQ ID NO: 388 - protein, 46 aa, Artificial Sequence
  Synthetic polypeptide
LACAKESANAELDSYGVSDFYKRLIDKAKTVEGVEALKDAILAALP

SEQ ID NO: 389 - protein, 46 aa, Artificial Sequence
  Synthetic polypeptide
LACAKSAANAELDSYGVSDFYKRLIDKAKTVEGVEALKDAILAALP

SEQ ID NO: 390 - protein, 46 aa, Artificial Sequence
  Synthetic polypeptide
LACAKSAANSELDSYGVSDFYKRLIDKAKTVEGVEALKDAILAALP

SEQ ID NO: 391 - protein, 46 aa, Artificial Sequence
  Synthetic polypeptide
LACAKEAANAELDAYGVSDFYKRLIDKAKTVEGVEALKDAILAALP

SEQ ID NO: 392 - protein, 46 aa, Artificial Sequence
  Synthetic polypeptide
LACAKEAANSELDAYGVSDFYKRLIDKAKTVEGVEALKDAILAALP

SEQ ID NO: 393 - protein, 46 aa, Artificial Sequence
  Synthetic polypeptide
LACAKESANSELDAYGVSDFYKRLIDKAKTVEGVEALKDAILAALP

SEQ ID NO: 394 - protein, 46 aa, Artificial Sequence
  Synthetic polypeptide
LACAKESANAELDAYGVSDFYKRLIDKAKTVEGVEALKDAILAALP

SEQ ID NO: 395 - protein, 46 aa, Artificial Sequence
  Synthetic polypeptide
LACAKSAANAELDAYGVSDFYKRLIDKAKTVEGVEALKDAILAALP

SEQ ID NO: 396 - protein, 46 aa, Artificial Sequence
  Synthetic polypeptide
LACAKSAANSELDAYGVSDFYKRLIDKAKTVEGVEALKDAILAALP

SEQ ID NO: 397 - protein, 46 aa, Artificial Sequence
  Synthetic polypeptide
LAQAKCAANAELDAYGVSDFYKRLIDKAKTVEGVEALKDAILAALP

SEQ ID NO: 398 - protein, 46 aa, Artificial Sequence
  Synthetic polypeptide
LAQAKCAANSELDAYGVSDFYKRLIDKAKTVEGVEALKDAILAALP

SEQ ID NO: 399 - protein, 46 aa, Artificial Sequence
  Synthetic polypeptide
LAQAKCSANSELDAYGVSDFYKRLIDKAKTVEGVEALKDAILAALP

SEQ ID NO: 400 - protein, 46 aa, Artificial Sequence
  Synthetic polypeptide
LAQAKCSANAELDAYGVSDFYKRLIDKAKTVEGVEALKDAILAALP

SEQ ID NO: 401 - protein, 46 aa, Artificial Sequence
  Synthetic polypeptide
LAQAKCAANAELDCYGVSDFYKRLIDKAKTVEGVEALKDAILAALP

SEQ ID NO: 402 - protein, 46 aa, Artificial Sequence
  Synthetic polypeptide
LAQAKCAANSELDCYGVSDFYKRLIDKAKTVEGVEALKDAILAALP

SEQ ID NO: 403 - protein, 46 aa, Artificial Sequence
  Synthetic polypeptide
LAQAKCAANAELDSYGVSDFYKRLIDKAKTVEGVEALKDAILAALP

SEQ ID NO: 404 - protein, 46 aa, Artificial Sequence
  Synthetic polypeptide
LAQAKCAANSELDSYGVSDFYKRLIDKAKTVEGVEALKDAILAALP

SEQ ID NO: 405 - protein, 46 aa, Artificial Sequence
  Synthetic polypeptide
LAQAKCSANSELDSYGVSDFYKRLIDKAKTVEGVEALKDAILAALP

SEQ ID NO: 406 - protein, 46 aa, Artificial Sequence
  Synthetic polypeptide
LAQAKCSANAELDSYGVSDFYKRLIDKAKTVEGVEALKDAILAALP

SEQ ID NO: 407 - protein, 46 aa, Artificial Sequence
  Synthetic polypeptide
LAQAKCAANAELDKYGVSDFYKRLIDKAKTVEGVEALKDAILAALP

SEQ ID NO: 408 - protein, 46 aa, Artificial Sequence
  Synthetic polypeptide
LAQAKCAANSELDKYGVSDFYKRLIDKAKTVEGVEALKDAILAALP

SEQ ID NO: 409 - protein, 46 aa, Artificial Sequence
  Synthetic polypeptide
LASAKCAANAELDAYGVSDFYKRLIDKAKTVEGVEALKDAILAALP

SEQ ID NO: 410 - protein, 46 aa, Artificial Sequence
  Synthetic polypeptide
LASAKCAANSELDAYGVSDFYKRLIDKAKTVEGVEALKDAILAALP

SEQ ID NO: 411 - protein, 46 aa, Artificial Sequence
  Synthetic polypeptide
LASAKCSANSELDAYGVSDFYKRLIDKAKTVEGVEALKDAILAALP

SEQ ID NO: 412 - protein, 46 aa, Artificial Sequence
  Synthetic polypeptide
LASAKCSANAELDAYGVSDFYKRLIDKAKTVEGVEALKDAILAALP

SEQ ID NO: 413 - protein, 46 aa, Artificial Sequence
  Synthetic polypeptide
LASAKCAANAELDCYGVSDFYKRLIDKAKTVEGVEALKDAILAALP

SEQ ID NO: 414 - protein, 46 aa, Artificial Sequence
  Synthetic polypeptide
LASAKCAANSELDCYGVSDFYKRLIDKAKTVEGVEALKDAILAALP

SEQ ID NO: 415 - protein, 46 aa, Artificial Sequence
  Synthetic polypeptide
LASAKCAANAELDSYGVSDFYKRLIDKAKTVEGVEALKDAILAALP

SEQ ID NO: 416 - protein, 46 aa, Artificial Sequence
  Synthetic polypeptide
LASAKCAANSELDSYGVSDFYKRLIDKAKTVEGVEALKDAILAALP

SEQ ID NO: 417 - protein, 46 aa, Artificial Sequence
  Synthetic polypeptide
LASAKCSANSELDSYGVSDFYKRLIDKAKTVEGVEALKDAILAALP

SEQ ID NO: 418 - protein, 46 aa, Artificial Sequence
  Synthetic polypeptide
LASAKCSANAELDSYGVSDFYKRLIDKAKTVEGVEALKDAILAALP

SEQ ID NO: 419 - protein, 46 aa, Artificial Sequence
  Synthetic polypeptide
LASAKCAANAELDKYGVSDFYKRLIDKAKTVEGVEALKDAILAALP

SEQ ID NO: 420 - protein, 46 aa, Artificial Sequence
  Synthetic polypeptide
LASAKCAANSELDKYGVSDFYKRLIDKAKTVEGVEALKDAILAALP

SEQ ID NO: 421 - protein, 46 aa, Artificial Sequence
  Synthetic polypeptide
LAEAKCAANAELDAYGVSDFYKRLIDKAKTVEGVEALKDAILAALP

SEQ ID NO: 422 - protein, 46 aa, Artificial Sequence
  Synthetic polypeptide
LAEAKCAANSELDAYGVSDFYKRLIDKAKTVEGVEALKDAILAALP

SEQ ID NO: 423 - protein, 46 aa, Artificial Sequence
  Synthetic polypeptide
LAEAKCSANSELDAYGVSDFYKRLIDKAKTVEGVEALKDAILAALP

SEQ ID NO: 424 - protein, 46 aa, Artificial Sequence
  Synthetic polypeptide
LAEAKCSANAELDAYGVSDFYKRLIDKAKTVEGVEALKDAILAALP

SEQ ID NO: 425 - protein, 46 aa, Artificial Sequence
  Synthetic polypeptide
LAEAKCAANAELDCYGVSDFYKRLIDKAKTVEGVEALKDAILAALP

SEQ ID NO: 426 - protein, 46 aa, Artificial Sequence
  Synthetic polypeptide
LAEAKCAANSELDCYGVSDFYKRLIDKAKTVEGVEALKDAILAALP

SEQ ID NO: 427 - protein, 46 aa, Artificial Sequence
  Synthetic polypeptide
LAEAKCAANAELDSYGVSDFYKRLIDKAKTVEGVEALKDAILAALP

SEQ ID NO: 428 - protein, 46 aa, Artificial Sequence
  Synthetic polypeptide
LAEAKCAANSELDSYGVSDFYKRLIDKAKTVEGVEALKDAILAALP

SEQ ID NO: 429 - protein, 46 aa, Artificial Sequence
  Synthetic polypeptide
LAEAKCSANSELDSYGVSDFYKRLIDKAKTVEGVEALKDAILAALP

SEQ ID NO: 430 - protein, 46 aa, Artificial Sequence
  Synthetic polypeptide
LAEAKCSANAELDSYGVSDFYKRLIDKAKTVEGVEALKDAILAALP

SEQ ID NO: 431 - protein, 46 aa, Artificial Sequence
  Synthetic polypeptide
LAEAKCAANAELDKYGVSDFYKRLIDKAKTVEGVEALKDAILAALP

SEQ ID NO: 432 - protein, 46 aa, Artificial Sequence
  Synthetic polypeptide
LAEAKCAANSELDKYGVSDFYKRLIDKAKTVEGVEALKDAILAALP

SEQ ID NO: 433 - protein, 46 aa, Artificial Sequence
  Synthetic polypeptide
LACAKCAANAELDAYGVSDFYKRLIDKAKTVEGVEALKDAILAALP

SEQ ID NO: 434 - protein, 46 aa, Artificial Sequence
  Synthetic polypeptide
LACAKCAANSELDAYGVSDFYKRLIDKAKTVEGVEALKDAILAALP

SEQ ID NO: 435 - protein, 46 aa, Artificial Sequence
  Synthetic polypeptide
LACAKCSANSELDAYGVSDFYKRLIDKAKTVEGVEALKDAILAALP

SEQ ID NO: 436 - protein, 46 aa, Artificial Sequence
  Synthetic polypeptide
LACAKCSANAELDAYGVSDFYKRLIDKAKTVEGVEALKDAILAALP

SEQ ID NO: 437 - protein, 46 aa, Artificial Sequence
  Synthetic polypeptide
LACAKCAANAELDCYGVSDFYKRLIDKAKTVEGVEALKDAILAALP

SEQ ID NO: 438 - protein, 46 aa, Artificial Sequence
  Synthetic polypeptide
LACAKCAANSELDCYGVSDFYKRLIDKAKTVEGVEALKDAILAALP

SEQ ID NO: 439 - protein, 46 aa, Artificial Sequence
  Synthetic polypeptide
LACAKCAANAELDSYGVSDFYKRLIDKAKTVEGVEALKDAILAALP

SEQ ID NO: 440 - protein, 46 aa, Artificial Sequence
  Synthetic polypeptide
LACAKCAANSELDSYGVSDFYKRLIDKAKTVEGVEALKDAILAALP

SEQ ID NO: 441 - protein, 46 aa, Artificial Sequence
  Synthetic polypeptide
LACAKCSANSELDSYGVSDFYKRLIDKAKTVEGVEALKDAILAALP

SEQ ID NO: 442 - protein, 46 aa, Artificial Sequence
  Synthetic polypeptide
LACAKCSANAELDSYGVSDFYKRLIDKAKTVEGVEALKDAILAALP

SEQ ID NO: 443 - protein, 46 aa, Artificial Sequence
  Synthetic polypeptide
LACAKCAANAELDKYGVSDFYKRLIDKAKTVEGVEALKDAILAALP

SEQ ID NO: 444 - protein, 46 aa, Artificial Sequence
  Synthetic polypeptide
LACAKCAANSELDKYGVSDFYKRLIDKAKTVEGVEALKDAILAALP

SEQ ID NO: 445 - protein, 49 aa, Artificial Sequence
  Synthetic polypeptide
GSLASAKEAANAELDSYGVSDFYKRLIDKAKTVEGVEALKDAILAALPG

SEQ ID NO: 446 - protein, 49 aa, Artificial Sequence
  Synthetic polypeptide
GSLAEAKEAANAELDSYGVSDFYKRLIDKAKTVEGVEALKDAILAALPG

SEQ ID NO: 447 - protein, 49 aa, Artificial Sequence
  Synthetic polypeptide
GSLASAKEAANAELDCYGVSDFYKRLIDKAKTVEGVEALKDAILAALPG

SEQ ID NO: 448 - protein, 49 aa, Artificial Sequence
  Synthetic polypeptide
GSLAEAKEAANAELDCYGVSDFYKRLIDKAKTVEGVEALKDAILAALPG

SEQ ID NO: 449 - protein, 50 aa, Artificial Sequence
  Synthetic polypeptide
GSLASAKEAANAELDSYGVSDFYKRLIDKAKTVEGVEALKDAILAALPCG

SEQ ID NO: 450 - protein, 50 aa, Artificial Sequence
  Synthetic polypeptide
GSLAEAKEAANAELDSYGVSDFYKRLIDKAKTVEGVEALKDAILAALPCG

SEQ ID NO: 451 - protein, 50 aa, Artificial Sequence
  Synthetic polypeptide
GCSLASAKEAANAELDKYGVSDFYKRLIDKAKTVEGVEALKDAILAALPG

SEQ ID NO: 452 - protein, 50 aa, Artificial Sequence
  Synthetic polypeptide
GCSLAEAKEAANAELDKYGVSDFYKRLIDKAKTVEGVEALKDAILAALPG

SEQ ID NO: 453 - protein, 47 aa, Artificial Sequence
  Synthetic polypeptide
GLAEAKVLANRELDKYGVSDYYKNLINNAKTVEGVKALIDEILAALP

SEQ ID NO: 454 - protein, 49 aa, Artificial Sequence
  Synthetic polypeptide
GSSLAEAKVLANRELDKYGVSDFYKRLINKAKTVEGVEALKLHILAALP

SEQ ID NO: 455 - protein, 49 aa, Artificial Sequence
  Synthetic polypeptide
GSSLASAKEAANAELDAYGVSDFYKRLIDKAKTVEGVEALKDAILAALP

SEQ ID NO: 456 - protein, 49 aa, Artificial Sequence
  Synthetic polypeptide
GSSLASAKEAANAELDSYGVSDFYKRLIDKAKTVEGVEALKDAILAALP

SEQ ID NO: 457 - protein, 47 aa, Artificial Sequence
  Synthetic polypeptide
GLASAKEAANAELDSYGVSDFYKRLIDKAKTVEGVEALKDAILAALP

SEQ ID NO: 458 - protein, 47 aa, Artificial Sequence
  Synthetic polypeptide
GLAEAKEAANAELDSYGVSDFYKRLIDKAKTVEGVEALKDAILAALP

SEQ ID NO: 459 - protein, 47 aa, Artificial Sequence
  Synthetic polypeptide
GLASAKEAANAELDCYGVSDFYKRLIDKAKTVEGVEALKDAILAALP

SEQ ID NO: 460 - protein, 47 aa, Artificial Sequence
  Synthetic polypeptide
ALASAKEAANAELDCYGVSDFYKRLIDKAKTVEGVEALKDAILAALP

SEQ ID NO: 461 - protein, 49 aa, Artificial Sequence
  Synthetic polypeptide
GSSLASAKEAANAELDKYGVSDFYKRLIDKAKTVEGVEALKDAILAALP

SEQ ID NO: 462 - protein, 48 aa, Artificial Sequence
  Synthetic polypeptide
GSLASAKEAANAELDSYGVSDFYKRLIDKAKTVEGVEALKDAILAALP

SEQ ID NO: 463 - protein, 48 aa, Artificial Sequence
  Synthetic polypeptide
GSLAEAKEAANAELDSYGVSDFYKRLIDKAKTVEGVEALKDAILAALP

SEQ ID NO: 464-499 - skipped (36 entries)

SEQ ID NO: 500 - protein, 47 aa, Artificial Sequence
  Synthetic polypeptide
SLAEAKEAANAELDSYGVSDFYKRLIDKAKTVEGVEALKDAILAALP

SEQ ID NO: 501 - protein, 47 aa, Artificial Sequence
  Synthetic polypeptide
LAEAKEAANAELDCYGVSDFYKRLIDKAKTVEGVEALKDAILAALPG

SEQ ID NO: 502 - protein, 48 aa, Artificial Sequence
  Synthetic polypeptide
SLAEAKEAANAELDCYGVSDFYKRLIDKAKTVEGVEALKDAILAALPG

SEQ ID NO: 503 - protein, 48 aa, Artificial Sequence
  Synthetic polypeptide
GSLAQAKEAANAELDSYGVSDFYKRLIDKAKTVEGVEALKDAILAALP

SEQ ID NO: 504 - protein, 48 aa, Artificial Sequence
  Synthetic polypeptide
GSLAEAKEAANRELDSYGVSDFYKRLIDKAKTVEGVEALKDAILAALP

SEQ ID NO: 505 - protein, 48 aa, Artificial Sequence
  Synthetic polypeptide
GSLAQAKEAANRELDSYGVSDFYKRLIDKAKTVEGVEALKDAILAALP

SEQ ID NO: 506 - protein, 48 aa, Artificial Sequence
  Synthetic polypeptide
GSLAEAKEAANRELDAYGVSDFYKRLIDKAKTVEGVEALKDAILAALP

SEQ ID NO: 507 - protein, 48 aa, Artificial Sequence
  Synthetic polypeptide
GSLAEAKVLANRELDKYGVSDFYKRLIDKAKTVEGVEALKDAILAALP

SEQ ID NO: 508 - protein, 47 aa, Artificial Sequence
  Synthetic polypeptide
SLAQAKEAANAELDSYGVSDFYKRLIDKAKTVEGVEALKDAILAALP

SEQ ID NO: 509 - protein, 47 aa, Artificial Sequence
  Synthetic polypeptide
SLAEAKEAANRELDSYGVSDFYKRLIDKAKTVEGVEALKDAILAALP

SEQ ID NO: 510 - protein, 47 aa, Artificial Sequence
  Synthetic polypeptide
SLAQAKEAANRELDSYGVSDFYKRLIDKAKTVEGVEALKDAILAALP

SEQ ID NO: 511 - protein, 47 aa, Artificial Sequence
  Synthetic polypeptide
SLAEAKEAANRELDAYGVSDFYKRLIDKAKTVEGVEALKDAILAALP

SEQ ID NO: 512 - protein, 47 aa, Artificial Sequence
  Synthetic polypeptide
SLAEAKVLANRELDKYGVSDFYKRLIDKAKTVEGVEALKDAILAALP

SEQ ID NO: 513 - protein, 46 aa, Artificial Sequence; identical to SEQ ID NO: 331
  Synthetic polypeptide

SEQ ID NO: 514 - protein, 46 aa, Artificial Sequence
  Synthetic polypeptide
LAEAKEAANRELDSYGVSDFYKRLIDKAKTVEGVEALKDAILAALP

SEQ ID NO: 515 - protein, 46 aa, Artificial Sequence
  Synthetic polypeptide
LAQAKEAANRELDSYGVSDFYKRLIDKAKTVEGVEALKDAILAALP

SEQ ID NO: 516 - protein, 46 aa, Artificial Sequence
  Synthetic polypeptide
LAEAKEAANRELDAYGVSDFYKRLIDKAKTVEGVEALKDAILAALP

SEQ ID NO: 517 - protein, 46 aa, Artificial Sequence
  Synthetic polypeptide
LAEAKVLANRELDKYGVSDFYKRLIDKAKTVEGVEALKDAILAALP

SEQ ID NO: 518 - protein, 48 aa, Artificial Sequence
  Synthetic polypeptide
GSLAQAKEAANAELDSYGVSDFYKRLIEKAKTVEGVEALKDAILAALP

SEQ ID NO: 519 - protein, 48 aa, Artificial Sequence
  Synthetic polypeptide
GSLAEAKEAANRELDSYGVSDFYKRLIEKAKTVEGVEALKDAILAALP

SEQ ID NO: 520 - protein, 48 aa, Artificial Sequence
  Synthetic polypeptide
GSLAQAKEAANRELDSYGVSDFYKRLIEKAKTVEGVEALKDAILAALP

SEQ ID NO: 521 - protein, 48 aa, Artificial Sequence
  Synthetic polypeptide
GSLAEAKEAANRELDAYGVSDFYKRLIEKAKTVEGVEALKDAILAALP

SEQ ID NO: 522 - protein, 48 aa, Artificial Sequence
  Synthetic polypeptide
GSLAEAKVLANRELDKYGVSDFYKRLIEKAKTVEGVEALKDAILAALP

SEQ ID NO: 523 - protein, 47 aa, Artificial Sequence
  Synthetic polypeptide
SLAQAKEAANAELDSYGVSDFYKRLIEKAKTVEGVEALKDAILAALP

SEQ ID NO: 524 - protein, 47 aa, Artificial Sequence
  Synthetic polypeptide
SLAEAKEAANRELDSYGVSDFYKRLIEKAKTVEGVEALKDAILAALP

SEQ ID NO: 525 - protein, 47 aa, Artificial Sequence
  Synthetic polypeptide
SLAQAKEAANRELDSYGVSDFYKRLIEKAKTVEGVEALKDAILAALP

SEQ ID NO: 526 - protein, 47 aa, Artificial Sequence
  Synthetic polypeptide
SLAEAKEAANRELDAYGVSDFYKRLIEKAKTVEGVEALKDAILAALP

SEQ ID NO: 527 - protein, 47 aa, Artificial Sequence
  Synthetic polypeptide
SLAEAKVLANRELDKYGVSDFYKRLIEKAKTVEGVEALKDAILAALP

SEQ ID NO: 528 - protein, 46 aa, Artificial Sequence
  Synthetic polypeptide
LAQAKEAANAELDSYGVSDFYKRLIEKAKTVEGVEALKDAILAALP

SEQ ID NO: 529 - protein, 46 aa, Artificial Sequence
  Synthetic polypeptide
LAEAKEAANRELDSYGVSDFYKRLIEKAKTVEGVEALKDAILAALP

SEQ ID NO: 530 - protein, 46 aa, Artificial Sequence
  Synthetic polypeptide
LAQAKEAANRELDSYGVSDFYKRLIEKAKTVEGVEALKDAILAALP

SEQ ID NO: 531 - protein, 46 aa, Artificial Sequence
  Synthetic polypeptide
LAEAKEAANRELDAYGVSDFYKRLIEKAKTVEGVEALKDAILAALP

SEQ ID NO: 532 - protein, 46 aa, Artificial Sequence
  Synthetic polypeptide
LAEAKVLANRELDKYGVSDFYKRLIEKAKTVEGVEALKDAILAALP

SEQ ID NO: 533 - protein, 48 aa, Artificial Sequence
  Synthetic polypeptide
GSLAQAKEAANAELDSYGVSDFYKRLIEKAKTVEGVEALKEAILAALP

SEQ ID NO: 534 - protein, 48 aa, Artificial Sequence
  Synthetic polypeptide
GSLAEAKEAANRELDSYGVSDFYKRLIEKAKTVEGVEALKEAILAALP

SEQ ID NO: 535 - protein, 48 aa, Artificial Sequence
  Synthetic polypeptide
GSLAQAKEAANRELDSYGVSDFYKRLIEKAKTVEGVEALKEAILAALP

SEQ ID NO: 536 - protein, 48 aa, Artificial Sequence
  Synthetic polypeptide
GSLAEAKEAANRELDAYGVSDFYKRLIEKAKTVEGVEALKEAILAALP

SEQ ID NO: 537 - protein, 48 aa, Artificial Sequence
  Synthetic polypeptide
GSLAEAKVLANRELDKYGVSDFYKRLIEKAKTVEGVEALKEAILAALP

SEQ ID NO: 538 - protein, 47 aa, Artificial Sequence
  Synthetic polypeptide
SLAQAKEAANAELDSYGVSDFYKRLIEKAKTVEGVEALKEAILAALP

SEQ ID NO: 539 - protein, 47 aa, Artificial Sequence
  Synthetic polypeptide
SLAEAKEAANRELDSYGVSDFYKRLIEKAKTVEGVEALKEAILAALP

SEQ ID NO: 540 - protein, 47 aa, Artificial Sequence
  Synthetic polypeptide
SLAQAKEAANRELDSYGVSDFYKRLIEKAKTVEGVEALKEAILAALP

SEQ ID NO: 541 - protein, 47 aa, Artificial Sequence
  Synthetic polypeptide
SLAEAKEAANRELDAYGVSDFYKRLIEKAKTVEGVEALKEAILAALP

SEQ ID NO: 542 - protein, 47 aa, Artificial Sequence
  Synthetic polypeptide
SLAEAKVLANRELDKYGVSDFYKRLIEKAKTVEGVEALKEAILAALP

SEQ ID NO: 543 - protein, 46 aa, Artificial Sequence
  Synthetic polypeptide
LAQAKEAANAELDSYGVSDFYKRLIEKAKTVEGVEALKEAILAALP

SEQ ID NO: 544 - protein, 46 aa, Artificial Sequence
  Synthetic polypeptide
LAEAKEAANRELDSYGVSDFYKRLIEKAKTVEGVEALKEAILAALP

SEQ ID NO: 545 - protein, 46 aa, Artificial Sequence
  Synthetic polypeptide
LAQAKEAANRELDSYGVSDFYKRLIEKAKTVEGVEALKEAILAALP

SEQ ID NO: 546 - protein, 46 aa, Artificial Sequence
  Synthetic polypeptide
LAEAKEAANRELDAYGVSDFYKRLIEKAKTVEGVEALKEAILAALP

SEQ ID NO: 547 - protein, 46 aa, Artificial Sequence
  Synthetic polypeptide
LAEAKVLANRELDKYGVSDFYKRLIEKAKTVEGVEALKEAILAALP

SEQ ID NO: 548 - protein, 48 aa, Artificial Sequence
  Synthetic polypeptide
GSLAQAKEAANAELDSYGVSDFYKRLIDKAKTVEGVEALKDAILASLP

SEQ ID NO: 549 - protein, 48 aa, Artificial Sequence
  Synthetic polypeptide
GSLAEAKEAANRELDSYGVSDFYKRLIDKAKTVEGVEALKDAILASLP

SEQ ID NO: 550 - protein, 48 aa, Artificial Sequence
  Synthetic polypeptide
GSLAQAKEAANRELDSYGVSDFYKRLIDKAKTVEGVEALKDAILASLP

SEQ ID NO: 551 - protein, 48 aa, Artificial Sequence
  Synthetic polypeptide
GSLAEAKEAANRELDAYGVSDFYKRLIDKAKTVEGVEALKDAILASLP

SEQ ID NO: 552 - protein, 48 aa, Artificial Sequence
  Synthetic polypeptide
GSLAEAKVLANRELDKYGVSDFYKRLIDKAKTVEGVEALKDAILASLP

SEQ ID NO: 553 - protein, 47 aa, Artificial Sequence
  Synthetic polypeptide
SLAQAKEAANAELDSYGVSDFYKRLIDKAKTVEGVEALKDAILASLP

SEQ ID NO: 554 - protein, 47 aa, Artificial Sequence
  Synthetic polypeptide
SLAEAKEAANRELDSYGVSDFYKRLIDKAKTVEGVEALKDAILASLP

SEQ ID NO: 555 - protein, 47 aa, Artificial Sequence
  Synthetic polypeptide
SLAQAKEAANRELDSYGVSDFYKRLIDKAKTVEGVEALKDAILASLP

SEQ ID NO: 556 - protein, 47 aa, Artificial Sequence
  Synthetic polypeptide
SLAEAKEAANRELDAYGVSDFYKRLIDKAKTVEGVEALKDAILASLP

SEQ ID NO: 557 - protein, 47 aa, Artificial Sequence
  Synthetic polypeptide
SLAEAKVLANRELDKYGVSDFYKRLIDKAKTVEGVEALKDAILASLP

SEQ ID NO: 558 - protein, 46 aa, Artificial Sequence
  Synthetic polypeptide
LAQAKEAANAELDSYGVSDFYKRLIDKAKTVEGVEALKDAILASLP

SEQ ID NO: 559 - protein, 46 aa, Artificial Sequence
  Synthetic polypeptide
LAEAKEAANRELDSYGVSDFYKRLIDKAKTVEGVEALKDAILASLP

SEQ ID NO: 560 - protein, 46 aa, Artificial Sequence
  Synthetic polypeptide
LAQAKEAANRELDSYGVSDFYKRLIDKAKTVEGVEALKDAILASLP

SEQ ID NO: 561 - protein, 46 aa, Artificial Sequence
  Synthetic polypeptide
LAEAKEAANRELDAYGVSDFYKRLIDKAKTVEGVEALKDAILASLP

SEQ ID NO: 562 - protein, 46 aa, Artificial Sequence
  Synthetic polypeptide
LAEAKVLANRELDKYGVSDFYKRLIDKAKTVEGVEALKDAILASLP

SEQ ID NO: 563 - protein, 48 aa, Artificial Sequence
  Synthetic polypeptide
GSLAQAKEAANAELDSYGVSDFYKRLIDKAKTVEGVEALKDAILAELP

SEQ ID NO: 564 - protein, 48 aa, Artificial Sequence
  Synthetic polypeptide
GSLAEAKEAANRELDSYGVSDFYKRLIDKAKTVEGVEALKDAILAELP

SEQ ID NO: 565 - protein, 48 aa, Artificial Sequence
  Synthetic polypeptide
GSLAQAKEAANRELDSYGVSDFYKRLIDKAKTVEGVEALKDAILAELP

SEQ ID NO: 566 - protein, 48 aa, Artificial Sequence
  Synthetic polypeptide
GSLAEAKEAANRELDAYGVSDFYKRLIDKAKTVEGVEALKDAILAELP

SEQ ID NO: 567 - protein, 48 aa, Artificial Sequence
  Synthetic polypeptide
GSLAEAKVLANRELDKYGVSDFYKRLIDKAKTVEGVEALKDAILAELP

SEQ ID NO: 568 - protein, 47 aa, Artificial Sequence
  Synthetic polypeptide
SLAQAKEAANAELDSYGVSDFYKRLIDKAKTVEGVEALKDAILAELP

SEQ ID NO: 569 - protein, 47 aa, Artificial Sequence
  Synthetic polypeptide
SLAEAKEAANRELDSYGVSDFYKRLIDKAKTVEGVEALKDAILAELP

SEQ ID NO: 570 - protein, 47 aa, Artificial Sequence
  Synthetic polypeptide
SLAQAKEAANRELDSYGVSDFYKRLIDKAKTVEGVEALKDAILAELP

SEQ ID NO: 571 - protein, 47 aa, Artificial Sequence
  Synthetic polypeptide
SLAEAKEAANRELDAYGVSDFYKRLIDKAKTVEGVEALKDAILAELP

SEQ ID NO: 572 - protein, 47 aa, Artificial Sequence
  Synthetic polypeptide
SLAEAKVLANRELDKYGVSDFYKRLIDKAKTVEGVEALKDAILAELP

SEQ ID NO: 573 - protein, 46 aa, Artificial Sequence
  Synthetic polypeptide
LAQAKEAANAELDSYGVSDFYKRLIDKAKTVEGVEALKDAILAELP

SEQ ID NO: 574 - protein, 46 aa, Artificial Sequence
  Synthetic polypeptide
LAEAKEAANRELDSYGVSDFYKRLIDKAKTVEGVEALKDAILAELP

SEQ ID NO: 575 - protein, 46 aa, Artificial Sequence
  Synthetic polypeptide
LAQAKEAANRELDSYGVSDFYKRLIDKAKTVEGVEALKDAILAELP

SEQ ID NO: 576 - protein, 46 aa, Artificial Sequence
  Synthetic polypeptide
LAEAKEAANRELDAYGVSDFYKRLIDKAKTVEGVEALKDAILAELP

SEQ ID NO: 577 - protein, 46 aa, Artificial Sequence
  Synthetic polypeptide
LAEAKVLANRELDKYGVSDFYKRLIDKAKTVEGVEALKDAILAELP

SEQ ID NO: 578 - protein, 48 aa, Artificial Sequence
  Synthetic polypeptide
GSLAQAKEAANAELDSYGVSDFYKRLIDKAKTVEGVEALKDAILKALP

SEQ ID NO: 579 - protein, 48 aa, Artificial Sequence
  Synthetic polypeptide
GSLAEAKEAANRELDSYGVSDFYKRLIDKAKTVEGVEALKDAILKALP

SEQ ID NO: 580 - protein, 48 aa, Artificial Sequence
  Synthetic polypeptide
GSLAQAKEAANRELDSYGVSDFYKRLIDKAKTVEGVEALKDAILKALP

SEQ ID NO: 581 - protein, 48 aa, Artificial Sequence
  Synthetic polypeptide
GSLAEAKEAANRELDAYGVSDFYKRLIDKAKTVEGVEALKDAILKALP

SEQ ID NO: 582 - protein, 48 aa, Artificial Sequence
  Synthetic polypeptide
GSLAEAKVLANRELDKYGVSDFYKRLIDKAKTVEGVEALKDAILKALP

SEQ ID NO: 583 - protein, 47 aa, Artificial Sequence
  Synthetic polypeptide
SLAQAKEAANAELDSYGVSDFYKRLIDKAKTVEGVEALKDAILKALP

SEQ ID NO: 584 - protein, 47 aa, Artificial Sequence
  Synthetic polypeptide
SLAEAKEAANRELDSYGVSDFYKRLIDKAKTVEGVEALKDAILKALP

SEQ ID NO: 585 - protein, 47 aa, Artificial Sequence
  Synthetic polypeptide
SLAQAKEAANRELDSYGVSDFYKRLIDKAKTVEGVEALKDAILKALP

SEQ ID NO: 586 - protein, 47 aa, Artificial Sequence
  Synthetic polypeptide
SLAEAKEAANRELDAYGVSDFYKRLIDKAKTVEGVEALKDAILKALP

SEQ ID NO: 587 - protein, 47 aa, Artificial Sequence
  Synthetic polypeptide
SLAEAKVLANRELDKYGVSDFYKRLIDKAKTVEGVEALKDAILKALP

SEQ ID NO: 588 - protein, 46 aa, Artificial Sequence
  Synthetic polypeptide
LAQAKEAANAELDSYGVSDFYKRLIDKAKTVEGVEALKDAILKALP

SEQ ID NO: 589 - protein, 46 aa, Artificial Sequence
  Synthetic polypeptide
LAEAKEAANRELDSYGVSDFYKRLIDKAKTVEGVEALKDAILKALP

SEQ ID NO: 590 - protein, 46 aa, Artificial Sequence
  Synthetic polypeptide
LAQAKEAANRELDSYGVSDFYKRLIDKAKTVEGVEALKDAILKALP

SEQ ID NO: 591 - protein, 46 aa, Artificial Sequence
  Synthetic polypeptide
LAEAKEAANRELDAYGVSDFYKRLIDKAKTVEGVEALKDAILKALP

SEQ ID NO: 592 - protein, 46 aa, Artificial Sequence
  Synthetic polypeptide
LAEAKVLANRELDKYGVSDFYKRLIDKAKTVEGVEALKDAILKALP

SEQ ID NO: 593 - protein, 46 aa, Artificial Sequence; identical to SEQ ID NO: 121
  Synthetic polypeptide
  MISC_FEATURE (6)..(6): Xaa is Val or Glu
  MISC_FEATURE (7)..(7): Xaa is Leu, Glu or Asp
  MISC_FEATURE (9)..(9): Xaa is Asn, Leu or Ile
  MISC_FEATURE (10)..(10): Xaa is Arg or Lys
  MISC_FEATURE (13)..(13): Xaa is Asp or Lys
  MISC_FEATURE (20)..(20): Xaa is Tyr or Phe
  MISC_FEATURE (23)..(23): Xaa is Asn, Arg or Ser
  MISC_FEATURE (24)..(24): Xaa is Val, Ile, Leu, Met, Phe or Tyr
  MISC_FEATURE (26)..(26): Xaa is Asn, Ser, Glu or Asp
  MISC_FEATURE (27)..(27): Xaa is Arg, Lys or Asn
  MISC_FEATURE (29)..(29): Xaa is Lys or Arg
  MISC_FEATURE (35)..(35): Xaa is Asp, Asn, Gln, Glu, His, Ser, Arg or Lys
  MISC_FEATURE (38)..(38): Xaa is Lys, Ile or Thr
  MISC_FEATURE (39)..(39): Xaa is Ala, Ser, Thr, Gly, His, Leu or Asp
  MISC_FEATURE (40)..(40): Xaa is His, Glu or Asp

SEQ ID NO: 594 - protein, 46 aa, Artificial Sequence
  Synthetic polypeptide
  MISC_FEATURE (3)..(3): Xaa is Glu, Ser, Gln or Cys
  MISC_FEATURE (6)..(6): Xaa is Glu, Ser or Cys
  MISC_FEATURE (7)..(7): Xaa is Ala or Ser
  MISC_FEATURE (10)..(10): Xaa is Ala, Ser or Arg
  MISC_FEATURE (14)..(14): Xaa is Ala, Ser, Cys or Lys
  MISC_FEATURE (45)..(45): Residues may be absent
  MISC_FEATURE (46)..(46): Residues may be absent
LAXAKXXANXELDXYGVSDFYKRLIDKAKTVEGVEALKDAILAALP

SEQ ID NO: 595 - protein, 49 aa, Artificial Sequence
  Synthetic polypeptide
SGSLAEAKEAANAELDSYGVSDFYKRLIDKAKTVEGVEALKDAILAALP

SEQ ID NO: 596 - protein, 48 aa, Artificial Sequence
  Synthetic polypeptide
SGSLAEAKEAANAELDSYGVSDFYKRLIDKAKTVEGVEALKDAILAAL

SEQ ID NO: 597 - protein, 48 aa, Artificial Sequence
  Synthetic polypeptide
SGSLAEAKEAANAELDSYGVSDFYKRLIDKAKTVEGVEALKDAILAAP

SEQ ID NO: 598 - protein, 47 aa, Artificial Sequence
  Synthetic polypeptide
SGSLAEAKEAANAELDSYGVSDFYKRLIDKAKTVEGVEALKDAILAA

SEQ ID NO: 599 - protein, 47 aa, Artificial Sequence
  Synthetic polypeptide
GSLAEAKEAANAELDSYGVSDFYKRLIDKAKTVEGVEALKDAILAAL

SEQ ID NO: 600 - protein, 47 aa, Artificial Sequence
  Synthetic polypeptide
GSLAEAKEAANAELDCYGVSDFYKRLIDKAKTVEGVEALKDAILAAL

SEQ ID NO: 601 - protein, 46 aa, Artificial Sequence
  Synthetic polypeptide
SLAEAKEAANAELDSYGVSDFYKRLIDKAKTVEGVEALKDAILAAL

SEQ ID NO: 602 - protein, 46 aa, Artificial Sequence
  Synthetic polypeptide
SLAEAKEAANAELDCYGVSDFYKRLIDKAKTVEGVEALKDAILAAL

SEQ ID NO: 603 - protein, 45 aa, Artificial Sequence
  Synthetic polypeptide
LAEAKEAANAELDSYGVSDFYKRLIDKAKTVEGVEALKDAILAAL

SEQ ID NO: 604 - protein, 45 aa, Artificial Sequence
  Synthetic polypeptide
LAEAKEAANAELDCYGVSDFYKRLIDKAKTVEGVEALKDAILAAL

SEQ ID NO: 605 - protein, 47 aa, Artificial Sequence
  Synthetic polypeptide
GSSLASAKEAANAELDAYGVSDFYKRLIDKAKTVEGVEALKDAILAA

SEQ ID NO: 606 - protein, 47 aa, Artificial Sequence
  Synthetic polypeptide
GSSLASAKEAANAELDSYGVSDFYKRLIDKAKTVEGVEALKDAILAA

SEQ ID NO: 607 - protein, 45 aa, Artificial Sequence
  Synthetic polypeptide
GLASAKEAANAELDSYGVSDFYKRLIDKAKTVEGVEALKDAILAA

SEQ ID NO: 608 - protein, 45 aa, Artificial Sequence
  Synthetic polypeptide
GLAEAKEAANAELDSYGVSDFYKRLIDKAKTVEGVEALKDAILAA

SEQ ID NO: 609 - protein, 45 aa, Artificial Sequence
  Synthetic polypeptide
GLASAKEAANAELDCYGVSDFYKRLIDKAKTVEGVEALKDAILAA

SEQ ID NO: 610 - protein, 45 aa, Artificial Sequence
  Synthetic polypeptide
ALASAKEAANAELDCYGVSDFYKRLIDKAKTVEGVEALKDAILAA

SEQ ID NO: 611 - protein, 47 aa, Artificial Sequence
  Synthetic polypeptide
GSSLASAKEAANAELDKYGVSDFYKRLIDKAKTVEGVEALKDAILAA

SEQ ID NO: 612 - protein, 46 aa, Artificial Sequence
  Synthetic polypeptide
GSLASAKEAANAELDSYGVSDFYKRLIDKAKTVEGVEALKDAILAA

SEQ ID NO: 613 - protein, 46 aa, Artificial Sequence
  Synthetic polypeptide
GSLAEAKEAANAELDSYGVSDFYKRLIDKAKTVEGVEALKDAILAA

SEQ ID NO: 614 - protein, 46 aa, Artificial Sequence
  Synthetic polypeptide
GSLAEAKEAANAELDCYGVSDFYKRLIDKAKTVEGVEALKDAILAA

SEQ ID NO: 615 - protein, 44 aa, Artificial Sequence
  Synthetic polypeptide
LAEAKEAANAELDSYGVSDFYKRLIDKAKTVEGVEALKDAILAA

SEQ ID NO: 616 - protein, 45 aa, Artificial Sequence
  Synthetic polypeptide
SLAEAKEAANAELDSYGVSDFYKRLIDKAKTVEGVEALKDAILAA

SEQ ID NO: 617 - protein, 44 aa, Artificial Sequence
  Synthetic polypeptide
LAEAKEAANAELDCYGVSDFYKRLIDKAKTVEGVEALKDAILAA

SEQ ID NO: 618 - protein, 45 aa, Artificial Sequence
  Synthetic polypeptide
SLAEAKEAANAELDCYGVSDFYKRLIDKAKTVEGVEALKDAILAA

SEQ ID NO: 619 - protein, 49 aa, Artificial Sequence
  Synthetic polypeptide
AGSLAEAKEAANAELDSYGVSDFYKRLIDKAKTVEGVEALKDAILAALP

SEQ ID NO: 620 - protein, 93 aa, Artificial Sequence
  Synthetic polypeptide
HGEGTFTSDLSKQLEEEAVRLFIEWLKQGGPSKEIISTGGGGSASGSLAEAKEAANAELDSYGVSDFYKRLIDKAKTVEGVEALKDAILAALP

SEQ ID NO: 621 - protein, 95 aa, Artificial Sequence
  Synthetic polypeptide
HGEGTFTSDLSKQLEEEAVRLFIEWLKNGGPSSGAPPKSTGGGGSASGSLAEAKEAANAELDSYGVSDFYKRLIDKAKTVEGVEALKDAILAALP

SEQ ID NO: 622 - protein, 94 aa, Artificial Sequence
  Synthetic polypeptide
HGEGTFTSDLSKQLEEEAVRLFIEWLKNGGPSSGAPPPSTGGGGSASGSLAEAKEAANAELDSYGVSDFYKRLIDKAKTVEGVEALKDAILAAL

SEQ ID NO: 623 - protein, 95 aa, Artificial Sequence
  Synthetic polypeptide
HGEGTFTSDLSKQMEEEAVRLFIEWLKNGGPSSGAPPPSTGGGGSASGSLAEAKEAANAELDSYGVSDFYKRLIDKAKTVEGVEALKDAILAALP

SEQ ID NO: 624 - protein, 88 aa, Artificial Sequence
  Synthetic polypeptide
HGEGTFTSDLSKQMEEEAVRLFIEWLKNGGPSSGAPPPSGGSLAEAKEAANAELDSYGVSDFYKRLIDKAKTVEGVEALKDAILAALP

SEQ ID NO: 625 - protein, 95 aa, Artificial Sequence
  Synthetic polypeptide
HGEGTFTSDLSKQLEEEAVRLFIEWLKNGGPSSGAPPPSTGGGGSASGSLAEAKEAANAELDSYGVSDFYKRLIDKAKTVEGVEALKDAILAALP

SEQ ID NO: 626 - protein, 88 aa, Artificial Sequence
  Synthetic polypeptide
HGEGTFTSDLSKQLEEEAVRLFIEWLKNGGPSSGAPPPSGGSLAEAKEAANAELDSYGVSDFYKRLIDKAKTVEGVEALKDAILAALP

SEQ ID NO: 627 - protein, 92 aa, Artificial Sequence
  Synthetic polypeptide
HGEGTFTSDLSKQLEEEAVRLFIEWLKQGGPSKEIISTGGGGSASGSLAEAKEAANAELDSYGVSDFYKRLIDKAKTVEGVEALKDAILAAL

SEQ ID NO: 628 - protein, 94 aa, Artificial Sequence
  Synthetic polypeptide
HGEGTFTSDLSKQLEEEAVRLFIEWLKNGGPSSGAPPKSTGGGGSASGSLAEAKEAANAELDSYGVSDFYKRLIDKAKTVEGVEALKDAILAAL

SEQ ID NO: 629 - protein, 93 aa, Artificial Sequence
  Synthetic polypeptide
HGEGTFTSDLSKQLEEEAVRLFIEWLKNGGPSSGAPPPSTGGGGSASGSLAEAKEAANAELDSYGVSDFYKRLIDKAKTVEGVEALKDAILAA

SEQ ID NO: 630 - protein, 94 aa, Artificial Sequence
  Synthetic polypeptide
HGEGTFTSDLSKQMEEEAVRLFIEWLKNGGPSSGAPPPSTGGGGSASGSLAEAKEAANAELDSYGVSDFYKRLIDKAKTVEGVEALKDAILAAL

SEQ ID NO: 631 - protein, 87 aa, Artificial Sequence
  Synthetic polypeptide
HGEGTFTSDLSKQMEEEAVRLFIEWLKNGGPSSGAPPPSGGSLAEAKEAANAELDSYGVSDFYKRLIDKAKTVEGVEALKDAILAAL

SEQ ID NO: 632 - protein, 87 aa, Artificial Sequence
  Synthetic polypeptide
HGEGTFTSDLSKQLEEEAVRLFIEWLKNGGPSSGAPPPSGGSLAEAKEAANAELDSYGVSDFYKRLIDKAKTVEGVEALKDAILAAL

SEQ ID NO: 633 - protein, 91 aa, Artificial Sequence
  Synthetic polypeptide
HGEGTFTSDLSKQLEEEAVRLFIEWLKQGGPSKEIISTGGGGSASGSLAEAKEAANAELDSYGVSDFYKRLIDKAKTVEGVEALKDAILAA

SEQ ID NO: 634 - protein, 93 aa, Artificial Sequence
  Synthetic polypeptide
HGEGTFTSDLSKQLEEEAVRLFIEWLKNGGPSSGAPPKSTGGGGSASGSLAEAKEAANAELDSYGVSDFYKRLIDKAKTVEGVEALKDAILAA

SEQ ID NO: 635 - protein, 93 aa, Artificial Sequence
  Synthetic polypeptide
HGEGTFTSDLSKQMEEEAVRLFIEWLKNGGPSSGAPPPSTGGGGSASGSLAEAKEAANAELDSYGVSDFYKRLIDKAKTVEGVEALKDAILAA

SEQ ID NO: 636 - protein, 86 aa, Artificial Sequence
  Synthetic polypeptide
HGEGTFTSDLSKQMEEEAVRLFIEWLKNGGPSSGAPPPSGGSLAEAKEAANAELDSYGVSDFYKRLIDKAKTVEGVEALKDAILAA

SEQ ID NO: 637 - protein, 86 aa, Artificial Sequence
  Synthetic polypeptide
HGEGTFTSDLSKQLEEEAVRLFIEWLKNGGPSSGAPPPSGGSLAEAKEAANAELDSYGVSDFYKRLIDKAKTVEGVEALKDAILAA

SEQ ID NO: 638 - protein, 92 aa, Artificial Sequence
  Synthetic polypeptide
HGEGTFTSDLSKQLEEEAVRLFIEWLKQGGPSKEIISTGGGGSASSLAEAKEAANAELDSYGVSDFYKRLIDKAKTVEGVEALKDAILAALP

SEQ ID NO: 639 - protein, 91 aa, Artificial Sequence
  Synthetic polypeptide
HGEGTFTSDLSKQLEEEAVRLFIEWLKQGGPSKEIISTGGGGSASSLAEAKEAANAELDSYGVSDFYKRLIDKAKTVEGVEALKDAILAAL

SEQ ID NO: 640 - protein, 91 aa, Artificial Sequence
  Synthetic polypeptide
HGEGTFTSDLSKQLEEEAVRLFIEWLKQGGPSKEIISTGGGGSASLAEAKEAANAELDSYGVSDFYKRLIDKAKTVEGVEALKDAILAALP

SEQ ID NO: 641 - protein, 90 aa, Artificial Sequence
  Synthetic polypeptide
HGEGTFTSDLSKQLEEEAVRLFIEWLKQGGPSKEIISTGGGGSASLAEAKEAANAELDSYGVSDFYKRLIDKAKTVEGVEALKDAILAAL

SEQ ID NO: 642 - protein, 88 aa, Artificial Sequence
  Synthetic polypeptide
HGEGTFTSDLSKQLEEEAVRLFIEWLKQGGPSKEIISGGGGSLAEAKEAANAELDSYGVSDFYKRLIDKAKTVEGVEALKDAILAALP

SEQ ID NO: 643 - protein, 87 aa, Artificial Sequence
  Synthetic polypeptide
HGEGTFTSDLSKQLEEEAVRLFIEWLKQGGPSKEIISGGGGSLAEAKEAANAELDSYGVSDFYKRLIDKAKTVEGVEALKDAILAAL

SEQ ID NO: 644 - protein, 87 aa, Artificial Sequence
  Synthetic polypeptide
HGEGTFTSDLSKQLEEEAVRLFIEWLKQGGPSKEIISGGGSLAEAKEAANAELDSYGVSDFYKRLIDKAKTVEGVEALKDAILAALP

SEQ ID NO: 645 - protein, 86 aa, Artificial Sequence
  Synthetic polypeptide
HGEGTFTSDLSKQLEEEAVRLFIEWLKQGGPSKEIISGGGSLAEAKEAANAELDSYGVSDFYKRLIDKAKTVEGVEALKDAILAAL

SEQ ID NO: 646 - protein, 86 aa, Artificial Sequence
  Synthetic polypeptide
HGEGTFTSDLSKQLEEEAVRLFIEWLKQGGPSKEIISGGGLAEAKEAANAELDSYGVSDFYKRLIDKAKTVEGVEALKDAILAALP

SEQ ID NO: 647 - protein, 85 aa, Artificial Sequence
  Synthetic polypeptide
HGEGTFTSDLSKQLEEEAVRLFIEWLKQGGPSKEIISGGGLAEAKEAANAELDSYGVSDFYKRLIDKAKTVEGVEALKDAILAAL

SEQ ID NO: 648 - protein, 86 aa, Artificial Sequence
  Synthetic polypeptide
HGEGTFTSDLSKQLEEEAVRLFIEWLKQGGPSKEIISGGSLAEAKEAANAELDSYGVSDFYKRLIDKAKTVEGVEALKDAILAALP

SEQ ID NO: 649 - protein, 85 aa, Artificial Sequence
  Synthetic polypeptide
HGEGTFTSDLSKQLEEEAVRLFIEWLKQGGPSKEIISGGSLAEAKEAANAELDSYGVSDFYKRLIDKAKTVEGVEALKDAILAAL

SEQ ID NO: 650 - protein, 85 aa, Artificial Sequence
  Synthetic polypeptide
HGEGTFTSDLSKQLEEEAVRLFIEWLKQGGPSKEIISGSLAEAKEAANAELDSYGVSDFYKRLIDKAKTVEGVEALKDAILAALP

SEQ ID NO: 651 - protein, 84 aa, Artificial Sequence
  Synthetic polypeptide
HGEGTFTSDLSKQLEEEAVRLFIEWLKQGGPSKEIISGSLAEAKEAANAELDSYGVSDFYKRLIDKAKTVEGVEALKDAILAAL

SEQ ID NO: 652 - protein, 84 aa, Artificial Sequence
  Synthetic polypeptide
HGEGTFTSDLSKQLEEEAVRLFIEWLKQGGPSKEIISGLAEAKEAANAELDSYGVSDFYKRLIDKAKTVEGVEALKDAILAALP

SEQ ID NO: 653 - protein, 83 aa, Artificial Sequence
  Synthetic polypeptide
HGEGTFTSDLSKQLEEEAVRLFIEWLKQGGPSKEIISGLAEAKEAANAELDSYGVSDFYKRLIDKAKTVEGVEALKDAILAAL

SEQ ID NO: 654 - protein, 94 aa, Artificial Sequence
  Synthetic polypeptide
HGEGTFTSDLSKQLEEEAVRLFIEWLKNGGPSSGAPPKSTGGGGSASSLAEAKEAANAELDSYGVSDFYKRLIDKAKTVEGVEALKDAILAALP

SEQ ID NO: 655 - protein, 93 aa, Artificial Sequence
  Synthetic polypeptide
HGEGTFTSDLSKQLEEEAVRLFIEWLKNGGPSSGAPPKSTGGGGSASSLAEAKEAANAELDSYGVSDFYKRLIDKAKTVEGVEALKDAILAAL

SEQ ID NO: 656 - protein, 93 aa, Artificial Sequence
  Synthetic polypeptide
HGEGTFTSDLSKQLEEEAVRLFIEWLKNGGPSSGAPPKSTGGGGSASLAEAKEAANAELDSYGVSDFYKRLIDKAKTVEGVEALKDAILAALP

SEQ ID NO: 657 - protein, 92 aa, Artificial Sequence
  Synthetic polypeptide
HGEGTFTSDLSKQLEEEAVRLFIEWLKNGGPSSGAPPKSTGGGGSASLAEAKEAANAELDSYGVSDFYKRLIDKAKTVEGVEALKDAILAAL

SEQ ID NO: 658 - protein, 90 aa, Artificial Sequence
  Synthetic polypeptide
HGEGTFTSDLSKQLEEEAVRLFIEWLKNGGPSSGAPPKSGGGGSLAEAKEAANAELDSYGVSDFYKRLIDKAKTVEGVEALKDAILAALP

SEQ ID NO: 659 - protein, 89 aa, Artificial Sequence
  Synthetic polypeptide
HGEGTFTSDLSKQLEEEAVRLFIEWLKNGGPSSGAPPKSGGGGSLAEAKEAANAELDSYGVSDFYKRLIDKAKTVEGVEALKDAILAAL

SEQ ID NO: 660 - protein, 89 aa, Artificial Sequence
  Synthetic polypeptide
HGEGTFTSDLSKQLEEEAVRLFIEWLKNGGPSSGAPPKSGGGSLAEAKEAANAELDSYGVSDFYKRLIDKAKTVEGVEALKDAILAALP

SEQ ID NO: 661 - protein, 88 aa, Artificial Sequence
  Synthetic polypeptide
HGEGTFTSDLSKQLEEEAVRLFIEWLKNGGPSSGAPPKSGGGSLAEAKEAANAELDSYGVSDFYKRLIDKAKTVEGVEALKDAILAAL

SEQ ID NO: 662 - protein, 88 aa, Artificial Sequence
  Synthetic polypeptide
HGEGTFTSDLSKQLEEEAVRLFIEWLKNGGPSSGAPPKSGGGLAEAKEAANAELDSYGVSDFYKRLIDKAKTVEGVEALKDAILAALP

SEQ ID NO: 663 - protein, 87 aa, Artificial Sequence
  Synthetic polypeptide
HGEGTFTSDLSKQLEEEAVRLFIEWLKNGGPSSGAPPKSGGGLAEAKEAANAELDSYGVSDFYKRLIDKAKTVEGVEALKDAILAAL

SEQ ID NO: 664 - protein, 88 aa, Artificial Sequence
  Synthetic polypeptide
HGEGTFTSDLSKQLEEEAVRLFIEWLKNGGPSSGAPPKSGGSLAEAKEAANAELDSYGVSDFYKRLIDKAKTVEGVEALKDAILAALP

SEQ ID NO: 665 - protein, 87 aa, Artificial Sequence
  Synthetic polypeptide
HGEGTFTSDLSKQLEEEAVRLFIEWLKNGGPSSGAPPKSGGSLAEAKEAANAELDSYGVSDFYKRLIDKAKTVEGVEALKDAILAAL

SEQ ID NO: 666 - protein, 87 aa, Artificial Sequence
  Synthetic polypeptide
HGEGTFTSDLSKQLEEEAVRLFIEWLKNGGPSSGAPPKSGSLAEAKEAANAELDSYGVSDFYKRLIDKAKTVEGVEALKDAILAALP

SEQ ID NO: 667 - protein, 86 aa, Artificial Sequence
  Synthetic polypeptide
HGEGTFTSDLSKQLEEEAVRLFIEWLKNGGPSSGAPPKSGSLAEAKEAANAELDSYGVSDFYKRLIDKAKTVEGVEALKDAILAAL

SEQ ID NO: 668 - protein, 86 aa, Artificial Sequence
  Synthetic polypeptide
HGEGTFTSDLSKQLEEEAVRLFIEWLKNGGPSSGAPPKSGLAEAKEAANAELDSYGVSDFYKRLIDKAKTVEGVEALKDAILAALP

SEQ ID NO: 669 - protein, 85 aa, Artificial Sequence
  Synthetic polypeptide
HGEGTFTSDLSKQLEEEAVRLFIEWLKNGGPSSGAPPKSGLAEAKEAANAELDSYGVSDFYKRLIDKAKTVEGVEALKDAILAAL

SEQ ID NO: 670 - protein, 95 aa, Artificial Sequence
  Synthetic polypeptide
HGEGTFTSDLSKQMEEEAVRLFIEWLKNGGPSSGAPPKSTGGGGSASGSLAEAKEAANAELDSYGVSDFYKRLIDKAKTVEGVEALKDAILAALP

SEQ ID NO: 671 - protein, 94 aa, Artificial Sequence
  Synthetic polypeptide
HGEGTFTSDLSKQMEEEAVRLFIEWLKNGGPSSGAPPKSTGGGGSASGSLAEAKEAANAELDSYGVSDFYKRLIDKAKTVEGVEALKDAILAAL

SEQ ID NO: 672 - protein, 94 aa, Artificial Sequence
  Synthetic polypeptide
HGEGTFTSDLSKQMEEEAVRLFIEWLKNGGPSSGAPPKSTGGGGSASSLAEAKEAANAELDSYGVSDFYKRLIDKAKTVEGVEALKDAILAALP

SEQ ID NO: 673 - protein, 93 aa, Artificial Sequence
  Synthetic polypeptide
HGEGTFTSDLSKQMEEEAVRLFIEWLKNGGPSSGAPPKSTGGGGSASSLAEAKEAANAELDSYGVSDFYKRLIDKAKTVEGVEALKDAILAAL

SEQ ID NO: 674 - protein, 93 aa, Artificial Sequence
  Synthetic polypeptide
HGEGTFTSDLSKQMEEEAVRLFIEWLKNGGPSSGAPPKSTGGGGSASLAEAKEAANAELDSYGVSDFYKRLIDKAKTVEGVEALKDAILAALP

SEQ ID NO: 675 - protein, 92 aa, Artificial Sequence
  Synthetic polypeptide
HGEGTFTSDLSKQMEEEAVRLFIEWLKNGGPSSGAPPKSTGGGGSASLAEAKEAANAELDSYGVSDFYKRLIDKAKTVEGVEALKDAILAAL

SEQ ID NO: 676 - protein, 90 aa, Artificial Sequence
  Synthetic polypeptide
HGEGTFTSDLSKQMEEEAVRLFIEWLKNGGPSSGAPPKSGGGGSLAEAKEAANAELDSYGVSDFYKRLIDKAKTVEGVEALKDAILAALP

SEQ ID NO: 677 - protein, 89 aa, Artificial Sequence
  Synthetic polypeptide
HGEGTFTSDLSKQMEEEAVRLFIEWLKNGGPSSGAPPKSGGGGSLAEAKEAANAELDSYGVSDFYKRLIDKAKTVEGVEALKDAILAAL

SEQ ID NO: 678 - protein, 89 aa, Artificial Sequence
  Synthetic polypeptide
HGEGTFTSDLSKQMEEEAVRLFIEWLKNGGPSSGAPPKSGGGSLAEAKEAANAELDSYGVSDFYKRLIDKAKTVEGVEALKDAILAALP

SEQ ID NO: 679 - protein, 88 aa, Artificial Sequence
  Synthetic polypeptide
HGEGTFTSDLSKQMEEEAVRLFIEWLKNGGPSSGAPPKSGGGSLAEAKEAANAELDSYGVSDFYKRLIDKAKTVEGVEALKDAILAAL

SEQ ID NO: 680 - protein, 88 aa, Artificial Sequence
  Synthetic polypeptide
HGEGTFTSDLSKQMEEEAVRLFIEWLKNGGPSSGAPPKSGGGLAEAKEAANAELDSYGVSDFYKRLIDKAKTVEGVEALKDAILAALP

SEQ ID NO: 681 - protein, 87 aa, Artificial Sequence
  Synthetic polypeptide
HGEGTFTSDLSKQMEEEAVRLFIEWLKNGGPSSGAPPKSGGGLAEAKEAANAELDSYGVSDFYKRLIDKAKTVEGVEALKDAILAAL

SEQ ID NO: 682 - protein, 88 aa, Artificial Sequence
  Synthetic polypeptide
HGEGTFTSDLSKQMEEEAVRLFIEWLKNGGPSSGAPPKSGGSLAEAKEAANAELDSYGVSDFYKRLIDKAKTVEGVEALKDAILAALP

SEQ ID NO: 683 - protein, 87 aa, Artificial Sequence
  Synthetic polypeptide
HGEGTFTSDLSKQMEEEAVRLFIEWLKNGGPSSGAPPKSGGSLAEAKEAANAELDSYGVSDFYKRLIDKAKTVEGVEALKDAILAAL

SEQ ID NO: 684 - protein, 87 aa, Artificial Sequence
  Synthetic polypeptide
HGEGTFTSDLSKQMEEEAVRLFIEWLKNGGPSSGAPPKSGSLAEAKEAANAELDSYGVSDFYKRLIDKAKTVEGVEALKDAILAALP

SEQ ID NO: 685 - protein, 86 aa, Artificial Sequence
  Synthetic polypeptide
HGEGTFTSDLSKQMEEEAVRLFIEWLKNGGPSSGAPPKSGSLAEAKEAANAELDSYGVSDFYKRLIDKAKTVEGVEALKDAILAAL

SEQ ID NO: 686 - protein, 86 aa, Artificial Sequence
  Synthetic polypeptide
HGEGTFTSDLSKQMEEEAVRLFIEWLKNGGPSSGAPPKSGLAEAKEAANAELDSYGVSDFYKRLIDKAKTVEGVEALKDAILAALP

SEQ ID NO: 687 - protein, 85 aa, Artificial Sequence
  Synthetic polypeptide
HGEGTFTSDLSKQMEEEAVRLFIEWLKNGGPSSGAPPKSGLAEAKEAANAELDSYGVSDFYKRLIDKAKTVEGVEALKDAILAAL

SEQ ID NO: 688 - protein, 94 aa, Artificial Sequence
  Synthetic polypeptide
HGEGTFTSDLSKQLEEEAVRLFIEWLKNGGPSSGAPPPSTGGGGSASSLAEAKEAANAELDSYGVSDFYKRLIDKAKTVEGVEALKDAILAALP

SEQ ID NO: 689 - protein, 93 aa, Artificial Sequence
  Synthetic polypeptide
HGEGTFTSDLSKQLEEEAVRLFIEWLKNGGPSSGAPPPSTGGGGSASSLAEAKEAANAELDSYGVSDFYKRLIDKAKTVEGVEALKDAILAAL

SEQ ID NO: 690 - protein, 93 aa, Artificial Sequence
  Synthetic polypeptide
HGEGTFTSDLSKQLEEEAVRLFIEWLKNGGPSSGAPPPSTGGGGSASLAEAKEAANAELDSYGVSDFYKRLIDKAKTVEGVEALKDAILAALP

SEQ ID NO: 691 - protein, 92 aa, Artificial Sequence
  Synthetic polypeptide
HGEGTFTSDLSKQLEEEAVRLFIEWLKNGGPSSGAPPPSTGGGGSASLAEAKEAANAELDSYGVSDFYKRLIDKAKTVEGVEALKDAILAAL

SEQ ID NO: 692 - protein, 90 aa, Artificial Sequence
  Synthetic polypeptide
HGEGTFTSDLSKQLEEEAVRLFIEWLKNGGPSSGAPPPSGGGGSLAEAKEAANAELDSYGVSDFYKRLIDKAKTVEGVEALKDAILAALP

SEQ ID NO: 693 - protein, 89 aa, Artificial Sequence
  Synthetic polypeptide
HGEGTFTSDLSKQLEEEAVRLFIEWLKNGGPSSGAPPPSGGGGSLAEAKEAANAELDSYGVSDFYKRLIDKAKTVEGVEALKDAILAAL

SEQ ID NO: 694 - protein, 89 aa, Artificial Sequence
  Synthetic polypeptide
HGEGTFTSDLSKQLEEEAVRLFIEWLKNGGPSSGAPPPSGGGSLAEAKEAANAELDSYGVSDFYKRLIDKAKTVEGVEALKDAILAALP

SEQ ID NO: 695 - protein, 88 aa, Artificial Sequence
  Synthetic polypeptide
HGEGTFTSDLSKQLEEEAVRLFIEWLKNGGPSSGAPPPSGGGSLAEAKEAANAELDSYGVSDFYKRLIDKAKTVEGVEALKDAILAAL

SEQ ID NO: 696 - protein, 88 aa, Artificial Sequence
  Synthetic polypeptide
HGEGTFTSDLSKQLEEEAVRLFIEWLKNGGPSSGAPPPSGGGLAEAKEAANAELDSYGVSDFYKRLIDKAKTVEGVEALKDAILAALP

SEQ ID NO: 697 - protein, 87 aa, Artificial Sequence
  Synthetic polypeptide
HGEGTFTSDLSKQLEEEAVRLFIEWLKNGGPSSGAPPPSGGGLAEAKEAANAELDSYGVSDFYKRLIDKAKTVEGVEALKDAILAAL

SEQ ID NO: 698 - protein, 87 aa, Artificial Sequence
  Synthetic polypeptide
HGEGTFTSDLSKQLEEEAVRLFIEWLKNGGPSSGAPPPSGSLAEAKEAANAELDSYGVSDFYKRLIDKAKTVEGVEALKDAILAALP

SEQ ID NO: 699 - protein, 86 aa, Artificial Sequence
  Synthetic polypeptide
HGEGTFTSDLSKQLEEEAVRLFIEWLKNGGPSSGAPPPSGSLAEAKEAANAELDSYGVSDFYKRLIDKAKTVEGVEALKDAILAAL

SEQ ID NO: 700 - protein, 86 aa, Artificial Sequence
  Synthetic polypeptide
HGEGTFTSDLSKQLEEEAVRLFIEWLKNGGPSSGAPPPSGLAEAKEAANAELDSYGVSDFYKRLIDKAKTVEGVEALKDAILAALP

SEQ ID NO: 701 - protein, 85 aa, Artificial Sequence
  Synthetic polypeptide
HGEGTFTSDLSKQLEEEAVRLFIEWLKNGGPSSGAPPPSGLAEAKEAANAELDSYGVSDFYKRLIDKAKTVEGVEALKDAILAAL

SEQ ID NO: 702 - protein, 94 aa, Artificial Sequence
  Synthetic polypeptide
HGEGTFTSDLSKQMEEEAVRLFIEWLKNGGPSSGAPPPSTGGGGSASSLAEAKEAANAELDSYGVSDFYKRLIDKAKTVEGVEALKDAILAALP

SEQ ID NO: 703 - protein, 93 aa, Artificial Sequence
  Synthetic polypeptide
HGEGTFTSDLSKQMEEEAVRLFIEWLKNGGPSSGAPPPSTGGGGSASSLAEAKEAANAELDSYGVSDFYKRLIDKAKTVEGVEALKDAILAAL

SEQ ID NO: 704 - protein, 93 aa, Artificial Sequence
  Synthetic polypeptide
HGEGTFTSDLSKQMEEEAVRLFIEWLKNGGPSSGAPPPSTGGGGSASLAEAKEAANAELDSYGVSDFYKRLIDKAKTVEGVEALKDAILAALP

SEQ ID NO: 705 - protein, 92 aa, Artificial Sequence
  Synthetic polypeptide
HGEGTFTSDLSKQMEEEAVRLFIEWLKNGGPSSGAPPPSTGGGGSASLAEAKEAANAELDSYGVSDFYKRLIDKAKTVEGVEALKDAILAAL

SEQ ID NO: 706 - protein, 90 aa, Artificial Sequence
  Synthetic polypeptide
HGEGTFTSDLSKQMEEEAVRLFIEWLKNGGPSSGAPPPSGGGGSLAEAKEAANAELDSYGVSDFYKRLIDKAKTVEGVEALKDAILAALP

SEQ ID NO: 707 - protein, 89 aa, Artificial Sequence
  Synthetic polypeptide
HGEGTFTSDLSKQMEEEAVRLFIEWLKNGGPSSGAPPPSGGGGSLAEAKEAANAELDSYGVSDFYKRLIDKAKTVEGVEALKDAILAAL

SEQ ID NO: 708 - protein, 89 aa, Artificial Sequence
  Synthetic polypeptide
HGEGTFTSDLSKQMEEEAVRLFIEWLKNGGPSSGAPPPSGGGSLAEAKEAANAELDSYGVSDFYKRLIDKAKTVEGVEALKDAILAALP

SEQ ID NO: 709 - protein, 88 aa, Artificial Sequence
  Synthetic polypeptide
HGEGTFTSDLSKQMEEEAVRLFIEWLKNGGPSSGAPPPSGGGSLAEAKEAANAELDSYGVSDFYKRLIDKAKTVEGVEALKDAILAAL

SEQ ID NO: 710 - protein, 88 aa, Artificial Sequence
  Synthetic polypeptide
HGEGTFTSDLSKQMEEEAVRLFIEWLKNGGPSSGAPPPSGGGLAEAKEAANAELDSYGVSDFYKRLIDKAKTVEGVEALKDAILAALP

SEQ ID NO: 711 - protein, 87 aa, Artificial Sequence
  Synthetic polypeptide
HGEGTFTSDLSKQMEEEAVRLFIEWLKNGGPSSGAPPPSGGGLAEAKEAANAELDSYGVSDFYKRLIDKAKTVEGVEALKDAILAAL

SEQ ID NO: 712 - protein, 87 aa, Artificial Sequence
  Synthetic polypeptide
HGEGTFTSDLSKQMEEEAVRLFIEWLKNGGPSSGAPPPSGSLAEAKEAANAELDSYGVSDFYKRLIDKAKTVEGVEALKDAILAALP

SEQ ID NO: 713 - protein, 86 aa, Artificial Sequence
  Synthetic polypeptide
HGEGTFTSDLSKQMEEEAVRLFIEWLKNGGPSSGAPPPSGSLAEAKEAANAELDSYGVSDFYKRLIDKAKTVEGVEALKDAILAAL

SEQ ID NO: 714 - protein, 86 aa, Artificial Sequence
  Synthetic polypeptide
HGEGTFTSDLSKQMEEEAVRLFIEWLKNGGPSSGAPPPSGLAEAKEAANAELDSYGVSDFYKRLIDKAKTVEGVEALKDAILAALP

SEQ ID NO: 715 - protein, 85 aa, Artificial Sequence
  Synthetic polypeptide
HGEGTFTSDLSKQMEEEAVRLFIEWLKNGGPSSGAPPPSGLAEAKEAANAELDSYGVSDFYKRLIDKAKTVEGVEALKDAILAAL

SEQ ID NO: 716 - protein, 100 aa, Artificial Sequence
  Synthetic polypeptide
HGEGTFTSDLSKQLEEEAVRLFIEWLKNGGPSSGAPPSKKKKKKTGGGGSASGSLAEAKEAANAELDSYGVSDFYKRLIDKAKTVEGVEALKDAILAALP

SEQ ID NO: 717 - protein, 99 aa, Artificial Sequence
  Synthetic polypeptide
HGEGTFTSDLSKQLEEEAVRLFIEWLKNGGPSSGAPPSKKKKKKTGGGGSASGSLAEAKEAANAELDSYGVSDFYKRLIDKAKTVEGVEALKDAILAAL

SEQ ID NO: 718 - protein, 99 aa, Artificial Sequence
  Synthetic polypeptide
HGEGTFTSDLSKQLEEEAVRLFIEWLKNGGPSSGAPPSKKKKKKTGGGGSASSLAEAKEAANAELDSYGVSDFYKRLIDKAKTVEGVEALKDAILAALP

SEQ ID NO: 719 - protein, 98 aa, Artificial Sequence
  Synthetic polypeptide
HGEGTFTSDLSKQLEEEAVRLFIEWLKNGGPSSGAPPSKKKKKKTGGGGSASSLAEAKEAANAELDSYGVSDFYKRLIDKAKTVEGVEALKDAILAAL

SEQ ID NO: 720 - protein, 98 aa, Artificial Sequence
  Synthetic polypeptide
HGEGTFTSDLSKQLEEEAVRLFIEWLKNGGPSSGAPPSKKKKKKTGGGGSASLAEAKEAANAELDSYGVSDFYKRLIDKAKTVEGVEALKDAILAALP

SEQ ID NO: 721 - protein, 97 aa, Artificial Sequence
  Synthetic polypeptide
HGEGTFTSDLSKQLEEEAVRLFIEWLKNGGPSSGAPPSKKKKKKTGGGGSASLAEAKEAANAELDSYGVSDFYKRLIDKAKTVEGVEALKDAILAAL

SEQ ID NO: 722 - protein, 95 aa, Artificial Sequence
  Synthetic polypeptide
HGEGTFTSDLSKQLEEEAVRLFIEWLKNGGPSSGAPPSKKKKKKGGGGSLAEAKEAANAELDSYGVSDFYKRLIDKAKTVEGVEALKDAILAALP

SEQ ID NO: 723 - protein, 94 aa, Artificial Sequence
  Synthetic polypeptide
HGEGTFTSDLSKQLEEEAVRLFIEWLKNGGPSSGAPPSKKKKKKGGGGSLAEAKEAANAELDSYGVSDFYKRLIDKAKTVEGVEALKDAILAAL

SEQ ID NO: 724 - protein, 94 aa, Artificial Sequence
  Synthetic polypeptide
HGEGTFTSDLSKQLEEEAVRLFIEWLKNGGPSSGAPPSKKKKKKGGGSLAEAKEAANAELDSYGVSDFYKRLIDKAKTVEGVEALKDAILAALP

SEQ ID NO: 725 - protein, 93 aa, Artificial Sequence
  Synthetic polypeptide
HGEGTFTSDLSKQLEEEAVRLFIEWLKNGGPSSGAPPSKKKKKKGGGSLAEAKEAANAELDSYGVSDFYKRLIDKAKTVEGVEALKDAILAAL

SEQ ID NO: 726 - protein, 93 aa, Artificial Sequence
  Synthetic polypeptide
HGEGTFTSDLSKQLEEEAVRLFIEWLKNGGPSSGAPPSKKKKKKGGGLAEAKEAANAELDSYGVSDFYKRLIDKAKTVEGVEALKDAILAALP

SEQ ID NO: 727 - protein, 92 aa, Artificial Sequence
  Synthetic polypeptide
HGEGTFTSDLSKQLEEEAVRLFIEWLKNGGPSSGAPPSKKKKKKGGGLAEAKEAANAELDSYGVSDFYKRLIDKAKTVEGVEALKDAILAAL

SEQ ID NO: 728 - protein, 93 aa, Artificial Sequence
  Synthetic polypeptide
HGEGTFTSDLSKQLEEEAVRLFIEWLKNGGPSSGAPPSKKKKKKGGSLAEAKEAANAELDSYGVSDFYKRLIDKAKTVEGVEALKDAILAALP

SEQ ID NO: 729 - protein, 92 aa, Artificial Sequence
  Synthetic polypeptide
HGEGTFTSDLSKQLEEEAVRLFIEWLKNGGPSSGAPPSKKKKKKGGSLAEAKEAANAELDSYGVSDFYKRLIDKAKTVEGVEALKDAILAAL

SEQ ID NO: 730 - protein, 92 aa, Artificial Sequence
  Synthetic polypeptide
HGEGTFTSDLSKQLEEEAVRLFIEWLKNGGPSSGAPPSKKKKKKGSLAEAKEAANAELDSYGVSDFYKRLIDKAKTVEGVEALKDAILAALP

SEQ ID NO: 731 - protein, 91 aa, Artificial Sequence
  Synthetic polypeptide
HGEGTFTSDLSKQLEEEAVRLFIEWLKNGGPSSGAPPSKKKKKKGSLAEAKEAANAELDSYGVSDFYKRLIDKAKTVEGVEALKDAILAAL

SEQ ID NO: 732 - protein, 91 aa, Artificial Sequence
  Synthetic polypeptide
HGEGTFTSDLSKQLEEEAVRLFIEWLKNGGPSSGAPPSKKKKKKGLAEAKEAANAELDSYGVSDFYKRLIDKAKTVEGVEALKDAILAALP

SEQ ID NO: 733 - protein, 90 aa, Artificial Sequence
  Synthetic polypeptide
HGEGTFTSDLSKQLEEEAVRLFIEWLKNGGPSSGAPPSKKKKKKGLAEAKEAANAELDSYGVSDFYKRLIDKAKTVEGVEALKDAILAAL

SEQ ID NO: 734 - protein, 113 aa, Artificial Sequence
  Synthetic polypeptide
MAHHHHHHVGTGSNENLYFQHGEGTFTSDLSKQLEEEAVRLFIEWLKQGGPSKEIISTGGGGSASGSLAEAKEAANAELDSYGVSDFYKRLIDKAKTVEGVEALKDAILAALP

SEQ ID NO: 735 - protein, 252 aa, Artificial Sequence
  Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQLEEEAVRLFIEWLKQGGPSKEIISTGGGGSASGSLAEAKEAANAELDSYGVSDFYKRLIDKAKTVEGVEALKDAILAALP

SEQ ID NO: 736 - protein, 115 aa, Artificial Sequence
  Synthetic polypeptide
MAHHHHHHVGTGSNENLYFQHGEGTFTSDLSKQLEEEAVRLFIEWLKNGGPSSGAPPPSTGGGGSASGSLAEAKEAANAELDSYGVSDFYKRLIDKAKTVEGVEALKDAILAALP

SEQ ID NO: 737 - protein, 254 aa, Artificial Sequence
  Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQLEEEAVRLFIEWLKNGGPSSGAPPPSTGGGGSASGSLAEAKEAANAELDSYGVSDFYKRLIDKAKTVEGVEALKDAILAALP

SEQ ID NO: 738 - protein, 115 aa, Artificial Sequence
  Synthetic polypeptide
MAHHHHHHVGTGSNENLYFQHGEGTFTSDLSKQMEEEAVRLFIEWLKNGGPSSGAPPPSTGGGGSASGSLAEAKEAANAELDSYGVSDFYKRLIDKAKTVEGVEALKDAILAALP

SEQ ID NO: 739 - protein, 254 aa, Artificial Sequence
  Synthetic polypeptide
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLAGSGSGHMHHHHHHSSGLVPRGSGMKETAAAKFERQHMDSPDLGTENLYFQHGEGTFTSDLSKQMEEEAVRLFIEWLKNGGPSSGAPPPSTGGGGSASGSLAEAKEAANAELDSYGVSDFYKRLIDKAKTVEGVEALKDAILAALP

SEQ ID NO: 740 - protein, 27 aa, Artificial Sequence
  Synthetic polypeptide
MGSSHHHHHHLQSSGVDLGTENLYFQG

SEQ ID NO: 741 - protein, 84 aa, Artificial Sequence
  Synthetic polypeptide
HGEGTFTSDLSKQLEEEAVRLFIEWLKNTGGGGSASGSLAEAKEAANAELDSYGVSDFYKRLIDKAKTVEGVEALKDAILAALP

(19) United States
(12) Patent Application Publication
(10) Pub. No.: US 2015/0133373 A1
(43) Pub. Date: May 14, 2015

(54) TRANSMUCOSAL DELIVERY OF ENGINEERED POLYPEPTIDES

(71) Applicants: AMYLIN PHARMACEUTICALS, LLC, San Diego, CA (US); ASTRAZENECA PHARMACEUTICALS LP, Wilmington, DE (US)

(72) Inventors: Steven Shijun Ren, San Diego, CA (US); Li Jin, San Diego, CA (US)

(21) Appl. No.: 14/388,510

(22) PCT Filed: Mar. 28, 2013
(86) PCT No.: PCT/US2013/034300

§ 371 Date: Sep. 26, 2014

Related U.S. Application Data
(60) Provisional application No. 61/616,961, filed on Mar. 28, 2012.

(51) Int. Cl. A61K 47/26; A61K 47/48; A61K 47/14; A61K 38/22

(52) U.S. Cl. CPC A61K 47/26; A61K 38/2278; A61K 47/48284; A61K 47/14
USPC 514/6.9; 514/13.1; 514/6.8

(57) ABSTRACT

Formulations are provided that comprise compounds having inter alia good duration of action, high potency and/or convenient dosing regimens, and a permeation enhancer for transmucosal administration. The compounds are engineered polypeptides which incorporate an albumin binding domain in combination with one or more biologically active polypeptides. The pharmaceutical compositions provided are suitable for methods of treatment for diseases and disorders including obesity and overweight, diabetes, dyslipidemia, hyperlipidemia, Alzheimer's disease, fatty liver disease, short bowel syndrome, Parkinson's disease, cardiovascular disease, and other and disorders of the central nervous system.

### CROSS-REFERENCE TO RELATED APPLICATIONS

This application claims priority to U.S. Provisional Patent Application No. 61/616,961, filed Mar. 28, 2012, the contents of which are incorporated herein by reference in its entirety and for all purposes.

### REFERENCE TO A “SEQUENCE LISTING,” A TABLE, OR A COMPUTER PROGRAM LISTING APPENDIX SUBMITTED AS AN ASCII TEXT FILE

The Sequence Listing written in file 92494-870568_ST25.TXT, created on Mar. 22, 2013, 399,949 bytes, machine format IBM-PC, MS-Windows operating system, is hereby incorporated by reference.

### BACKGROUND OF THE INVENTION

The present application relates to formulations for non-invasive, transmucosal delivery of compounds having good duration of action, high potency and/or convenient dosing regimens including oral administration, and method of use thereof. There are provided herein formulations of engineered polypeptides (Exendin ABD) which incorporate an albumin binding domain in combination with a biologically active peptide. Without wishing to be bound by any theory, it is believed that because the engineered polypeptides described herein can bind albumin with high affinity, the compounds can be sequestered (e.g., bound to albumin) while in the circulation leading to increased duration of action, due for example to decreased renal clearance and/or degradation. Surprisingly, the compounds are active while bound to circulating serum albumin. Because the compounds also comprise a sequence with exendin-4 activity, diseases amendable to such treatment include obesity and overweight, diabetes, dyslipidemia, hyperlipidemia, short bowel syndrome, Alzheimer's disease, fatty liver disease, NASH, Parkinson's disease, cardiovascular disease, and other disorders of the central nervous system, or combinations thereof.

There remains a need for methods and formulations for delivery of polypeptides useful in the above described metabolic diseases, conditions and disorders. Accordingly, it is an object of the present invention to provide formulations for non-invasive transmucosal delivery of engineered polypeptides with extended half-lives useful to treat the above conditions.

Each patent, patent application, and publication cited herein is hereby incorporated herein by reference in its entirety and for all purposes.

### BRIEF SUMMARY OF THE INVENTION

Provided are compositions and improved compositions for non-invasive, transmucosal delivery comprising an engineered polypeptide (Exendin ABD polypeptide), as defined herein, and at least one transmucosal permeation enhancer. The permeation enhancer or combination of permeation enhancers provides formulations for non-invasive, transmucosal delivery, or improved transmucosal delivery. Further improvement can be achieved by the incorporation of additional agents as described herein. The permeation enhancer can enhance paracellular permeation, open cell tight junctions, enhance transcellular permeation, inhibit an intestinal protease, enhance solubility of a different permeation enhancer and/or be mucoadhesive. The composition can optionally, but preferably, further comprise a second permeation enhancer, wherein the second permeation enhancer enhances paracellular permeation, opens cell tight junctions, enhances transcellular permeation, inhibits an intestinal protease, enhances solubility of a different permeation enhancer in the composition and/or is a mucoadhesive. The composition can further optionally but preferably comprise a third permeation enhancer, wherein the third permeation enhancer enhances paracellular permeation, opens cell tight junctions, enhances transcellular permeation, inhibits an intestinal protease, enhances solubility of a different permeation enhancer in the composition and/or is a mucoadhesive. The composition can optionally further comprise (c) an inhibitor of an intestinal protease, a mucoadhesive, a surfactant, an oil, an emulsifier or a mixture thereof, to further improve bioavailability of the delivered engineered polypeptide (Exendin ABD). The composition can optionally further comprise (d) a pH lowering agent to further improve bioavailability of the delivered engineered polypeptide (Exendin ABD) by decreasing activity of proteases at the site of delivery. The composition can optionally further comprise conventional formulation agents (e) including a bulking agent, a polypeptide stabilizing agent, or other excipient, or a mixture thereof.

In a preferred formulation embodiment, the composition comprises an Exendin ABD formulated with a permeation enhancer that is a non-conjugated bile acid or salt and a permeation enhancer that is an aromatic alcohol. Preferably the non-conjugated bile acid or salt, in addition to enhancing permeation, enhances the solubility of the aromatic acid at the site of absorption, e.g. small intestine.

The Exendin ABD compounds are engineered polypeptides which include an Albumin Binding Domain (ABD) polypeptide as defined herein capable of binding albumin, and a hormone domain (HD1) polypeptide as defined herein, which HD1 polypeptides can be biologically active and can elicit a beneficial biological response, in covalent linkage with the ABD. The hormone domain includes a polypeptide which is an exendin, a fragment of an exendin, or analog of an exendin. Any of the ABD or HD1 polypeptides described herein can be optionally covalently bonded in the engineered polypeptide through a linker L, for example L1 as described herein. Without wishing to be bound by any theory, it is believed that because the engineered polypeptides described herein can bind albumin, the compounds can be sequestered in a subject leading to increased duration of action in the subject. In a first aspect, there is provided a formulation of an engineered polypeptide as described herein. The engineered polypeptide includes an Albumin Binding Domain polypeptide (ABD), either an ABD1 type ABD or an ABD2 type ABD as described herein, and a hormone domain (HD1). The hormone domain includes a polypeptide which is an exendin, a fragment of an exendin, or analog of an exendin.

In another aspect, there is provided a method for treating a disease or disorder in a subject in need of treatment using the formulations of the invention. The method includes administering an engineered polypeptide formulated as described herein to the subject.

In yet another aspect, there is provided a method for making a pharmaceutical composition described herein.

One advantage of the present invention is that the formulations contain engineered polypeptides that can be synthesized completely by recombinant methods, avoiding complex or additional synthetic or chemical steps and associated reactive reagents and catalysts. Consequently, the engineered polypeptides used in the present invention can be much less expensive to synthesize than chemically derivatized compounds of prolonged duration of action. In addition to a long duration of action (e.g., at least one week in a human subject, albeit once daily delivery can be provided as the Exendin ABD provide long action of longer than a day), a further advantage is relatively small size, which can allow for oral delivery, or other non-invasive delivery routes, as demonstrated herein to improve patient compliance.

The compounds disclosed herein demonstrate surprising efficacy in an OGTT DOA (oral glucose tolerance test for duration of action) test of at least 24 hours and even longer to 2 days in mice, which translates to 7 days or longer in humans, a robust glycemic control and body weight loss in diabetic obese (ob/ob) mice, and provide a dose-dependent reduction of food intake over at least two days in mice. In normal rats, compound exposure lasts for several days (even as long as 4-5 days, which translates to at least once a week in humans) after subcutaneous and intravenous dosing. The compounds also provide body weight reduction activity. Compounds are stable in plasma and to plasma proteases, are active while bound to serum albumin, and surprisingly provide greater maximal in vivo efficacy than exendin-4 as shown herein. In one aspect the compounds contain an ABD that is deimmunized (referred to as an ABD2 type ABD), providing less immunogenicity than a non-deimmunized ABD (referred to as an ABD1 type ABD). Even more surprisingly the compounds are suitable for oral delivery. Even more surprising is the improved uptake and bioavailability, with retention of duration and biological activity, of the Exendin ABD as formulated for transmucosal delivery herein.

### BRIEF DESCRIPTION OF THE DRAWINGS

FIG. 1A: Blood glucose level (BGL) data histogram prior to gavage at 1-day post dosage of Cmpd 15 in OGTT DOA test. Vehicle mean pre-gavage glucose: 117 mg/dL. Legend (left to right): vehicle (open), 2 nmol/kg (diagonal upper left to lower right); 25 nmol/kg (diagonal lower left to upper right); 250 nmol/kg (fine diagonal). FIG. 1B: Change in blood glucose at 30 min. Vehicle mean pre-gavage glucose: 117 mg/dL. Legend: same as in FIG. 1A. * p<0.5 vs. vehicle control; ANOVA, Dunnett's test.

FIG. 2A: Blood glucose level (BGL) data histogram prior to gavage at 2-day post dosage of Cmpd 15 in OGTT DOA test. Vehicle mean pre-gavage glucose: 135 mg/dL. Legend (left to right): vehicle (open), 25 nmol/kg (vertical lines); 250 nmol/kg (diagonal lines). FIG. 2B: Change in blood glucose at 30 min. Vehicle mean pre-gavage glucose: 135 mg/dL. Legend: same as FIG. 2A. * p<0.5 vs. vehicle control; ANOVA, Dunnett's test.

FIG. 3A: Blood glucose level (BGL) data histogram prior to gavage at 1-day post dosage of Cmpd 15 and Cmpd 8 in OGTT DOA test. Vehicle mean pre-gavage glucose: 117 mg/dL. Legend (left to right): vehicle (open); 2 nmol/kg Cmpd 15 (diagonal upper left to lower right); 25 nmol/kg Cmpd 15 (diagonal lower left to upper right); 250 nmol/kg Cmpd 15 (fine diagonal); 2 nmol/kg Cmpd 8 (tiled); 25 nmol/kg Cmpd 8 (horizontal lines); 250 nmol/kg Cmpd 8 (dotted). FIG. 3B: Change in blood glucose at 30 min. Vehicle mean pre-gavage glucose: 117 mg/dL. Legend: same as FIG. 1A. * p<0.5 vs. vehicle control; ANOVA, Dunnett's test.

FIG. 4: Effect of Cmpd 15 in HSD fed anesthetized rats. FIG. 4A: Glucose time course after intravenous glucose tolerance test (IVGTT). Legend: vehicle (Triangle tip up); Cmpd 15 at 240 nmol/kg (box). FIG. 4B: Histogram depicting glucose (AUC, 0-60 min) after IVGTT. Legend: vehicle (left); Cmpd 15 (right). FIG. 4C: Time course of insulin after IVGTT. Legend: As in FIG. 4A. FIG. 4D: Histogram depicting change in insulin (AUC, 0-30 min) Legend: As in FIG. 4B. FIG. 4E: Time course of change in body weight after sc injection of Cmpd 15. Legend: As in FIG. 4A. FIG. 4F: Histogram of daily food intake after sc injection of Cmpd 15. Legend: for each day, histogram depicts vehicle and Cmpd 15 (240 nmol/kg) in order left to right; n=5. *p<0.05 vs. vehicle control; Dunnett's test.

FIG. 5: Effect of Cmpd 15 in ob/ob mice. FIG. 5A: Time course of change in body weight (0-10 days) after injection of Cmpd 15 at 250 nmol/kg. Legend: Vehicle (square); Cmpd 15 (triangle). FIG. 5B: Time course of change in blood glucose after dosage as described for FIG. 5A. Legend: As in FIG. 5A. FIG. 5C: Time course of change in HbA_{1c} after dosage as described for FIG. 5A. Legend: As in FIG. 5A. * p<0.5 vs. vehicle control; ANOVA, Dunnett's test.

FIG. 6: Effects of Cmpd 15 in Zucker Diabetic Fatty rats. FIG. 6A: Time course of change in body weight after treatment of Zucker Diabetic Fatty rats with Cmpd 15. FIG. 6B: Time course of plasma glucose (mg/dL) after treatment with Cmpd 15. Legend: Vehicle (solid box); Cmpd 15 (0.17 mg/kg) (triangle tip up); Cmpd 15 (0.5 mg/kg) (triangle tip down).

FIG. 7: Comparison in OGTT DOA. Effects of Cmpds 15, 8 and 10, compared with exendin-4, were evaluated as the change in blood glucose at 30 min (% pre-gavage). Legend: compounds in order left to right of histogram: vehicle; Cmpd 15 at 2 nmol/kg; Cmpd 15 at 25 nmol/kg; Cmpd 15 at 250 nmol/kg; Cmpd 8 at 2 nmol/kg; Cmpd 8 at 25 nmol/kg; Cmpd 8 at 250 nmol/kg; Cmpd 10 at 25 nmol/kg; Cmpd 10 at 250 nmol/kg; exendin-4 at 250 nmol/kg. * p<0.5 vs. vehicle control; ANOVA, Dunnett's test.

FIG. 8: Presents a time profile of percent of compound remaining in human plasma over a 5 hour time course. Legend: Peptide (SEQ ID NO:4) (closed box); Cmpd 7 (open box); Cmpd 31 (cross); Cmpd 15 (open diamond); GLP-1(7-36)amide (closed diamond).

FIG. 9: Blood glucose level (BGL) data histogram prior to gavage at 1-day post dosage of Cmpd 31. Vehicle mean pre-gavage glucose: 126 mg/dL. Legend: vehicle (open), Cmpd 31 (25 nmol/kg; closed). Legend: same as FIG. 1A. * p<0.5 vs. vehicle control; ANOVA, Dunnett's test.

FIG. 10: FIG. 10A demonstrates time course of effect of Cmpd 31 on inhibiting food intake in normal mice over 6 hours. Legend: vehicle (box); Cmpd 31 at 1 nmol/kg (diamond); Cmpd 31 at 10 nmol/kg (cross); Cmpd 31 at 30 nmol/kg (circle); Cmpd 31 at 100 nmol/kg (star). FIG. 10B depicts histogram of results of effect of Cmpd 31 on inhibiting food intake in normal mice over 54 hours. Legend (left to right for each time period): vehicle (open); [¹⁴Leu]exendin-4 at 1 nmol/kg (verticle lines); [¹⁴Leu]exendin-4 at 10 nmol/kg (diagonal lines, upper left to lower right); [¹⁴Leu]exendin-4 at 30 nmol/kg (diagonal lines, lower left to upper right); [¹⁴Leu]exendin-4 at 100 nmol/kg (fine diagonal lines); Cmpd 31 at 1 nmol/kg (vertical lines); Cmpd 31 at 10 nmol/kg (light dots); Cmpd 31 at 30 nmol/kg (heavy dots); Cmpd 31 at 100 nmol/kg (checkered).

FIG. 11: FIG. 11A (Cmpd 15) and FIG. 11B (Cmpd 21) depict time course of changes in blood glucose compared to liraglutide, all given twice weekly (BIW). Legend (FIGS. 11A-11B): vehicle (box); liraglutide at 250 nmol/kg BIW (closed triangle); test compound at 25 nmol/kg BIW (open triangle); test compound at 250 nmol/kg BIW (diamond). FIG. 11C depicts histogram showing lowering of HbA1c (% change from baseline) for Cmpd 15 and Cmpd 21 given twice weekly (BIW), compared to exendin-4 given by continuous subcutaneous infusion (CSI). Legend (left to right): vehicle (open); Cmpd 15 at 25 nmol/kg BIW (fine checkered); Cmpd 15 at 250 nmol/kg BIW (dotted); Cmpd 21 at 25 nmol/kg BIW (diagonal crosshatching); Cmpd 21 at 250 nmol/kg BIW (vertical-horizontal crosshatching); exendin-4 at 7.2 nmol/kg/day CSI (dark tiling); exendin-4 at 100 nmol/kg/day CSI (light tiling). FIG. 11D depicts reduction in body weight (% change from baseline) for Cmpd 15 and Cmpd 21 given twice weekly (BIW), compared to exendin-4 given by continuous subcutaneous infusion (CSI). Legend (left to right): as in FIG. 11C.

FIG. 12: FIGS. 12A-12C depict pharmacokinetic (PK) profile and biological activity of exemplary engineered polypeptides Cmpd 15 and Cmpd 21 dosed subcutaneously in normal Harlan Sprague-Dawley (HSD) rats. FIG. 12A depicts effect of compounds to reduce food intake. FIG. 12B depicts effect of compounds to reduce body weight. FIG. 12C depicts a PK profile of the compounds after a single dose. Legend: vehicle (box); Cmpd 21 (triangle); Cmpd 15 (diamond).

FIG. 13: FIGS. 13A-13C depict pharmacokinetic (PK) profile and biological activity of an exemplary engineered polypeptide Cmpd 31 compared to unconjugated exendin analog dosed intravenously in normal Harlan Sprague-Dawley (HSD) rats. FIG. 13A depicts effect of compounds to reduce food intake. FIG. 13B depicts effect of compounds to reduce body weight. FIG. 13C depicts a PK profile of the compounds after a single dose. Inset: Tabulation of time versus PK results (pg/mL) for [¹⁴Leu]exendin-4 at 2 nmol/kg IV and Cmpd 31 at 2 nmol/kg IV. Legend: vehicle (diamond); [¹⁴Leu]exendin-4 at 2 nmol/kg IV (box); Cmpd 31 at 2 nmol/kg IV (circle).

FIG. 14: This figure depicts a biological activity time course of an exemplary engineered polypeptide (Cmpd 15) compared to unconjugated exendin analog to lower blood glucose after oral delivery. See Example 18. Mean pre-treatment glucose: ˜623 mg/dL. Legend: vehicle (closed box); exendin-4 analog (open box); Cmpd 15 (diamond).

FIG. 15 shows the result of binding analysis performed in a Biacore instrument for investigating the binding of the albumin binding polypeptide PEP07912 (SEQ ID NO:456) to human serum albumin. Three different concentrations of purified protein (40 nM, fat gray line; 10 nM, black line; and 2.5 nM, gray line) were injected over a surface with 955 RU of immobilized human serum albumin.

FIG. 16A-16C show the result of binding analysis performed by ELISA for investigating the binding of the albumin binding polypeptides PEP07913 (SEQ ID NO:453), PEP06923 (SEQ ID NO:454), PEP07271 (SEQ ID NO:455), PEP07912 (SEQ ID NO:457), PEP07554 (SEQ ID NO:456), PEP07914 (SEQ ID NO:458), PEP07968 (i.e. DOTA conjugated to PEP07911 (SEQ ID NO:459)) and PEP07844 (SEQ ID NO:461), to IgG molecules present in 126 individual normal human sera, where A) shows the average OD-value, B) shows the percentage of negative sera (defined as OD<0.15), and C) shows the percentage of positive sera (defined as OD>1.0).

FIG. 17A-17C are diagrams showing an immunogenicity assessment of albumin binding polypeptides PEP07913 (SEQ ID NO:453), PEP07912 (SEQ ID NO:457), PEP07914 (SEQ ID NO:458) and PEP07968 (i.e. DOTA conjugated to PEP07911 (SEQ ID NO:459)) in a CD3+CD4+ T cell proliferation assay. A) shows the number of individuals responding to the albumin binding polypeptides compared to recombinant human albumin in a cohort of 52 Caucasian donors. B) shows the average stimulation indices (SI) for PEP07913, PEP07912, PEP07914 and PEP07968 compared to the negative control containing recombinant human albumin. C) shows the number of responding individuals against all proteins in the study as compared to the buffer control.

FIG. 18A-18C. Pharmacokinetic (PK) profile and biological activity of an exemplary engineered polypeptide Cmpd 2-11 dosed in normal Harlan Sprague-Dawley (HSD) rats. FIG. 18A depicts effect of compounds to reduce food intake. FIG. 18B depicts effect of compounds to reduce body weight. FIG. 18C depicts a PK profile of the compound after a single dose. In the figures, vehicle is solid square and engineered polypeptide is open inverted triangle.

FIG. 19A-19C. Pharmacokinetic (PK) profile and biological activity of an exemplary engineered polypeptide Cmpd 2-9 dosed in normal Harlan Sprague-Dawley (HSD) rats. FIG. 19A depicts effect of compounds to reduce food intake. FIG. 19B depicts effect of compounds to reduce body weight. FIG. 19C depicts a PK profile of the compound after a single dose. In the figures, vehicle is solid square and engineered polypeptide is closed triangle.

FIG. 20A-20C. FIGS. 20A-20C depict pharmacokinetic (PK) profile and biological activity of an exemplary engineered polypeptide Cmpd 2-11 compared to an unconjugated exendin analog dosed intravenously in normal Harlan Sprague-Dawley (HSD) rats. FIG. 20A depicts effect of compound to reduce food intake. FIG. 20B depicts effect of compound to reduce body weight. FIG. 20C depicts a PK profile of the exemplary compound after a single intravenous dose. Results presented as picomolar plasma levels. Legend FIGS. 20A and 20B: vehicle (diamond); [¹⁴Leu]exendin-4 at 2 nmol/kg IV (closed triangle); Cmpd 2-11 at 2 nmol/kg IV (square). Legend: FIG. 20C: [¹⁴Leu]exendin-4 at 2 nmol/kg IV (square); exendin-4 at 2 nmol/kg IV (open circle); Cmpd 2-11 at 2 nmol/kg IV (closed triangle).

FIG. 21A-21F. FIGS. 21A-21F depict pharmacokinetic (PK) profile and biological activity of an exemplary engineered polypeptide Cmpd 2-11 administered sub-chronically either daily or twice weekly. Cmpd 2-11 was subcutaneously administered at 25 nmol/kg over 14 days, either twice weekly (BIW; open inverted trangles) as indicated by the down arrows or daily (QD; open square) and compared to vehicle (closed circle). FIG. 21A depicts cumulative food intake. FIG. 21B depicts percent change in daily food intake. FIG. 21C depicts percent change in cumulative food intake. FIG. 21D depicts total body weight. FIG. 21E depicts percent change in body weight. FIG. 21F depicts a PK profile of Cmpd 2-11 given BIW or QD.

FIG. 22A-22I depict results comparing intra-duodenal administration of exemplary Exendin ABD compounds Cmpd 103 (Cmpd 31) and Cmpd 202 (Cmpd 2-11) and of comparator compound Cmpd X, formulated in OF1, OF2, OF3, OF4 and PBS (ID-PBS) as described herein for intestinal transmucosal uptake in rats. Results for intravenous administration in PBS (IV-PBS) are also shown. FIG. 22A depicts blood plasma exposure as AUC at 120 minutes after delivery of the formulations described herein for Cmpd 103. FIG. 22B presents a pharmacokinetic profile over 120 minutes for the formulations of Cmpd 103. FIG. 22C presents profiles of blood pressure after delivery of the formulations of Cmpd 103. FIG. 22D depicts blood plasma exposure as AUC at 120 minutes after delivery of the formulations described herein for Cmpd 202. FIG. 22E presents a pharmacokinetic profile over 120 minutes for the formulations of Cmpd 202. FIG. 22F presents blood pressure profiles after delivery of the formulations of Cmpd 202. FIG. 22G depicts blood plasma exposure as AUC at 120 minutes after delivery of the formulations described herein for Cmpd X. FIG. 22H presents a pharmacokinetic profile over 120 minutes for the formulations of Cmpd X. FIG. 22I presents profiles of blood pressure after delivery of the formulations of Cmpd X.

FIG. 23 depicts blood plasma exposure over time of exemplary engineered polypeptides formulated in OF1 administered intra-jejunally to rats. Cmpd 101 is also referred to as Cmpd 15. Cmpd 102 is also referred to as Cmpd 21. Cmpd 103 is also referred to as Cmpd 31. Cmpd 201 is also referred to as Cmpd 2-9. Cmpd 202 is also referred to as Cmpd 2-11.

FIG. 24 is a pharmacokinetic profile in blood plasma of exemplary engineered polypeptide Cmpd 103 (Cmpd 15) formulated in OF1 after a single oral administration to beagle dogs.

FIG. 25 is a pharmacokinetic profiles in blood plasma of exemplary engineered polypeptide Cmpd 103 (Cmpd 15) and Cmpd X formulated in OF1 after a single oral administration to a cynomolgous monkey.

### DETAILED DESCRIPTION OF THE INVENTION

### I. Definitions

“Obesity” and “overweight” refer to mammals having a weight greater than normally expected, and may be determined by, e.g., physical appearance, body mass index (BMI) as known in the art, waist-to-hip circumference ratios, skinfold thickness, waist circumference, and the like. The Centers for Disease Control and Prevention (CDC) define overweight as an adult human having a BMI of 25 to 29.9; and define obese as an adult human having a BMI of 30 or higher. Additional metrics for the determination of obesity exist. For example, the CDC states that a person with a waist-to-hip ratio greater than 1.0 is overweight.

“Lean body mass” refers to the fat-free mass of the body, i.e., total body weight minus body fat weight is lean body mass. Lean body mass can be measured by methods such as hydrostatic weighing, computerized chambers, dual-energy X-ray absorptiometry, skin calipers, magnetic resonance imaging (MRI) and bioelectric impedance analysis (BIA) as known in the art.

“Mammal” refers to warm-blooded animals that generally have fur or hair, that give live birth to their progeny, and that feed their progeny with milk. Mammals include humans; companion animals (e.g., dogs, cats); farm animals (e.g., cows, horses, sheep, pigs, goats); wild animals; and the like. In one embodiment, the mammal is a female. In one embodiment, the mammal is a female human. In one embodiment, the mammal is a cat or dog. In one embodiment, the mammal is a diabetic mammal, e.g., a human having type 2 diabetes. In one embodiment, the mammal is an obese diabetic mammal, e.g., an obese mammal having type 2 diabetes. The term “subject” in the context of methods described herein refers to a mammal.

“Fragment” in the context of polypeptides refers herein in the customary chemical sense to a portion of a polypeptide. For example, a fragment can result from N-terminal deletion or C-terminal deletion of one or more residues of a parent polypeptide, and/or a fragment can result from internal deletion of one or more residues of a parent polypeptide. “Fragment” in the context of an antibody refers to a portion of an antibody which can be linked to a biologically active molecule to modulate solubility, distribution within a subject, and the like. For example, exendin-4(1-30) describes a biologically active fragment of exendin-4 where the exendin C-terminal “tail” of amino acids 31-39 is deleted. The term “parent” in the context of polypeptides refers, in the customary sense, to a polypeptide which serves as a reference structure prior to modification, e.g., insertion, deletion and/or substitution. The term “conjugate” in the context of engineered polypeptides described herein refers to covalent linkage between component polypeptides, e.g., ABD, HD1 and the like. The term “fusion” in the context of engineered polypeptides described herein refers to covalent linkage between component polypeptides, e.g., ABD, HD1 and the like, via either or both terminal amino or carboxy functional group of the peptide backbone. Engineered polypeptides can be synthetically or recombinantly made. Typically, fusions are made using recombinant biotechnology, however, can also be made by chemical synthesis and conjugation methods.

“Analog” as used herein in the context of polypeptides refers to a compound that has insertions, deletions and/or substitutions of amino acids relative to a parent compound. “Analog sequence” as used herein in the context of polypeptides refers to an amino acid sequence that has insertions, deletions and/or substitutions of amino acids relative to a parent amino acid sequence (e.g., wild-type sequence, native sequence). An analog may have superior stability, solubility, efficacy, half-life, and the like. In some embodiments, an analog is a compound having at least 50%, for example 50%, 55%, 60%, 65%, 70%, 75%, 80%, 85%, 90%, 95%, 98%, or even higher, sequence identity to the parent compound. In a preferred embodiment the analog has from 1 to 5 amino acid modifications selected independently from any one or combination of an insertion, deletion, addition and substitution. In any of the embodiments herein, the exendin analog can have from 1 to 5 amino acid modifications selected independently from any one or combination of an insertion, deletion, addition and substitution, and preferably retains at least 50%, for example 50%, 55%, 60%, 65%, 70%, 75%, 80%, 85%, 90%, 95%, 98%, or even higher, sequence identity to the parent compound, and even more preferably at least 80%, 85%, 90%, 95%, 98%, or even higher, sequence identity to the parent compound, and preferably the parent compound is exendin-4, exendin-4(1-38), exendin-4(1-37), exendin-4(1-36), exendin-4(1-35), exendin-4(1-34), exendin-4(1-33), exendin-4(1-32), exendin-4(1-31), exendin-4(1-30), exendin-4(1-29) or exendin-4(1-28), or their Leu-14 substitution counterparts, e.g. Leu14 exendin-4(1-38), Leu14 exendin-4(1-38), Leu14 exendin-4(1-37), Leu14 exendin-4(1-36), Leu14 exendin 4(135), Leu14 exendin-4(1-34), Leu14 exendin 4(1-33), Leu14 exendin-4(1-32), Leu14 exendin-4(1-31), Leu14 exendin-4(1-30), Leu14 exendin-4(1-29) or Leu14 exendin-4(1-28), and most preferably the parent compound has the sequence of exendin-4 or Leu14 exendin-4. In a preferred embodiment the exendin analog fragment is not an exendin-4(1-28) or its amino acid substitution analog such as Leu14 exendin-4(1-28). Preferably the exendin analog fragment is at least 29 amino acids in length.and most preferably the parent compound has the sequence of exendin-4. and most preferably the parent compound has the sequence of exendin-4. In one embodiment at least amino acids corresponding to positions 1, 4, 6, 7 and 9 of exendin-4 are those as in native exendin-4, and further the one to five modifications are conservative amino acid substitutions at positions other than positions 1, 4, 6, 7 and 9 of exendin-4. For example, in yet a further embodiment of the embodiments herein, an exendin analog retains the amino acid at least as found in position 3, 4, 6, 5, 7, 8, 9, 10, 11, 13, 15, 18, 19, 22, 23, 25, 26, and/or 30 of exendin-4, and further preferably has no more than 1 to 5 of the remaining positions substituted with another amino acid, most preferably a chemically conservative amino acid. In all of the analogs herein, any substitution or modification at positions 1 and/or 2 will retain resistance to DPP-IV cleavage while retaining or improving insulinotropic activity as is known in the art for exendin-4 analogs, such as desamino-histidyl-exendin-4. As customary in the art, the term “conservative” in the context of amino acid substitutions refers to substitution which maintains properties of charge type (e.g., anionic, cationic, neutral, polar and the like), hydrophobicity or hydrophilicity, bulk (e.g., van der Waals contacts and the like), and/or functionality (e.g., hydroxy, amine, sulfhydryl and the like). The term “non-conservative” refers to an amino acid substitution which is not conservative.

“Identity,” “sequence identity” and the like in the context of comparing two or more nucleic acids or polypeptide sequences, refer to two or more sequences or subsequences that are the same or have a specified percentage of amino acid residues or nucleotides that are the same (i.e., about 50% identity, preferably 50%, 55%, 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a sequence comparison algorithms as known in the art, for example BLAST or BLAST 2.0. This definition includes sequences that have deletions and/or additions, as well as those that have substitutions, as well as naturally occurring, e.g., polymorphic or allelic variants, and man-made variants. In preferred algorithms, account is made for gaps and the like, as known in the art. For sequence comparison, typically one sequence acts as a reference sequence, to which test sequences are compared. When using a sequence comparison algorithm, test and reference sequences are entered into a computer, subsequence coordinates are designated if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection. See e.g., Current Protocols in Molecular Biology (Ausubel et al., eds. 1995 supplement)). Preferred examples of algorithms that are suitable for determining percent sequence identity and sequence similarity include the BLAST and BLAST 2.0 algorithms, which are described in Altschul et al., 1977, Nuci. Acids Res. 25:3389-3402 and Altschul et al., 1990, J. Mol. Biol. 215:403-410. BLAST and BLAST 2.0 are used, as known in the art, to determine percent sequence identity for the nucleic acids and proteins of the invention. Software for performing BLAST analyses is publicly available through the web site of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., Id.). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, 1989, Proc. Natl. Acad. Sci. USA 89:10915) alignments (B) of 50, expectation (E) of 10, M=5, N=−4, and a comparison of both strands.

The term “about” in the context of a numeric value refers to +/−10% of the numeric value.

The terms “peptide” and “polypeptide” in the context of components of the engineered polypeptides described herein are synonymous.

### II. Compounds of the Formulations

In a first aspect, engineered polypeptide compounds, Exendin ABD, are provided with sequence which includes an Albumin Binding Domain (ABD) polypeptide sequence and at least one polypeptide hormone domain (HD1) sequence. The terms “Albumin Binding Domain,” “ABD” and the like refer to polypeptides capable of binding albumin as described herein. The terms “hormone domain,” “hormone domain polypeptide” and the like refer to a GLP-1 receptor agonist polypeptide capable of eliciting a biological response in a subject. Exemplary hormone domains include, but are not limited to, an exendin, an exendin fragment, or an exendin analog. The term ABD as used herein is meant to include an ABD of the ABD1 type as defined herein and a de-immunized ABD referred to as an ABD of the ABD2 type as defined herein, or may it may refer to one of the two types as is evident from the context in which the term ABD is used. Exendin ABD as used herein is used interchangeably with “engineered polypeptide” or “improved engineered polypeptide” and is meant to include compounds having HD1 as defined herein that comprise the ABD of the ABD1 or ABD2 type, and may refer to one of the two types as evident from the context in which the term Exendin ABD is used.

It was surprisingly found that an exendin, exendin analog or active fragment can be fused to an very-high-affinity Albumin Binding Domain (ABD), of either the ABD1 or ABD2 type, derived from and having substantial amino acid sequence identity to the albumin-binding domains of bacterial protein G of Streptococcus strain G148, while retaining sufficient exendin-4 biological activity and having an extended duration of action, for example of at least 3 days and even 5 days in a rodent, which translates to at least a one week duration or longer in a human subject. “Duration of action” refers in the customary sense to allowing for more infrequent dosing in a therapeutical regimen. Thus, a prolonged duration of action will allowed for less frequent and/or more convenient dosing schedules. This was surprising in part because such ABD peptides have not been extensively demonstrated to be a robust platform as a therapeutic protein carrier, they are relatively hydrophobic which could interact adversely with an attached therapeutic peptide, and were not able to act as a carrier for at least one family of peptide hormones. Specifically, rat amylin when conjugated or fused to the ABDs described herein did not display any significant or long-acting in vivo activity in the same rodent models in which various exendin-ABD constructs were found to be active and with long duration of action. Without wishing to be bound by theory, it is believed the rat amylin sequence interacted with and disrupted the secondary structure and folding of the ABD.

Furthermore, for the fusions with the less immunoreactive de-immunized ABDs, the ABD2 type, the therapeutic conjugate or fusion compounds herein surprisingly have retained albumin binding affinity and specificity while having lower immunogenicity and exendin-4 therapeutic activity. The compounds are surprisingly active despite the absence of a plasma-protease cleavage site between the exendin and the ABD. Further surprising, the therapeutic compounds are believed active even when bound to albumin. The ABD2 compounds described herein provide albumin binding affinity and specificity while having lower immunogenicity than previously described ABD1 compounds, which were based on the albumin binding region of Streptococcal protein G strain 148 (G148) and in Jonsson et al. (Protein Eng. Design & Selection, 2008, 21:515-527). Recently, a few T- and B-cell epitopes were experimentally identified within the albumin binding region of Streptococcal protein G strain 148 (G148) (Goetsch et al, Clin. Diagn. Lab. Immunol. 10:125-32, 2003). The authors were interested in utilizing the T-cell epitopes of G148 in vaccines, i.e. to utilize the inherent immune-stimulatory property of the albumin binding region. Goetsch et al. additionally found a B-cell epitope, i.e. a region bound by antibodies after immunization, in the sequence of G148. In pharmaceutical compositions for human administration no immune-response is desired. Therefore, the albumin binding domain G148 is as such not preferred for use in such compositions due to its abovementioned immune-stimulatory properties. Without wishing to be bound by any theory, it is believed that fusion of an ABD2 Albumin Binding Domain with a hormone domain HD1 as described herein, can provide decreased immunogenicity as judged by a reduction in immune response relative to the hormone domain without ABD2 fusion.

As used herein “ABD sequence” is a sequence of an ABD compound that is monovalent or divalent, as appropriate, that forms part of an engineered polypeptide disclosed herein. “Peptide hormone domain (HD1) sequence” is a sequence of a peptide hormone domain (HD1) compound that is monovalent or divalent, as appropriate, that forms part of an engineered polypeptide disclosed herein. “Exendin sequence” is a sequence of an exendin compound that is monovalent or divalent, as appropriate, that forms part of an engineered polypeptide disclosed herein. “Exendin analog sequence” is a sequence of an exendin analog compound that is monovalent or divalent, as appropriate, that forms part of an engineered polypeptide disclosed herein. “Exendin active fragment sequence” is a sequence of an exendin active fragment compound that is monovalent or divalent, as appropriate, that forms part of an engineered polypeptide disclosed herein. “Exendin analog active fragment sequence” is a sequence of an exendin analog active fragment compound that is monovalent or divalent, as appropriate, that forms part of an engineered polypeptide disclosed herein. “Albumin binding motif (ABM) sequence” is a sequence of an ABM that is monovalent or divalent, as appropriate, that forms part of an engineered polypeptide disclosed herein. Unless stated otherwise, it is understood that where an engineered polypeptide “comprises” a compound (e.g., an ABD or HD1), the sequence of the engineered polypeptide includes the sequence of the compound (e.g. an ABD sequence or an HD1 sequence).

Biologically Active Components.

Biologically active compound components contemplated for use in the compounds and methods described herein include the exendins. The terms “biologically active compound” and the like refer in the customary sense to compounds, e.g., polypeptides and the like, which can elicit a biological response.

Exendins.

The exendins are peptides that are found in the salivary secretions of the Gila monster and the Mexican Bearded Lizard, reptiles that are endogenous to Arizona and Northern Mexico. Exendin-3 is present in the salivary secretions of Heloderma horridum (Mexican Beaded Lizard), and exendin-4 is present in the salivary secretions of Heloderma suspectum (Gila monster). See Eng et al, 1990, J. Biol. Chem., 265:20259-62; Eng et al, 1992, J. Biol. Chem., 267:7402-7405. The sequences of exendin-3 and exendin-4, respectively, follow:

(SEQ ID NO: 1)

HSDGTFTSDLSKQMEEEAVRLFIEWLKNGGPSSGAPPPS-NH₂;

(SEQ ID NO: 2)

HGEGTFTSDLSKQMEEEAVRLFIEWLKNGGPSSGAPPPS-NH₂.

Hargrove et al. (Regulatory Peptides, 2007, 141:113-119) reported an exendin-4 peptide analog that is a full-length C-terminally amidated exendin-4 peptide analog with a single nucleotide difference at position 14 compared to native exendin-4. The sequence of [¹⁴Leu]Exendin-4 is as follows: HGEGTFTSDLSKQLEEEAVRLFIEWLKNGGPS SGAPPPS-NH₂ (SEQ ID NO:3). The Leu14 exendin-4 is a preferred analog for use in the engineered polypeptides and their uses described herein. Another exendin-4 peptide analog is a chimera of the first 32 amino acids of exendin-4 having amino acid substitutions at positions 14 and 28 followed by a 5 amino acid sequence from the C-terminus of a non-mammalian (frog) GLP 1: [Leu¹⁴,Gln²⁸]Exendin-4(1-32)-fGLP-1(33-37). This compound has the following sequence: HGEGTFTSDLSKQLEEEAVRLFIEWLKQGGPSKEIIS (SEQ ID NO:4). Also known in the art are C-terminally truncated, biologically active forms of exendin-4, such as exendin-4(1-28), exendin-4(1-29), exendin-4(1-30), exendin-4(1-31), exendin-4(1-32) and their amidated forms. All of these exendin analogs are suitable as components of the engineered polypeptides of the present invention. As is customary in the art, square brackets (i.e., “[ ]”) in a peptidic compound name indicates substitution of the residue or chemical feature within the square brackets. For example, [¹⁴Leu]Exendin-4, [¹⁴Leu]Ex-4, and the like refer to exendin-4 having leucine at position 14. The numeric position of an amino acid can be indicated by prepended or postpended numbers in a variety of ways routinely employed in the art. For example, the terms ¹⁴Leu, Leu14, 14Leu, Leu¹⁴ and the like, are synonymous in referring to leucine at position 14.

It is understood that in some embodiments a C-terminal amide, or other C-terminal capping moiety can be present in compounds described herein.

Although the exendins have some sequence similarity to several members of the glucagon-like peptide family, with the highest homology, 53%, being to GLP-1(7-36)NH₂ (Goke et al, 1993, J. Biol. Chem., 268:19650-55) [sequence of GLP-1(7-37)NH₂: HAEGTFTSDVSSYLEGQAAKEFIAWLVKGRG (SEQ ID NO:5], also sometimes referred to as “GLP-1”) which has an insulinotropic effect stimulating insulin secretion from pancreatic beta-cells, exendins are not GLP-1 homologs.

Pharmacological studies have led to reports that exendin-4 can act at GLP-1 receptors in vitro on certain insulin-secreting cells, however, it has also been reported that exendin-4 may act at receptors not acted upon by GLP-1. Further, exendin-4 shares some but not all biological properties in vivo with GLP-1, and it has a significantly longer duration of action than GLP-1. Based on their insulinotropic activities, the use of exendin-3 and exendin-4 for the treatment of diabetes mellitus and the prevention of hyperglycemia has been proposed (Eng, U.S. Pat. No. 5,424,286, incorporated herein by reference in its entirety and for all purposes), and in fact, exendin-4 has been approved in the United States and in Europe for use as a therapeutic for treating type 2 diabetes.

Indeed, it is believed that exendins are not the species homolog of mammalian GLP-1 as was reported by Chen and Drucker who cloned the exendin gene from the Gila monster (J. Biol. Chem. 272:4108-15 (1997)). The observation that the Gila monster also has separate genes for proglucagons (from which GLP-1 is processed), that are more similar to mammalian proglucagon than exendin, indicated that exendins are not merely species homologs of GLP-1.

Methods for regulating gastrointestinal motility using exendin agonists are described in U.S. Pat. No. 6,858,576 (i.e., based on U.S. application Ser. No. 08/908,867 filed Aug. 8, 1997, which is a continuation-in-part of U.S. application Ser. No. 08/694,954 filed Aug. 8, 1996). Methods for reducing food intake using exendin agonists are described in U.S. Pat. No. 6,956,026 (i.e., based on U.S. application Ser. No. 09/003,869, filed Jan. 7, 1998, which claims the benefit of U.S. Application Nos. 60/034,905 filed Jan. 7, 1997, 60/055,404 filed Aug. 7, 1997, 60/065,442 filed Nov. 14, 1997, and 60/066,029 filed Nov. 14, 1997.

Novel exendin agonist compound sequences useful in the engineered polypeptides described herein are described in WO 99/07404 (i.e., PCT/US98/16387 filed Aug. 6, 1998), in WO 99/25727 (i.e., PCT/US98/24210, filed Nov. 13, 1998), in WO 99/25728 (i.e., PCT/US98/24273, filed Nov. 13, 1998), in WO 99/40788, in WO 00/41546, and in WO 00/41548, which are incorporated herein by reference and for all purposes along with their granted U.S. patent counterparts. Methods to assay for exendin activities in vitro and in vivo, as known in the art, including insulinotropic, food intake inhibition activity and weight loss activity, are described herein and also in the above references and other references recited herein.

Certain exemplary exendins, exendin agonists, and exendin analog agonists include: exendin fragments exendin-4 (1-30) (His Gly Glu Gly Thr Phe Thr Ser Asp Leu Ser Lys Gln Met Glu Glu Glu Ala Val Arg Leu Phe Ile Glu Trp Leu Lys Asn Gly Gly); exendin-4(1-28), exendin-4(1-29), exendin-4(1-30), exendin-4(1-31) and exendin-4(1-32). Analogs include substitution at the ¹⁴Met position (i.e., ¹⁴Met) with a non-oxidizing amino acid such as leucine. Examples include [¹⁴Leu]exendin-4, [¹⁴Leu]exendin-4(1-30), [¹⁴Leu]exendin-4(1-28) and [¹⁴Leu,²⁵Phe]exendin-4.

Exendin analog agonists for use in the engineered polypeptides described herein include those described in U.S. Pat. No. 7,223,725 (incorporated herein by reference and for all purposes), such as compounds of the formula: Xaa₁ Xaa₂ Xaa₃ Gly Xaa₅ Xaa₆ Xaa₇ Xaa₈ Xaa₉ Xaa₁₀ Xaa₁₁ Xaa₁₂ Xaa₁₃ Xaa₁₄ Xaa₁₅ Xaa₁₆ Xaa₁₇ Ala Xaa₁₉ Xaa₂₀ Xaa₂₁ Xaa₂₂Xaa₂₃ Xaa₂₄ Xaa₂₅ Xaa₂₆ Xaa₂₇ Xaa₂₈-Z₁; wherein Xaa₁ is His, Arg or Tyr; Xaa₂ is Ser, Gly, Ala or Thr; Xaa₃ is Ala, Asp or Glu; Xaa₅ is Ala or Thr; Xaa₆ is Ala, Phe, Tyr; Xaa₇ is Thr or Ser; Xaa₈ is Ala, Ser or Thr; Xaa₉ is Asp or Glu; Xaa₁₀ is Ala, Leu, Ile, Val, or Met; Xaa₁₁ is Ala or Ser; Xaa₁₂ is Ala or Lys; Xaa₁₃ is Ala or Gln; Xaa₁₄ is Ala, Leu, Ile, Val or Met; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Ala, Phe, Tyr; Xaa₂₃ is Ile, Val, Leu, or Met; Xaa₂₄ is Ala, Glu or Asp; Xaa₂₅ is Ala, Trp, Phe, Tyr; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂ or Gly Gly Xaa₃₁ Ser Ser Gly Ala Xaa₃₆ Xaa₃₇ Xaa₃₈-Z₂; Xaa₃₁, Xaa₃₆, Xaa₃₇ and Xaa₃₈ are independently Pro or are absent; and Z₂ is —OH or —NH₂. In any and each of the exendin analogs described above, also specifically contemplated are those wherein a replacement for the histidine corresponding to Xaa1 is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. Further specifically contemplated herein are exendin analogs described herein wherein a replacement for the glycine at Xaa2 is made with any of D-Ala, Val, Leu, Lys, Aib, (1-amino cyclopropyl) carboxylic acid, (1-aminocyclobutyl)carboxylic acid, 1-aminocyclopentyl)carboxylic acid, (1-aminocyclohexyl)carboxylic acid, (1-aminocycloheptyl)carboxylic acid, or (1-amino cyclooctyl)carboxylic acid.

According to one embodiment, exemplary compounds include those of the above formula wherein: Xaa₁ is His or Arg; Xaa₂ is Gly or Ala; Xaa₃ is Asp or Glu; Xaa₅ is Ala or Thr; Xaa₆ is Ala or Phe; Xaa₇ is Thr or Ser; Xaa₈ is Ala, Ser or Thr; Xaa₉ is Asp or Glu; Xaa₁₀ is Ala, or Leu; Xaa₁₁ is Ala or Ser; Xaa₁₂ is Ala or Lys; Xaa₁₃ is Ala or Gln; Xaa₁₄ is Ala or Leu; Xaa₁₅ is Ala or Glu; Xaa₁₆ is Ala or Glu; Xaa₁₇ is Ala or Glu; Xaa₁₉ is Ala or Val; Xaa₂₀ is Ala or Arg; Xaa₂₁ is Ala or Leu; Xaa₂₂ is Phe; Xaa₂₃ is Ile, Val; Xaa₂₄ is Ala, Glu or Asp; Xaa₂₅ is Ala, Trp or Phe; Xaa₂₆ is Ala or Leu; Xaa₂₇ is Ala or Lys; Xaa₂₈ is Ala or Asn; Z₁ is —OH, —NH₂, Gly-Z₂, Gly Gly-Z₂, Gly Gly Xaa₃₁-Z₂, Gly Gly Xaa₃₁ Ser-Z₂, Gly Gly Xaa₃₁ Ser Ser-Z₂, Gly Gly Xaa₃₁ Ser Ser Gly-Z₂, Gly Gly Xaa₃₁ Ser Ser Gly Ala-Z₂, Gly Gly Xaa₃₁ Ser Ser Gly Ala Xaa₃₆-Z₂, Gly Gly Xaa₃₁ Ser Ser Gly Ala Xaa₃₆ Xaa₃₇-Z₂, Gly Gly Xaa₃₁ Ser Ser Gly Ala Xaa₃₆ Xaa₃₇ Xaa₃₈-Z₂; Xaa₃₁, Xaa₃₆, Xaa₃₇ and Xaa₃₈ being independently Pro or is absent and Z₂ being —OH or —NH₂; provided that no more than three of Xaa₃, Xaa₅, Xaa₆, Xaa₈, Xaa₁₀, Xaa₁₁, Xaa₁₂, Xaa₁₃, Xaa₁₄, Xaa₁₅, Xaa₁₆, Xaa₁₇, Xaa₁₉, Xaa₂₀, Xaa₂₁, Xaa₂₄, Xaa₂₅, Xaa₂₆, Xaa₂₇ and Xaa₂₈ are Ala. In any and each of the exendin analogs described above, also specifically contemplated are those wherein a replacement for the histidine corresponding to position Xaa1 is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. Further specifically contemplated herein are exendin analogs described herein wherein a replacement for the glycine at Xaa 2 is made with any of D-Ala, Val, Leu, Lys, Aib, (1-aminocyclopropyl) carboxylic acid, (1-amino cyclobutyl)carboxylic acid, 1-aminocyclopentyl)carboxylic acid, (1-aminocyclohexyl)carboxylic acid, (1-amino cycloheptyl)carboxylic acid, or (1-aminocyclooctyl)carboxylic acid.

Other exemplary compounds include those set forth in WO 99/25727 identified therein as compounds 2-23. According to another embodiment, provided are compounds where Xaa₁₄ is Leu, Ile, or Val more preferably Leu, and/or Xaa₂₅ is Trp, Phe or Tyr, more preferably Trp or Phe. These compounds will be less susceptive to oxidative degradation, both in vitro and in vivo, as well as during synthesis of the compound.

Additional examples of exendin analogs suitable for use in the present fusion polypeptides include those described in U.S. Pat. No. 6,528,486 published Mar. 4, 2003 (incorporated herein by reference and for all purposes). Specifically, exendin analogs include those consisting of an exendin or exendin analog having at least 90% homology to exendin-4 having optionally between one and five deletions at positions 34-39, and a C-terminal extension of a peptide sequence of 4-20 amino acid units covalently bound to said exendin wherein each amino acid unit in said peptide extension sequence is selected from the group consisting of Ala, Leu, Ser, Thr, Tyr, Asn, Gln, Asp, Glu, Lys, Arg, His, and Met. More preferably the extension is a peptide sequence of 4-20 amino acid residues, e.g., in the range of 4-15, more preferably in the range of 4-10 in particular in the range of 4-7 amino acid residues, e.g., of 4, 5, 6, 7, 8 or 10 amino acid residues, where 6 amino acid residues are preferred. Most preferably, according to U.S. Pat. No. 6,528,486 the extension peptide contains at least one Lys residue, and is even more preferably from 3 to 7 lysines and even most preferably 6 lysines.

For example, one analog is HGEGTFTSDLSKQMEEEAVRLFIEWLKNGG PSSGAPPSKKKKKK (SEQ ID NO:118) (also designated ([des-³⁶Pro]exendin-4(1-39)-Lys₆). Additional exemplary analogs include Lys₆-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Ser-(Lys)₆ (H-Lys₆-des Pro ³⁶exendin-4(1-39)-Lys₆) (SEQ ID NO:184); His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Trp-Leu-Lys-Asn-Gly-Gly-Pro-Ser-Ser-Gly-Ala-Ser (H-[des ³⁶Pro, ^{37,38}Pro]exendin-4(1-39)-NH₂) (SEQ ID NO:185); Lys-Lys-Lys-Lys-Lys-Lys-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser (H-(Lys)₆-[des ³⁶Pro, ^{37,38}Pro]exendin-4(1-39) (SEQ ID NO:186); Asn-Glu-Glu-Glu-Glu-Glu-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser (H-Asn-(Glu)₅-[des ³⁶Pro, ^{37,38}Pro]exendin-4(1-39) (SEQ ID NO:187); His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser-(Lys)₆ ([des ³⁶Pro, ^{37,38}Pro]exendin-4(1-39)-(Lys)₆) (SEQ ID NO:188); (Lys)₆-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser-(Lys)₆ (H-(Lys)₆-[des ³⁶Pro, ^{37,38}Pro]exendin-4(1-39)-(Lys)₆) (SEQ ID NO:189); and Asp-Glu-Glu-Glu-Glu-Glu-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Ser-(Lys)₆ (Asn-(Glu)₅-[des ³⁶Pro, ^{37,38}Pro]exendin-4(1-39)-(Lys)₆) (SEQ ID NO:190). As customary in the art, repetition of an amino acid can be indicated by a subscripted number setting forth the number of repetitions; i.e., Lys₆, (Lys)₆ and the like refer to hexalysyl (SEQ ID NO:191). In any and each of the exendin analogs described above, specifically contemplated are those wherein a replacement for the histidine corresponding to position 1 is made with any of D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (or imidazopropionyl), beta-hydroxy-imidazopropionyl, N-dimethyl-histidyl or beta-carboxy-imidazopropionyl. Further specifically contemplated herein are exendin analogs described herein wherein a replacement for the glycine at position 2 is made with any of D-Ala, Val, Leu, Lys, Aib, (1-aminocyclopropyl)carboxylic acid, (1-aminocyclobutyl)carboxylic acid, 1-aminocyclopentyl)carboxylic acid, (1-amino cyclohexyl)carboxylic acid, (1-aminocycloheptyl) carboxylic acid, or (1-aminocyclooctyl) carboxylic acid.

Further examples of exendin analogs suitable for use in the engineered polypeptide constructs are those described in published PCT application WO2004035623 (incorporated herein by reference and for all purposes), particularly those comprised of naturally-occurring amino acids, which describes exendin analogs having at least one modified amino acid residue particularly at positions ¹³Gln, ¹⁴Met, ²⁵Trp or ²⁸Asn with reference to the corresponding positions of exendin-4(1-39). According to that publication are additional such analogs further comprising a 1-7 amino acid C-terminal extension that comprises at least one Lys amino acid and more preferably at least five Lys amino acid units such as six or seven Lys amino acid units.

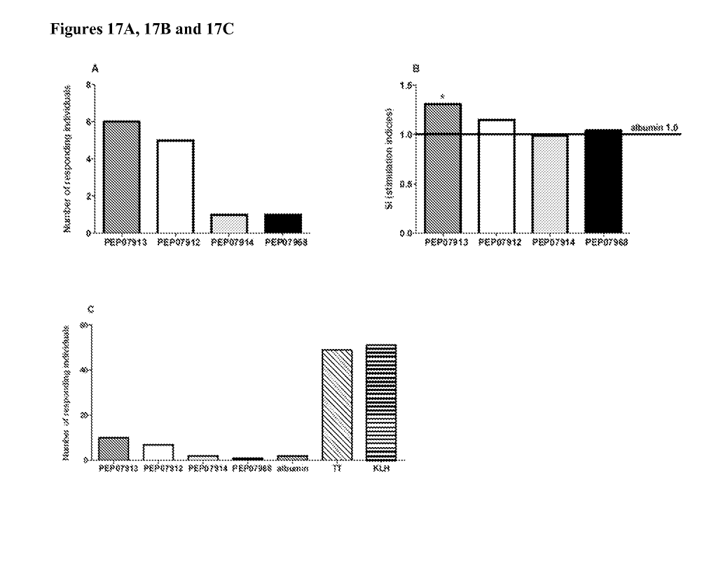

Yet further examples of exendin analogs suitable for use in the engineered polypeptide constructs are those described in published PCT application WO/2010/120476, entitled “N-Terminus Conformationally Constrained GLP-1 Receptor Agonist Compounds” (incorporated herein by reference and for all purposes), which describes exendin analogs having modified amino acid residues in the N-terminal portion of an exendin or exendin analog to create a high beta-turn characteristic in that region. For example, analogs are designed to mimic amino acid residues His1 Gly2 Glu3 by creating a conformationally constrained region, include exendin analogs containing a thiazolidine-proline peptide mimetic at His1 Gly2 Glu3 (see for example compounds described in FIGS. 17A-F therein), which can be used as a modification in exendin-4, lixisenatide, or other analogs described herein.

In any and each of the exendins, e.g. exendin-4, the exendin analogs and formulas described herein, specifically contemplated are those wherein a replacement for the histidine corresponding to position 1 is made with any of L-histidine, D-histidine, desamino-histidine, 2-amino-histidine, beta-hydroxy-histidine, homohistidine. N-alpha-acetyl-histidine, alpha-fluoromethyl-histidine, alpha-methyl-histidine, 3-pyridylalanine, 2-pyridylalanine, 4-pyridylalanine, 4-imidazoacetyl, des-amino-histidyl (imidazopropionyl), beta-hydroxy-imidazopropionyl, N-dimethyl-histidyl or beta-carboxy-imidazopropionyl. For example, preferred exendin analogs for use in engineered polypeptide conjugates as described herein wherein the His1 position is modified are (4-imidazoacetyl) exendin-4, (des-amino-histidyl) exendin-4 (or (imidazopropionyl) exendin-4), (beta-hydroxy-imidazopropionyl) exendin-4, (N-dimethyl-histidyl) exendin-4 and (beta-carboxy-imidazopropionyl) exendin-4. Further specifically contemplated herein are exendins or exendin analogs described herein wherein a replacement for the glycine at position 2 is made with any of D-Ala, Val, Leu, Lys, Aib, (1-aminocyclopropyl)carboxylic acid, (1-aminocyclobutyl)carboxylic acid, 1-aminocyclopentyl)carboxylic acid, (1-amino cyclohexyl)carboxylic acid, (1-aminocycloheptyl)carboxylic acid, or (1-aminocyclooctyl) carboxylic acid. Thus for example, such an engineered polypeptide would include (4-imidazoacetyl)exendin-4-Gly-PEP07986, where the exendin-4 analog is linked via a peptide bound at its C-terminal alpha carboxy group to a glycine as linker via a peptide bond to the N-terminus of the PEP07986 sequence.

Any of the above exendin analogs or their active fragments are suitable for use in the present engineered polypeptides, with or without a linker to the ABD.

Albumin Binding Domain (ABD) Peptides.

The engineered polypeptide (Exendin ABD) includes an Albumin Binding Domain polypeptide (ABD), either an ABD1 type ABD or an ABD2 type ABD as described herein. The ABD2 sequences are improved upon the ABD1 sequence by having amino acid substitutions that reduce immunogenicity of the ABD but retain its other properties such as high affinity albumin binding and long duration of circulation in blood.

Because of the presence of an albumin binding motif, the ABD peptide described herein for use in the Exendin ABD binds to albumin with a K_{D} value of the interaction that is at most 1×10⁻⁶ M and even more preferably at most 1×10⁻⁹ M (even tighter affinity). More preferably the K_{D} value of the interaction that is at most 1×10⁻¹⁰ M, even more preferably is at most 1×10⁻¹¹ M, yet even more preferably is at most 1×10⁻¹² M, and even further is at most 1×10⁻¹³ M. The values are most preferably for affinity to human serum albumin (“HSA”).

In one embodiment, the ABD according to this aspect binds to albumin such that the k_{off} value of the interaction is at most 5×10⁻⁵ s⁻¹, such as at most 5×10⁻⁶ s⁻¹.

The terms “albumin binding” and “binding affinity for albumin” as used herein refer to a property of a polypeptide which may be tested for example by the use of surface plasmon resonance technology, such as in a Biacore instrument as known in the art. For example, as described in the examples below, albumin binding affinity may be tested in an experiment in which albumin, or a fragment thereof, is immobilized on a sensor chip of the instrument, and the sample containing the polypeptide to be tested is passed over the chip. Alternatively, the polypeptide to be tested is immobilized on a sensor chip of the instrument, and a sample containing albumin, or a fragment thereof, is passed over the chip. Albumin may, in this regard, be a serum albumin from a mammal, such as human serum albumin. The skilled person may then interpret the results obtained by such experiments to establish at least a qualitative measure of the binding affinity of the polypeptide for albumin. If a quantitative measure is desired, for example to determine a K_{D} value for the interaction, surface plasmon resonance methods may also be used. Binding values may for example be defined in a Biacore2000 instrument (GE Healthcare). Albumin is suitably immobilized on a sensor chip of the measurement, and samples of the polypeptide whose affinity is to be determined are prepared by serial dilution and injected. K_{D} values may then be calculated from the results using for example the 1:1 Langmuir binding model of the BIAevaluation 4.1 software provided by the instrument manufacturer (GE Healthcare).

Albumin Binding Domain (ABD) Peptides of the ABD1 Type.

Albumin Binding Domain (ABD) peptides of the ABD1 type for use in the invention are those with comparably high affinity for albumin and derive from and have substantial amino acid sequence identity to the albumin-binding domains of bacterial protein G of Streptococcus strain G148. As such, ABD1 peptides contemplated for the engineered polypeptides described herein include those having the albumin binding motifs as described by Jonsson et al. (Protein Eng. Design & Selection, 2008, 21:515-527) as well as the ABD peptides described therein, and those motifs and ABD peptides further described in PCT Published Appl. No. WO2009/016043, as well as analogs thereof, particularly those having at least 85% amino acid identity. In one embodiment the ABD1 peptide can include an albumin binding motif (“ABM”) that includes the amino acid sequence

(SEQ ID NO: 119)

GVSD X₅ YK X₈ X₉ I X₁₁ X₁₂ A X₁₄ TVEGV X₂₀ AL X₂₃
X₂₄ X₂₅ I

wherein, independently of each other,

X₅ is selected from Y and F;

X₈ is selected from N, R and S;

X₉ is selected from V, I, L, M, F and Y;

X₁₁ is selected from N, S, E and D;

X₁₂ is selected from R, K and N;

X₁₄ is selected from K and R;

X₂₀ is selected from D, N, Q, E, H, S, R and K;

X₂₃ is selected from K, I and T;

X₂₄ is selected from A, S, T, G, H, L and D; and

X₂₅ is selected from H, E and D.

Preferably the ABD1 peptide binds to albumin with a K_{D} value of the interaction that is at most 1×10⁻⁶ M, and even more preferably at most 1×10⁻⁹ M (even tighter affinity). The term “K_{D}” refers to a dissociation constant, as customary in the art. More preferably the K_{D} value of the interaction that is at most 1×10⁻¹⁰ M, even more preferably is at most 1×10⁻¹¹ M, yet even more preferably is at most 1×10⁻¹² M, and even further is at most 1×10⁻¹³ M. For example, a Kd value of 1×10⁻¹⁴ M is a K_{D} value of the interaction that is at most 1×10⁻¹³ M. The K_{D} values can be determined as described in PCT Published Appl. No. WO 2009/016043, preferably to human serum albumin. In one embodiment is contemplated the above genus with the proviso that the amino acid sequence is not GVSDYYKNLINNAKTVEGVKALIDEI (SEQ ID NO:120).

As demonstrated herein and in the cited references, the albumin binding capacity of the ABD1 peptide can be retained despite amino acid changes so long as such changes retain sufficient tertiary structure of the ABD peptide. Such changes include, for example, a substitution where an amino acid residue belonging to a certain functional grouping of amino acid residues (e.g. hydrophobic, hydrophilic, polar etc.) is exchanged for another amino acid residue from the same functional group. Accordingly, in one such embodiment of the ABD1 peptide, the motif X5 is Y. In one embodiment of the ABD X₈ is selected from N and R, and may in particular be R. In one embodiment X₉ is L. In one embodiment X₁₁ is selected from N and S, and may in particular be N. In one embodiment X₁₂ is selected from R and K, such as X₁₂ being R or X₁₂ being K. In one embodiment X₁₄ is K. In one embodiment X₂₀ is selected from D, N, Q, E, H, S and R, and may in particular be E. In one embodiment X₂₃ is selected from K and I, and may in particular be K. In one embodiment X₂₄ is selected from A, S, T, G, H and L. In a more specific embodiment X₂₄ is L. In an even more specific embodiment “X₂₃ X₂₄” is KL. In another even more specific embodiment “X₂₃ X₂₄” is TL. In one embodiment X₂₄ is selected from A, S, T, G and H. In a more specific embodiment X₂₄ is selected from A, S, T, G and H and X₂₃ is I. In one embodiment X₂₅ is H.

The sequences of individual albumin binding motifs within the above formula include those presented as SEQ ID NOs:1-257 in PCT Published Appl. No. WO 2009/016043, incorporated herein by reference and for all purposes. In certain embodiments of the albumin binding polypeptide the albumin binding motif consists of an amino acid sequence selected from SEQ ID NO:1-257. In a more specific embodiment of this aspect of the invention, the motif sequence is selected from SEQ ID NO:2, SEQ ID NO:3, SEQ ID NO:9, SEQ ID NO:15, SEQ ID NO:25, SEQ ID NO:27, SEQ ID NO:46, SEQ ID NO:49, SEQ ID NO:53, SEQ ID NO:54, SEQ ID NO:55, SEQ ID NO:155, SEQ ID NO:239, SEQ ID NO:240, SEQ ID NO:241, SEQ ID NO:242, SEQ ID NO:243, SEQ ID NO:244 and SEQ ID NO:245 of PCT Published Appl. No. WO 2009/016043. In yet more specific embodiments of this aspect of the invention, the motif sequence is selected from SEQ ID NO:3, SEQ ID NO:53 and SEQ ID NO:239 of PCT Published Appl. No. WO 2009/016043. ABD1 type ABD peptides, containing these albumin binding motifs and thus suitable for conjugation or fusion to a hormone domain HD1 as described herein are further described herein and below and exemplified in Table 1A and the Examples. Not to be bound by theory but it is believed that the albumin binding motif can form part of a three-helix bundle protein domain. For example, the motif may essentially constitute or form part of two alpha helices with an interconnecting loop, within the three-helix bundle protein domain. Accordingly, in particular embodiments of the invention, such a three-helix bundle protein domain is selected from the group of three-helix domains of bacterial receptor protein G from Streptococcus strain G148. In different variants of this embodiment, the three-helix bundle protein domain of which the motif forms a part is selected from the group of domain GA1, domain GA2 and domain GA3 of protein G from Streptococcus strain G148, in particular domain GA3.

In embodiments of the present invention wherein the motif “forms part of a three-helix bundle protein domain,” this is understood to mean that the sequence of the albumin binding motif is “inserted” into or “grafted” onto or “fused” to the sequence of the naturally occurring (or otherwise original) three-helix bundle domain, such that the motif replaces a similar structural motif in the original domain. For example and without wishing to be bound by theory, the motif is thought to constitute two of the three helices of a three-helix bundle, and can replace such a two-helix motif within any three-helix bundle. The replacement of two helices of the three-helix bundle domain by the two motif helices disclosed herein is performed so as not to affect the basic structure of the polypeptide. That is, the overall folding of the backbone of the polypeptide according to this embodiment of the invention will be substantially the same as that of the three-helix bundle protein domain of which it forms a part, e.g. having the same elements of secondary structure in the same order etc. Thus, a motif useful to the engineered polypeptides herein can “form part” of a three-helix bundle domain if the polypeptide according to this embodiment has the same fold as the original domain, implying that the basic structural properties are shared, those properties e.g. resulting in similar CD spectra.

Accordingly, in one embodiment the ABD1 is a three-helix bundle protein domain, which includes the albumin binding motif as defined above and additional sequences making up the remainder of the three-helix configuration. To such an albumin binding domain polypeptide can be fused an exendin or analogs or active fragments thereof to create the engineered polypeptides (Exendin ABD) as described herein. An ABD1 peptide suitable for conjugation or fusion to an exendin compound can includes the amino acid sequence: LAEAK Xₐ X_{b} A X_{c} X_{d} EL Xₑ KY (SEQ ID NO:182) covalently linked to an albumin binding motif (ABM) which is further covalently linked to the amino acid sequence LAALP (SEQ ID NO:183), wherein ABM is an albumin binding motif as defined herein, Xₐ is selected from V and E; X_{b} is selected from L, E and D; X_{c} is selected from N, L and I; X_{d} is selected from R and K; and Xₑ is selected from D and K. In some embodiments, an albumin binding domain polypeptide suitable for conjugation or fusion to an exendin compound is the amino acid sequence: LAEAK Xₐ X_{b} A X_{c} X_{d} EL Xₑ KY (SEQ ID NO:182) covalently linked to an albumin binding motif (ABM) which is further covalently linked to the amino acid sequence LAALP (SEQ ID NO:183), as described above.

In some embodiments, the ABD1 includes the amino acid sequence LAEAK Xₐ X_{b} A X_{c} X_{d} EL Xₑ KY GVSD X₅ YK X₈ X₉ I X₁₁ X₁₂ A X₁₄ TVEGV X₂₀ AL X₂₃ X₂₄ X₂₅ I LAALP (SEQ ID NO:121), wherein Xₐ is selected from V and E; X_{b} is selected from L, E and D; X_{c} is selected from N, L and I; X_{d} is selected from R and K; Xₑ is selected from D and K; X₅ is selected from Y and F; X₈ is selected from N, R and S;

X₉ is selected from V, I, L, M, F and Y; X₁₁ is selected from N, S, E and D; X₁₂ is selected from R, K and N; X₁₄ is selected from K and R; X₂₀ is selected from D, N, Q, E, H, S, R and K; X₂₃ is selected from K, I and T; X₂₄ is selected from A, S, T, G, H, L and D; and X₂₅ is selected from H, E and D.

Further for each of the embodiments herein of the ABD1 sequence, the C-terminal proline (corresponding to position 46 above) can be optionally absent. Even further for each embodiment of the ABD1 sequence, the leucine at position 45 can be optionally present or absent.

In one embodiment of this ABD1 Xₐ is V. In one embodiment of this polypeptide X_{b} is L. In one embodiment of this polypeptide X_{c} is N. In one embodiment of this ABD1 X_{d} is R. In one embodiment of this polypeptide Xₑ is D.

In certain embodiments, Xₐ is E. In certain embodiments X_{b} is D. In certain embodiments, X_{c} is I. In certain embodiments, X_{d} is K. In certain embodiments, Xₐ independently is E, and/or independently X_{b} is D, and/or independently X_{c} is I, and/or independently X_{d} is K. In certain embodiments, the ABD1 is LAEAKEDAIKELDKYGVSDYYKRLISKAKTVEGVKALISEILAALP (SEQ ID NO:122). In certain embodiments, the ABD1 sequence is LAEAKEDAIKELDKYGVSDYYKNLINKAKTVEGVEALTLHILAALP (SEQ ID NO:123). In certain embodiments, the ABD1 sequence is LAEAKEDAIKELDKYGVSDYYKNLINKAKTVEGVEALISEILAALP (SEQ ID NO:124).

Sequences of individual ABD1 polypeptides suitable for fusion with the active hormone domain peptides as described herein are presented in Jonsson et al. (Id.) and as SEQ ID NOs:258-514 in PCT Published Appl. No. WO 2009/016043, incorporated herein by reference.

Selected ABD1 sequences are disclosed in Table 1A below. Also encompassed by the present invention is an Albumin Binding polypeptide having an amino acid sequence with 85% or greater identity to a sequence selected from SEQ ID NOs: 258-514 in PCT Published Appl. No. WO 2009/016043. In particular embodiments, the sequence of the ABD1 is selected from SEQ ID NO:259, SEQ ID NO:260, SEQ ID NO:266, SEQ ID NO:272, SEQ ID NO:282, SEQ ID NO:284, SEQ ID NO:303, SEQ ID NO:306, SEQ ID NO:310, SEQ ID NO:311, SEQ ID NO:312, SEQ ID NO:412, SEQ ID NO:496, SEQ ID NO:497, SEQ ID NO:498, SEQ ID NO:499, SEQ ID NO:500, SEQ ID NO:501 and SEQ ID NO:502 in PCT Published Appl. No. WO 2009/016043, and sequences having 85% or greater identity thereto. In more specific embodiments of this aspect of the invention, the sequence of ABD1 is selected from SEQ ID NO:260, SEQ ID NO:310 and SEQ ID NO:496 in PCT Published Appl. No. WO 2009/016043 and sequences having 85% or greater identity thereto. In yet further embodiments, the sequence of the ABD1 is selected from SEQ ID NO:260, SEQ ID NO:270, SEQ ID NO:272, SEQ ID NO:291, SEQ ID NO:294, SEQ ID NO:298, SEQ ID NO:299, SEQ ID NO:300, SEQ ID NO:400, SEQ ID NO:484, SEQ ID NO:485, SEQ ID NO:486, SEQ ID NO:487, SEQ ID NO:488, SEQ ID NO:489 and SEQ ID NO:490 in PCT Published Appl. No. WO 2009/016043, and sequences having 85% or greater identity thereto.

Exemplary ABD1 species include, but are not limited to, the compounds with sequence set forth in Table 1A following and the Examples. See also PCT Published Appl. No. WO 2009/016043, incorporated herein by reference in its entirety and for all purposes. An ABD peptide sequence useful in compounds, methods and pharmaceuticals compositions described herein can be a fragment or analog of an ABD1 peptide sequence disclosed herein or known in the art so long as it contains an albumin binding motif sequence and binds albumin with the affinity described herein.

TABLE 1A
Selected ABD peptides of the ABD1 type

| | SEQ |
|---|---|
| | ID |
| ABD1 peptide sequence | NO: |
| LAEAKVLANRELDKYGVSDYYKNLINNAKTVEGVKALIDEILAALP | 23 |
| LAEAKVLANRELDKYGVSDFYKSYINRAKTVEGVHTLIGHILAALP | 24 |
| LAEAKVLANRELDKYGVSDFYKRLINKAKTVEGVNALTHHILAALP | 25 |
| LAEAKVLANRELDKYGVSDYYKNLINRARTVEGVHALIDHILAALP | 26 |
| LAEAKVLANRELDKYGVSDYYKNIINRAKTVEGVRALKLHILAALP | 27 |
| LAEAKVLANRELDKYGVSDFYKNLINRAKTVEGVSSLKGHILAALP | 28 |
| LAEAKVLANRELDKYGVSDYYKNLINKAKTVEGVEALTLHILAALP | 29 |
| LAEAKVLANRELDKYGVSDFYKNLINRAKTVEGVDALIAHILAALP | 30 |
| LAEAKVLANRELDKYGVSDFYKSLINRAKTVEGVDALTSHILAALP | 31 |
| LAEAKVLANRELDKYGVSDFYKNLINRAKTVEGVNSLTSHILAALP | 32 |
| LAEAKVLANRELDKYGVSDFYKNVINKAKTVEGVEALIADILAALP | 33 |
| LAEAKVLANRELDKYGVSDYYKNLINKAKTVEGVQALIAHILAALP | 34 |
| LAEAKVLANRELDKYGVSDFYKRLINKAKTVEGVEALKLHILAALP | 35 |
| LAEAKEDAIKELDKYGVSDYYKRLISKAKTVEGVKALISEILAALP | 122 |
| LAEAKEDAIKELDKYGVSDYYKNLINKAKTVEGVEALTLHILAALP | 123 |
| LAEAKEDAIKELDKYGVSDYYKNLINKAKTVEGVEALISEILAALP | 124 |

In another preferred embodiment of the ABD used in the engineered polypeptides described herein, the amino acid sequence of the albumin binding portion of the engineered polypeptide includes an ABD1 selected from any one of the sequences described herein, including those from Table 1A or the listing herein and further including their des-Pro46 forms.

In one embodiment, the ABD1 according to this aspect further includes one or more additional amino acid residues positioned at the N- and/or the C-terminal of the ABD1 sequence defined or exemplified herein. These additional amino acid residues may play a role in further enhancing the binding of albumin by the polypeptide, and improving the conformational stability of the folded albumin binding domain, but may equally well serve other purposes, related for example to one or more of production, purification, stabilization in vivo or in vitro, coupling, labeling or detection of the polypeptide, as well as any combination thereof. Such additional amino acid residues may include one or more amino acid residue(s) added for purposes of chemical coupling, e.g. to the HD1.

For example, the amino acids directly preceding or following the alpha helix at the N- or C-terminus of the ABD1 amino acid sequence may thus in one embodiment affect the conformational stability. One example of an amino acid residue which may contribute to improved conformational stability is a serine residue positioned at the N-terminal of the ABD1 amino acid sequence as defined above. The N-terminal serine residue may in some cases form a canonical S-X-X-E capping box, by involving hydrogen bonding between the gamma oxygen of the serine side chain and the polypeptide backbone NH of the glutamic acid residue. This N-terminal capping may contribute to stabilization of the first alpha helix of the three helix domain constituting the albumin binding polypeptide according to the first aspect of the disclosure.

Thus, in one embodiment, the additional amino acids include at least one serine residue at the N-terminal of the polypeptide. The ABD1 amino acid sequence is in other words preceded by one or more serine residue(s). In another embodiment of the albumin binding polypeptide, the additional amino acids include a glycine residue at the N-terminal of the ABD sequence. It is understood that the ABD1 amino acid sequence may be preceded by one, two, three, four or any suitable number of amino acid residues. Thus, the ABD amino acid sequence may be preceded by a single serine residue, a single glycine residue or a combination of the two, such as a glycine-serine (GS) combination or a glycine-serine-serine (GSS) combination. An example of one such ABD1 having a N-terminal serine is SLAEAKVLANRELDKYGVSDFYKRLINKAKTVEGVEALKLHILAALP (SEQ ID NO:176). The corresponding des-proline form would be SLAEAKVLANRELDKYGVSDFYKRLINKAKTVEGVEALKLHILAAL (SEQ ID NO:177).

In yet another embodiment, the additional amino acid residues include an alanine acid at the N-terminal of the ABD1 polypeptide defined herein, or in combination with serine as an alanine-serine sequence at the N-terminal of the ABD1 sequences above. In yet another embodiment, the additional amino acid residues include a glutamic acid at the N-terminal of the ABD polypeptide defined herein.

Similarly, C-terminal capping may be exploited to improve stability of the third alpha helix of the three helix domain constituting the ABD1. The C-terminal proline residue present at the C-terminal of the ABD1 amino acid sequence defined above may at least partly function as a capping residue. A lysine residue following the proline residue at the C-terminal may contribute to further stabilization of the third helix of the albumin binding polypeptide, by hydrogen bonding between the epsilon amino group of the lysine residue and the carbonyl groups of the amino acids located two and three residues before the lysine in the polypeptide backbone, e.g. the carbonyl groups of the leucine and alanine residues of the ABD1 amino acid sequence defined above. Thus, in one embodiment, the additional amino acids include a lysine residue at the C-terminal of the polypeptide.

As discussed above, the additional amino acids may be related to the production of the albumin binding polypeptide. In particular, one or more optional amino acid residues following the C-terminal proline may provide advantages when the albumin binding polypeptide according to the first aspect is produced by chemical peptide synthesis. Such additional amino acid residues may for example prevent formation of undesired substances, such as diketopiperazine at the dipeptide stage of the synthesis. One example of such an amino acid residue is glycine. Thus, in one embodiment, the additional amino acids of an ABD1 include a glycine residue at the C-terminal of the polypeptide, directly following the proline residue or following an additional lysine and/or glycine residue as accounted for above. Alternatively, polypeptide production may benefit from amidation of the C-terminal proline residue of the ABD amino acid sequence. In this case, the C-terminal proline includes an additional amine group at the carboxyl carbon.

The skilled person is aware of methods for accomplishing C-terminal modification, such as by different types of pre-made matrices for peptide synthesis.

In another embodiment, the additional amino acid residues includes a cysteine residue at the N- and/or C-terminal of the polypeptide. Such a cysteine residue may directly precede and/or follow the ABD amino acid sequence as defined herein or may precede and/or follow any other additional amino acid residues as described above. By the addition of a cysteine residue to the polypeptide chain, a thiol group for site directed conjugation of the albumin binding polypeptide may be obtained. Alternatively, a selenocysteine residue may be introduced at the C-terminal of the ABD1 polypeptide chain, in a similar fashion as for the introduction of a cysteine residue, to facilitate site-specific conjugation (Cheng et al, Nat Prot 1:2, 2006).

In one embodiment, the ABD1 includes no more than two cysteine residues. In another embodiment, the ABD1 includes no more than one cysteine residue.

In another embodiment, the additional amino acid residues of the ABD1 includes a “tag” for purification or detection of the polypeptide, such as a hexahistidyl (His₆) tag, or a “myc” (“c-Myc”) tag or a “FLAG” tag for interaction with antibodies specific to the tag and/or to be used in purification. The skilled person is aware of other alternatives.

For example, in preferred engineered polypeptide embodiments the ABD1 includes LAEAKVLANRELDKYGVSDFYKRLINKAKTVEGVEALKLHILAALP (SEQ ID NO:35), and its N-terminally extended ABD1 sequence forms including SLAEAKVLANRELDKYGVSDFYKRLINKAKTVEGVEALKLHILAALP (SEQ ID NO:176) and GSLAEAKVLANRELDKYGVSDFYKRLINKAKTVEGVEALKLHILAALP (SEQ ID NO:178). The serine in position 2 is capping the sequence, raising Tm approximately 2° C. compared to having a glycine or an alanine in this position. An alanine can also immediately precede the serine as in ASLAEAKVLANRELDKYGVSDFYKR LINKAKTVEGVEALKLHILAALP (SEQ ID NO:179). Also preferred are the corresponding polypeptides where the C-terminal proline, glycine or both is absent in each of the above ABD1 sequences. Accordingly, also preferred are sequences where the ABD1 includes the des-proline forms, which can improve yields compared to the parent forms LAEAKVLANRELDKYGVSDFYKRLINKAKTVEGVEALKLHILAAL (SEQ ID NO:35), and its N-terminally extended ABD1 sequence forms including SLAEAKVLANRELD KYGVSDFYKRLINKAKTVEGVEALKLHILAAL (SEQ ID NO:177) and GSLAEAKVLANRELDKYGVSDFYKRLINKAKTVEGVEALKLHILAAL (SEQ ID NO:180) and ASLAEAKVLANRELDKYGVSDFYKRLINKAKTVEGVEALKLHILAAL (SEQ ID NO:181).

Albumin Binding Domain (ABD) Peptides of the ABD2 Type.

For ABD1 type ABD, for previously disclosed albumin binding domains derived from Streptococcal protein G strain 148 (G148) and for some variants having a high affinity to albumin, e.g. WO09/016043, the higher affinity was achieved at the cost of reduced thermal stability. In addition, it has been reported that T- and B-cell epitopes were experimentally identified within the albumin binding region of G148 (Goetsch et al, Clin Diagn Lab Immunol 10:125-32, 2003). The authors behind the study were interested in utilizing the T-cell epitopes of G148 in vaccines, i.e. to utilize the inherent immune-stimulatory property of the albumin binding region. Goetsch et al. additionally found a B-cell epitope, i.e. a region bound by antibodies after immunization, in the sequence of G148. Therefore, the albumin binding domain G148 and polypeptides derived from G148, and thus fusion/conjugates containing them, risk the abovementioned immune-stimulatory properties. In pharmaceutical compositions for human administration no (or reduced) immune-response is desired.

The above drawbacks and deficiencies of such fusions and/or conjugates are overcome or reduced by the use of the improved Albumin Binding Domain (ABD) peptides of the ABD2 type disclosed herein for use in the engineered polypeptides (Exendin ABD) described herein. Such ABD2 type of ABD are those with comparably high affinity for albumin and derive from albumin-binding domain of bacterial protein G of Streptococcus strain G148 and have substantial amino acid sequence identity thereto, yet are modified as described herein to further provide desirable immunological properties, e.g. reduced immunogenicity (also referred to as “deimmunized” ABD). Accordingly, the Albumin Binding Domain polypeptide comprising the long-duration engineered polypeptide conjugate or fusion described herein is a three-helix bundle protein domain, which comprises an albumin binding motif and additional sequences comprising the three-helix configuration. The ABD2 type ABD for use in the present compounds include those “deimmunized” ABD sequences described in PCT Published Appl. No. WO2012/004384, which is herein incorporated by reference for its ABD2 sequences and ABD2 assays. The ABD2 peptides described herein and contemplated for use in the engineered polypeptides described herein are superior to those having the albumin binding sequence as described by Jonsson et al. (Protein Eng. Design & Selection, 2008, 21:515-527) as well as the ABD peptides described therein, and those ABD peptides further described in PCT Published Appl. No. WO2009/016043 (also referred herein as ABD1 type ABD). To the ABD2 polypeptide described herein is fused an HD1 comprising an exendin or analog or active fragment thereof to create the engineered polypeptide (Exendin ABD) described herein. An Albumin Binding Domain polypeptide suitable for conjugation or fusion to an exendin compound can comprise the improved ABD2 amino acid sequence which comprises a sequence selected from:

formula (i)

(SEQ ID NO: 300)

LA X3 AK X6 X7 AN X10 ELD X14 YGVSDF YKRLI X26
KAKTVEGVEALK X39 X40 IL X43 X44 LP

wherein independently of each other

X3 is selected from E, S, Q and C;

X6 is selected from E, S and C;

X7 is selected from A and S;

X10 is selected from A, S and R;

X14 is selected from A, S, C and K;

X26 is selected from D and E;

X39 is selected from D and E;

X40 is selected from A and E;

X43 is selected from A and K;

X44 is selected from A, S and E;

the leucine at position 45 is present or absent; and

the proline at position 46 is present or absent; and

formula (ii) an amino acid sequence which has at least 95% identity to the sequence defined in (i),

with the proviso that X₇ is not L, E or D;

or alternatively,

with the proviso that the amino acid sequence is not defined by the following sequence, as defined in PCT Published Application No. WO 2009/016043: LAEAK Xₐ X_{b} A X_{c} X_{d} EL Xₑ KY GVSD X₅ YK X₈ X₉ I X₁₁ X₁₂ A X₁₄ TVEGV X₂₀ AL X₂₃ X₂₄ X₂₅ ILAALP (SEQ ID NO:593) wherein independently of each other,

Xₐ is selected from V and E;

X_{b} is selected from L, E and D;

X_{c} is selected from N, L and I;

X_{d} is selected from R and K;

Xₑ is selected from D and K; and

X₅ is selected from Y and F;

X₈ is selected from N, R and S;

X₉ is selected from V, I, L, M, F and Y;

X₁₁ is selected from N, S, E and D;

X₁₂ is selected from R, K and N;

X₁₄ is selected from K and R;

X₂₀ is selected from D, N, Q, E, H, S, R and K;

X₂₃ is selected from K, I and T;

X₂₄ is selected from A, S, T, G, H, L and D; and

X₂₅ is selected from H, E and D.

In a further embodiment of the ABD2 according to the first aspect above—the formula (i) or (ii), X6 is E. In another embodiment of the ABD2 according to this aspect, X6 is S. In another embodiment of the ABD2 according to this aspect, X3 is S. In another embodiment of the ABD2 according to this aspect, X3 is E. In another embodiment of the ABD2 according to this aspect, X7 is A. In another embodiment of the ABD2 according to this aspect, X7 is S. In another embodiment of the ABD2 according to this aspect, X10 is A. In another embodiment of the ABD2 according to this aspect, X10 is S. In another embodiment of the ABD2 according to this aspect, X10 is R. In another embodiment of the ABD2 according to this aspect, X14 is S. In another embodiment of the ABD2 according to this aspect, X14 is C. In another embodiment of the ABD2 according to this aspect, X14 is A. In another embodiment of the ABD2 according to this aspect, X14 is KIn another embodiment of the ABD2 according to this aspect, X26 is D. In another embodiment of the ABD2 according to this aspect, X26 is E. In another embodiment of the ABD2 according to this aspect X39 is D. In another embodiment of the ABD2 according to this aspect X39 is E. In another embodiment of the ABD2 according to this aspect X40 is A. In another embodiment of the ABD2 according to this aspect X40 is E. In another embodiment of the ABD2 according to this aspect X43 is A. In another embodiment of the ABD2 according to this aspect X43 is K. In another embodiment of the ABD2 according to this aspect X44 is A. In another embodiment of the ABD2 according to this aspect X44 is S. In another embodiment of the ABD2 according to this aspect X44 is E. In another embodiment of the ABD2 according to this aspect leucine at position 45 is present. In another embodiment of the ABD2 according to this aspect leucine at position 45 is absent. In a further embodiment the proline at position 46 is present. In a further embodiment the proline at position 46 at is absent.

In a further preferred embodiment albumin binding domain polypeptide suitable for conjugation or fusion to an exendin compound can comprise the improved ABD amino acid sequence selected from:

formula (iii)

(SEQ ID NO: 594)

LA X3 AK X6 X7 AN X10 ELD X14 YGVSDF YKRLIDKAKT
VEGVEALKDA ILAALP

wherein independently of each other

X3 is selected from E, S, Q and C;

X6 is selected from E, S and C;

X7 is selected from A and S;

X10 is selected from A, S and R;

X14 is selected from A, S, C and K;

the leucine at position 45 is present or absent; and

the proline at position 46 is present or absent; and

formula (iv) an amino acid sequence which has at least 95% identity to the sequence defined in (iii),

with the proviso that X₇ is not L, E or D;

or alternatively, with the proviso that the amino acid sequence is not defined by the following sequence, as defined in PCT Published Application No. WO 2009/016043: LAEAK Xₐ X_{b} A X_{c} X_{d} EL Xₑ KY GVSD X₅ YK X₈ X₉ I X₁₁ X₁₂ A X₁₄ TVEGV X₂₀ AL X₂₃ X₂₄ X₂₅ ILAALP (SEQ ID NO:593) wherein independently of each other,

Xₐ is selected from V and E;

X_{b} is selected from L, E and D;

X_{c} is selected from N, L and I;

X_{d} is selected from R and K;

Xₑ is selected from D and K; and

X₅ is selected from Y and F;

X₈ is selected from N, R and S;

X₉ is selected from V, I, L, M, F and Y;

X₁₁ is selected from N, S, E and D;

X₁₂ is selected from R, K and N;

X₁₄ is selected from K and R;

X₂₀ is selected from D, N, Q, E, H, S, R and K;

X₂₃ is selected from K, I and T;

X₂₄ is selected from A, S, T, G, H, L and D; and

X₂₅ is selected from H, E and D.

In a further embodiment of the ABD2 according to this aspect—formula (iii) or (iv), X6 is E. In another embodiment of the ABD2 according to this aspect, X6 is S. In another embodiment of the ABD2 according to this aspect, X3 is S. In another embodiment of the ABD2 according to this aspect, X3 is E. In another embodiment of the ABD2 according to this aspect, X7 is A. In another embodiment of the ABD2 according to this aspect, X7 is S. In another embodiment of the ABD2 according to this aspect, X10 is A. In another embodiment of the ABD2 according to this aspect, X10 is S. In another embodiment of the ABD2 according to this aspect, X10 is R. In another embodiment of the ABD2 according to this aspect, X14 is S. In another embodiment of the ABD2 according to this aspect, X14 is C. In another embodiment of the ABD2 according to this aspect, X14 is A. In another embodiment of the ABD2 according to this aspect, X14 is K. In another embodiment of the ABD2 according to this aspect leucine at position 45 is present. In another embodiment of the ABD2 according to this aspect leucine at position 45 is absent. In a further embodiment the proline at position 46 is present. In a further embodiment the proline at position 46 at is absent.

In a further embodiment of any one of the formulas (i) to (iv) the ABD comprises a one or more N-terminal helix-capping amino acids, and in a further embodiment the helix-capping amino acid may be serine, or may be glycine-serine. Accordingly for each albumin binding domain sequence disclosed herein, including those in the figures and sequenced listing, also specifically contemplated for all aspects as disclosed herein in the engineered polypeptide, are albumin binding domains corresponding to the ABD of any one of the formulas (i) to (iv) contained therein, their Ser-ABD, Gly-Ser-ABD, Gly-ABD, Ala-ABD and their des-C-terminal-proline sequences.

Thus, modified variants of (i) or (iii), which are such that the resulting sequence is at least 95% identical to a sequence belonging to the class defined by (i) or (iii), are also encompassed. For example, it is possible that an amino acid residue belonging to a certain functional grouping of amino acid residues (e.g. hydrophobic, hydrophilic, polar etc) could be exchanged for another amino acid residue from the same functional group.

The above defined class of sequence related ABD2 polypeptides having a binding affinity for albumin is derived from a common parent polypeptide sequence, which folds into a three alpha helix bundle domain. More specifically, the polypeptides as described above are derived from a model building based on a structure of a complex between serum albumin and the albumin binding domain G148-GA3 (Lejon et al, J. Biol. Chem. 279:42924-8, 2004), as well as analyses of binding and structural properties of a number of mutational variants of the common parent polypeptide sequence. The above defined amino acid sequence of any one of formulas (i) to (iv) comprises amino acid substitutions, as compared to the parent polypeptide sequence, that result in a class of polypeptides which are expected to fold into an almost identical three helix bundle domain. While the parent polypeptide sequence already comprises a binding surface for interaction with albumin, that binding surface is modified by some of the substitutions according to the above definition. The substitutions according to the above definition provide an improved albumin binding ability as compared to the parent polypeptide sequence. Importantly and surprisingly, the substitutions according to the above definition provide enhanced immunological properties, in addition to retaining and/or improving strong affinity for albumin.

Accordingly, the improved ABD polypeptides, ABD2, exhibit a set of characteristics, which, for example, make them suitable for use as fusion or conjugate partners for therapeutic molecules for human administration. Importantly and surprisingly, the improved ABD according to the present disclosure demonstrate, for example in comparison with related albumin binding polypeptides such as the albumin binding domain G148-GA3 and the albumin binding polypeptides disclosed in WO09/016043, and the ABD1 herein, at least five of the following six characteristics:

(1) The ABD2 polypeptides display a different surface compared to, for example, G148-GA3 and other bacterially derived albumin binding domains. The difference may decrease or eliminate any risk for antibody reactions in a subject, such as a human, which has been previously exposed to such bacterial proteins.

(2) The ABD2 polypeptides comprise fewer potential T-epitopes than, for example, G148-GA3 and other related, but different, mutational variants of the common parent polypeptide sequence, and hence exhibit low and/or lower immunogenicity when administered to a subject, such as a human.

(3) The polypeptides display lower reactivity with circulating antibodies when administered to a subject, such as a human. Thus, by amino acid substitutions in the surface of the polypeptides exposed to circulating antibodies, i.e. in the polypeptide surface not involved in the binding interaction with albumin, antibody cross-reactivity is reduced as compared to, for example, antibody cross-reactivity caused by G148-GA3 as measured in a test set of human sera.

(4) The polypeptides have a high albumin binding ability, both in terms of a higher binding affinity, as defined by a K_{D} value, and in terms of a slower off-rate, as defined by a koff value, than, for example, known naturally occurring albumin binding polypeptides, such as the albumin binding domains derived from bacterial proteins.

(5) The polypeptides comprise fewer amino acid residues that are associated with stability problems of polypeptides than, for example, known naturally occurring albumin binding polypeptides, such as the albumin binding domains derived from bacterial proteins. Thus, the polypeptides comprise, for example, no oxidation-prone methionines or tryptophans and only one asparagine.

(6) The polypeptides have a higher structural stability, as defined by a melting point of above 55° C., than previous albumin binding polypeptides, such as those disclosed in WO09/016043.

In one embodiment, the ABD2 of the conjugate/fusions display all six of the above listed characteristics. In another embodiment, the ABD2 according to the first aspect displays, when bound to albumin, a more hydrophilic profile than, for example, previous albumin binding polypeptides, such as those disclosed in WO09/016043. The surface of the ABD2 which is exposed to the surroundings when the polypeptide interacts with albumin comprises fewer amino acid residues that confer surface hydrophobicity.

Further for each of the embodiments herein of the ABD2 sequence, the C-terminal proline (corresponding to position 46 above) can be optionally absent. Even further for each embodiment of the ABD2 sequence, the leucine at position 45 can be optionally present or absent.

In another embodiment, the amino acid sequence of the ABD2 is selected from any one of SEQ ID NO:301-344. More specifically, the amino acid sequence is selected from SEQ ID NO:304-305, SEQ ID NO:307-308, SEQ ID NO:310-311, SEQ ID NO:313-314, SEQ ID NO:316-317, SEQ ID NO:319-320, SEQ ID NO:322-323, SEQ ID NO:325-326, SEQ ID NO:328-329, SEQ ID NO:331-332, SEQ ID NO:334-335, SEQ ID NO:337-338, SEQ ID NO:341-342 and SEQ ID NO:349-350.

In another preferred embodiment of the ABD used in the engineered polypeptides described herein, the amino acid sequence of the ABD2 portion of an engineered polypeptide includes an ABD selected from any one of the sequences described herein, including those from Table 1B or Table 1C, the sequence listing herein and further including their des-Pro46 and/or des-Leu45 forms.

In one embodiment, the ABD2 according to this aspect further includes one or more additional amino acid residues positioned at the N- and/or the C-terminal of the ABD sequence defined in (i) or (iii). These additional amino acid residues may play a role in further enhancing the binding of albumin by the polypeptide, and improving the conformational stability of the folded albumin binding domain, but may equally well serve other purposes, related for example to one or more of production, purification, stabilization in vivo or in vitro, coupling, labeling or detection of the polypeptide, as well as any combination thereof. Such additional amino acid residues may include one or more amino acid residue(s) added for purposes of chemical coupling, e.g. to the HD1.

For example, the amino acids directly preceding or following the alpha helix at the N- or C-terminus of the ABD2 amino acid sequence (i) or (iii) may thus in one embodiment affect the conformational stability. One example of an amino acid residue which may contribute to improved conformational stability is a serine residue positioned at the N-terminal of the ABD2 amino acid sequence (i) or (iii) as defined above. The N-terminal serine residue may in some cases form a canonical S-X-X-E capping box, by involving hydrogen bonding between the gamma oxygen of the serine side chain and the polypeptide backbone NH of the glutamic acid residue. This N-terminal capping may contribute to stabilization of the first alpha helix of the three helix domain constituting the ABD2 according to the first aspect of the disclosure.

Thus, in one embodiment, the additional amino acids include at least one serine residue at the N-terminal of the polypeptide. The ABD2 amino acid sequence is in other words preceded by one or more serine residue(s). In another embodiment of the albumin binding polypeptide, the additional amino acids include a glycine residue at the N-terminal of the ABD2 sequence. It is understood that the ABD2 amino acid sequence (i) or (iii) may be preceded by one, two, three, four or any suitable number of amino acid residues. Thus, the ABD2 amino acid sequence may be preceded by a single serine residue, a single glycine residue or a combination of the two, such as a glycine-serine (GS) combination or a glycine-serine-serine (GSS) combination. Examples of ABD2 comprising additional amino residues at the N-terminal are set out in SEQ ID NO:445-463, such as in SEQ ID NO:445-448 and SEQ ID NO:462-463, and in Table 1B and Table 1C. In yet another embodiment, the additional amino acid residues comprise a serine at the N-terminal of the polypeptide as defined by the sequence formula (i) or (iii). An example of one such ABD2 having a N-terminal serine is SGSLAEAKEAANAELDSYGVSDFYKRLIDKAKTVEGVEALKDAILAALP (SEQ ID NO. 595). The corresponding des-proline form is SGSLAEAKEAANAELDSYGVSDFYKRLIDKAKTVEGVEALKDAILAAL (SEQ ID NO:596). The corresponding des-Leu form is SGSLAEAKEAANAELDSYGVSDFYKRLIDKAKTVEGVEALKDAILAAP (SEQ ID NO:597). The corresponding des-Pro des-Leu form is SGSLAEAKEAANAELDSYGVSDFYKRLIDKAKTVEGVEALKDAILAA (SEQ ID NO:598).

In yet another embodiment, the additional amino acid residue or residues include an alanine acid at the N-terminal of the ABD2 polypeptide defined herein, or in combination with serine as an alanine-serine sequence at the N-terminal of the ABD2 sequences above. In yet another embodiment, the additional amino acid residue or residues include a glutamic acid at the N-terminal of the ABD2 polypeptide defined herein. In yet another embodiment, the additional amino acid residue or residues includes a cysteine at the N-terminal of the ABD2 polypeptide defined herein. Such additional residues when present are preferably from 1 to 5 amino acids.

Similarly, C-terminal capping may be exploited to improve stability of the third alpha helix of the three helix domain constituting the albumin binding polypeptide. The C-terminal proline residue present at the C-terminal of the ABD2 amino acid sequence defined in (i) or (iii) may at least partly function as a capping residue. A lysine residue following the proline residue at the C-terminal may contribute to further stabilization of the third helix of the albumin binding polypeptide, by hydrogen bonding between the epsilon amino group of the lysine residue and the carbonyl groups of the amino acids located two and three residues before the lysine in the polypeptide backbone, e.g. the carbonyl groups of the leucine and alanine residues of the ABD2 amino acid sequence defined in (i) or (iii). Thus, in one embodiment, the additional amino acids include a lysine residue at the C-terminal of the polypeptide. Such additional residues when present are preferably from 1 to 5 amino acids.

As discussed above, the additional amino acids may be related to the production of the ABD2. In particular, one or more optional amino acid residues following the C-terminal proline may provide advantages when the ABD2 according to the first aspect is produced by chemical peptide synthesis. Such additional amino acid residues may for example prevent formation of undesired substances, such as diketopiperazine at the dipeptide stage of the synthesis. One example of such an amino acid residue is glycine. Thus, in one embodiment, the additional amino acids include a glycine residue at the C-terminal of the polypeptide, directly following the proline residue or following an additional lysine and/or glycine residue as accounted for above. Alternatively, polypeptide production may benefit from amidation of the C-terminal proline residue of the ABD2 amino acid sequence (i) or (iii). In this case, the C-terminal proline includes an additional amine group at the carboxyl carbon.

Examples of ABD2 comprising additional amino acid residues at the C-terminal are set out in SEQ ID NO:445-452, such as in SEQ ID NO:449-450, and in Table 1B and Table 1C. The skilled person is aware of methods for accomplishing C-terminal modification, such as by different types of pre-made matrices for peptide synthesis.

In another embodiment, the additional amino acid residues includes a cysteine residue at the N- and/or C-terminal of the ABD2 polypeptide. Such a cysteine residue may directly precede and/or follow the ABD amino acid sequence as defined in (i) or (iii) or may precede and/or follow any other additional amino acid residues as described above. Examples of albumin binding polypeptides comprising a cysteine residue at the N- and/or C-terminal of the polypeptide chain are set out in SEQ ID NO:449-450 (C-terminal) and SEQ ID NO:451-452 (N-terminal), and in Table 1B and Table 1C. By the addition of a cysteine residue to the polypeptide chain, a thiol group for site directed conjugation of the ABD2 may be obtained. Alternatively, a selenocysteine residue may be introduced at the C-terminal of the polypeptide chain, in a similar fashion as for the introduction of a cysteine residue, to facilitate site-specific conjugation (Cheng et al, Nat Prot 1:2, 2006).

In one embodiment, the ABD2 includes no more than two cysteine residues. In another embodiment, the ABD2 includes no more than one cysteine residue.

In another embodiment, the additional amino acid residues of the ABD2 includes a “tag” for purification or detection of the polypeptide, such as a hexahistidyl (His₆) tag, or a “myc” (“c-Myc”) tag or a “FLAG” tag for interaction with antibodies specific to the tag and/or to be used in purification. The skilled person is aware of other alternatives.

Exemplary ABD species of the ABD2 sequence type (improved “deimmunized”) include, but are not limited to, the compounds set forth in Table 1B following and the Examples.

TABLE 1B
Selected ABD sequences of the ABD2 type

| Designation | ABD2 peptide sequence | SEQ ID NO: |
|---|---|---|
| PEP07271 | GSSLASAKEAANAELDAYGVSDFYKRLIDKAKTVEGVEALK | 455 |
| | DAILAALP | |
| PEP07554 | GSSLASAKEAANAELDSYGVSDFYKRLIDKAKTVEGVEALK | 456 |
| | DAILAALP | |
| PEP07912 | GLASAKEAANAELDSYGVSDFYKRLIDKAKTVEGVEALKD | 457 |
| | AILAALP | |
| PEP07914 | GLAEAKEAANAELDSYGVSDFYKRLIDKAKTVEGVEALKD | 458 |
| | AILAALP | |
| PEP07911 | GLASAKEAANAELDCYGVSDFYKRLIDKAKTVEGVEALKD | 459 |
| | AILAALP | |
| PEP07834 | ALASAKEAANAELDCYGVSDFYKRLIDKAKTVEGVEALKD | 460 |
| | AILAALP | |
| PEP07844 | GSSLASAKEAANAELDKYGVSDFYKRLIDKAKTVEGVEALK | 461 |
| | DAILAALP | |
| PEP07983 | GSLASAKEAANAELDSYGVSDFYKRLIDKAKTVEGVEALKD | 462 |
| | AILAALP | |
| PEP07986 | GSLAEAKEAANAELDSYGVSDFYKRLIDKAKTVEGVEALKD | 463 |
| | AILAALP | |
| PEP08185 | GSLAEAKEAANAELDCYGVSDFYKRLIDKAKTVEGVEALK | 448 |
| | DAILAALPG | |
| | LAEAKEAANAELDSYGVSDFYKRLIDKAKTVEGVEALKDAI | 313 |
| | LAALP | |
| | SLAEAKEAANAELDSYGVSDFYKRLIDKAKTVEGVEALKDA | 500 |
| | ILAALP | |
| | LAEAKEAANAELDCYGVSDFYKRLIDKAKTVEGVEALKDAI | 501 |
| | LAALPG | |
| | SLAEAKEAANAELDCYGVSDFYKRLIDKAKTVEGVEALKD | 502 |
| | AILAALPG | |
| (des C- | GSLAEAKEAANAELDSYGVSDFYKRLIDKAKTVEGVEALKD | 599 |
| terminal | AILAAL | |
| Pro) | | |
| PEP07986 | | |
| (des C- | GSLAEAKEAANAELDCYGVSDFYKRLIDKAKTVEGVEALK | 600 |
| terminal | DAILAAL | |
| Pro-Gly) | | |
| PEP08185 | | |
| | SLAEAKEAANAELDSYGVSDFYKRLIDKAKTVEGVEALKDA | 601 |
| | ILAAL | |
| | SLAEAKEAANAELDCYGVSDFYKRLIDKAKTVEGVEALKD | 602 |
| | AILAAL | |
| | LAEAKEAANAELDSYGVSDFYKRLIDKAKTVEGVEALKDAI | 603 |
| | LAAL | |
| | LAEAKEAANAELDCYGVSDFYKRLIDKAKTVEGVEALKDAI | 604 |
| | LAAL | |
| | GSSLASAKEAANAELDAYGVSDFYKRLIDKAKTVEGVEALK | 605 |
| | DAILAA | |
| | GSSLASAKEAANAELDSYGVSDFYKRLIDKAKTVEGVEALK | 606 |
| | DAILAA | |
| | GLASAKEAANAELDSYGVSDFYKRLIDKAKTVEGVEALKD | 607 |
| | AILAA | |
| | GLAEAKEAANAELDSYGVSDFYKRLIDKAKTVEGVEALKD | 608 |
| | AILAA | |
| | GLASAKEAANAELDCYGVSDFYKRLIDKAKTVEGVEALKD | 609 |
| | AILAA | |
| | ALASAKEAANAELDCYGVSDFYKRLIDKAKTVEGVEALKD | 610 |
| | AILAA | |
| | GSSLASAKEAANAELDKYGVSDFYKRLIDKAKTVEGVEALK | 611 |
| | DAILAA | |
| | GSLASAKEAANAELDSYGVSDFYKRLIDKAKTVEGVEALKD | 612 |
| | AILAA | |
| | GSLAEAKEAANAELDSYGVSDFYKRLIDKAKTVEGVEALKD | 613 |
| | AILAA | |
| | GSLAEAKEAANAELDCYGVSDFYKRLIDKAKTVEGVEALK | 614 |
| | DAILAA | |
| | LAEAKEAANAELDSYGVSDFYKRLIDKAKTVEGVEALKDAI | 615 |
| | LAA | |
| | SLAEAKEAANAELDSYGVSDFYKRLIDKAKTVEGVEALKDA | 616 |
| | ILAA | |
| | LAEAKEAANAELDCYGVSDFYKRLIDKAKTVEGVEALKDAI | 617 |
| | LAA | |
| | SLAEAKEAANAELDCYGVSDFYKRLIDKAKTVEGVEALKD | 618 |
| | AILAA | |

Table 1C is a listing of the amino acid sequences of examples of albumin binding polypeptides of the ABD2 type (SEQ ID NO:301-452, SEQ ID NO:455-461) useful in the engineered polypeptides disclosed herein, the GA3 domain from protein G of Streptococcus strain G148 (SEQ ID NO:453) extended by a N-terminal glycine residue and an albumin binding polypeptide derived from G148-GA3 as previously described by Jonsson et al (Protein Eng. Design & Selection, 2008, 21:515-527); SEQ ID NO:454).

TABLE 1C
Selected ABD peptides of the ABD2 type

| | | SEQ ID |
|---|---|---|
| ABD | Amino acid sequence | NO: |
| PP001 | LASAKEAANAELDAYGVSDFYKRLIDKAKTVEGVEALKDAILAALP | 301 |
| PP002 | LASAKEAANSELDAYGVSDFYKRLIDKAKTVEGVEALKDAILAALP | 302 |
| PP003 | LASAKESANSELDAYGVSDFYKRLIDKAKTVEGVEALKDAILAALP | 303 |
| PP004 | LASAKESANAELDAYGVSDFYKRLIDKAKTVEGVEALKDAILAALP | 304 |
| PP005 | LASAKSAANAELDAYGVSDFYKRLIDKAKTVEGVEALKDAILAALP | 305 |
| PP006 | LASAKSAANSELDAYGVSDFYKRLIDKAKTVEGVEALKDAILAALP | 306 |
| PP007 | LASAKEAANAELDSYGVSDFYKRLIDKAKTVEGVEALKDAILAALP | 307 |
| PP008 | LASAKEAANSELDSYGVSDFYKRLIDKAKTVEGVEALKDAILAALP | 308 |
| PP009 | LASAKESANSELDSYGVSDFYKRLIDKAKTVEGVEALKDAILAALP | 309 |
| PP010 | LASAKESANAELDSYGVSDFYKRLIDKAKTVEGVEALKDAILAALP | 310 |
| PP011 | LASAKSAANAELDSYGVSDFYKRLIDKAKTVEGVEALKDAILAALP | 311 |
| PP012 | LASAKSAANSELDSYGVSDFYKRLIDKAKTVEGVEALKDAILAALP | 312 |
| PP013 | LAEAKEAANAELDSYGVSDFYKRLIDKAKTVEGVEALKDAILAALP | 313 |
| PP014 | LAEAKEAANSELDSYGVSDFYKRLIDKAKTVEGVEALKDAILAALP | 314 |
| PP015 | LAEAKESANSELDSYGVSDFYKRLIDKAKTVEGVEALKDAILAALP | 315 |
| PP016 | LAEAKESANAELDSYGVSDFYKRLIDKAKTVEGVEALKDAILAALP | 316 |
| PP017 | LAEAKSAANAELDSYGVSDFYKRLIDKAKTVEGVEALKDAILAALP | 317 |
| PP018 | LAEAKSAANSELDSYGVSDFYKRLIDKAKTVEGVEALKDAILAALP | 318 |
| PP019 | LAEAKEAANAELDAYGVSDFYKRLIDKAKTVEGVEALKDAILAALP | 319 |
| PP020 | LAEAKEAANSELDAYGVSDFYKRLIDKAKTVEGVEALKDAILAALP | 320 |
| PP021 | LAEAKESANSELDAYGVSDFYKRLIDKAKTVEGVEALKDAILAALP | 321 |
| PP022 | LAEAKESANAELDAYGVSDFYKRLIDKAKTVEGVEALKDAILAALP | 322 |
| PP023 | LAEAKSAANAELDAYGVSDFYKRLIDKAKTVEGVEALKDAILAALP | 323 |
| PP024 | LAEAKSAANSELDAYGVSDFYKRLIDKAKTVEGVEALKDAILAALP | 324 |
| PP025 | LAQAKEAANAELDAYGVSDFYKRLIDKAKTVEGVEALKDAILAALP | 325 |
| PP026 | LAQAKEAANSELDAYGVSDFYKRLIDKAKTVEGVEALKDAILAALP | 326 |
| PP027 | LAQAKESANSELDAYGVSDFYKRLIDKAKTVEGVEALKDAILAALP | 327 |
| PP028 | LAQAKESANAELDAYGVSDFYKRLIDKAKTVEGVEALKDAILAALP | 328 |
| PP029 | LAQAKSAANAELDAYGVSDFYKRLIDKAKTVEGVEALKDAILAALP | 329 |
| PP030 | LAQAKSAANSELDAYGVSDFYKRLIDKAKTVEGVEALKDAILAALP | 330 |
| PP031 | LAQAKEAANAELDSYGVSDFYKRLIDKAKTVEGVEALKDAILAALP | 331 |
| PP032 | LAQAKEAANSELDSYGVSDFYKRLIDKAKTVEGVEALKDAILAALP | 332 |
| PP033 | LAQAKESANSELDSYGVSDFYKRLIDKAKTVEGVEALKDAILAALP | 333 |
| PP034 | LAQAKESANAELDSYGVSDFYKRLIDKAKTVEGVEALKDAILAALP | 334 |
| PP035 | LAQAKSAANAELDSYGVSDFYKRLIDKAKTVEGVEALKDAILAALP | 335 |
| PP036 | LAQAKSAANSELDSYGVSDFYKRLIDKAKTVEGVEALKDAILAALP | 336 |
| PP037 | LASAKEAANAELDCYGVSDFYKRLIDKAKTVEGVEALKDAILAALP | 337 |
| PP038 | LASAKEAANSELDCYGVSDFYKRLIDKAKTVEGVEALKDAILAALP | 338 |
| PP039 | LASAKESANSELDCYGVSDFYKRLIDKAKTVEGVEALKDAILAALP | 339 |
| PP040 | LASAKESANAELDCYGVSDFYKRLIDKAKTVEGVEALKDAILAALP | 340 |
| PP041 | LASAKSAANAELDCYGVSDFYKRLIDKAKTVEGVEALKDAILAALP | 341 |
| PP042 | LASAKSAANSELDCYGVSDFYKRLIDKAKTVEGVEALKDAILAALP | 342 |
| PP043 | LASAKEAANAELDKYGVSDFYKRLIDKAKTVEGVEALKDAILAALP | 343 |
| PP044 | LASAKEAANSELDKYGVSDFYKRLIDKAKTVEGVEALKDAILAALP | 344 |
| PP045 | LASAKESANSELDKYGVSDFYKRLIDKAKTVEGVEALKDAILAALP | 345 |
| PP046 | LASAKESANAELDKYGVSDFYKRLIDKAKTVEGVEALKDAILAALP | 346 |
| PP047 | LASAKSAANAELDKYGVSDFYKRLIDKAKTVEGVEALKDAILAALP | 347 |
| PP048 | LASAKSAANSELDKYGVSDFYKRLIDKAKTVEGVEALKDAILAALP | 348 |
| PP049 | LAEAKEAANAELDCYGVSDFYKRLIDKAKTVEGVEALKDAILAALP | 349 |
| PP050 | LAEAKEAANSELDCYGVSDFYKRLIDKAKTVEGVEALKDAILAALP | 350 |
| PP051 | LAEAKESANSELDCYGVSDFYKRLIDKAKTVEGVEALKDAILAALP | 351 |
| PP052 | LAEAKESANAELDCYGVSDFYKRLIDKAKTVEGVEALKDAILAALP | 352 |
| PP053 | LAEAKSAANAELDCYGVSDFYKRLIDKAKTVEGVEALKDAILAALP | 353 |
| PP054 | LAEAKSAANSELDCYGVSDFYKRLIDKAKTVEGVEALKDAILAALP | 354 |
| PP055 | LAEAKEAANAELDKYGVSDFYKRLIDKAKTVEGVEALKDAILAALP | 355 |
| PP056 | LAEAKEAANSELDKYGVSDFYKRLIDKAKTVEGVEALKDAILAALP | 356 |
| PP057 | LAEAKESANSELDKYGVSDFYKRLIDKAKTVEGVEALKDAILAALP | 357 |
| PP058 | LAEAKESANAELDKYGVSDFYKRLIDKAKTVEGVEALKDAILAALP | 358 |
| PP059 | LAEAKSAANAELDKYGVSDFYKRLIDKAKTVEGVEALKDAILAALP | 359 |
| PP060 | LAEAKSAANSELDKYGVSDFYKRLIDKAKTVEGVEALKDAILAALP | 360 |
| PP061 | LAQAKEAANAELDCYGVSDFYKRLIDKAKTVEGVEALKDAILAALP | 361 |
| PP062 | LAQAKEAANSELDCYGVSDFYKRLIDKAKTVEGVEALKDAILAALP | 362 |
| PP063 | LAQAKESANSELDCYGVSDFYKRLIDKAKTVEGVEALKDAILAALP | 363 |
| PP064 | LAQAKESANAELDCYGVSDFYKRLIDKAKTVEGVEALKDAILAALP | 364 |
| PP065 | LAQAKSAANAELDCYGVSDFYKRLIDKAKTVEGVEALKDAILAALP | 365 |
| PP066 | LAQAKSAANSELDCYGVSDFYKRLIDKAKTVEGVEALKDAILAALP | 366 |
| PP067 | LAQAKEAANAELDKYGVSDFYKRLIDKAKTVEGVEALKDAILAALP | 367 |
| PP068 | LAQAKEAANSELDKYGVSDFYKRLIDKAKTVEGVEALKDAILAALP | 368 |
| PP069 | LAQAKESANSELDKYGVSDFYKRLIDKAKTVEGVEALKDAILAALP | 369 |
| PP070 | LAQAKESANAELDKYGVSDFYKRLIDKAKTVEGVEALKDAILAALP | 370 |
| PP071 | LAQAKSAANAELDKYGVSDFYKRLIDKAKTVEGVEALKDAILAALP | 371 |
| PP072 | LAQAKSAANSELDKYGVSDFYKRLIDKAKTVEGVEALKDAILAALP | 372 |
| PP073 | LACAKEAANAELDCYGVSDFYKRLIDKAKTVEGVEALKDAILAALP | 373 |
| PP074 | LACAKEAANSELDCYGVSDFYKRLIDKAKTVEGVEALKDAILAALP | 374 |
| PP075 | LACAKESANSELDCYGVSDFYKRLIDKAKTVEGVEALKDAILAALP | 375 |
| PP076 | LACAKESANAELDCYGVSDFYKRLIDKAKTVEGVEALKDAILAALP | 376 |
| PP077 | LACAKSAANAELDCYGVSDFYKRLIDKAKTVEGVEALKDAILAALP | 377 |
| PP078 | LACAKSAANSELDCYGVSDFYKRLIDKAKTVEGVEALKDAILAALP | 378 |
| PP079 | LACAKEAANAELDKYGVSDFYKRLIDKAKTVEGVEALKDAILAALP | 379 |
| PP080 | LACAKEAANSELDKYGVSDFYKRLIDKAKTVEGVEALKDAILAALP | 380 |
| PP081 | LACAKESANSELDKYGVSDFYKRLIDKAKTVEGVEALKDAILAALP | 381 |
| PP082 | LACAKESANAELDKYGVSDFYKRLIDKAKTVEGVEALKDAILAALP | 382 |
| PP083 | LACAKSAANAELDKYGVSDFYKRLIDKAKTVEGVEALKDAILAALP | 383 |
| PP084 | LACAKSAANSELDKYGVSDFYKRLIDKAKTVEGVEALKDAILAALP | 384 |
| PP085 | LACAKEAANAELDSYGVSDFYKRLIDKAKTVEGVEALKDAILAALP | 385 |
| PP086 | LACAKEAANSELDSYGVSDFYKRLIDKAKTVEGVEALKDAILAALP | 386 |
| PP087 | LACAKESANSELDSYGVSDFYKRLIDKAKTVEGVEALKDAILAALP | 387 |
| PP088 | LACAKESANAELDSYGVSDFYKRLIDKAKTVEGVEALKDAILAALP | 388 |
| PP089 | LACAKSAANAELDSYGVSDFYKRLIDKAKTVEGVEALKDAILAALP | 389 |
| PP090 | LACAKSAANSELDSYGVSDFYKRLIDKAKTVEGVEALKDAILAALP | 390 |
| PP091 | LACAKEAANAELDAYGVSDFYKRLIDKAKTVEGVEALKDAILAALP | 391 |
| PP092 | LACAKEAANSELDAYGVSDFYKRLIDKAKTVEGVEALKDAILAALP | 392 |
| PP093 | LACAKESANSELDAYGVSDFYKRLIDKAKTVEGVEALKDAILAALP | 393 |
| PP094 | LACAKESANAELDAYGVSDFYKRLIDKAKTVEGVEALKDAILAALP | 394 |
| PP095 | LACAKSAANAELDAYGVSDFYKRLIDKAKTVEGVEALKDAILAALP | 395 |
| PP096 | LACAKSAANSELDAYGVSDFYKRLIDKAKTVEGVEALKDAILAALP | 396 |
| PP097 | LAQAKCAANAELDAYGVSDFYKRLIDKAKTVEGVEALKDAILAALP | 397 |
| PP098 | LAQAKCAANSELDAYGVSDFYKRLIDKAKTVEGVEALKDAILAALP | 398 |
| PP099 | LAQAKCSANSELDAYGVSDFYKRLIDKAKTVEGVEALKDAILAALP | 399 |
| PP100 | LAQAKCSANAELDAYGVSDFYKRLIDKAKTVEGVEALKDAILAALP | 400 |
| PP101 | LAQAKCAANAELDCYGVSDFYKRLIDKAKTVEGVEALKDAILAALP | 401 |
| PP102 | LAQAKCAANSELDCYGVSDFYKRLIDKAKTVEGVEALKDAILAALP | 402 |
| PP103 | LAQAKCAANAELDSYGVSDFYKRLIDKAKTVEGVEALKDAILAALP | 403 |
| PP104 | LAQAKCAANSELDSYGVSDFYKRLIDKAKTVEGVEALKDAILAALP | 404 |
| PP105 | LAQAKCSANSELDSYGVSDFYKRLIDKAKTVEGVEALKDAILAALP | 405 |
| PP106 | LAQAKCSANAELDSYGVSDFYKRLIDKAKTVEGVEALKDAILAALP | 406 |
| PP107 | LAQAKCAANAELDKYGVSDFYKRLIDKAKTVEGVEALKDAILAALP | 407 |
| PP108 | LAQAKCAANSELDKYGVSDFYKRLIDKAKTVEGVEALKDAILAALP | 408 |
| PP109 | LASAKCAANAELDAYGVSDFYKRLIDKAKTVEGVEALKDAILAALP | 409 |
| PP110 | LASAKCAANSELDAYGVSDFYKRLIDKAKTVEGVEALKDAILAALP | 410 |
| PP111 | LASAKCSANSELDAYGVSDFYKRLIDKAKTVEGVEALKDAILAALP | 411 |
| PP112 | LASAKCSANAELDAYGVSDFYKRLIDKAKTVEGVEALKDAILAALP | 412 |
| PP113 | LASAKCAANAELDCYGVSDFYKRLIDKAKTVEGVEALKDAILAALP | 413 |
| PP114 | LASAKCAANSELDCYGVSDFYKRLIDKAKTVEGVEALKDAILAALP | 414 |
| PP115 | LASAKCAANAELDSYGVSDFYKRLIDKAKTVEGVEALKDAILAALP | 415 |
| PP116 | LASAKCAANSELDSYGVSDFYKRLIDKAKTVEGVEALKDAILAALP | 416 |
| PP117 | LASAKCSANSELDSYGVSDFYKRLIDKAKTVEGVEALKDAILAALP | 417 |
| PP118 | LASAKCSANAELDSYGVSDFYKRLIDKAKTVEGVEALKDAILAALP | 418 |
| PP119 | LASAKCAANAELDKYGVSDFYKRLIDKAKTVEGVEALKDAILAALP | 419 |
| PP120 | LASAKCAANSELDKYGVSDFYKRLIDKAKTVEGVEALKDAILAALP | 420 |
| PP121 | LAEAKCAANAELDAYGVSDFYKRLIDKAKTVEGVEALKDAILAALP | 421 |
| PP122 | LAEAKCAANSELDAYGVSDFYKRLIDKAKTVEGVEALKDAILAALP | 422 |
| PP123 | LAEAKCSANSELDAYGVSDFYKRLIDKAKTVEGVEALKDAILAALP | 423 |
| PP124 | LAEAKCSANAELDAYGVSDFYKRLIDKAKTVEGVEALKDAILAALP | 424 |
| PP125 | LAEAKCAANAELDCYGVSDFYKRLIDKAKTVEGVEALKDAILAALP | 425 |
| PP126 | LAEAKCAANSELDCYGVSDFYKRLIDKAKTVEGVEALKDAILAALP | 426 |
| PP127 | LAEAKCAANAELDSYGVSDFYKRLIDKAKTVEGVEALKDAILAALP | 427 |
| PP128 | LAEAKCAANSELDSYGVSDFYKRLIDKAKTVEGVEALKDAILAALP | 428 |
| PP129 | LAEAKCSANSELDSYGVSDFYKRLIDKAKTVEGVEALKDAILAALP | 429 |
| PP130 | LAEAKCSANAELDSYGVSDFYKRLIDKAKTVEGVEALKDAILAALP | 430 |
| PP131 | LAEAKCAANAELDKYGVSDFYKRLIDKAKTVEGVEALKDAILAALP | 431 |
| PP132 | LAEAKCAANSELDKYGVSDFYKRLIDKAKTVEGVEALKDAILAALP | 432 |
| PP133 | LACAKCAANAELDAYGVSDFYKRLIDKAKTVEGVEALKDAILAALP | 433 |
| PP134 | LACAKCAANSELDAYGVSDFYKRLIDKAKTVEGVEALKDAILAALP | 434 |
| PP135 | LACAKCSANSELDAYGVSDFYKRLIDKAKTVEGVEALKDAILAALP | 435 |
| PP136 | LACAKCSANAELDAYGVSDFYKRLIDKAKTVEGVEALKDAILAALP | 436 |
| PP137 | LACAKCAANAELDCYGVSDFYKRLIDKAKTVEGVEALKDAILAALP | 437 |
| PP138 | LACAKCAANSELDCYGVSDFYKRLIDKAKTVEGVEALKDAILAALP | 438 |
| PP139 | LACAKCAANAELDSYGVSDFYKRLIDKAKTVEGVEALKDAILAALP | 439 |
| PP140 | LACAKCAANSELDSYGVSDFYKRLIDKAKTVEGVEALKDAILAALP | 440 |
| PP141 | LACAKCSANSELDSYGVSDFYKRLIDKAKTVEGVEALKDAILAALP | 441 |
| PP142 | LACAKCSANAELDSYGVSDFYKRLIDKAKTVEGVEALKDAILAALP | 442 |
| PP143 | LACAKCAANAELDKYGVSDFYKRLIDKAKTVEGVEALKDAILAALP | 443 |
| PP144 | LACAKCAANSELDKYGVSDFYKRLIDKAKTVEGVEALKDAILAALP | 444 |
| PP145 | GSLASAKEAANAELDSYGVSDFYKRLIDKAKTVEGVEALKDAILAALPG | 445 |
| PP146 | GSLAEAKEAANAELDSYGVSDFYKRLIDKAKTVEGVEALKDAILAALPG | 446 |
| PP147 | GSLASAKEAANAELDCYGVSDFYKRLIDKAKTVEGVEALKDAILAALPG | 447 |
| PEP08185 | GSLAEAKEAANAELDCYGVSDFYKRLIDKAKTVEGVEALKDAILAALPG | 448 |
| PP149 | GSLASAKEAANAELDSYGVSDFYKRLIDKAKTVEGVEALKDAILAALPCG | 449 |
| PP150 | GSLAEAKEAANAELDSYGVSDFYKRLIDKAKTVEGVEALKDAILAALPCG | 450 |
| PP151 | GCSLASAKEAANAELDKYGVSDFYKRLIDKAKTVEGVEALKDAILAALPG | 451 |
| PP152 | GCSLAEAKEAANAELDKYGVSDFYKRLIDKAKTVEGVEALKDAILAALPG | 452 |
| PEP07913 | GLAEAKVLANRELDKYGVSDYYKNLINNAKTVEGVKALIDEILAALP | 453 |
| PEP06923 | GSSLAEAKVLANRELDKYGVSDFYKRLINKAKTVEGVEALKLHILAALP | 454 |
| PEP07271 | GSSLASAKEAANAELDAYGVSDFYKRLIDKAKTVEGVEALKDAILAALP | 455 |
| PEP07554 | GSSLASAKEAANAELDSYGVSDFYKRLIDKAKTVEGVEALKDAILAALP | 456 |
| PEP07912 | GLASAKEAANAELDSYGVSDFYKRLIDKAKTVEGVEALKDAILAALP | 457 |
| PEP07914 | GLAEAKEAANAELDSYGVSDFYKRLIDKAKTVEGVEALKDAILAALP | 458 |
| PEP07911 | GLASAKEAANAELDCYGVSDFYKRLIDKAKTVEGVEALKDAILAALP | 459 |
| PEP07834 | ALASAKEAANAELDCYGVSDFYKRLIDKAKTVEGVEALKDAILAALP | 460 |
| PEP07844 | GSSLASAKEAANAELDKYGVSDFYKRLIDKAKTVEGVEALKDAILAALP | 461 |
| PEP07983 | GSLASAKEAANAELDSYGVSDFYKRLIDKAKTVEGVEALKDAILAALP | 462 |
| PEP07986 | GSLAEAKEAANAELDSYGVSDFYKRLIDKAKTVEGVEALKDAILAALP | 463 |
| PP013 + NtermS | SLAEAKEAANAELDSYGVSDFYKRLIDKAKTVEGVEALKDAILAALP | 500 |
| 501 | LAEAKEAANAELDCYGVSDFYKRLIDKAKTVEGVEALKDAILAALPG | 501 |
| 501 + NtermS | SLAEAKEAANAELDCYGVSDFYKRLIDKAKTVEGVEALKDAILAALPG | 502 |
| | GSLAQAKEAANAELDSYGVSDFYKRLIDKAKTVEGVEALKDAILAALP | 503 |
| | GSLAEAKEAANRELDSYGVSDFYKRLIDKAKTVEGVEALKDAILAALP | 504 |
| | GSLAQAKEAANRELDSYGVSDFYKRLIDKAKTVEGVEALKDAILAALP | 505 |
| | GSLAEAKEAANRELDAYGVSDFYKRLIDKAKTVEGVEALKDAILAALP | 506 |
| | GSLAEAKVLANRELDKYGVSDFYKRLIDKAKTVEGVEALKDAILAALP | 507 |
| | SLAQAKEAANAELDSYGVSDFYKRLIDKAKTVEGVEALKDAILAALP | 508 |
| | SLAEAKEAANRELDSYGVSDFYKRLIDKAKTVEGVEALKDAILAALP | 509 |
| | SLAQAKEAANRELDSYGVSDFYKRLIDKAKTVEGVEALKDAILAALP | 510 |
| | SLAEAKEAANRELDAYGVSDFYKRLIDKAKTVEGVEALKDAILAALP | 511 |
| | SLAEAKVLANRELDKYGVSDFYKRLIDKAKTVEGVEALKDAILAALP | 512 |
| | LAQAKEAANAELDSYGVSDFYKRLIDKAKTVEGVEALKDAILAALP | 513 |
| | LAEAKEAANRELDSYGVSDFYKRLIDKAKTVEGVEALKDAILAALP | 514 |
| | LAQAKEAANRELDSYGVSDFYKRLIDKAKTVEGVEALKDAILAALP | 515 |
| | LAEAKEAANRELDAYGVSDFYKRLIDKAKTVEGVEALKDAILAALP | 516 |
| | LAEAKVLANRELDKYGVSDFYKRLIDKAKTVEGVEALKDAILAALP | 517 |
| | GSLAQAKEAANAELDSYGVSDFYKRLIEKAKTVEGVEALKDAILAALP | 510 |
| | GSLAEAKEAANRELDSYGVSDFYKRLIEKAKTVEGVEALKDAILAALP | 519 |
| | GSLAQAKEAANRELDSYGVSDFYKRLIEKAKTVEGVEALKDAILAALP | 520 |
| | GSLAEAKEAANRELDAYGVSDFYKRLIEKAKTVEGVEALKDAILAALP | 521 |
| | GSLAEAKVLANRELDKYGVSDFYKRLIEKAKTVEGVEALKDAILAALP | 522 |
| | SLAQAKEAANAELDSYGVSDFYKRLIEKAKTVEGVEALKDAILAALP | 523 |
| | SLAEAKEAANRELDSYGVSDFYKRLIEKAKTVEGVEALKDAILAALP | 524 |
| | SLAQAKEAANRELDSYGVSDFYKRLIEKAKTVEGVEALKDAILAALP | 525 |
| | SLAEAKEAANRELDAYGVSDFYKRLIEKAKTVEGVEALKDAILAALP | 526 |
| | SLAEAKVLANRELDKYGVSDFYKRLIEKAKTVEGVEALKDAILAALP | 527 |
| | LAQAKEAANAELDSYGVSDFYKRLIEKAKTVEGVEALKDAILAALP | 528 |
| | LAEAKEAANRELDSYGVSDFYKRLIEKAKTVEGVEALKDAILAALP | 529 |
| | LAQAKEAANRELDSYGVSDFYKRLIEKAKTVEGVEALKDAILAALP | 530 |
| | LAEAKEAANRELDAYGVSDFYKRLIEKAKTVEGVEALKDAILAALP | 531 |
| | LAEAKVLANRELDKYGVSDFYKRLIEKAKTVEGVEALKDAILAALP | 532 |
| | GSLAQAKEAANAELDSYGVSDFYKRLIEKAKTVEGVEALKEAILAALP | 533 |
| | GSLAEAKEAANRELDSYGVSDFYKRLIEKAKTVEGVEALKEAILAALP | 534 |
| | GSLAQAKEAANRELDSYGVSDFYKRLIEKAKTVEGVEALKEAILAALP | 535 |
| | GSLAEAKEAANRELDAYGVSDFYKRLIEKAKTVEGVEALKEAILAALP | 536 |
| | GSLAEAKVLANRELDKYGVSDFYKRLIEKAKTVEGVEALKEAILAALP | 537 |
| | SLAQAKEAANAELDSYGVSDFYKRLIEKAKTVEGVEALKEAILAALP | 538 |
| | SLAEAKEAANRELDSYGVSDFYKRLIEKAKTVEGVEALKEAILAALP | 539 |
| | SLAQAKEAANRELDSYGVSDFYKRLIEKAKTVEGVEALKEAILAALP | 540 |
| | SLAEAKEAANRELDAYGVSDFYKRLIEKAKTVEGVEALKEAILAALP | 541 |
| | SLAEAKVLANRELDKYGVSDFYKRLIEKAKTVEGVEALKEAILAALP | 542 |
| | LAQAKEAANAELDSYGVSDFYKRLIEKAKTVEGVEALKEAILAALP | 543 |
| | LAEAKEAANRELDSYGVSDFYKRLIEKAKTVEGVEALKEAILAALP | 544 |
| | LAQAKEAANRELDSYGVSDFYKRLIEKAKTVEGVEALKEAILAALP | 545 |
| | LAEAKEAANRELDAYGVSDFYKRLIEKAKTVEGVEALKEAILAALP | 546 |
| | LAEAKVLANRELDKYGVSDFYKRLIEKAKTVEGVEALKEAILAALP | 547 |
| | GSLAQAKEAANAELDSYGVSDFYKRLIDKAKTVEGVEALKDAILASLP | 548 |
| | GSLAEAKEAANRELDSYGVSDFYKRLIDKAKTVEGVEALKDAILASLP | 549 |
| | GSLAQAKEAANRELDSYGVSDFYKRLIDKAKTVEGVEALKDAILASLP | 550 |
| | GSLAEAKEAANRELDAYGVSDFYKRLIDKAKTVEGVEALKDAILASLP | 551 |
| | GSLAEAKVLANRELDKYGVSDFYKRLIDKAKTVEGVEALKDAILASLP | 552 |
| | SLAQAKEAANAELDSYGVSDFYKRLIDKAKTVEGVEALKDAILASLP | 553 |
| | SLAEAKEAANRELDSYGVSDFYKRLIDKAKTVEGVEALKDAILASLP | 554 |
| | SLAQAKEAANRELDSYGVSDFYKRLIDKAKTVEGVEALKDAILASLP | 555 |
| | SLAEAKEAANRELDAYGVSDFYKRLIDKAKTVEGVEALKDAILASLP | 556 |
| | SLAEAKVLANRELDKYGVSDFYKRLIDKAKTVEGVEALKDAILASLP | 557 |
| | LAQAKEAANAELDSYGVSDFYKRLIDKAKTVEGVEALKDAILASLP | 558 |
| | LAEAKEAANRELDSYGVSDFYKRLIDKAKTVEGVEALKDAILASLP | 559 |
| | LAQAKEAANRELDSYGVSDFYKRLIDKAKTVEGVEALKDAILASLP | 560 |
| | LAEAKEAANRELDAYGVSDFYKRLIDKAKTVEGVEALKDAILASLP | 561 |
| | LAEAKVLANRELDKYGVSDFYKRLIDKAKTVEGVEALKDAILASLP | 562 |
| | GSLAQAKEAANAELDSYGVSDFYKRLIDKAKTVEGVEALKDAILAELP | 563 |
| | GSLAEAKEAANRELDSYGVSDFYKRLIDKAKTVEGVEALKDAILAELP | 564 |
| | GSLAQAKEAANRELDSYGVSDFYKRLIDKAKTVEGVEALKDAILAELP | 565 |
| | GSLAEAKEAANRELDAYGVSDFYKRLIDKAKTVEGVEALKDAILAELP | 566 |
| | GSLAEAKVLANRELDKYGVSDFYKRLIDKAKTVEGVEALKDAILAELP | 567 |
| | SLAQAKEAANAELDSYGVSDFYKRLIDKAKTVEGVEALKDAILAELP | 568 |
| | SLAEAKEAANRELDSYGVSDFYKRLIDKAKTVEGVEALKDAILAELP | 569 |
| | SLAQAKEAANRELDSYGVSDFYKRLIDKAKTVEGVEALKDAILAELP | 570 |
| | SLAEAKEAANRELDAYGVSDFYKRLIDKAKTVEGVEALKDAILAELP | 571 |
| | SLAEAKVLANRELDKYGVSDFYKRLIDKAKTVEGVEALKDAILAELP | 572 |
| | LAQAKEAANAELDSYGVSDFYKRLIDKAKTVEGVEALKDAILAELP | 573 |
| | LAEAKEAANRELDSYGVSDFYKRLIDKAKTVEGVEALKDAILAELP | 574 |
| | LAQAKEAANRELDSYGVSDFYKRLIDKAKTVEGVEALKDAILAELP | 575 |
| | LAEAKEAANRELDAYGVSDFYKRLIDKAKTVEGVEALKDAILAELP | 576 |
| | LAEAKVLANRELDKYGVSDFYKRLIDKAKTVEGVEALKDAILAELP | 577 |
| | GSLAQAKEAANAELDSYGVSDFYKRLIDKAKTVEGVEALKDAILKALP | 578 |
| | GSLAEAKEAANRELDSYGVSDFYKRLIDKAKTVEGVEALKDAILKALP | 579 |
| | GSLAQAKEAANRELDSYGVSDFYKRLIDKAKTVEGVEALKDAILKALP | 580 |
| | GSLAEAKEAANRELDAYGVSDFYKRLIDKAKTVEGVEALKDAILKALP | 581 |
| | GSLAEAKVLANRELDKYGVSDFYKRLIDKAKTVEGVEALKDAILKALP | 582 |
| | SLAQAKEAANAELDSYGVSDFYKRLIDKAKTVEGVEALKDAILKALP | 583 |
| | SLAEAKEAANRELDSYGVSDFYKRLIDKAKTVEGVEALKDAILKALP | 584 |
| | SLAQAKEAANRELDSYGVSDFYKRLIDKAKTVEGVEALKDAILKALP | 585 |
| | SLAEAKEAANRELDAYGVSDFYKRLIDKAKTVEGVEALKDAILKALP | 586 |
| | SLAEAKVLANRELDKYGVSDFYKRLIDKAKTVEGVEALKDAILKALP | 587 |
| | LAQAKEAANAELDSYGVSDFYKRLIDKAKTVEGVEALKDAILKALP | 588 |
| | LAEAKEAANRELDSYGVSDFYKRLIDKAKTVEGVEALKDAILKALP | 589 |
| | LAQAKEAANRELDSYGVSDFYKRLIDKAKTVEGVEALKDAILKALP | 590 |
| | LAEAKEAANRELDAYGVSDFYKRLIDKAKTVEGVEALKDAILKALP | 591 |
| | LAEAKVLANRELDKYGVSDFYKRLIDKAKTVEGVEALKDAILKALP | 592 |

For example, in preferred engineered polypeptide embodiments the ABD2 comprises LAEAKEAANAELDSYGVSDFYKRLIDKAKTVEGVEALKDAILAALP (SEQ ID NO:313), and its N-terminally extended ABD2 sequence forms including SLAEAKEAANAELDSYGVSDFYKRLIDKAKTVEGVEALKDAILAALP (SEQ ID NO:500) and GSLAEAKEAANAELDSYGVSDFYKRLIDKAKTVEGVEALKDAILAALP (SEQ ID NO:463; PEP07986). The serine in position 2 is capping the sequence, raising Tm approximately 2° C. compared to having a glycine or an alanine in this position. An alanine can also immediately precede the serine as in AGSLAEAKEAANAELDSYGVSDFYKRLIDKAKTVEGVEALKDAILAALP (SEQ ID NO. 619). Also preferred are the corresponding polypeptides where the C-terminal proline, glycine or both is absent in each of the above ABD2 sequences. Accordingly, also preferred are sequences where the ABD2 includes the des-proline forms, which can improve yields compared to the parent forms: LAEAKEAANAELDSYGVSDFYKRLIDKAKTVEGVEALKDAILAAL (SEQ ID NO:313), and its N-terminally extended ABD sequence forms including SLAEAKEAANAELDSYGVSDFYKRLIDKAKTVEGVEALKDAILAAL (SEQ ID NO:601) and GSLAEAKEAANAELDSYGVSDFYKRLIDKAKTVEGVEALKDAILAAL (SEQ ID NO:599). Also preferred is the des-Leu45 form of each ABD.

In preferred engineered polypeptide embodiments where Cys-conjugation is desired the preferred ABD2 can comprise LAEAKEAANAELDCYGVSDFYKRLIDKAKTVEGVEALKDAILAALPG (SEQ ID NO:501) and its N-terminally extended ABD2 sequence forms including SLAEAKEAANAELDCYGVSDFYKRLIDKAKTVEGVEALKDAILAALPG (SEQ ID NO:502) and GSLAEAKEAANAELDCYGVSDFYKRLIDKAKTVEGVEALKDAILAALPG (SEQ ID NO:448; PEP08185). Also preferred are the polypeptides where the C-terminal proline or glycine or both are absent in each of the above ABD2 sequences.

Binding to Albumin.

Serum albumin is the most abundant protein in mammalian sera (40 g/L; approximately 0.7 mM in humans) where it binds a variety of molecules including but not limited to lipids and bilirubin (Peters T, 1985, Advances in Protein Chemistry 37:161). It has been observed that the half-life of serum albumin is directly proportional to the size of the animal, where for example human serum albumin (HSA) has a half-life of 19 days and rabbit serum albumin has a half-life of about 5 days (McCurdy T R et al., J. Lab. Clin. Med. 143:115, 2004). Human serum albumin is widely distributed throughout the body, in particular in the intestinal and blood compartments, where it is mainly involved in the maintenance of osmolarity. Structurally, albumins are single-chain proteins including three homologous domains and totaling 584 or 585 amino acids (Dugaiczyk L et al., 1982, Proc. Natl. Acad. Sci. USA 79:71). Albumins contain 17 disulfide bridges and a single reactive thiol, C34, but lack N-linked and O-linked carbohydrate moieties (Peters, 1985, Id.; Nicholson J P et al., 2000, Br J Anaesth 85:599). The lack of glycosylation simplifies recombinant expression of albumin. This property of albumin, together with the fact that its three-dimensional structure is known (He, X M and Carter, D C, Nature 358:209 1992), has made it an attractive candidate for use in recombinant fusion proteins. Such fusion proteins generally combine a therapeutic protein (which would be rapidly cleared from the body upon administration of the protein per se) and a plasma protein (which exhibits a natural slow clearance) in a single polypeptide chain (Sheffield W P, Curr. Drug Targets Cardiovacs. Haematol. Disord. 1:1 2001). Such fusion proteins may provide clinical benefits in requiring less frequent injection and higher levels of therapeutic protein in vivo. However, the engineered polypeptides herein are not conjugated to albumin, but instead contain motifs that allow non-covalent binding to albumin.

Albumin Half-Life.

It has been observed that the half-life of albumin in different species generally adheres to allometric scaling based on animal weight. For example, the albumin half-life in mouse, rat, rabbit and human has been estimated as 1, 1.9, 5.6 and 19 days, respectively. Indeed, power fitting analysis (Davies & Morris, 1993, Pharm. Res. (N.Y.) 10:1093-1095) provides the equation:

Albumin half-life (days)=3.75*body weight(kg)^{0.368}

### Further Embodiments

It is understood that each of the polypeptides disclosed herein are also contemplated to include a methionine at the N-terminus in frame with the naturally-occurring first amino acid thereof, e.g., Met-exendin-4, which is exendin-4 with an added N-terminal methionine. It is further understood that where a C-terminal Gly appears in a engineered polypeptide sequence set forth herein, the residue may be lost during subsequent amidation. Some embodiments are intermediates in synthesis, for example, such as those having a “His tag” which is used for affinity purification as is known in the art, and that can optionally be subsequently removed to yield a mature engineered polypeptide suitable for therapeutic use.

In some embodiments of any of the engineered polypeptides described herein, an exendin analog can have at least 70%, for example 70%, 75%, 80%, 85%, 90%, 95%, 98% or even higher, sequence identity relative to a parent exendin sequence. In some embodiments, the parent exendin is exendin-4, and the exendin analog may have at least 70%, for example 70%, 75%, 80%, 85%, 90%, 95%, 98% or even higher, sequence identity relative to exendin-4. As known the art, GLP-1 (glucagon-like peptide 1) is not an exendin; and the sequence of GLP-1 is specifically excluded from exendin sequences suitable for the engineered polypeptides described herein.

In some embodiments, compounds are provided having a linker, for example L1, as described herein, covalently linking a polypeptide hormone domain with an ABD peptide. In some embodiments, a first linker (L1) covalently links HD1 within the engineered polypeptide. In some embodiments, L1 is a bond. In some embodiments, the polypeptide hormone domain (e.g., HD1 as described herein) can be covalently linked to the ABD peptide via a peptide linker. Any linker is optional; i.e., any linker may simply be a bond. When present the chemical structure of a linker is not critical because it serves mainly a spacer function. In one embodiment the linker includes from 1 to 30 or less amino acids linked by peptide bonds. The amino acids can be selected from the 20 naturally occurring (i.e., physiological) amino acids. Alternatively, non-natural amino acids can be incorporated either by chemical synthesis, post-translational chemical modification or by in vivo incorporation by recombinant expression in a host cell. Some of these amino acids may be glycosylated. In another embodiment the 1 to 30 or less amino acids are selected from glycine, alanine, proline, asparagine, glutamine, and lysine, and further from aspartate and glutamate. In a further embodiment the linker is made up of a majority of amino acids that are sterically unhindered, such as glycine, alanine and/or serine. “Sterically unhindered” refers, in the customary sense, to a amino acid having a small side chain, e.g., 0-2 non-hydrogen atoms, such that steric hinderance is minimized relative to amino acids having larger side chains, e.g., Leu, Trp, Tyr, Phe, and the like. Polyglycines are particularly useful, e.g. (Gly)₃, (Gly)₄ (SEQ ID NO:125), (Gly)₅ (SEQ ID NO:126), as are polyalanines, poly(Gly-Ala) and poly(Gly-Ser). Charged polyglycines can be useful, and include e.g., poly (Glyₙ-Glu) (SEQ ID NO:127), poly(Glyₙ-Lys) (SEQ ID NO:128), poly(Glyₙ-Asp) (SEQ ID NO:129), and poly(Glyₙ-Arg) (SEQ ID NO:130) motifs (where n can be 1 to 6). Other specific examples of linkers are (Gly)₃Lys(Gly)₄ (SEQ ID NO:131); (Gly)₃AsnGlySer(Gly)₂ (SEQ ID NO:132); (Gly)₃Cys(Gly)₄ (SEQ ID NO:133); and GlyProAsnGlyGly (SEQ ID NO:134). Combinations of Gly and Ala are particularly useful as are combination of Gly and Ser. Thus, in a further embodiment the peptide linker is selected from the group consisting of a glycine rich peptide, e.g., Gly-Gly-Gly; the sequences [Gly-Ser]ₙ (SEQ ID NO:135), [Gly-Gly-Ser]ₙ (SEQ ID NO:136), [Gly-Gly-Gly-Ser]ₙ (SEQ ID NO:137) and [Gly-Gly-Gly-Gly-Ser]ₙ (SEQ ID NO:138), where n is 1, 2, 3, 4, 5 or 6, for example [Gly-Gly-Gly-Gly Ser]₃. “Glycine rich peptide” refers to a polypeptide which includes a plurality of glycine residues, preferably a majority of glycine residues, more preferably a preponderance of glycine residues.

In one aspect with any of the ABD sequences disclosed herein, i.e. both ABD1 and ABD2, the linker to exendin-4 or exendin analog sequence is a glycine including linker as disclosed herein, for example G, GGG, GGS, GGGS (SEQ ID NO:192), TGGGGAS (SEQ ID NO:193), TGGGGGAS (SEQ ID NO:194), or TGGGGSAS (SEQ ID NO:195).

In certain embodiments, charged linkers may be used. Such charges linkers may be contain a significant number of acidic residues (e.g., Asp, Glu, and the like), or may contain a significant number of basic residues (e.g., Lys, Arg, and the like), such that the linker has a pI lower than 7 or greater than 7, respectively. As understood by the artisan, and all other things being equal, the greater the relative amount of acidic or basic residues in a given linker, the lower or higher, respectively, the pI of that linker will be. Such linkers may impart advantageous properties to the engineered polypeptides disclosed herein, such as modifying the peptides pI (isoelectric point) which can in turn improve solubility and/or stability characteristics of such polypeptides at a particular pH, such as at physiological pH (e.g., between pH 7.2 and pH 7.6, inclusive), or in a pharmaceutical composition including such polypeptides. As is known in the art, solubility for a peptide can be improved by formulation in a composition having a pH that is at least or more than plus or minus one pH unit from the pI of the peptide.

For example, an “acidic linker” is a linker that has a pI of less than 7; between 6 and 7, inclusive; between 5 and 6, inclusive; between 4 and 5, inclusive; between 3 and 4, inclusive; between 2 and 3, inclusive; or between 1 and 2, inclusive. Similarly, a “basic linker” is a linker that has a pI of greater than 7; between 7 and 8, inclusive; between 8 and 9, inclusive; between 9 and 10, inclusive; between 10 and 11, inclusive; between 11 and 12 inclusive, or between 12 and 13, inclusive. In certain embodiments, an acidic linker will contain a sequence that is selected from the group of [Gly-Glu]ₙ (SEQ ID NO:139); [Gly-Gly-Glu]ₙ (SEQ ID NO:140); [Gly-Gly-Gly-Glu]ₙ (SEQ ID NO:141); [Gly-Gly-Gly-Gly-Glu]ₙ (SEQ ID NO:142), [Gly-Asp]ₙ (SEQ ID NO:143); [Gly-Gly-Asp]ₙ (SEQ ID NO:144); [Gly-Gly-Gly-Asp]ₙ (SEQ ID NO:145); [Gly-Gly-Gly-Gly-Asp]ₙ (SEQ ID NO:146), where n is 1, 2, 3, 4, 5, 6, 7, 8, 9, 10, or more; for example, [Gly-Gly-Glu]₆. In certain embodiments, a basic linker will contain a sequence that is selected from the group of [Gly-Lys]ₙ (SEQ ID NO:147); [Gly-Gly-Lys]ₙ (SEQ ID NO:148); [Gly-Gly-Gly-Lys]ₙ (SEQ ID NO:149); [Gly-Gly-Gly-Gly-Lys]ₙ (SEQ ID NO:150), [Gly-Arg]ₙ (SEQ ID NO:151); [Gly-Gly-Arg]ₙ (SEQ ID NO:152); [Gly-Gly-Gly-Arg]ₙ (SEQ ID NO:153); [Gly-Gly-Gly-Gly-Arg]ₙ (SEQ ID NO:154) where n is 1, 2, 3, 4, 5, 6, 7, 8, 9, 10, or more; for example, [Gly-Gly-Lys]₆.

Additionally, linkers may be prepared which possess certain structural motifs or characteristics, such as an alpha helix. For example, such a linker may contain a sequence that is selected from the group of [Glu-Ala-Ala-Ala-Lys]ₙ (SEQ ID NO:155), where n is 1, 2, 3, 4, 5, 6, 7, 8, 9, 10, or more; for example, [Glu-Ala-Ala-Ala-Lys]₃, [Glu-Ala-Ala-Ala-Lys]₄, or [Glu-Ala-Ala-Ala-Lys]₅. One in the art can readily determine helix content of any particular linker sequence.

A biocompatible linker other than a peptide linker may be used to covalently attach the C-terminus of an exendin to the N-terminus of the ABD or ABM sequence. The linker can be a biocompatible polymer, preferably water soluble, and more preferably about 50 kD to about 5000 kD, or about 50 KD to 500 kD, or about 100 kD to 500 kD. An exemplary biocompatible, water soluble polymer linker is a PEG linker, such as —(CH₂—CH₂—O)ₙ— where n is such that the PEG linker can have a molecular weight of 100 to 5000 kD, preferably 100 to 500 kD. Such a linker may be —NH—CH₂—CH₂—(O—CH₂—CH₂)ₙ—O—CH₂—CO—, where n is such that the PEG linker molecular weight is 100 kD to 5000 kD, preferably 10 kD to 500 kD. Other biocompatible polymers can be used, such as including but not limited to polysaccharides, polypropylene glycol, and co-polymers of propylene and ethylene glycols. Typically such a linker will include a reactive group at each end that can be the same or different reactive group. Such linkers with reactive groups are known and available. Preferably the reactive group is reactive with either an N-terminal amino or C-terminal carboxy group of a peptide. For example, a reactive group can be an a butylaldehyde, a propionaldehyde, an aldehyde, a succinimide or a maleimide moiety, as is known in the art. Less preferred are alkyl linkers such as —NH—(CH₂)ₙ—C(O)—, wherein n=2-20, and which can be further substituted by any group that does not sterically-hinder peptide function, such as a lower alkyl (e.g., C₁-C₆), lower acyl, halogen, CN, and NH₂.

It is also to be understood that linkers suitable for use in accordance with the invention may possess one or more of the characteristics and motifs described above and herein. For example, a linker may include an acidic linker as well as a structural motif, such as an alpha helix. Similarly, a linker may include a basic linker and a structural motif, such as an alpha helix. A linker may include an acidic linker, a basic linker, and a structural motif, such as an alpha helix. Additionally, it is also to be understood that engineered polypeptides in accordance with the invention may possess more than one linker, and each such linker may possess one or more of the characteristics described herein.

The linkers described herein are exemplary, and linkers within the scope of this invention may be much longer and may include other residues. In one embodiment, expressly excluded are engineered polypeptides in which the exendin sequence is linked directly to the ABD sequence without a linker.

In some embodiments, the engineered polypeptide includes an ABD sequence at the C-terminal, and a HD1 sequence at the N-terminal. In certain preferred embodiments, the N-terminal is an exendin sequence, an exendin fragment sequence or an exendin analog sequence. Further to embodiments which include an ABD and a HD1, the engineered polypeptide can have the structure HD1-ABD.

It is understood that absent an express indication of the N-terminus and/or C-terminus of a engineered polypeptide set forth herein, the engineered polypeptide is to be read in the N-terminus to C-terminus orientation. For example, where HD1 has the sequence of an exendin compound or analog thereof, the terms HD1-ABD, HD1-L1-ABD, HD1-ABD, and the like mean, in the absence of an express indication of the N-terminus and/or the C-terminus, that the exendin sequence or analog thereof resides at the N-terminus of the engineered polypeptide, and the ABD resides at the C-terminus Conversely, if the N-terminus and/or C-terminus is expressly indicated, then the engineered polypeptide is to be read according to the express indication of the termini. For example, the terms HD1_{C-term}-ABD, HD1-L1-ABD_{N-term} and the like mean that the ABD resides at the N-terminus of the engineered polypeptide, and HD1 resides at the C-terminus

In some embodiments, the engineered polypeptide described herein has an affinity for serum albumin which is different than the affinity of the ABD polypeptide alone, i.e., in the absence of a fused hormone domain. In order to obtain effective association, the engineered polypeptide can have a binding affinity for serum albumin such that the dissociation constant K_{D} is, for example, less than about 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M or even 10⁻¹⁵ M. In some embodiments, the affinity is not excessively tight such that the engineered polypeptide can dissociate from the albumin and elicit a biological response, for example binding to a receptor, for example, an GLP-1 receptor. The affinity can be measured as described in PCT Published Appl. No. WO 2009/016043, preferably to human serum albumin, which is incorporated herein by reference in its entirety and for all purposes, including without limitation assays and synthesis methods.

In some embodiments, a engineered polypeptide described herein is superior to a corresponding compound having a different moiety that can extend plasma half-life (e.g., PEG or of Fc or albumin) conjugated with a hormone domain(s). In this context, the term “superior” refers to a variety of functional properties which could be weighed in the evaluation of a treatment for a disease or disorder. For example, the engineered polypeptide described herein could require less biologically active (hormone domain) component, for example 1×, 2×, 3×, 4×, 5×, or even less, than the corresponding compound having a different moiety conjugated with the hormone domain(s). For further example, the engineered polypeptide described herein could have higher potency, for example, 1.5×, 2×, 3×, 4×, 5×, 10×, 20×, 50×, or even higher potency.

Engineered polypeptide compounds contemplated herein include the compounds as set forth in Table 2 following. One preferred compound is Cmpd 31.

TABLE 2
Selected exemplary engineered polypeptides
having an ABD1

| Cmpd | Sequence |
|---|---|
| 5 | HGEGTFTSDLSKQLEEEAVRLFIEWLKNGGPSSGAPPPSTGGGGS |
| | ASLAEAKVLANRELDKYGVSDFYKRLINKAKTVEGVEALKLHILA |
| | ALP (SEQ ID NO: 40) |
| 6 | HGEGTFTSDLSKQLEEEAVRLFIEWLKNGGPSSGAPPPSTGGGGS |
| | GGGSGGGSGGGSASLAEAKVLANRELDKYGVSDFYKRLINKAKTV |
| | EGVEALKLHILAALP (SEQ ID NO: 41) |
| 7 | HGEGTFTSDLSKQLEEEAVRLFIEWLKQGGPSKEIISTGGGGGAS |
| | LAEAKVLANRELDKYGVSDFYKRLINKAKTVEGVEALKLHILAAL |
| | P (SEQ ID NO: 42) |
| 8 | HGEGTFTSDLSKQLEEEAVRLFIEWLKQGGPSKEIISTGGGGSGG |
| | GSGGGSGGGSASLAEAKVLANRELDKYGVSDFYKRLINKAKTVEG |
| | VEALKLHILAALP (SEQ ID NO: 43) |
| 10 | HGEGTFTSDLSKQMEEEAVRLFIEWLKNTGGGGSASLAEAKVLAN |
| | RELDKYGVSDFYKRLINKAKTVEGVEALKLHILAALP (SEQ ID |
| | NO: 51) |
| 15 | HGEGTFTSDLSKQLEEEAVRLFIEWLKQGGPSKEIISTGGGGSAS |
| | LAEAKVLANRELDKYGVSDFYKRLINKAKTVEGVEALKLHILAAL |
| | P (SEQ ID NO: 163) |
| 21 | HGEGTFTSDLSKQMEEEAVRLFIEWLKNGGPSSGAPPPSGGSLAE |
| | AKVLANRELDKYGVSDFYKRLINKAKTVEGVEALKLHILAALP |
| | (SEQ ID NO: 99) |
| 23 | HGEGTFTSDLSKQMEEEAVRLFIEWLKNGGPSSGAPPPSGGSLSE |
| | AKEMAIRELDANGVSDFYKDKIDDAKTVEGVVALKDLILNSLP |
| | (SEQ ID NO: 169) |
| 24 | HGEGTFTSDLSKQMEEEAVRLFIEWLKNGGPSSGAPPPSGGSLAK |
| | AKADAIEILKKYGIGDYYIKLINNGKTAEGVTALKDEILASLP |
| | (SEQ ID NO: 170) |
| 31 | HGEGTFTSDLSKQLEEEAVRLFIEWLKNGGPSSGAPPPSGGSLAE |
| | AKVLANRELDKYGVSDFYKRLINKAKTVEGVEALKLHILAALP |
| | (SEQ ID NO: 95) |
| 32 | HGEGTFTSDLSKQMEEEAVRLFIEWLKNGGSLAEAKVLANRELDK |
| | YGVSDFYKRLINKAKTVEGVEALKLHILAALP (SEQ ID NO: |
| | 97) |
| 33 | HGEGTFTSDLSKQLEEEAVRLFIEWLKQGGPSKEIISGGSLAEAK |
| | VLANRELDKYGVSDFYKRLINKAKTVEGVEALKLHILAALP |
| | (SEQ ID NO: 96) |
| 34 | HGEGTFTSDLSKQMEEEAVRLFIEWLKNGGPSSGAPPPSTGGGGS |
| | ASLAEAKVLANRELDKYGVSDFYKRLINKAKTVEGVEALKLHILA |
| | ALP (SEQ ID NO: 55); |

Additional polypeptide compounds contemplated herein include the compounds as set forth in Table 3A following:

TABLE 3A
Selected exemplary engineered polypeptides
with an ABD1

| Cmpd | Sequence |
|---|---|
| 9 | HGEGTFTSDLSKQMEEEAVRLFIEWLKNTGGGGSGGGSGGGSGGG |
| | SASLAEAKVLANRELDKYGVSDFYKRLINKAKTVEGVEALKLHIL |
| | AALP (SEQ ID NO: 53) |
| 11 | HGEGTFTSDLSKQMEEEAVRLFIEWLKNTGGGGSGGGSGGGSGGG |
| | SASLAEAKVLANRELDKYGVSDYYKNIINRAKTVEGVRALKLHIL |
| | AALP (SEQ ID NO: 62) |
| 12 | HGEGTFTSDLSKQMEEEAVRLFIEWLKNTGGGGSASLAEAKVLAN |
| | RELDKYGVSDYYKNIINRAKTVEGVRALKLHILAALP (SEQ ID |
| | NO: 67) |
| 19 | HGEGTFTSDLSKQMEEEAVRLFIEWLKNGGPSSGAPPPSTGGGGS |
| | ASYGVSDFYKRLINKAKTVEGVEALKLHILAALP (SEQ ID |
| | NO: 166) |
| 20 | HGEGTFTSDLSKQMEEEAVRLFIEWLKNGGPSSGAPPPSGYGVSD |
| | FYKRLINKAKTVEGVEALKLHILAALP (SEQ ID NO: 167) |

Additional engineered polypeptide compounds contemplated herein, having a variety of HD1, L1 and ABD components, include the compounds having the structure of any of the engineered polypeptides of the tables and listing herein, including those disclosed in Table 3B following.

TABLE 3B
Selected exemplary engineered polypeptides
having an ABD1
Sequence

HGEGTFTSDLSKQMEEEAVRLFIEWLKNTGGGGSASLAEAKVLANRELDK
YGVSDFYKRLINKAKTVEGVEALKLHILAALP (SEQ ID NO: 51)
HGEGTFTSDLSKQLEEEAVRLFIEWLKNTGGGGSASLAEAKVLANRELDK
YGVSDFYKRLINKAKTVEGVEALKLHILAALP (SEQ ID NO: 52)
HGEGTFTSDLSKQMEEEAVRLFIEWLKNTGGGGSGGGSGGGSGGGSASLA
EAKVLANRELDKYGVSDFYKRLINKAKTVEGVEALKLHILAALP (SEQ
ID NO: 53)
HGEGTFTSDLSKQLEEEAVRLFIEWLKNTGGGGSGGGSGGGSGGGSASLA
EAKVLANRELDKYGVSDFYKRLINKAKTVEGVEALKLHILAALP (SEQ
ID NO: 54)
HGEGTFTSDLSKQMEEEAVRLFIEWLKNGGPSSGAPPPSTGGGGSASLAE
AKVLANRELDKYGVSDFYKRLINKAKTVEGVEALKLHILAALP (SEQ
ID NO: 55)
HGEGTFTSDLSKQMEEEAVRLFIEWLKNGGPSSGAPPPSTGGGGSGGGSG
GGSGGGSASLAEAKVLANRELDKYGVSDFYKRLINKAKTVEGVEALKLHI
LAALP (SEQ ID NO: 56)
HGEGTFTSDLSKQMEEEAVRLFIEWLKQGGPSKEIISTGGGGSASLAEAK
VLANRELDKYGVSDFYKRLINKAKTVEGVEALKLHILAALP (SEQ ID
NO: 57)
HGEGTFTSDLSKQMEEEAVRLFIEWLKQGGPSKEIISTGGGGSGGGSGGG
SGGGSASLAEAKVLANRELDKYGVSDFYKRLINKAKTVEGVEALKLHILA
ALP (SEQ ID NO: 58)
HGEGTFTSDLSKQMEEEAVRLFIEWLKNGGPSSGAPPSKKKKKKTGGGGS
ASLAEAKVLANRELDKYGVSDFYKRLINKAKTVEGVEALKLHILAALP
(SEQ ID NO: 59)
HGEGTFTSDLSKQMEEEAVRLFIEWLKNGGPSSGAPPSKKKKKKTGGGGS
GGGSGGGGGGSASLAEAKVLANRELDKYGVSDFYKRLINKAKTVEGVEAL
KLHILAALP (SEQ ID NO: 60)
HGEGTFTSDLSKQLEEEAVRLFIEWLKNGGPSSGAPPPSGGGLAEAKVLA
NRELDKYGVSDFYKRLINKAKTVEGVEALKLHILAALP (SEQ ID NO:
61)
HGEGTFTSDLSKQMEEEAVRLFIEWLKNTGGGGSGGGSGGGSGGGSASLA
EAKVLANRELDKYGVSDYYKNIINRAKTVEGVRALKLHILAALP (SEQ
ID NO: 62)
HGEGTFTSDLSKQLEEEAVRLFIEWLKNGGPSSGAPPPSTGGGGSASLAE
AKVLANRELDKYGVSDYYKNIINRAKTVEGVRALKLHILAALP (SEQ
ID NO: 63)
HGEGTFTSDLSKQLEEEAVRLFIEWLKNGGPSSGAPPPSTGGGGSGGGSG
GGSGGGSASLAEAKVLANRELDKYGVSDYYKNIINRAKTVEGVRALKLHI
LAALP (SEQ ID NO: 64)
HGEGTFTSDLSKQLEEEAVRLFIEWLKQGGPSKEIISTGGGGSASLAEAK
VLANRELDKYGVSDYYKNIINRAKTVEGVRALKLHILAALP (SEQ ID
NO: 65)
HGEGTFTSDLSKQLEEEAVRLFIEWLKQGGPSKEIISTGGGGSGGGSGGG
SGGGSASLAEAKVLANRELDKYGVSDYYKNIINRAKTVEGVRALKLHILA
ALP (SEQ ID NO: 66)
HGEGTFTSDLSKQMEEEAVRLFIEWLKNTGGGGSASLAEAKVLANRELDK
YGVSDYYKNIINRAKTVEGVRALKLHILAALP (SEQ ID NO: 67)
HGEGTFTSDLSKQLEEEAVRLFIEWLKNTGGGGSASLAEAKVLANRELDK
YGVSDYYKNIINRAKTVEGVRALKLHILAALP (SEQ ID NO: 68)
HGEGTFTSDLSKQLEEEAVRLFIEWLKNTGGGGSGGGSGGGSGGGSASLA
EAKVLANRELDKYGVSDYYKNIINRAKTVEGVRALKLHILAALP (SEQ
ID NO: 70)
HGEGTFTSDLSKQMEEEAVRLFIEWLKNGGPSSGAPPPSTGGGGSASLAE
AKVLANRELDKYGVSDYYKNIINRAKTVEGVRALKLHILAALP (SEQ
ID NO: 71)
HGEGTFTSDLSKQMEEEAVRLFIEWLKNGGPSSGAPPPSTGGGGSGGGSG
GGSGGGSASLAEAKVLANRELDKYGVSDYYKNIINRAKTVEGVRALKLHI
LAALP (SEQ ID NO: 72)
HGEGTFTSDLSKQMEEEAVRLFIEWLKQGGPSKEIISTGGGGSASLAEAK
VLANRELDKYGVSDYYKNIINRAKTVEGVRALKLHILAALP (SEQ ID
NO: 73)
HGEGTFTSDLSKQMEEEAVRLFIEWLKQGGPSKEIISTGGGGSGGGSGGG
SGGGSASLAEAKVLANRELDKYGVSDYYKNIINRAKTVEGVRALKLHILA
ALP (SEQ ID NO: 74)
HGEGTFTSDLSKQMEEEAVRLFIEWLKNGGPSSGAPPSKKKKKKTGGGGS
ASLAEAKVLANRELDKYGVSDYYKNIINRAKTVEGVRALKLHILAALP
(SEQ ID NO: 75)
HGEGTFTSDLSKQMEEEAVRLFIEWLKNGGPSSGAPPSKKKKKKTGGGGS
GGGSGGGSGGGSASLAEAKVLANRELDKYGVSDYYKNIINRAKTVEGVRA
LKLHILAALP (SEQ ID NO: 76)
HGEGTFTSDLSKQLEEEAVRLFIEWLKNGGPSSGAPPPSGGGLAEAKVLA
NRELDKYGVSDYYKNIINRAKTVEGVRALKLHILAALP (SEQ ID NO:
77)
HGEGTFTSDLSKQLEEEAVRLFIEWLKNGGPSSGAPPPSGGGLAEAKVLA
NRELDKYGVSDFYKRLINKAKTVEGVEALKLHILAALP (SEQ ID NO:
78)
HGEGTFTSDLSKQLEEEAVRLFIEWLKQGGPSKEIISGGGLAEAKVLANR
ELDKYGVSDFYKRLINKAKTVEGVEALKLHILAALP (SEQ ID NO:
79)
HGEGTFTSDLSKQMEEEAVRLFIEWLKNGGGLAEAKVLANRELDKYGVSD
FYKRLINKAKTVEGVEALKLHILAALP (SEQ ID NO: 80)
HGEGTFTSDLSKQLEEEAVRLFIEWLKNGGGLAEAKVLANRELDKYGVSD
FYKRLINKAKTVEGVEALKLHILAALP (SEQ ID NO: 81)
HGEGTFTSDLSKQMEEEAVRLFIEWLKNGGPSSGAPPPSGGGLAEAKVLA
NRELDKYGVSDFYKRLINKAKTVEGVEALKLHILAALP (SEQ ID NO:
82)
HGEGTFTSDLSKQMEEEAVRLFIEWLKQGGPSKEIISGGGLAEAKVLANR
ELDKYGVSDFYKRLINKAKTVEGVEALKLHILAALP (SEQ ID NO:
83)
HGEGTFTSDLSKQMEEEAVRLFIEWLKNGGPSSGAPPSKKKKKKGGGLAE
AKVLANRELDKYGVSDFYKRLINKAKTVEGVEALKLHILAALP (SEQ
ID NO: 84)
HGEGTFTSDLSKQLEEEAVRLFIEWLKNGGPSSGAPPPSGGGLAEAKVLA
NRELDKYGVSDFYKRLINKAKTVEGVEALKLHILAALP (SEQ ID NO:
85)
HGEGTFTSDLSKQMEEEAVRLFIEWLKNGGGLAEAKVLANRELDKYGVSD
YYKNIINRAKTVEGVRALKLHILAALP (SEQ ID NO: 86)
HGEGTFTSDLSKQLEEEAVRLFIEWLKNGGPSSGAPPPSGGGLAEAKVLA
NRELDKYGVSDYYKNIINRAKTVEGVRALKLHILAALP (SEQ ID NO:
87)
HGEGTFTSDLSKQLEEEAVRLFIEWLKQGGPSKEIISGGGLAEAKVLANR
ELDKYGVSDYYKNIINRAKTVEGVRALKLHILAALP (SEQ ID NO:
88)
HGEGTFTSDLSKQLEEEAVRLFIEWLKNGGGLAEAKVLANRELDKYGVSD
YYKNIINRAKTVEGVRALKLHILAALP (SEQ ID NO: 89)
HGEGTFTSDLSKQLEEEAVRLFIEWLKNGGGLAEAKVLANRELDKYGVSD
YYKNIINRAKTVEGVRALKLHILAALP (SEQ ID NO: 90)
HGEGTFTSDLSKQMEEEAVRLFIEWLKNGGPSSGAPPPSGGGLAEAKVLA
NRELDKYGVSDYYKNIINRAKTVEGVRALKLHILAALP (SEQ ID NO:
91)
HGEGTFTSDLSKQMEEEAVRLFIEWLKQGGPSKEIISGGGLAEAKVLANR
ELDKYGVSDYYKNIINRAKTVEGVRALKLHILAALP (SEQ ID NO:
92)
HGEGTFTSDLSKQMEEEAVRLFIEWLKNGGPSSGAPPSKKKKKKGGGLAE
AKVLANRELDKYGVSDYYKNIINRAKTVEGVRALKLHILAALP (SEQ
ID NO: 93)
HGEGTFTSDLSKQLEEEAVRLFIEWLKNGGPSSGAPPPSGGSLAEAKVLA
NRELDKYGVSDYYKNIINRAKTVEGVRALKLHILAALP (SEQ ID NO:
94)
HGEGTFTSDLSKQLEEEAVRLFIEWLKNGGPSSGAPPPSGGSLAEAKVLA
NRELDKYGVSDFYKRLINKAKTVEGVEALKLHILAALP (SEQ ID NO:
95)
HGEGTFTSDLSKQLEEEAVRLFIEWLKQGGPSKEIISGGSLAEAKVLANR
ELDKYGVSDFYKRLINKAKTVEGVEALKLHILAALP (SEQ ID NO:
96)
HGEGTFTSDLSKQMEEEAVRLFIEWLKNGGSLAEAKVLANRELDKYGVSD
FYKRLINKAKTVEGVEALKLHILAALP (SEQ ID NO: 97)
HGEGTFTSDLSKQLEEEAVRLFIEWLKNGGSLAEAKVLANRELDKYGVSD
FYKRLINKAKTVEGVEALKLHILAALP (SEQ ID NO: 98)
HGEGTFTSDLSKQMEEEAVRLFIEWLKNGGPSSGAPPPSGGSLAEAKVLA
NRELDKYGVSDFYKRLINKAKTVEGVEALKLHILAALP (SEQ ID NO:
99)
HGEGTFTSDLSKQMEEEAVRLFIEWLKQGGPSKEIISGGSLAEAKVLANR
ELDKYGVSDFYKRLINKAKTVEGVEALKLHILAALP (SEQ ID NO:
100)
HGEGTFTSDLSKQMEEEAVRLFIEWLKNGGPSSGAPPSKKKKKKGGSLAE
AKVLANRELDKYGVSDFYKRLINKAKTVEGVEALKLHILAALP (SEQ
ID NO: 101)
HGEGTFTSDLSKQMEEEAVRLFIEWLKNGGSLAEAKVLANRELDKYGVSD
YYKNIINRAKTVEGVRALKLHILAALP (SEQ ID NO: 102)
HGEGTFTSDLSKQLEEEAVRLFIEWLKNGGPSSGAPPPSGGSLAEAKVLA
NRELDKYGVSDYYKNIINRAKTVEGVRALKLHILAALP (SEQ ID NO:
103)
HGEGTFTSDLSKQLEEEAVRLFIEWLKQGGPSKEIISGGSLAEAKVLANR
ELDKYGVSDYYKNIINRAKTVEGVRALKLHILAALP (SEQ ID NO:
104)
HGEGTFTSDLSKQLEEEAVRLFIEWLKNGGSLAEAKVLANRELDKYGVSD
YYKNIINRAKTVEGVRALKLHILAALP (SEQ ID NO: 105)
HGEGTFTSDLSKQLEEEAVRLFIEWLKNGGSLAEAKVLANRELDKYGVSD
YYKNIINRAKTVEGVRALKLHILAALP (SEQ ID NO: 106)
HGEGTFTSDLSKQMEEEAVRLFIEWLKNGGPSSGAPPPSGGSLAEAKVLA
NRELDKYGVSDYYKNIINRAKTVEGVRALKLHILAALP (SEQ ID NO:
107)
HGEGTFTSDLSKQMEEEAVRLFIEWLKQGGPSKEIISGGSLAEAKVLANR
ELDKYGVSDYYKNIINRAKTVEGVRALKLHILAALP (SEQ ID NO:
108)
HGEGTFTSDLSKQMEEEAVRLFIEWLKNGGPSSGAPPSKKKKKKGGSLAE
AKVLANRELDKYGVSDYYKNIINRAKTVEGVRALKLHILAALP (SEQ
ID NO: 109)

Engineered polypeptide compounds contemplated herein include the compounds as set forth in Table 3C following. Preferred compounds are Cmpd 2-5, Cmpd 2-9 and Cmpd 2-11.

TABLE 3C
Selected exemplary engineered polypeptides
containing ABD sequences of the ABD2
sequence type.

| Cmpd | Sequence |
|---|---|
| 2-5 | HGEGTFTSDLSKQLEEEAVRLFIEWLKQGGPSKEIISTGGGGSAS |
| | GSLAEAKEAANAELDSYGVSDFYKRLIDKAKTVEGVEALKDAILA |
| | ALP (SEQ ID NO: 620) |
| 2-6 | HGEGTFTSDLSKQLEEEAVRLFIEWLKNGGPSSGAPPKSTGGGGS |
| | ASGSLAEAKEAANAELDSYGVSDFYKRLIDKAKTVEGVEALKDAI |
| | LAALP (SEQ ID NO: 621) |
| 2-7 | HGEGTFTSDLSKQLEEEAVRLFIEWLKNGGPSSGAPPPSTGGGGS |
| | ASGSLAEAKEAANAELDSYGVSDFYKRLIDKAKTVEGVEALKDAI |
| | LAAL (SEQ ID NO: 622) |
| 2-8 | HGEGTFTSDLSKQMEEEAVRLFIEWLKNGGPSSGAPPPSTGGGGS |
| | ASGSLAEAKEAANAELDSYGVSDFYKRLIDKAKTVEGVEALKDAI |
| | LAALP (SEQ ID NO: 623) |
| 2-9 | HGEGTFTSDLSKQMEEEAVRLFIEWLKNGGPSSGAPPPSGGSLAE |
| | AKEAANAELDSYGVSDFYKRLIDKAKTVEGVEALKDAILAALP |
| | (SEQ ID NO: 624) |
| 2-10 | HGEGTFTSDLSKQLEEEAVRLFIEWLKNGGPSSGAPPPSTGGGGS |
| | ASGSLAEAKEAANAELDSYGVSDFYKRLIDKAKTVEGVEALKDAI |
| | LAALP (SEQ ID NO: 625) |
| 2-11 | HGEGTFTSDLSKQLEEEAVRLFIEWLKNGGPSSGAPPPSGGSLAE |
| | AKEAANAELDSYGVSDFYKRLIDKAKTVEGVEALKDAILAALP |
| | (SEQ ID NO: 626) |
| | HGEGTFTSDLSKQLEEEAVRLFIEWLKQGGPSKEIISTGGGGSAS |
| | GSLAEAKEAANAELDSYGVSDFYKRLIDKAKTVEGVEALKDAILA |
| | AL (SEQ ID NO: 627) |
| | HGEGTFTSDLSKQLEEEAVRLFIEWLKNGGPSSGAPPKSTGGGGS |
| | ASGSLAEAKEAANAELDSYGVSDFYKRLIDKAKTVEGVEALKDAI |
| | LAAL (SEQ ID NO: 628) |
| | HGEGTFTSDLSKQLEEEAVRLFIEWLKNGGPSSGAPPPSTGGGGS |
| | ASGSLAEAKEAANAELDSYGVSDFYKRLIDKAKTVEGVEALKDAI |
| | LAA (SEQ ID NO: 629) |
| | HGEGTFTSDLSKQMEEEAVRLFIEWLKNGGPSSGAPPPSTGGGGS |
| | ASGSLAEAKEAANAELDSYGVSDFYKRLIDKAKTVEGVEALKDAI |
| | LAAL (SEQ ID NO: 630) |
| | HGEGTFTSDLSKQMEEEAVRLFIEWLKNGGPSSGAPPPSGGSLAE |
| | AKEAANAELDSYGVSDFYKRLIDKAKTVEGVEALKDAILAAL |
| | (SEQ ID NO: 631) |
| | HGEGTFTSDLSKQLEEEAVRLFIEWLKNGGPSSGAPPPSGGSLAE |
| | AKEAANAELDSYGVSDFYKRLIDKAKTVEGVEALKDAILAAL |
| | (SEQ ID NO: 632) |
| | HGEGTFTSDLSKQLEEEAVRLFIEWLKQGGPSKEIISTGGGGSAS |
| | GSLAEAKEAANAELDSYGVSDFYKRLIDKAKTVEGVEALKDAILA |
| | A (SEQ ID NO: 633) |
| | HGEGTFTSDLSKQLEEEAVRLFIEWLKNGGPSSGAPPKSTGGGGS |
| | ASGSLAEAKEAANAELDSYGVSDFYKRLIDKAKTVEGVEALKDAI |
| | LAA (SEQ ID NO: 634) |
| | HGEGTFTSDLSKQMEEEAVRLFIEWLKNGGPSSGAPPPSTGGGGS |
| | ASGSLAEAKEAANAELDSYGVSDFYKRLIDKAKTVEGVEALKDAI |
| | LAA (SEQ ID NO: 635) |
| | HGEGTFTSDLSKQMEEEAVRLFIEWLKNGGPSSGAPPPSGGSLAE |
| | AKEAANAELDSYGVSDFYKRLIDKAKTVEGVEALKDAILAA |
| | (SEQ ID NO: 636) |
| | HGEGTFTSDLSKQLEEEAVRLFIEWLKNGGPSSGAPPPSGGSLAE |
| | AKEAANAELDSYGVSDFYKRLIDKAKTVEGVEALKDAILAA |
| | (SEQ ID NO: 637) |

Additional engineered polypeptide compounds specifically contemplated herein as if set forth specifically, have any on an HD1 and an ABD component, optionally with any of the L1 sequences disclosed herein, and include the compounds having the structure of any of the engineered polypeptides of the tables and listing herein, including those disclosed in Table 3D following:

TABLE 3D
Selected exemplary engineered polypeptides containing
ABD sequences of the ABD2 type.

| Cmpd | Sequence |
|---|---|
| 2-5 | HGEGTFTSDLSKQLEEEAVRLFIEWLKQGGPSKEIISTGGGGSASGSLAE |
| | AKEAANAELDSYGVSDFYKRLIDKAKTVEGVEALKDAILAALP (SEQ ID |
| | NO: 620) |
| | HGEGTFTSDLSKQLEEEAVRLFIEWLKQGGPSKEIISTGGGGSASGSLAE |
| | AKEAANAELDSYGVSDFYKRLIDKAKTVEGVEALKDAILAAL (SEQ ID |
| | NO: 627) |
| | HGEGTFTSDLSKQLEEEAVRLFIEWLKQGGPSKEIISTGGGGSASSLAEA |
| | KEAANAELDSYGVSDFYKRLIDKAKTVEGVEALKDAILAALP (SEQ ID |
| | NO: 638) |
| | HGEGTFTSDLSKQLEEEAVRLFIEWLKQGGPSKEIISTGGGGSASGSLAE |
| | AKEAANAELDSYGVSDFYKRLIDKAKTVEGVEALKDAILAA (SEQ ID |
| | NO: 633) |
| | HGEGTFTSDLSKQLEEEAVRLFIEWLKQGGPSKEIISTGGGGSASSLAEA |
| | KEAANAELDSYGVSDFYKRLIDKAKTVEGVEALKDAILAAL (SEQ ID |
| | NO: 639) |
| | HGEGTFTSDLSKQLEEEAVRLFIEWLKQGGPSKEIISTGGGGSASLAEAK |
| | EAANAELDSYGVSDFYKRLIDKAKTVEGVEALKDAILAALP (SEQ ID |
| | NO: 640) |
| | HGEGTFTSDLSKQLEEEAVRLFIEWLKQGGPSKEIISTGGGGSASLAEAK |
| | EAANAELDSYGVSDFYKRLIDKAKTVEGVEALKDAILAAL (SEQ ID |
| | NO: 641) |
| | HGEGTFTSDLSKQLEEEAVRLFIEWLKQGGPSKEIISGGGGSLAEAKEAA |
| | NAELDSYGVSDFYKRLIDKAKTVEGVEALKDAILAALP (SEQ ID |
| | NO: 642) |
| | HGEGTFTSDLSKQLEEEAVRLFIEWLKQGGPSKEIISGGGGSLAEAKEAA |
| | NAELDSYGVSDFYKRLIDKAKTVEGVEALKDAILAAL (SEQ ID NO: 643) |
| | HGEGTFTSDLSKQLEEEAVRLFIEWLKQGGPSKEIISGGGSLAEAKEAAN |
| | AELDSYGVSDFYKRLIDKAKTVEGVEALKDAILAALP (SEQ ID NO: 644) |
| | HGEGTFTSDLSKQLEEEAVRLFIEWLKQGGPSKEIISGGGSLAEAKEAAN |
| | AELDSYGVSDFYKRLIDKAKTVEGVEALKDAILAAL (SEQ ID NO: 645) |
| | HGEGTFTSDLSKQLEEEAVRLFIEWLKQGGPSKEIISGGGLAEAKEAANA |
| | ELDSYGVSDFYKRLIDKAKTVEGVEALKDAILAALP (SEQ ID NO: 646) |
| | HGEGTFTSDLSKQLEEEAVRLFIEWLKQGGPSKEIISGGGLAEAKEAANA |
| | ELDSYGVSDFYKRLIDKAKTVEGVEALKDAILAAL (SEQ ID NO: 647) |
| | HGEGTFTSDLSKQLEEEAVRLFIEWLKQGGPSKEIISGGSLAEAKEAANA |
| | ELDSYGVSDFYKRLIDKAKTVEGVEALKDAILAALP (SEQ ID NO: 648) |
| | HGEGTFTSDLSKQLEEEAVRLFIEWLKQGGPSKEIISGGSLAEAKEAANA |
| | ELDSYGVSDFYKRLIDKAKTVEGVEALKDAILAAL (SEQ ID NO: 649) |
| | HGEGTFTSDLSKQLEEEAVRLFIEWLKQGGPSKEIISGSLAEAKEAANAE |
| | LDSYGVSDFYKRLIDKAKTVEGVEALKDAILAALP (SEQ ID NO: 650) |
| | HGEGTFTSDLSKQLEEEAVRLFIEWLKQGGPSKEIISGSLAEAKEAANAE |
| | LDSYGVSDFYKRLIDKAKTVEGVEALKDAILAAL (SEQ ID NO: 651) |
| | HGEGTFTSDLSKQLEEEAVRLFIEWLKQGGPSKEIISGLAEAKEAANAEL |
| | DSYGVSDFYKRLIDKAKTVEGVEALKDAILAALP (SEQ ID NO: 652) |
| | HGEGTFTSDLSKQLEEEAVRLFIEWLKQGGPSKEIISGLAEAKEAANAEL |
| | DSYGVSDFYKRLIDKAKTVEGVEALKDAILAAL (SEQ ID NO: 653) |
| 2-6 | HGEGTFTSDLSKQLEEEAVRLFIEWLKNGGPSSGAPPKSTGGGGSASGSL |
| | AEAKEAANAELDSYGVSDFYKRLIDKAKTVEGVEALKDAILAALP (SEQ |
| | ID NO: 621) |
| | HGEGTFTSDLSKQLEEEAVRLFIEWLKNGGPSSGAPPKSTGGGGSASGSL |
| | AEAKEAANAELDSYGVSDFYKRLIDKAKTVEGVEALKDAILAAL (SEQ |
| | ID NO: 628) |
| | HGEGTFTSDLSKQLEEEAVRLFIEWLKNGGPSSGAPPKSTGGGGSASSLA |
| | EAKEAANAELDSYGVSDFYKRLIDKAKTVEGVEALKDAILAALP (SEQ |
| | ID NO: 654) |
| | HGEGTFTSDLSKQLEEEAVRLFIEWLKNGGPSSGAPPKSTGGGGSASSLA |
| | EAKEAANAELDSYGVSDFYKRLIDKAKTVEGVEALKDAILAAL (SEQ ID |
| | NO: 655) |
| | HGEGTFTSDLSKQLEEEAVRLFIEWLKNGGPSSGAPPKSTGGGGSASLAE |
| | AKEAANAELDSYGVSDFYKRLIDKAKTVEGVEALKDAILAALP (SEQ ID |
| | NO: 656) |
| | HGEGTFTSDLSKQLEEEAVRLFIEWLKNGGPSSGAPPKSTGGGGSASLAE |
| | AKEAANAELDSYGVSDFYKRLIDKAKTVEGVEALKDAILAAL (SEQ ID |
| | NO: 657) |
| | HGEGTFTSDLSKQLEEEAVRLFIEWLKNGGPSSGAPPKSGGGGSLAEAK |
| | EAANAELDSYGVSDFYKRLIDKAKTVEGVEALKDAILAALP (SEQ ID |
| | NO: 658) |
| | HGEGTFTSDLSKQLEEEAVRLFIEWLKNGGPSSGAPPKSGGGGSLAEAK |
| | EAANAELDSYGVSDFYKRLIDKAKTVEGVEALKDAILAAL (SEQ ID |
| | NO: 659) |
| | HGEGTFTSDLSKQLEEEAVRLFIEWLKNGGPSSGAPPKSGGGSLAEAKE |
| | AANAELDSYGVSDFYKRLIDKAKTVEGVEALKDAILAALP (SEQ ID |
| | NO: 660) |
| | HGEGTFTSDLSKQLEEEAVRLFIEWLKNGGPSSGAPPKSGGGSLAEAKE |
| | AANAELDSYGVSDFYKRLIDKAKTVEGVEALKDAILAAL (SEQ ID |
| | NO: 661) |
| | HGEGTFTSDLSKQLEEEAVRLFIEWLKNGGPSSGAPPKSGGGLAEAKEA |
| | ANAELDSYGVSDFYKRLIDKAKTVEGVEALKDAILAALP (SEQ ID |
| | NO: 662) |
| | HGEGTFTSDLSKQLEEEAVRLFIEWLKNGGPSSGAPPKSGGGLAEAKEA |
| | ANAELDSYGVSDFYKRLIDKAKTVEGVEALKDAILAAL (SEQ ID |
| | NO: 663) |
| | HGEGTFTSDLSKQLEEEAVRLFIEWLKNGGPSSGAPPKSGGSLAEAKEA |
| | ANAELDSYGVSDFYKRLIDKAKTVEGVEALKDAILAALP (SEQ ID |
| | NO: 664) |
| | HGEGTFTSDLSKQLEEEAVRLFIEWLKNGGPSSGAPPKSGGSLAEAKEA |
| | ANAELDSYGVSDFYKRLIDKAKTVEGVEALKDAILAAL (SEQ ID |
| | NO: 665) |
| | HGEGTFTSDLSKQLEEEAVRLFIEWLKNGGPSSGAPPKSGSLAEAKEAA |
| | NAELDSYGVSDFYKRLIDKAKTVEGVEALKDAILAALP (SEQ ID |
| | NO: 666) |
| | HGEGTFTSDLSKQLEEEAVRLFIEWLKNGGPSSGAPPKSGSLAEAKEAA |
| | NAELDSYGVSDFYKRLIDKAKTVEGVEALKDAILAAL (SEQ ID NO: 667) |
| | HGEGTFTSDLSKQLEEEAVRLFIEWLKNGGPSSGAPPKSGLAEAKEAAN |
| | AELDSYGVSDFYKRLIDKAKTVEGVEALKDAILAALP (SEQ ID NO: 668) |
| | HGEGTFTSDLSKQLEEEAVRLFIEWLKNGGPSSGAPPKSGLAEAKEAAN |
| | AELDSYGVSDFYKRLIDKAKTVEGVEALKDAILAAL (SEQ ID NO: 669) |
| | HGEGTFTSDLSKQMEEEAVRLFIEWLKNGGPSSGAPPKSTGGGGSASGS |
| | LAEAKEAANAELDSYGVSDFYKRLIDKAKTVEGVEALKDAILAALP |
| | (SEQ ID NO: 670) |
| | HGEGTFTSDLSKQMEEEAVRLFIEWLKNGGPSSGAPPKSTGGGGSASGS |
| | LAEAKEAANAELDSYGVSDFYKRLIDKAKTVEGVEALKDAILAAL (SEQ |
| | ID NO: 671) |
| | HGEGTFTSDLSKQMEEEAVRLFIEWLKNGGPSSGAPPKSTGGGGSASSL |
| | AEAKEAANAELDSYGVSDFYKRLIDKAKTVEGVEALKDAILAALP (SEQ |
| | ID NO: 672) |
| | HGEGTFTSDLSKQMEEEAVRLFIEWLKNGGPSSGAPPKSTGGGGSASSL |
| | AEAKEAANAELDSYGVSDFYKRLIDKAKTVEGVEALKDAILAAL (SEQ |
| | ID NO: 673) |
| | HGEGTFTSDLSKQMEEEAVRLFIEWLKNGGPSSGAPPKSTGGGGSASLA |
| | EAKEAANAELDSYGVSDFYKRLIDKAKTVEGVEALKDAILAALP (SEQ |
| | ID NO: 674) |
| | HGEGTFTSDLSKQMEEEAVRLFIEWLKNGGPSSGAPPKSTGGGGSASLA |
| | EAKEAANAELDSYGVSDFYKRLIDKAKTVEGVEALKDAILAAL (SEQ ID |
| | NO: 675) |
| | HGEGTFTSDLSKQMEEEAVRLFIEWLKNGGPSSGAPPKSGGGGSLAEAK |
| | EAANAELDSYGVSDFYKRLIDKAKTVEGVEALKDAILAALP (SEQ ID |
| | NO: 676) |
| | HGEGTFTSDLSKQMEEEAVRLFIEWLKNGGPSSGAPPKSGGGGSLAEAK |
| | EAANAELDSYGVSDFYKRLIDKAKTVEGVEALKDAILAAL (SEQ ID |
| | NO: 677) |
| | HGEGTFTSDLSKQMEEEAVRLFIEWLKNGGPSSGAPPKSGGGSLAEAKE |
| | AANAELDSYGVSDFYKRLIDKAKTVEGVEALKDAILAALP (SEQ ID |
| | NO: 678) |
| | HGEGTFTSDLSKQMEEEAVRLFIEWLKNGGPSSGAPPKSGGGSLAEAKE |
| | AANAELDSYGVSDFYKRLIDKAKTVEGVEALKDAILAAL (SEQ ID |
| | NO: 679) |
| | HGEGTFTSDLSKQMEEEAVRLFIEWLKNGGPSSGAPPKSGGGLAEAKEA |
| | ANAELDSYGVSDFYKRLIDKAKTVEGVEALKDAILAALP (SEQ ID |
| | NO: 680) |
| | HGEGTFTSDLSKQMEEEAVRLFIEWLKNGGPSSGAPPKSGGGLAEAKEA |
| | ANAELDSYGVSDFYKRLIDKAKTVEGVEALKDAILAAL (SEQ ID |
| | NO: 681) |
| | HGEGTFTSDLSKQMEEEAVRLFIEWLKNGGPSSGAPPKSGGSLAEAKEA |
| | ANAELDSYGVSDFYKRLIDKAKTVEGVEALKDAILAALP (SEQ ID |
| | NO: 682) |
| | HGEGTFTSDLSKQMEEEAVRLFIEWLKNGGPSSGAPPKSGGSLAEAKEA |
| | ANAELDSYGVSDFYKRLIDKAKTVEGVEALKDAILAAL (SEQ ID |
| | NO: 683) |
| | HGEGTFTSDLSKQMEEEAVRLFIEWLKNGGPSSGAPPKSGSLAEAKEAA |
| | NAELDSYGVSDFYKRLIDKAKTVEGVEALKDAILAALP (SEQ ID |
| | NO: 684) |
| | HGEGTFTSDLSKQMEEEAVRLFIEWLKNGGPSSGAPPKSGSLAEAKEAA |
| | NAELDSYGVSDFYKRLIDKAKTVEGVEALKDAILAAL (SEQ ID NO: 685) |
| | HGEGTFTSDLSKQMEEEAVRLFIEWLKNGGPSSGAPPKSGLAEAKEAAN |
| | AELDSYGVSDFYKRLIDKAKTVEGVEALKDAILAALP (SEQ ID NO: 686) |
| | HGEGTFTSDLSKQMEEEAVRLFIEWLKNGGPSSGAPPKSGLAEAKEAAN |
| | AELDSYGVSDFYKRLIDKAKTVEGVEALKDAILAAL (SEQ ID NO: 687) |
| 2-10 | HGEGTFTSDLSKQLEEEAVRLFIEWLKNGGPSSGAPPPSTGGGGSASGSL |
| | AEAKEAANAELDSYGVSDFYKRLIDKAKTVEGVEALKDAILAALP (SEQ |
| | ID NO: 625) |
| | HGEGTFTSDLSKQLEEEAVRLFIEWLKNGGPSSGAPPPSTGGGGSASSLA |
| | EAKEAANAELDSYGVSDFYKRLIDKAKTVEGVEALKDAILAALP (SEQ |
| | ID NO: 688) |
| | HGEGTFTSDLSKQLEEEAVRLFIEWLKNGGPSSGAPPPSTGGGGSASGSL |
| | AEAKEAANAELDSYGVSDFYKRLIDKAKTVEGVEALKDAILAA (SEQ ID |
| | NO: 629) |
| | HGEGTFTSDLSKQLEEEAVRLFIEWLKNGGPSSGAPPPSTGGGGSASSLA |
| | EAKEAANAELDSYGVSDFYKRLIDKAKTVEGVEALKDAILAAL (SEQ ID |
| | NO: 689) |
| | HGEGTFTSDLSKQLEEEAVRLFIEWLKNGGPSSGAPPPSTGGGGSASLAE |
| | AKEAANAELDSYGVSDFYKRLIDKAKTVEGVEALKDAILAALP (SEQ ID |
| | NO: 690) |
| | HGEGTFTSDLSKQLEEEAVRLFIEWLKNGGPSSGAPPPSTGGGGSASLAE |
| | AKEAANAELDSYGVSDFYKRLIDKAKTVEGVEALKDAILAAL (SEQ ID |
| | NO: 691) |
| | HGEGTFTSDLSKQLEEEAVRLFIEWLKNGGPSSGAPPPSGGGGSLAEAKE |
| | AANAELDSYGVSDFYKRLIDKAKTVEGVEALKDAILAALP (SEQ ID |
| | NO: 692) |
| | HGEGTFTSDLSKQLEEEAVRLFIEWLKNGGPSSGAPPPSGGGGSLAEAKE |
| | AANAELDSYGVSDFYKRLIDKAKTVEGVEALKDAILAAL (SEQ ID |
| | NO: 693) |
| | HGEGTFTSDLSKQLEEEAVRLFIEWLKNGGPSSGAPPPSGGGSLAEAKEA |
| | ANAELDSYGVSDFYKRLIDKAKTVEGVEALKDAILAALP (SEQ ID |
| | NO: 694) |
| | HGEGTFTSDLSKQLEEEAVRLFIEWLKNGGPSSGAPPPSGGGSLAEAKEA |
| | ANAELDSYGVSDFYKRLIDKAKTVEGVEALKDAILAAL (SEQ ID |
| | NO: 695) |
| | HGEGTFTSDLSKQLEEEAVRLFIEWLKNGGPSSGAPPPSGGGLAEAKEA |
| | ANAELDSYGVSDFYKRLIDKAKTVEGVEALKDAILAALP (SEQ ID |
| | NO: 696) |
| | HGEGTFTSDLSKQLEEEAVRLFIEWLKNGGPSSGAPPPSGGGLAEAKEA |
| | ANAELDSYGVSDFYKRLIDKAKTVEGVEALKDAILAAL (SEQ ID |
| | NO: 697) |
| 2-11 | HGEGTFTSDLSKQLEEEAVRLFIEWLKNGGPSSGAPPPSGGSLAEAKEAA |
| | NAELDSYGVSDFYKRLIDKAKTVEGVEALKDAILAALP (SEQ ID |
| | NO: 626) |
| | HGEGTFTSDLSKQLEEEAVRLFIEWLKNGGPSSGAPPPSGGSLAEAKEAA |
| | NAELDSYGVSDFYKRLIDKAKTVEGVEALKDAILAAL (SEQ ID NO: 632) |
| | HGEGTFTSDLSKQLEEEAVRLFIEWLKNGGPSSGAPPPSGSLAEAKEAAN |
| | AELDSYGVSDFYKRLIDKAKTVEGVEALKDAILAALP (SEQ ID NO: 698) |
| | HGEGTFTSDLSKQLEEEAVRLFIEWLKNGGPSSGAPPPSGSLAEAKEAAN |
| | AELDSYGVSDFYKRLIDKAKTVEGVEALKDAILAAL (SEQ ID NO: 699) |
| | HGEGTFTSDLSKQLEEEAVRLFIEWLKNGGPSSGAPPPSGLAEAKEAAN |
| | AELDSYGVSDFYKRLIDKAKTVEGVEALKDAILAALP (SEQ ID NO: 700) |
| | HGEGTFTSDLSKQLEEEAVRLFIEWLKNGGPSSGAPPPSGGSLAEAKEAA |
| | NAELDSYGVSDFYKRLIDKAKTVEGVEALKDAILAA (SEQ ID NO: 637) |
| | HGEGTFTSDLSKQLEEEAVRLFIEWLKNGGPSSGAPPPSGLAEAKEAAN |
| | AELDSYGVSDFYKRLIDKAKTVEGVEALKDAILAAL (SEQ ID NO: 701) |
| 2-8 | HGEGTFTSDLSKQMEEEAVRLFIEWLKNGGPSSGAPPPSTGGGGSASGSL |
| | AEAKEAANAELDSYGVSDFYKRLIDKAKTVEGVEALKDAILAALP (SEQ |
| | ID NO: 623) |
| | HGEGTFTSDLSKQMEEEAVRLFIEWLKNGGPSSGAPPPSTGGGGSASGSL |
| | AEAKEAANAELDSYGVSDFYKRLIDKAKTVEGVEALKDAILAAL (SEQ |
| | ID NO: 630) |
| | HGEGTFTSDLSKQMEEEAVRLFIEWLKNGGPSSGAPPPSTGGGGSASSLA |
| | EAKEAANAELDSYGVSDFYKRLIDKAKTVEGVEALKDAILAALP (SEQ |
| | ID NO: 702) |
| | HGEGTFTSDLSKQMEEEAVRLFIEWLKNGGPSSGAPPPSTGGGGSASSLA |
| | EAKEAANAELDSYGVSDFYKRLIDKAKTVEGVEALKDAILAAL (SEQ ID |
| | NO: 703) |
| | HGEGTFTSDLSKQMEEEAVRLFIEWLKNGGPSSGAPPPSTGGGGSASLAE |
| | AKEAANAELDSYGVSDFYKRLIDKAKTVEGVEALKDAILAALP (SEQ ID |
| | NO: 704) |
| | HGEGTFTSDLSKQMEEEAVRLFIEWLKNGGPSSGAPPPSTGGGGSASLAE |
| | AKEAANAELDSYGVSDFYKRLIDKAKTVEGVEALKDAILAAL (SEQ ID |
| | NO: 705) |
| | HGEGTFTSDLSKQMEEEAVRLFIEWLKNGGPSSGAPPPSGGGGSLAEAK |
| | EAANAELDSYGVSDFYKRLIDKAKTVEGVEALKDAILAALP (SEQ ID |
| | NO: 706) |
| | HGEGTFTSDLSKQMEEEAVRLFIEWLKNGGPSSGAPPPSGGGGSLAEAK |
| | EAANAELDSYGVSDFYKRLIDKAKTVEGVEALKDAILAAL (SEQ ID |
| | NO: 707) |
| | HGEGTFTSDLSKQMEEEAVRLFIEWLKNGGPSSGAPPPSGGGSLAEAKE |
| | AANAELDSYGVSDFYKRLIDKAKTVEGVEALKDAILAALP (SEQ ID |
| | NO: 708) |
| | HGEGTFTSDLSKQMEEEAVRLFIEWLKNGGPSSGAPPPSGGGSLAEAKE |
| | AANAELDSYGVSDFYKRLIDKAKTVEGVEALKDAILAAL (SEQ ID |
| | NO: 709) |
| | HGEGTFTSDLSKQMEEEAVRLFIEWLKNGGPSSGAPPPSGGGLAEAKEA |
| | ANAELDSYGVSDFYKRLIDKAKTVEGVEALKDAILAALP (SEQ ID |
| | NO: 710) |
| | HGEGTFTSDLSKQMEEEAVRLFIEWLKNGGPSSGAPPPSGGGLAEAKEA |
| | ANAELDSYGVSDFYKRLIDKAKTVEGVEALKDAILAAL (SEQ ID |
| | NO: 711) |
| 2-9 | HGEGTFTSDLSKQMEEEAVRLFIEWLKNGGPSSGAPPPSGGSLAEAKEA |
| | ANAELDSYGVSDFYKRLIDKAKTVEGVEALKDAILAALP (SEQ ID |
| | NO: 624) |
| | HGEGTFTSDLSKQMEEEAVRLFIEWLKNGGPSSGAPPPSGGSLAEAKEA |
| | ANAELDSYGVSDFYKRLIDKAKTVEGVEALKDAILAAL (SEQ ID |
| | NO: 631) |
| | HGEGTFTSDLSKQMEEEAVRLFIEWLKNGGPSSGAPPPSGGSLAEAKEA |
| | ANAELDSYGVSDFYKRLIDKAKTVEGVEALKDAILAA (SEQ ID NO: 636) |
| | HGEGTFTSDLSKQMEEEAVRLFIEWLKNGGPSSGAPPPSGSLAEAKEAA |
| | NAELDSYGVSDFYKRLIDKAKTVEGVEALKDAILAALP (SEQ ID |
| | NO: 712) |
| | HGEGTFTSDLSKQMEEEAVRLFIEWLKNGGPSSGAPPPSGSLAEAKEAA |
| | NAELDSYGVSDFYKRLIDKAKTVEGVEALKDAILAAL (SEQ ID NO: 713) |
| | HGEGTFTSDLSKQMEEEAVRLFIEWLKNGGPSSGAPPPSGLAEAKEAAN |
| | AELDSYGVSDFYKRLIDKAKTVEGVEALKDAILAALP (SEQ ID NO: 714) |
| | HGEGTFTSDLSKQMEEEAVRLFIEWLKNGGPSSGAPPPSGLAEAKEAAN |
| | AELDSYGVSDFYKRLIDKAKTVEGVEALKDAILAAL (SEQ ID NO: 715) |
| | HGEGTFTSDLSKQLEEEAVRLFIEWLKNGGPSSGAPPSKKKKKKTGGGG |
| | SASGSLAEAKEAANAELDSYGVSDFYKRLIDKAKTVEGVEALKDAILAA |
| | LP (SEQ ID NO: 716) |
| | HGEGTFTSDLSKQLEEEAVRLFIEWLKNGGPSSGAPPSKKKKKKTGGGG |
| | SASGSLAEAKEAANAELDSYGVSDFYKRLIDKAKTVEGVEALKDAILAA |
| | L (SEQ ID NO: 717) |
| | HGEGTFTSDLSKQLEEEAVRLFIEWLKNGGPSSGAPPSKKKKKKTGGGG |
| | SASSLAEAKEAANAELDSYGVSDFYKRLIDKAKTVEGVEALKDAILAAL |
| | P (SEQ ID NO: 718) |
| | HGEGTFTSDLSKQLEEEAVRLFIEWLKNGGPSSGAPPSKKKKKKTGGGG |
| | SASSLAEAKEAANAELDSYGVSDFYKRLIDKAKTVEGVEALKDAILAAL |
| | (SEQ ID NO: 719) |
| | HGEGTFTSDLSKQLEEEAVRLFIEWLKNGGPSSGAPPSKKKKKKTGGGG |
| | SASLAEAKEAANAELDSYGVSDFYKRLIDKAKTVEGVEALKDAILAALP |
| | (SEQ ID NO: 720) |
| | HGEGTFTSDLSKQLEEEAVRLFIEWLKNGGPSSGAPPSKKKKKKTGGGG |
| | SASLAEAKEAANAELDSYGVSDFYKRLIDKAKTVEGVEALKDAILAAL |
| | (SEQ ID NO: 721) |
| | HGEGTFTSDLSKQLEEEAVRLFIEWLKNGGPSSGAPPSKKKKKKGGGGS |
| | LAEAKEAANAELDSYGVSDFYKRLIDKAKTVEGVEALKDAILAALP |
| | (SEQ ID NO: 722) |
| | HGEGTFTSDLSKQLEEEAVRLFIEWLKNGGPSSGAPPSKKKKKKGGGGS |
| | LAEAKEAANAELDSYGVSDFYKRLIDKAKTVEGVEALKDAILAAL (SEQ |
| | ID NO: 723) |
| | HGEGTFTSDLSKQLEEEAVRLFIEWLKNGGPSSGAPPSKKKKKKGGGSL |
| | AEAKEAANAELDSYGVSDFYKRLIDKAKTVEGVEALKDAILAALP (SEQ |
| | ID NO: 724) |
| | HGEGTFTSDLSKQLEEEAVRLFIEWLKNGGPSSGAPPSKKKKKKGGGSL |
| | AEAKEAANAELDSYGVSDFYKRLIDKAKTVEGVEALKDAILAAL (SEQ |
| | ID NO: 725) |
| | HGEGTFTSDLSKQLEEEAVRLFIEWLKNGGPSSGAPPSKKKKKKGGGLA |
| | EAKEAANAELDSYGVSDFYKRLIDKAKTVEGVEALKDAILAALP (SEQ |
| | ID NO: 726) |
| | HGEGTFTSDLSKQLEEEAVRLFIEWLKNGGPSSGAPPSKKKKKKGGGLA |
| | EAKEAANAELDSYGVSDFYKRLIDKAKTVEGVEALKDAILAAL (SEQ ID |
| | NO: 727) |
| | HGEGTFTSDLSKQLEEEAVRLFIEWLKNGGPSSGAPPSKKKKKKGGSLA |
| | EAKEAANAELDSYGVSDFYKRLIDKAKTVEGVEALKDAILAALP (SEQ |
| | ID NO: 728) |
| | HGEGTFTSDLSKQLEEEAVRLFIEWLKNGGPSSGAPPSKKKKKKGGSLA |
| | EAKEAANAELDSYGVSDFYKRLIDKAKTVEGVEALKDAILAAL (SEQ ID |
| | NO: 729) |
| | HGEGTFTSDLSKQLEEEAVRLFIEWLKNGGPSSGAPPSKKKKKKGSLAE |
| | AKEAANAELDSYGVSDFYKRLIDKAKTVEGVEALKDAILAALP (SEQ ID |
| | NO: 730) |
| | HGEGTFTSDLSKQLEEEAVRLFIEWLKNGGPSSGAPPSKKKKKKGSLAE |
| | AKEAANAELDSYGVSDFYKRLIDKAKTVEGVEALKDAILAAL (SEQ ID |
| | NO: 731) |
| | HGEGTFTSDLSKQLEEEAVRLFIEWLKNGGPSSGAPPSKKKKKKGLAEA |
| | KEAANAELDSYGVSDFYKRLIDKAKTVEGVEALKDAILAALP (SEQ ID |
| | NO: 732) |
| | HGEGTFTSDLSKQLEEEAVRLFIEWLKNGGPSSGAPPSKKKKKKGLAEA |
| | KEAANAELDSYGVSDFYKRLIDKAKTVEGVEALKDAILAAL (SEQ ID |
| | NO: 733) |

Specifically contemplated are compounds of the above Exendin ABD sequences in which any N-terminal methionine is absent. The N-terminal methionine can be present primarily as a convenience for bacterial expression. However, engineered peptides of the present invention can be expressed in a eukaryotic host cell (e.g. yeast (e.g. Pichia), mammalian, baculovirus) or other host cell having post-translational N-terminal proteolytic processing to yield an N-terminal amino acid as found in a naturally occurring mature peptide counterpart of the desired hormone or ABD sequence, i.e. without the added methionine or other leader sequence. Alternatively, an N-terminal sequence used for expression and/or secretion (and even purification) can be one that can be removed post-translationally, e.g. as by use of a protease such as TEV.

### III. Methods of Design and Production

### Design of Constructs

The engineered polypeptides described herein can be designed at the amino acid level. These sequences can then be back translated using a variety of software products known in the art such that the nucleotide sequence is optimized for the desired expression host, e.g. based protein expression, codon optimization, restriction site content. For example, the nucleotide sequence can be optimized for E. coli based protein expression and for restriction site content. Based on the nucleotide sequence of interest, overlapping oligonucleotides can be provided for multistep PCR, as known in the art. These oligonucleotides can be used in multiple PCR reactions under conditions well known in the art to build the cDNA encoding the protein of interest. For one example is 1× Amplitaq Buffer, 1.3 mM MgCl₂, 200 uM dNTPs, 4 U Amplitaq Gold, 0.2 uM of each primer (AmpliTaq Gold, ABI), with cycling parameters: (94 C:30 s, 58 C:1 mM, 72 C:1 min), 35 cycles.

Restriction sites can be added to the ends of the PCR products for use in vector ligation as known in the art. Specific sites can include Nde1 and Xho1, such that the cDNA can then be in the proper reading frame in a pET45b expression vector (Novagen). By using these sites, any N-terminal His Tag that are in this vector can be removed as the translation start site would then be downstream of the tag. Once expression constructs are completed, verification can be conduct by sequencing using e.g., T7 promoter primer, T7 terminator primer and standard ABI BigDye Term v3.1 protocols as known in the art. Sequence information can be obtained from e.g., an ABI 3730 DNA Analyzer and can be analyzed using Vector NTI v.10 software (Invitrogen). Expression constructs can be designed in a modular manner such that linker sequences can be easily cut out and changed, as known in the art.

Protease recognition sites, known in the art or described herein, can be incorporated into constructs useful for the design, construction, manipulation and production of recombinant engineering polypeptides described herein.

Exemplary Constructs.

Constructs useful in the production of engineered polypeptides contemplated herein include constructs encoding the polypeptides having an ABD1 as set forth in Table 4A following.

TABLE 4A
Selected exemplary constructs for recombinant
production of engineered polypeptides

| Cmpd | Sequence |
|---|---|
| P1 | MAHHHHHHVGTGSNENLYFQHGEGTFTSDLSKQMEEEAVRLFIEW |
| | LKNTGGGGSGGGSGGGSGGGSASLAEAKVLANRELDKYGVSDFY |
| | KRLINKAKTVEGVEALKLHILAALP (SEQ ID NO: 156) |
| P2 | MAHHHHHHVGTGSNENLYFQHGEGTFTSDLSKQMEEEAVRLFIEW |
| | LKNTGGGGSASLAEAKVLANRELDKYGVSDFYKRLINKAKTVEGV |
| | EALKLHILAALP (SEQ ID NO: 157) |
| P3 | MAHHHHHHVGTGSNENLYFQHGEGTFTSDLSKQMEEEAVRLFIEW |
| | LKNTGGGGSGGGSGGGSGGGSASLAEAKVLANRELDKYGVSDYYK |
| | NIINRAKTVEGVRALKLHILAALP (SEQ ID NO: 158) |
| P4 | MAHHHHHHVGTGSNENLYFQHGEGTFTSDLSKQMEEEAVRLFIEW |
| | LKNTGGGGSASLAEAKVLANRELDKYGVSDYYKNIINRAKTVEGV |
| | RALKLHILAALP (SEQ ID NO: 159) |
| P5 | MSDKIIHLTDDSFDTDVLKADGAILVDFWAEWCGPCKMIAPILDE |
| | IADEYQGKLTVAKLNIDQNPGTAPKYGIRGIPTLLLFKNGEVAAT |
| | KVGALSKGQLKEFLDANLAGSGSGHMHHHHHHSSGLVPRGSGMK |
| | ETAAAKFERQHMDSPDLGTENLYFQHGEGTFTSDLSKQLEEEAVR |
| | LFIEWLKNGGPSSGAPPPSTGGGGSGGGSGGGSGGGSASLAEAKV |
| | LANRELDKYGVSDFYKRLINKAKTVEGVEALKLHILAALP |
| | (SEQ ID NO: 160) |
| P6 | MSDKIIHLTDDSFDTDVLKADGAILVDFWAEWCGPCKMIAPILD |
| | EIADEYQGKLTVAKLNIDQNPGTAPKYGIRGIPTLLLFKNGEVAA |
| | TKVGALSKGQLKEFLDANLAGSGSGHMHHHHHHSSGLVPRGSGM |
| | KETAAAKFERQHMDSPDLGTENLYFQHGEGTFTSDLSKQMEEEA |
| | VRLFIEWLKNTGGGGSGGGSGGGSGGGSASLAEAKVLANRELD |
| | KYGVSDYYKNIINRAKTVEGVRALKLHILAALP |
| | (SEQ ID NO: 161) |
| P7 | MSDKIIHLTDDSFDTDVLKADGAILVDFWAEWCGPCKMIAPILDE |
| | IADEYQGKLTVAKLNIDQNPGTAPKYGIRGIPTLLLFKNGEVAAT |
| | KVGALSKGQLKEFLDANLAGSGSGHMHHHHHHSSGLVPRGSGM |
| | KETAAAKFERQHMDSPDLGTENLYFQHGEGTFTSDLSKQMEEEA |
| | VRLFIEWLKNTGGGGSASLAEAKVLANRELDKYGVSDYYKNIINR |
| | AKTVEGVRALKLHILAALP (SEQ ID NO: 162) |

Exemplary Constructs.

Constructs useful in the production of engineered polypeptides contemplated herein include constructs encoding the polypeptides set forth in Table 4B following.

TABLE 4B
Selected exemplary constructs for recombinant
production of engineeredpolypeptides containing
ABD sequences of the ABD2 type.

MAHHHHHHVGTGSNENLYFQHGEGTFTSDLSKQLEEEAVRLFIEWLKQGG
PSKEIISTGGGGSASGSLAEAKEAANAELDSYGVSDFYKRLIDKAKTVEG
VEALKDAILAALP (SEQ ID NO: 734)
MSDKIIHLTDDSFDTDVLKADGAILVDFWAEWCGPCKMIAPILDEIADEY
QGKLTVAKLNIDQNPGTAPKYGIRGIPTLLLFKNGEVAATKVGALSKGQL
KEFLDANLAGSGSGHMHHHHHHSSGLVPRGSGMKETAAAKFERQHMDSPD
LGTENLYFQHGEGTFTSDLSKQLEEEAVRLFIEWLKQGGPSKEIISTGGG
GSASGSLAEAKEAANAELDSYGVSDFYKRLIDKAKTVEGVEALKDAILAA
LP (SEQ ID NO: 735)
MAHHHHHHVGTGSNENLYFQHGEGTFTSDLSKQLEEEAVRLFIEWLKNGG
PSSGAPPPSTGGGGSASGSLAEAKEAANAELDSYGVSDFYKRLIDKAKTV
EGVEALKDAILAALP (SEQ ID NO: 736)
MSDKIIHLTDDSFDTDVLKADGAILVDFWAEWCGPCKMIAPILDEIADEY
QGKLTVAKLNIDQNPGTAPKYGIRGIPTLLLFKNGEVAATKVGALSKGQL
KEFLDANLAGSGSGHMHHHHHHSSGLVPRGSGMKETAAAKFERQHMDSPD
LGTENLYFQHGEGTFTSDLSKQLEEEAVRLFIEWLKNGGPSSGAPPPSTG
GGGSASGSLAEAKEAANAELDSYGVSDFYKRLIDKAKTVEGVEALKDAIL
AALP (SEQ ID NO: 737)
MAHHHHHHVGTGSNENLYFQHGEGTFTSDLSKQMEEEAVRLFIEWLKNGG
PSSGAPPPSTGGGGSASGSLAEAKEAANAELDSYGVSDFYKRLIDKAKTV
EGVEALKDAILAALP (SEQ ID NO: 738)
MSDKIIHLTDDSFDTDVLKADGAILVDFWAEWCGPCKMIAPILDEIADEY
QGKLTVAKLNIDQNPGTAPKYGIRGIPTLLLFKNGEVAATKVGALSKGQL
KEFLDANLAGSGSGHMHHHHHHSSGLVPRGSGMKETAAAKFERQHMDSPD
LGTENLYFQHGEGTFTSDLSKQMEEEAVRLFIEWLKNGGPSSGAPPPSTG
GGGSASGSLAEAKEAANAELDSYGVSDFYKRLIDKAKTVEGVEALKDAIL
AALP (SEQ ID NO: 739)

The Exendin ABD compounds display only a 10-100 fold loss of GLP-R activation in an in vitro assay, as described herein, which is more than compensated by the long duration of action in plasma. The Exendin ABD compounds as exemplified herein retain albumin binding similar to the parent ABD sequence itself, in the picomolar affinity. The Exendin ABD are stable in human plasma for at least 6 hours (as determined by in vitro assay). The Exendin ABD are more stable in pancreatin mix a acidic pH than at neutral pH. The Exendin ABD are stable in the formulations exemplified herein. In vivo the Exendin ABD demonstrate only about a 3-fold lower glucose lowering activity in a acute mouse OGTT assay. In a duration of action assay for glucose lowering in a mouse, the Exendin ABD display duration greater than 24 hours, particularly at doses of 25 nmol/kg. The Leu14 exendin-4 is not active in this assay. The Exendin ABD demonstrate reduction of food intake activity with a ED50 only about 5-fold higher than Leu14-exendin. As shown herein, the Exendin ABD are effective to lower glucose and reduce body weight, for example in an diabetes and obese diabetes system. In a rat, IV dosed Exendin ABD indicated a t½ of at least 11 hours, whereas Leu14 exendin-4 is about 30 minutes. Using formulations for transmucosal administration as described herein, intestinal delivery to a rat provided an exposure greater than 24 hours, to a beagle dog of greater than 5 days, and to a primate greater than 2 weeks. As the turnover of albumin in the cynomolgus monkey is about 11-13 days, and about 20 days in humans, the formulations will provide once weekly, twice weekly and even once monthly delivery in a human subject. The long duration profiles were accompanied by relatively low Cmax and a desirable smooth profile, even after a single dose. When formulated as provided herein relatively rapid uptake was observed demonstrating that a sustained release formulation is not required. After about one hour, therapeutic levels were achieved and were sustained over the time course. The Exendin ABD compounds appear to circulate at least as long if not longer than the serum albumin of the recipient species. A flatter, more even, patient-tolerable PK profile was observed, which provides continuous sustained exposure at therapeutic levels, which when combined with a Cmax that is relatively low compared to the average sustained plasma concentration, will enhance patient tolerability, compliance, and acceptance enabling increased therapy effectiveness, particularly for human patients.

General Methods of Production.

The engineered polypeptide compounds described herein may be prepared using biological, chemical, and/or recombinant DNA techniques that are known in the art. Exemplary methods are described herein and in U.S. Pat. No. 6,872,700; WO 2007/139941; WO 2007/140284; WO 2008/082274; WO 2009/011544; and US Publication No. 2007/0238669, the disclosures of which are incorporated herein by reference in their entireties and for all purposes. Other methods for preparing the compounds are set forth herein.

The engineered polypeptides compounds described herein may be prepared using standard solid-phase peptide synthesis techniques, such as an automated or semiautomated peptide synthesizer. Briefly and generally, the ABD and therapeutic hormonal peptide can be made separately and then conjugated together or can be made as a single polypeptide. Thus, the albumin binding polypeptide, therapeutic hormone or engineered polypeptide may alternatively be produced by non-biological peptide synthesis using amino acids and/or amino acid derivatives having reactive side-chains protected, the non-biological peptide synthesis including step-wise coupling of the amino acids and/or the amino acid derivatives to form a polypeptide according to the first aspect having reactive side-chains protected, removing the protecting groups from the reactive side-chains of the polypeptide, and folding of the polypeptide in aqueous solution. Thus, normal amino acids (e.g. glycine, alanine, phenylalanine, isoleucine, leucine and valine) and pre-protected amino acid derivatives are used to sequentially build a polypeptide sequence, in solution or on a solid support in an organic solvent. When a complete polypeptide sequence is built, the protecting groups are removed and the polypeptide is allowed to fold in an aqueous solution.

Each polypeptide according to the present disclosure reversibly folds, with the ABD domain reversibly folding into a three helix bundle domain without added factors, and hence folds spontaneously. The engineered conjugate may be produced by a method including producing an albumin binding polypeptide according to any method, e.g. as described herein, such as by non-biological peptide synthesis, and conjugating the produced ABD polypeptide with the therapeutic hormone defined herein. The ABDs herein fold completely reversibly. This was assessed by circular dichroism spectra analysis; one spectrum taken at 20° C. and a second spectrum after heating to 90° C. followed by return to 20° C. During this procedure the Tm, as known in the art, was determined and found to be unchanged after the folding of the denatured polypeptide.

Typically, using such techniques, an alpha-N-carbamoyl protected amino acid and an amino acid attached to the growing peptide chain on a resin are coupled at RT in an inert solvent (e.g., dimethylformamide, N-methylpyrrolidinone, methylene chloride, and the like) in the presence of coupling agents (e.g., dicyclohexylcarbodiimide, 1-hydroxybenzo-triazole, and the like) in the presence of a base (e.g., diisopropylethylamine, and the like). The alpha-N-carbamoyl protecting group is removed from the resulting peptide-resin using a reagent (e.g., trifluoroacetic acid, piperidine, and the like) and the coupling reaction repeated with the next desired N-protected amino acid to be added to the peptide chain. Suitable N-protecting groups are well known in the art, such as t-butyloxycarbonyl (tBoc) fluorenylmethoxycarbonyl (Fmoc), and the like. The solvents, amino acid derivatives and 4-methylbenzhydryl-amine resin used in the peptide synthesizer may be purchased from Applied Biosystems Inc. (Foster City, Calif.).

For chemical synthesis solid phase peptide synthesis can be used for the engineered polypeptides, since in general solid phase synthesis is a straightforward approach with excellent scalability to commercial scale, and is generally compatible with relatively long engineered polypeptides. Solid phase peptide synthesis may be carried out with an automatic peptide synthesizer (Model 430A, Applied Biosystems Inc., Foster City, Calif.) using the NMP/HOBt (Option 1) system and tBoc or Fmoc chemistry (See APPLIED BIOSYSTEMS USER'S MANUAL FOR THE ABI 430A PEPTIDE SYNTHESIZER, Version 1.3B Jul. 1, 1988, section 6, pp. 49-70, Applied Biosystems, Inc., Foster City, Calif.) with capping. Boc-peptide-resins may be cleaved with HF (−5° C. to 0° C., 1 hour). The peptide may be extracted from the resin with alternating water and acetic acid, and the filtrates lyophilized. The Fmoc-peptide resins may be cleaved according to standard methods (e.g., Introduction to Cleavage Techniques, Applied Biosystems, Inc., 1990, pp. 6-12). Peptides may also be assembled using an Advanced Chem Tech Synthesizer (Model MPS 350, Louisville, Ky.).

A chemical synthesis method that provided better yields is exemplified as follows for Cmpd 2-11. Solid phase synthesis was performed using a Prelude 6 channel peptide synthesizer (Protein Technologies, Inc., Tucson, Ariz., USA) using Fomc-Pro-Novasyn TGT resin (0.2 mmole/g) using default “double coupling” settings. However for VS (amino acid positions 59-60) and KT (amino acid positions 71-72) sequences pseudoproline double coupling was used, and for amino acids V19, R20, I23, and P37 triple coupling was used. For the exendin portion from His1 to Ser39, HATU/DIEA double or triple coupling (˜60 min each, 6× excess of reagents) was performed unless otherwise indicated with deblocking with 20% piperidine 2×15 min. For the linker and ABD portion, HATU/DIEA double coupling was performed unless otherwise indicated (˜60 min each, 3× excess of reagents) with deblocking with 20% piperidine 2×15 min. Polypeptide purification was performed using RP-HPLC purification on a C5 column using acetonitrile as solvent, with eluted samples identified by analysis on an analytical RP-HPLC on a C18 column using acetonitrile as solvent, followed by preparative RP-HPLC on a C18 column using a more narrow gradient than in the first RP-HPLC and acetonitrile as solvent. Fractions containing desired engineered polypeptide were pooled and lyophilized.

In one embodiment of the engineered polypeptides described herein, particularly those ending at its C-terminus with proline or other amino acid known to racemize during peptide synthesis, a glycine can be added to the C-terminus to counter potential problems with racemization of the C-terminal amino acid residue. Alternatively the C-terminal amino acid can in its (alpha-amino group) amidated form, e.g. proline versus proline amide, rather than ending with a glycine. However, if the amidated polypeptide is desired to be produced by recombinant rather than chemical synthesis, then amidation of the C-terminal amino acid can be performed by several methods known in the art, e.g. use of amidating PAM enzyme. An engineered polypeptide obtainable by recombinant production is preferred.

The ABD herein fold completely reversibly, that is they can be denatured and will refold spontaneously to the desired active tertiary structure. This was assessed by circular dichroism spectra analysis, for example of ABD2 SEQ ID NO:463, where one compares spectrum taken at 20° C. (folded state) and a second spectrum taken after heating to 90° C. (heat denaturation) a third spectrum taken following return to 20° C. (refolded state). During this procedure the Tm can be determined

The compounds (exendins, ABDs, linkers, engineered polypeptides) described herein may also be prepared using recombinant DNA techniques using methods known in the art, such as Sambrook et al., 1989, MOLECULAR CLONING: A LABORATORY MANUAL, 2d Ed., Cold Spring Harbor. Non-peptide compounds may be prepared by art-known methods. For example, phosphate-containing amino acids and peptides containing such amino acids, may be prepared using methods known in the art, such as described in Bartlett et al, 1986, Biorg. Chem., 14:356-377. Compounds can be conjugated using art methods or as described herein

The engineered polypeptides may alternatively be produced by recombinant techniques well known in the art. See, e.g., Sambrook et al., 1989 (Id.). These engineered polypeptides produced by recombinant technologies may be expressed from a polynucleotide. One skilled in the art will appreciate that the polynucleotides, including DNA and RNA, that encode such engineered polypeptides may be obtained from the wild-type cDNA, e.g. exendin-4, taking into consideration the degeneracy of codon usage, and may further engineered as desired to incorporate the indicated substitutions. These polynucleotide sequences may incorporate codons facilitating transcription and translation of mRNA in microbial hosts. Such manufacturing sequences may readily be constructed according to the methods well known in the art. See, e.g., WO 83/04053, incorporated herein by reference in its entirety and for all purposes. The polynucleotides above may also optionally encode an N-terminal methionyl residue. Non-peptide compounds useful in the present invention may be prepared by art-known methods. For example, phosphate-containing amino acids and peptides containing such amino acids may be prepared using methods known in the art. See, e.g., Bartlett and Landen, 1986, Bioorg. Chem. 14: 356-77.

A variety of expression vector/host systems may be utilized to contain and express a engineered polypeptide coding sequence. These include but are not limited to microorganisms such as bacteria transformed with recombinant bacteriophage, plasmid or cosmid DNA expression vectors; yeast transformed with yeast expression vectors; insect cell systems infected with virus expression vectors (e.g., baculovirus); plant cell systems transfected with virus expression vectors (e.g., cauliflower mosaic virus, CaMV; tobacco mosaic virus, TMV) or transformed with bacterial expression vectors (e.g., Ti or pBR322 plasmid); or animal cell systems. Mammalian cells that are useful in recombinant protein productions include but are not limited to VERO cells, HeLa cells, Chinese hamster ovary (CHO) cell lines, COS cells (such as COS-7), WI 38, BHK, HepG2, 3T3, RIN, MDCK, A549, PC12, K562 and 293 cells. Exemplary protocols for the recombinant expression of the protein are described herein and/or are known in the art.

As such, polynucleotide sequences are useful in generating new and useful viral and plasmid DNA vectors, new and useful transformed and transfected procaryotic and eucaryotic host cells (including bacterial, yeast, and mammalian cells grown in culture), and new and useful methods for cultured growth of such host cells capable of expression of the present engineered polypeptides. The polynucleotide sequences encoding engineered polypeptides herein may be useful for gene therapy in instances where underproduction of engineered polypeptides would be alleviated, or the need for increased levels of such would be met.

The present invention also provides for processes for recombinant DNA production of the present engineered polypeptides. Provided is a process for producing the engineered polypeptides from a host cell containing nucleic acids encoding the engineered polypeptide including: (a) culturing the host cell containing polynucleotides encoding the engineered polypeptide under conditions facilitating the expression of the DNA molecule; and (b) obtaining the engineered polypeptides.

Host cells may be prokaryotic or eukaryotic and include bacteria, mammalian cells (such as Chinese Hamster Ovary (CHO) cells, monkey cells, baby hamster kidney cells, cancer cells or other cells), yeast cells, and insect cells.

Mammalian host systems for the expression of the recombinant protein also are well known to those of skill in the art. Host cell strains may be chosen for a particular ability to process the expressed protein or produce certain post-translation modifications that will be useful in providing protein activity. Such modifications of the polypeptide include, but are not limited to, acetylation, carboxylation, glycosylation, phosphorylation, lipidation and acylation. Post-translational processing, which cleaves a “prepro” form of the protein, may also be important for correct insertion, folding and/or function. Different host cells, such as CHO, HeLa, MDCK, 293, WI38, and the like, have specific cellular machinery and characteristic mechanisms for such post-translational activities, and may be chosen to ensure the correct modification and processing of the introduced foreign protein.

Alternatively, a yeast system may be employed to generate the engineered polypeptides of the present invention. The coding region of the engineered polypeptides DNA is amplified by PCR. A DNA encoding the yeast pre-pro-alpha leader sequence is amplified from yeast genomic DNA in a PCR reaction using one primer containing nucleotides 1-20 of the alpha mating factor gene and another primer complementary to nucleotides 255-235 of this gene (Kurjan and Herskowitz, 1982, Cell, 30: 933-43). The pre-pro-alpha leader coding sequence and engineered polypeptide coding sequence fragments are ligated into a plasmid containing the yeast alcohol dehydrogenase (ADH2) promoter, such that the promoter directs expression of a fusion protein consisting of the pre-pro-alpha factor fused to the mature engineered polypeptide. As taught by Rose and Broach, (Rose & Broach, 1990, Meth. Enz., 185: 234-79, Goeddel ed., Academic Press, Inc., San Diego, Calif.), the vector further includes an ADH2 transcription terminator downstream of the cloning site, the yeast “2-micron” replication origin, the yeast leu-2d gene, the yeast REP1 and REP2 genes, the E. coli beta-lactamase gene, and an E. coli origin of replication. The beta-lactamase and leu-2d genes provide for selection in bacteria and yeast, respectively. The leu-2d gene also facilitates increased copy number of the plasmid in yeast to induce higher levels of expression. The REP1 and REP2 genes encode proteins involved in regulation of the plasmid copy number.

The DNA construct described in the preceding paragraph is transformed into yeast cells using a known method, e.g., lithium acetate treatment (Steams et al., 1990,. Meth. Enz. 185: 280-297). The ADH2 promoter is induced upon exhaustion of glucose in the growth media (Price et al., 1987, Gene 55:287). The pre-pro-alpha sequence effects secretion of the fusion protein from the cells. Concomitantly, the yeast KEX2 protein cleaves the pre-pro sequence from the mature engineered polypeptides (Bitter et al., 1984, Proc. Natl. Acad. Sci. USA 81:5330-5334).

Engineered polypeptides of the invention may also be recombinantly expressed in yeast, e.g. Pichia, using a commercially available expression system, e.g., the Pichia Expression System (Invitrogen, San Diego, Calif.), following the manufacturer's instructions. This system also relies on the pre-pro-alpha sequence to direct secretion, but transcription of the insert is driven by the alcohol oxidase (AOX1) promoter upon induction by methanol. The secreted engineered polypeptide is purified from the yeast growth medium by, e.g., the methods used to purify said engineered polypeptide from bacterial and mammalian cell supernatants.

Alternatively, the DNA encoding a engineered polypeptide may be cloned into a baculovirus expression vector, e.g. pVL1393 (PharMingen, San Diego, Calif.). This engineered-polypeptide-encoding vector is then used according to the manufacturer's directions (PharMingen) or known techniques to infect Spodoptera frugiperda cells, grown for example in sF9 protein-free media, and to produce recombinant protein. The protein is purified and concentrated from the media using methods known in the art, e.g. a heparin-Sepharose column (Pharmacia, Piscataway, N.J.) and sequential molecular sizing columns (Amicon, Beverly, Mass.), and resuspended in appropriate solution, e.g. PBS. SDS-PAGE analysis can be used to characterize the protein, for example by showing a single band that confirms the size of the desired engineered polypeptide, as can full amino acid amino acid sequence analysis, e.g. Edman sequencing on a Proton 2090 Peptide Sequencer, or confirmation of its N-terminal sequence.

For example, the DNA sequence encoding the predicted mature engineered polypeptide may be cloned into a plasmid containing a desired promoter and, optionally, a leader sequence (see, e.g., Better et al., 1988, Science 240:1041-1043). The sequence of this construct may be confirmed by automated sequencing. The plasmid is then transformed into E. coli, strain MC1061, using standard procedures employing CaCl2 incubation and heat shock treatment of the bacteria (Sambrook et al., Id.). The transformed bacteria are grown in LB medium supplemented with carbenicillin, and production of the expressed protein is induced by growth in a suitable medium. If present, the leader sequence will affect secretion of the mature engineered polypeptide and be cleaved during secretion. The secreted recombinant engineered polypeptide is purified from the bacterial culture media by the method described herein.

Alternatively, the engineered polypeptides may be expressed in an insect system. Insect systems for protein expression are well known to those of skill in the art. In one such system, Autographa californica nuclear polyhedrosis virus (AcNPV) is used as a vector to express foreign genes in Spodoptera frugiperda cells or in Trichoplusia larvae. The engineered polypeptide coding sequence is cloned into a nonessential region of the virus, such as the polyhedrin gene, and placed under control of the polyhedrin promoter. Successful insertion of a engineered polypeptide will render the polyhedrin gene inactive and produce recombinant virus lacking coat protein coat. The recombinant viruses are then used to infect S. frugiperda cells or Trichoplusia larvae in which engineered polypeptide of the present invention is expressed (Smith et al., 1983, J. Virol. 46:584; Engelhard et al., 1994, Proc. Natl. Acad. Sci. USA 91:3224-3227).

In another example, the DNA sequence encoding the engineered polypeptides may be amplified by PCR and cloned into an appropriate vector, for example, pGEX-3X (Pharmacia, Piscataway, N.J.). The pGEX vector is designed to produce a fusion protein including glutathione-S-transferase (GST), encoded by the vector, and a protein encoded by a DNA fragment inserted into the vector's cloning site. The primers for the PCR may be generated to include, for example, an appropriate cleavage site. The recombinant fusion protein may then be cleaved from the GST portion of the fusion protein. The pGEX-3X/engineered polypeptide construct is transformed into E. coli XL-1 Blue cells (Stratagene, La Jolla, Calif.), and individual transformants are isolated and grown at 37 degrees C. in LB medium (supplemented with carbenicillin) to an optical density at wavelength 600 nm of 0.4, followed by further incubation for 4 hours in the presence of 0.5 mM Isopropyl beta-D-Thiogalactopyranoside (Sigma Chemical Co., St. Louis, Mo.). Plasmid DNA from individual transformants is purified and partially sequenced using an automated sequencer to confirm the presence of the desired engineered polypeptide-encoding gene insert in the proper orientation.

The fusion protein, when expected to be produced as an insoluble inclusion body in the bacteria, may be purified as described above or as follows. Cells are harvested by centrifugation; washed in 0.15 M NaCl, 10 mM Tris, pH 8, 1 mM EDTA; and treated with 0.1 mg/mL lysozyme (Sigma Chemical Co.) for 15 min. at RT. The lysate is cleared by sonication, and cell debris is pelleted by centrifugation for 10 min. at 12,000×g. The fusion protein-containing pellet is resuspended in 50 mM Tris, pH 8, and 10 mM EDTA, layered over 50% glycerol, and centrifuged for 30 min. at 6000×g. The pellet is resuspended in standard phosphate buffered saline solution (PBS) free of Mg⁺⁺ and Ca⁺⁺. The fusion protein is further purified by fractionating the resuspended pellet in a denaturing SDS polyacrylamide gel (Sambrook et al., supra). The gel is soaked in 0.4 M KCl to visualize the protein, which is excised and electroeluted in gel-running buffer lacking SDS. If the GST/engineered polypeptide fusion protein is produced in bacteria as a soluble protein, it may be purified using the GST Purification Module (Pharmacia Biotech).

The fusion protein may be subjected to digestion to cleave the GST from the mature engineered polypeptide. The digestion reaction (20-40 μg fusion protein, 20-30 units human thrombin (4000 U/mg (Sigma) in 0.5 mL PBS) is incubated 16-48 hrs. at RT and loaded on a denaturing SDS-PAGE gel to fractionate the reaction products. The gel is soaked in 0.4 M KCl to visualize the protein bands. The identity of the protein band corresponding to the expected molecular weight of the engineered polypeptide may be confirmed by partial amino acid sequence analysis using an automated sequencer (Applied Biosystems Model 473A, Foster City, Calif.).

In a particularly exemplary method of recombinant expression of the engineered polypeptides of the present invention, mammalian 293 cells may be co-transfected with plasmids containing the engineered polypeptides cDNA in the pCMV vector (5′ CMV promoter, 3′ HGH poly A sequence) and pSV2neo (containing the neo resistance gene) by the calcium phosphate method. In one embodiment, the vectors should be linearized with ScaI prior to transfection. Similarly, an alternative construct using a similar pCMV vector with the neo gene incorporated can be used. Stable cell lines are selected from single cell clones by limiting dilution in growth media containing 0.5 mg/mL G418 (neomycin-like antibiotic) for 10-14 days. Cell lines are screened for engineered polypeptides expression by ELISA or Western blot, and high-expressing cell lines are expanded for large scale growth.

It is preferable that the transformed cells are used for long-term, high-yield protein production and as such stable expression is desirable. Once such cells are transformed with vectors that contain selectable markers along with the desired expression cassette, the cells may be allowed to grow for 1-2 days in an enriched media before they are switched to selective media. The selectable marker is designed to confer resistance to selection, and its presence allows growth and recovery of cells that successfully express the introduced sequences. Resistant clumps of stably transformed cells can be proliferated using tissue culture techniques appropriate to the cell.

A number of selection systems may be used to recover the cells that have been transformed for recombinant protein production. Such selection systems include, but are not limited to, HSV thymidine kinase, hypoxanthine-guanine phosphoribosyltransferase and adenine phosphoribosyltransferase genes, in tk-, hgprt- or aprt-cells, respectively. Also, anti-metabolite resistance can be used as the basis of selection for dhfr, that confers resistance to methotrexate; gpt, that confers resistance to mycophenolic acid; neo, that confers resistance to the aminoglycoside, G418; also, that confers resistance to chlorsulfuron; and hygro, that confers resistance to hygromycin. Additional selectable genes that may be useful include trpB, which allows cells to utilize indole in place of tryptophan, or hisD, which allows cells to utilize histinol in place of histidine. Markers that give a visual indication for identification of transformants include anthocyanins, beta-glucuronidase and its substrate, GUS, and luciferase and its substrate, luciferin.

The engineered polypeptides of the present invention may be produced using a combination of both automated peptide synthesis and recombinant techniques. For example, either or both the exendin compound and the ABD, and optionally a linker, can be made synthetically or recombinantly and then ligated together using methods known in the art, such as “native chemical ligation” and known variations thereof in which an amide bond is formed joining the parent compounds. See, e.g., U.S. Pat. No. 6,326,468, which is incorporated herein by reference and for all purposes. Alternatively, for example, an engineered polypeptide of the present invention may contain a combination of modifications including deletion, substitution, insertion and derivatization by PEGylation (or other moiety, e.g. polymer, fatty acyl chain, C-terminal amidation). Such an engineered polypeptide may be produced in stages. In the first stage, an intermediate engineered polypeptide containing the modifications of deletion, substitution, insertion, and any combination thereof, may be produced by recombinant techniques as described. Then after an optional purification step as described herein, the intermediate engineered polypeptide is PEGylated (or subjected to other chemical derivatization, e.g., acylation, C-terminal amidation) through chemical modification with an appropriate PEGylating reagent (e.g., from NeKtar Transforming Therapeutics, San Carlos, Calif.) to yield the desired engineered polypeptide derivative. One skilled in the art will appreciate that the above-described procedure may be generalized to apply to a engineered polypeptide containing a combination of modifications selected from deletion, substitution, insertion, derivation, and other means of modification well known in the art and contemplated by the present invention.

C-terminal amidation can be achieved by use of a glycine amino acid-C-terminally extended precursor, synthesized for example in yeast (e.g. Pichia) as alpha-factor fusion protein that will be secreted into culture medium. After purification, the C-terminal glycine of the engineered polypeptide precursor can be converted to amide by enzymatic amidation, e.g. peptidylglycine alpha-amidating monooxygenase (PAM). See e.g., Cooper et al., 1989, Biochem. Biophys. Acta, 1014:247-258. See also U.S. Pat. No. 6,319,685, which is incorporated herein by reference in its entirety and for all purposes, which teaches methods for enzymatic amidation, including an alpha-amidating enzyme from rat being sufficiently pure in alpha-amidating enzyme to exhibit a specific activity of at least about 25 mU per mg of protein, and being sufficiently free of proteolytic impurities to be suitable for use with substrates purified from natural sources or produced by recombinant DNA techniques.

Peptides may be purified by any number of methods known in the art, including as described herein. In one method peptides are purified by RP-HPLC (preparative and analytical) using a Waters Delta Prep 3000 system. A C4, C8 or C18 preparative column (10 micron, 2.2×25 cm; Vydac, Hesperia, Calif.) may be used to isolate peptides, and purity may be determined using a C4, C8 or C18 analytical column (5 micron, 0.46×25 cm; Vydac). Solvents (A=0.1% TFA/water and B=0.1% TFA/CH₃CN) may be delivered to the analytical column at a flow rate of 1.0 ml/min and to the preparative column at 15 ml/min. Amino acid analyses may be performed on the Waters Pico Tag system and processed using the Maxima program. Peptides may be hydrolyzed by vapor-phase acid hydrolysis (115° C., 20-24 h). Hydrolysates may be derivatized and analyzed by standard methods (Cohen et al, THE PICO TAG METHOD: A MANUAL OF ADVANCED TECHNIQUES FOR AMINO ACID ANALYSIS, pp. 11-52, Millipore Corporation, Milford, Mass. (1989)). Fast atom bombardment analysis may be carried out by M-Scan, Incorporated (West Chester, Pa.). Mass calibration may be performed using cesium iodide or cesium iodide/glycerol. Plasma desorption ionization analysis using time of flight detection may be carried out on an Applied Biosystems Bio-Ion 20 mass spectrometer.

Engineered Polypeptide Expression Assay.

Methods are available for assaying the level of protein expression by a host cell. Procedures useful for assaying the level of protein expression by a host cell are exemplified in the following typical protocol. About 25 ul BL21 E. coli cells are transformed with 2 ul plasmid DNA (expression vector for the engineered polynucleotide). Cells can be plated and incubated overnight at 37 degrees C. or at room temperature (RT) over a 48-hr period. A single colony can be selected and used to grow starter culture in 4 ml LB media with appropriate antibiotic for ˜6 hrs. Glycerol stocks can be prepared by adding 100 ul 80% sterile glycerol to 900 ul stock, which can then be mixed gently and stored at −80 C. A 250 ul sample can be removed for TCP uninduced sample. An aliquot, for example, 2 ml of Magic media containing appropriate antibiotic can be inoculated with 5 ul starter culture, which can then be incubated overnight (up to 24 hrs) at 37 C, 300 rpm. As known in the art, Magic Media is autoinducing. Alternatively, 60 ml Magic Media containing appropriate antibiotic can be inoculated with 60 ul starter culture in a 250 ml or 125 ml Thompson flask, which can then be incubated overnight (up to 24 hrs) at 30 C, 300 rpm. After incubation, 250 ul culture can be removed from each tube and the cells pelleted. The cell can be resuspended in 1 ml 50 mM Tris pH 8, 150 mM NaCl, to which can be added 0.1 volumes (100 ul) POP culture reagent and 1 ul r-lysozyme (1:750 dilution in r-lysozyme buffer). The mixture can be mixed well and incubated at least 10 min at RT. The preparation can then be centrifuge 10 min at 14000×G. The supernatant (soluble fraction) can be removed and retained, and samples can be prepared for gel analysis (15 ul+5 ul LDS). The remaining inclusion body pellet can be resuspended in 1 ml 1% SDS with sonication. The sample can be prepared for gel analysis (15 ul+5 ul LDS). For uninduced samples, 1.0 volumes POP culture reagent and 1 ul r-lysozyme (1:750 dilution in r-lysozyme buffer) can be added. The mixture can be mixed well and incubated at least 10 min at RT. These samples may not need to be centrifuged. The sample can then be prepared for gel analysis (15 ul+5 ul LDS). NU-PAGE gels (4-12%) non-reduced in 1×MES buffer can be run and stained with SimplyBlue microwave protocol. Destaining can be conducted overnight, as known in the art. A gel image can be retained, and analyzed to determine protein expression levels.

Engineered polypeptides can be and were expressed and isolated as follows. A protein sequence of the desired engineered polypeptide was designed and back translated using commercial software to a DNA sequence for cloning into an E. coli expression vector. Nucleic acid sequences were either obtained as oligonucleotides and ligated using standard PCR amplification techniques, or were digested from existing expression constructs using standard restriction enzymes and then ligated together. Sequences expressing the protein of interest were placed in plasmid pET45 with a T7 promoter for inducible expression. After constructs were verified by sequencing, the vector DNA was purified and transformed into an expression host, typically BL21(DE3). A single colony was selected to grow a starter culture in 4 ml LB media for ˜6 hrs. Glycerol stocks were prepared by adding 100 ul 80% glycerol to 900 ul stock and stored at −80 C. Optionally, 500 ul of un-induced sample was retained for gel analysis. A 60 ml culture (e.g. MagicMedia™ E. coli Expression Medium; Invitrogen, USA; see Glenn et al., J. Biol. Chem. 2008, 283(19):12717-29) was inoculated using 60 ul starter culture in a 125 ml Thompson flask and incubated at 30 degrees C. overnight. Removed 250 ul sample for analysis. The cells were collected as a pellet by centrifuging, and frozen for later processing. Preparation of cell extract and first pass purification with Nickel resin was performed as follows. E. coli cell pellets were completely resuspended in a volume of lysis buffer (50 mM TrisHCl, 150 mM NaCl, pH 8.0) equal to the starting culture volume. Cells were then subjected to a microfluidizer (Microfluidics, MA) at 100 psi for three times. Cell extracts were centrifuged for 30 minutes at 16,000×g to remove debris. EGTA (150 mM stock) was added to the cell extract to a final concentration of 3 mM EGTA. The lysate was then applied to a Ni-NTA Superflow column that had been washed and pre-equilibrated. Protein bound to the column was then washed with lysis buffer plus EGTA (50 mM TrisHCl, 150 mM NaCl, pH8.0, 3 mM EGTA) before the bound protein was eluted with 50 mL of elution buffer (25 mM TrisHCl, 50 mM NaCl, 250 mM Imidazol, pH8.0). Cleavage of His-Tag and subsequent purification was as follows. The eluted protein was concentrated with Amicon-Ultra15 centrifugal filter unit (Millipore, USA) and then diluted with 25 mM TrisHCl, pH8.0, 50 mM NaCl to prepare for protease digestion which removes the HisTag from the N-terminus of the desired protein. Added was 0.1% of β-mercaptoethanol and 1% of Turbo TEV protease (2 mg/mL, 10,000 units/mg; Excellgen, USA) to the protein solution, which was mixed and incubated at room temperature for 4 hours and then at 4° C. over night. An Ni-NTA Superflow column (Qiagen, USA) was pre-equilibrated with 50 mM TrisHCl, 100 mM NaCl, 45 mM imidazole, pH8.0. The TEV digest reaction was diluted 2-fold with 50 mM TrisHCl, 150 mM NaCl, pH8.0. The diluted digest reaction was carefully applied to the top of Ni-NTA column and flow-through was collected. To the column was added 10 mL of 50 mM trisHCl, 100 mM NaCl, 45 mM imidazole, pH8.0 to elute any unbound protein. The eluted proteins from the column were collected and combined, and then polished using size exclusion chromatography (2× with Superdex 75 HiLoad 26/60 column; GE Healthcare Biosciences, USA). Any remaining bacterial endotoxin was removed using EndoTrap Red (Lonza, Switzerland) according to manufacturer's instructions.

Inclusion Body Preparation.

For engineered polypeptides that are found in the inclusion body fraction, the following procedure can be beneficial. The cell pellet can be resuspended in a minimum of 100 ml Lysis buffer for each 50 ml culture. Upon the addition of 30 ml, a 10 ml pipette can be used to resuspend, then the tube can be washed out with an additional 70 ml. The resuspended cell solution can be multiply run, e.g., 4 passes, through a microfluidizer@ 100 PSI (min) taking care to keep chamber in ice water through the entire process. The fluidized slurry can be centrifuged at 14000×g, 20 min (e.g., JLA 10.5, 10,000 rpm, using 250 ml nalgene bottles). The inclusion body pellet can be resuspended on ice in chilled lysis buffer with stir bar and stir plate for 1 hour at 4 C after disruption with pipette tip. The pellet can be resuspended a second time in distilled H₂O with stir bar and stir plate for 1 hour at 4 C after disruption with pipette tip, followed by centrifugation at 14000×g, 15 min. The supernatant can be removed and discarded. The resultant can be stored at −80 C.

Protein Purification.

As described herein, numerous methods are known for isolation of expressed polypeptides. Preferred are secreted engineered polypeptides. However, the following is one example if inclusion bodies are formed. Inclusion body pellets can be solubilized in appropriate volume of solubilization buffer (8M urea or 8M guanidine, 50 mM Tris, 10 mM DTT, pH 7.75) for 1 hour at RT. The solubilized pellets can be centrifuged for 20 min at 27 000 g. Filtered (e.g., 0.4 um) supernatant can be transferred drop by drop into appropriate volume of refolding buffer (50 mM Tris-HCl, 1 M urea, 0.8 M arginine, 4 mM cysteine, 1 mM cystamine; pH 8) at RT. The result can then be placed at 4° C. overnight or longer with gentle mixing. Samples can be concentrated and run on a gel filtration column (Superdex75 26/60) at 1-2 ml/min in 4 C environment using a GE Healthsciences AKTA FPLC. Appropriate protein containing fractions can be identified via SDS-PAGE, pooled and run through a second gel filtration column. Pooled protein can then be concentrated in Amicon filter to appropriate concentration and assayed for endotoxin levels using, e.g., Endosafe PTS Reader (Charles River), as known in the art. Once a protein sample has passed the endotoxin criteria, it can be sterile filtered, dispensed into aliquots and run through quality control assays. Quality control assays can include analytical HPLC-SEC, non reducing SDS PAGE and RP HPLC-MS to obtain approximate mass. Proteins can be obtained in 1×PBS (137 mM sodium chloride, 2.7 mM potassium chloride, 4.3 mM disodium phosphate, 1.4 mM monopotassium phosphate, pH7.2), distributed into aliquots and flash frozen for storage at −70 to −80° C.

### IV. Methods of Use and Treating Disease

### Indications

A variety of diseases and disorders are contemplated to be beneficially treated by the polypeptide compounds and methods described herein, primarily based upon those amenable to treatment by interaction with the GLP-1 receptor, such as by exendin-4.

Obesity and Overweight.

Obesity and its associated disorders including overweight are common and serious public health problems in the United States and throughout the world. Upper body obesity is the strongest risk factor known for type 2 diabetes mellitus and is a strong risk factor for cardiovascular disease. Obesity is a recognized risk factor for hypertension, atherosclerosis, congestive heart failure, stroke, gallbladder disease, osteoarthritis, sleep apnea, reproductive disorders such as polycystic ovarian syndrome, cancers of the breast, prostate, and colon, and increased incidence of complications of general anesthesia. See, e.g., Kopelman, 2000, Nature 404:635-43.

Obesity reduces life-span and carries a serious risk of the co-morbidities listed above, as well disorders such as infections, varicose veins, acanthosis nigricans, eczema, exercise intolerance, insulin resistance, hypertension hypercholesterolemia, cholelithiasis, orthopedic injury, and thromboembolic disease. See e.g., Rissanen et al, 1990, Br. Med. J., 301:835-7. Obesity is also a risk factor for the group of conditions called insulin resistance syndrome, or “Syndrome X” and metabolic syndrome. The worldwide medical cost of obesity and associated disorders is enormous.

The pathogenesis of obesity is believed to be multi-factoral. A problem is that, in obese subjects, nutrient availability and energy expenditure do not come into balance until there is excess adipose tissue. The central nervous system (CNS) controls energy balance and coordinates a variety of behavioral, autonomic and endocrine activities appropriate to the metabolic status of the animal. The mechanisms or systems that control these activities are broadly distributed across the forebrain (e.g., hypothalamus), hindbrain (e.g., brainstem), and spinal cord. Ultimately, metabolic (i.e., fuel availability) and cognitive (i.e., learned preferences) information from these systems is integrated and the decision to engage in appetitive (food seeking) and consummatory (ingestion) behaviors is either turned on (meal procurement and initiation) or turned off (meal termination). The hypothalamus is thought to be principally responsible for integrating these signals and then issuing commands to the brainstem. Brainstem nuclei that control the elements of the consummatory motor control system (e.g., muscles responsible for chewing and swallowing). As such, these CNS nuclei have literally been referred to as constituting the “final common pathway” for ingestive behavior.

Neuroanatomical and pharmacological evidence support that signals of energy and nutritional homeostasis integrate in forebrain nuclei and that the consummatory motor control system resides in brainstem nuclei, probably in regions surrounding the trigeminal motor nucleus. There are extensive reciprocal connection between the hypothalamus and brainstem. A variety of CNS-directed anti-obesity therapeutics (e.g., small molecules and peptides) focus predominantly upon forebrain substrates residing in the hypothalamus and/or upon hindbrain substrates residing in the brainstem.

Obesity remains a poorly treatable, chronic, essentially intractable metabolic disorder. Accordingly, a need exists for new therapies useful in weight reduction and/or weight maintenance in a subject. Such therapies would lead to a profound beneficial effect on the subject's health.

Diabetes and Cardiovascular Disease.

Diabetes mellitus is recognized as a complex, chronic disease in which 60% to 70% of all case fatalities among diabetic patients are a result of cardiovascular complications. Diabetes is not only considered a coronary heart disease risk equivalent but is also identified as an independent predictor of adverse events, including recurrent myocardial infarction, congestive heart failure, and death following a cardiovascular incident. The adoption of tighter glucose control and aggressive treatment for cardiovascular risk factors would be expected to reduce the risk of coronary heart disease complications and improve overall survival among diabetic patients. Yet, diabetic patients are two to three times more likely to experience an acute myocardial infarction than non-diabetic patients, and diabetic patients live eight to thirteen years less than non-diabetic patients.

Understanding the high risk nature of diabetic/acute myocardial infarction patients, the American College of Cardiology/American Heart Association (“ACC/AHA”) clinical practice guidelines for the management of hospitalized patients with unstable angina or non-ST-elevation myocardial infarction (collectively referred to as “ACS”) recently recognized that hospitalized diabetic patients are a special population requiring aggressive management of hyperglycemia. Specifically, the guidelines state that glucose-lowering therapy for hospitalized diabetic/ACS patients should be targeted to achieve preprandial glucose less than 10 mg/dL, a maximum daily target than 180 mg/dL, and a post-discharge hemoglobin Alc less than 7%.

In a nationwide sample of elderly ACS patients, it was demonstrated that an increase in 30-day mortality in diabetic patients corresponded with the patients having higher glucose values upon admission to the hospital. See “Diabetic Coronary Artery Disease & Intervention,” Coronary Therapeutics 2002, Oak Brook, Ill., Sep. 20, 2002. There is increasing evidence that sustained hyperglycemia rather than transient elevated glucose upon hospital admission is related to serious adverse events. Although the ideal metric for hyperglycemia and vascular risk in patients is not readily known, it appears that the mean glucose value during hospitalization is most predictive of mortality. In a separate study of ACS patients form over forty hospitals in the United States, it was found that persistent hyperglycemia, as opposed to random glucose values upon admission to the hospital, was more predictive of in-hospital mortality. See Acute Coronary Syndrome Summit: A State of the Art Approach, Kansas City, Mo., Sep. 21, 2002. Compared with glucose values upon admission, a logistic regression model of glucose control over the entire hospitalization was most predictive of mortality. There was nearly a two-fold increased risk of mortality during hospitalization for each 10 mg/dL increase in glucose over 120 mg/dL. In a smaller cohort of consecutive diabetic/ACS patients, there was a graded increase in mortality at one year with increasing glucose levels upon hospital admission. In the hospital setting, the ACC/AHA guidelines suggest initiation of aggressive insulin therapy to achieve lower blood glucose during hospitalization.

Lipid Regulation Diseases.

Dyslipidemia is a disruption in the normal lipid component in the blood. It is believed that prolonged elevation of insulin levels can lead to dyslipidemia. Hyperlipidemia is the presence of raised or abnormal levels of lipids and/or lipoproteins in the blood. Fatty liver disease, e.g., nonalcoholic fatty liver disease (NAFLD) refers to a wide spectrum of liver disease ranging from simple fatty liver (steatosis), to nonalcoholic steatohepatitis (NASH), to cirrhosis (irreversible, advanced scarring of the liver). All of the stages of NAFLD have in common the accumulation of fat (fatty infiltration) in the liver cells (hepatocytes).

Additionally, without wishing to be bound by any theory, it is believed that relative insulin deficiency in type 2 diabetes, glucose toxicity, and increased hepatic free fatty acid burden through elevated delivery from intra-abdominal adipose tissue via the portal vein, are implicated as possible causes in fatty liver disorders. Indeed, it has been hypothesized that eating behavior is the key factor driving the metabolic syndrome of obesity with its many corollaries, including NASH. Accordingly, treatments aimed at decreasing food intake and increasing the number of small meals, as has already been demonstrated in type 2 diabetes, may effectively treat and prevent NASH. Drugs that promote insulin secretion and weight loss, and delay gastric emptying are also effective at improving glucose tolerance and thus may improve fatty liver with its attendant hyperinsulinemia. Thus, use of exendins, exendin analog agonists, exendin derivative agonists, particularly exendin-4, can be well suited as a treatment modality for this condition. Accordingly, engineered polypeptides described herein which include an exendin or biologically active (hormone domain) peptide component, or fragment or analog thereof, can be useful in the treatment of fatty liver disorders.

Alzheimer's Disease.

Alzheimer's disease (AD), as known in the art, is associated with plaques and tangles in the brain which include dysregulation of the A-beta protein. Stimulation of neuronal GLP-1 receptors has been reported to play an important role in regulating neuronal plasticity and cell survival. Stimulation of GLP-1 receptor has been reported to induce neurite outgrowth and to protect against excitotoxic cell death and oxidative injury in cultured neuronal cells. GLP-1 and exendin-4 were reported to reduce endogenous levels of amyloid-beta peptide (A-beta protein) in mouse brain and to reduce levels of beta-amyloid precursor protein (beta-APP) in neurons. See, e.g., Perry et al., 2004, Curr. Drug Targets 5(6):565-571. Treatment with the engineered compounds disclosed herein can provide benefit to the therapeutic targets associated with Alzheimer's disease.

Parkinson's Disease.

Parkinson's disease (PD) is the synonym of “primary parkinsonism”, i.e. isolated parkinsonism due to a neurodegenerative process without any secondary systemic cause. Parkinsonism is characterized by symptoms of tremor, stiffness, and slowing of movement caused by loss of dopamine Without wishing to be bound by any theory, it is believed that exendin-4 may act as a survival factor for dopaminergic neurons by functioning as a microglia-deactivating factor and suggest that exendin-4 may be a valuable therapeutic agent for neurodegenerative diseases such as PD.

Metabolic Syndrome X.

Metabolic Syndrome X is characterized by insulin resistance, dyslipidemia, hypertension, and visceral distribution of adipose tissue, and plays a pivotal role in the pathophysiology of type 2 diabetes. It has also been found to be strongly correlated with NASH, fibrosis, and cirrhosis of the liver. Accordingly, engineered polypeptides described herein can be useful in the treatment of metabolic syndrome X.

Steroid Induced Diabetes.

Glucocorticoids are well known to affect carbohydrate metabolism. In response to exogenous glucocorticoid administration, increased hepatic glucose production and reduced insulin secretion and insulin-stimulated glucose uptake in peripheral tissues is observed. Furthermore, glucocorticoid treatment alters the proinsulin(P1)/immunoreactive insulin(IRI) ratio, as known in the art. Typical characteristics of the hyperglycemia induced by glucocorticoids in subjects without diabetes include a minimal elevation of fasting blood glucose, exaggerated postprandial hyperglycemia, insensitivity to exogenous insulin, and non-responsiveness to metformin or sulfonylurea therapy. Accordingly, engineered polypeptides described herein which include an exendin biologically active (hormone domain) peptide component, or fragment or analog thereof, can be useful in the treatment of steroid induced diabetes.

Human Immunodeficiency Virus (HIV) Treatment-Induced Diabetes.

Shortly after the introduction of human immunodeficiency virus (HIV)-1 protease inhibitors (PIs) into routine clinical use, reports linking PT use with the development of hyperglycemia began to appear. While approximately 1% to 6% of HIV-infected subjects who are treated with PIs will develop diabetes mellitus, a considerably larger proportion will develop insulin resistance and impaired glucose tolerance. Accordingly, engineered polypeptides described herein which include an exendin biologically active (hormone domain) peptide component, or fragment or analog thereof, can be useful in the treatment of HIV treatment-induced diabetes.

Latent Autoimmune Diabetes in Adults (LADA).

Progressive autoimmune diabetes, also known as latent autoimmune diabetes in adults (LADA), is thought to be present in approximately 10% of patients diagnosed with type 2 diabetes. LADA patients have circulating antibodies to either islet cell cytoplasmic antigen or, more frequently, glutamic acid decarboxylase. These subjects exhibit clinical features characteristic of both type 1 and type 2 diabetes. Although insulin secretion is better preserved in the slowly progressing than in the rapidly progressing form of autoimmune diabetes, insulin secretion tends to deteriorate with time in LADA subjects. Accordingly, engineered polypeptides described herein which include an exendin biologically active (hormone domain) peptide component, or fragment or analog thereof, can be useful in the treatment of LADA.

Hypoglycemia Unawareness (HU).

Defective glucose counterregulation can occur even after only a single recent episode of hypoglycemia. Subjects who experience repeated episodes of hypoglycemia often lose their capacity to recognize the symptoms typically associated with hypoglycemia or impending insulin shock, a condition called “hypoglycemia unawareness”. Because the-patient doesn't appreciate his or her own status, blood glucose levels can then fall so low that serious neurological problems ensue, including coma and seizure. Accordingly, engineered polypeptides described herein which include an exendin biologically active (hormone domain) peptide component, or fragment or analog thereof, can be useful in the treatment of HU.

Restrictive Lung Disease.

GLP 1 receptor has been localized in the lung. Exendins can elicit a biological response via GLP-1 receptor. In particular, sarcoidosis is a systemic granulomatous disease that frequently involves the lung. Although classically thought of as a restrictive lung disease, airway obstruction has become a recognized feature of the disease in the past years. Sarcoidosis can affect the airway at any level and when the involvement includes small airways, it can resemble more common obstructive airway diseases, such as asthma and chronic bronchitis. Accordingly, engineered polypeptides described herein which include an exendin biologically active (hormone domain) peptide component, or fragment or analog thereof, can be useful in the treatment of restrictive lung disease because such hormone domain peptide can improve elasticity of lung or delay rigidity.

Short Bowel Syndrome (SBS).

Exendin-4 has been reported as effective for the treatment of short bowel syndrome. See Kunkel et al. Neurogastroenterol. Motil. (2011). SBS is a serious clinical disorder characterized by diarrhea and nutritional deprivation. Glucagon-like peptide-1 (GLP-1), produced by L-cells in the ileum, regulates proximal gut transit. When extensive ileal resection occurs, as in SBS, GLP-1 levels may be deficient. Exenatide improved the nutritional state and intestinal symptoms of patients with SBS. Accordingly, SBS patients are amenable to treatment with the engineered polypeptides described herein. Improvement in bowel frequency and form and obtaining bowel movements that are no longer meal-related can be achieved. An additional benefit is that total parenteral nutrition can be stopped. These compounds herein will provide substantial improvement in the bowel habits, nutritional status and quality of life of SBS patients, and further may reduce the need for parenteral nutrition and small bowel transplant.

Accordingly, in one aspect, there is provided a method for treating a disease or disorder in a subject. The subject is in need of treatment for the disease or disorder. In some embodiments, the subject is need of treatment is obese. The disease or disorder is diabetes, overweight, obesity, Alzheimer's disease, fatty liver disease, dyslipidemia, coronary artery disease, stroke, SBS or hyperlipidemia, or other diseases discussed herein. Diabetes can include type I, type II, gestational or pre-diabetes as well as HIV or steroid induced diabetes. The method of treatment includes administration to the subject of a engineered polypeptide as described herein in an amount effective to treatment the disease or disorder. Particularly useful for these diseases are compounds described herein having glucose lowering activity (e.g. exendin-4 or its fragments or analogs linked to an ABD), having reduction of body weight or reduction of food intake activity, lowering of HbA1c, delaying of gastric emptying, lowering of plasma glucagon, and/or intestinal motility benefit.

In some embodiments, the disease or disorder is diabetes, overweight or obesity, or dyslipidemia or hyperlipidemia. The engineered polypeptide can include ABD and HD1 polypeptides, and optionally a linker L1, where HD1 is an exendin or fragment or analog thereof. Accordingly, the engineered polypeptide can have one of the following structures: HD1-ABD or HD1-L1-ABD. In some embodiments, the exendin is preferably exendin-4 or Leu14 exendin-4.

In some embodiments, the disease or disorder is diabetes, overweight, obesity, dyslipidemia, Alzheimer's disease, fatty liver disease, SBS or hyperlipidemia. The engineered polypeptide may include an exendin or fragment or analog thereof. Accordingly, the engineered polypeptide can have one of the following structures: HD1-ABD or HD1-L1-ABD. In some embodiments, the exendin in the engineered polypeptide is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4. Particularly useful for these diseases are compounds described herein having glucose lowering activity (e.g. exendin-4 or its fragments or analogs linked to an ABD), having reduction of body weight or reduction of food intake activity, lowering of HbA1c, delaying of gastric emptying, lowering of plasma glucagon, or intestinal motility benefit.

In some embodiments, the disease or disorder is diabetes, overweight, obesity, dyslipidemia, Alzheimer's disease, fatty liver disease, SBS or hyperlipidemia. The engineered polypeptide may include an exendin or fragment or analog thereof. Accordingly, the engineered polypeptide can have one of the following structures: HD1 ABD or HD1 L1 ABD. In some embodiments, the exendin is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4. Particularly useful for these diseases are compounds described herein having glucose lowering activity (e.g. exendin-4 or its fragments or analogs linked to an ABD), having reduction of body weight or reduction of food intake activity, delaying of gastric emptying, lowering of plasma glucagon, or intestinal motility benefit.

The disease or disorder can be diabetes, overweight, obesity, dyslipidemia, Alzheimer's disease, fatty liver disease, SBS, hyperlipidemia, Parkinson's disease or cardiovascular disease or other diseases described herein. The engineered polypeptide may include an exendin or fragment or analog thereof. Accordingly, the engineered polypeptide can have one of the following structures: HD1-ABD or HD1-L1-ABD. In some embodiments, the exendin is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4. Particularly useful for these diseases are compounds described herein having glucose lowering activity (e.g. exendin-4 or its fragments or analogs linked to an ABD), having reduction of body weight or reduction of food intake activity, a lowering of HbA1c, delaying of gastric emptying, lowering of plasma glucagon, or intestinal motility benefit.

Additional diseases and disorders which can be treated by the compounds and methods described herein include steroid-induced diabetes, HIV treatment-induced diabetes, latent autoimmune diabetes in adults (LADA), Nonalcoholic steatohepatitis (NASH) and nonalcoholic fatty liver disease (NAFLD), hypoglycemia unawareness (HU), restrictive lung disease including sarcoidosis, and metabolic syndrome X. The engineered polypeptide may include an exendin or fragment or analog thereof. Accordingly, the engineered polypeptide can have one of the following structures: HD1-ABD or HD1-L1-ABD. In some embodiments, the exendin is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4. Particularly useful for these diseases are compounds described herein having glucose lowering activity (e.g. exendin-4 or its fragments or analogs linked to an ABD), having reduction of body weight or reduction of food intake activity, delaying of gastric emptying, lowering of HbA1c, lowering of plasma glucagon, or intestinal motility benefit. The engineered polypeptide can include only exendin, or analog or fragment thereof, as a hormone domain. The disease or disorder can be diabetes, overweight, obesity, dyslipidemia, Alzheimer's disease, fatty liver disease, SBS, hyperlipidemia, Parkinson's disease or cardiovascular disease or other diseases described herein. The engineered polypeptide may include an exendin or fragment or analog thereof, preferably exendin-4 or Leu14 exendin-4, linked to an ABD. Accordingly, the engineered polypeptide can have one of the following structures: HD1-ABD or HD1-L1-ABD. Preferably the compounds are having reduction of body weight or reduction of food intake activity, delaying of gastric emptying, lowering of plasma glucagon, or intestinal motility benefit.

Additional diseases and disorders which can be treated by the compounds and methods described herein include steroid-induced diabetes, HIV treatment-induced diabetes, latent autoimmune diabetes in adults (LADA), Nonalcoholic steatohepatitis (NASH) and nonalcoholic fatty liver disease (NAFLD), hypoglycemia unawareness (HU), restrictive lung disease including sarcoidosis, and metabolic syndrome X. The engineered polypeptide preferably has one of the following structures: HD1-analogs linked to an ABD or HD1-L1-ABD. In some embodiments, the exendin is preferably exendin-4 or its analog Leu14 exendin-4. In some embodiments, the exendin fragment is a fragment of exendin-4. In some embodiments, the exendin analog has at least 70%, for example 70%, 75%, 80%, 85%, 90%, 95% or even higher, identity with exendin-4.

### V. Assays

Methods for production and assay of engineered polypeptides described herein are generally available to the skilled artisan. Further, specific methods are described herein as well as in the patent publications and other references cited herein, which are incorporated by reference for this additional purpose.

GLP-1 Receptor Binding and Functional Assays:

GLP-1 receptor binding activity and affinity may be measured in any number of known methods. For example, in one method binding activity is measured using a binding displacement assay in which the receptor source is RINm5F cell membranes, and the ligand is [¹²⁵I]GLP-1 or iodinated exendin(1-39) or iodinated exendin(9-39). Homogenized RINm5F cell membranes are incubated in 20 mM HEPES buffer with 40,000 cpm [¹²⁵I]GLP-1 (or exendin) tracer, and varying concentrations of test compound for 2 hours at 23° C. with constant mixing. Reaction mixtures are filtered through glass filter pads presoaked with 0.3% PEI solution and rinsed with ice-cold phosphate buffered saline. Bound counts are determined using a scintillation counter. Binding affinities are calculated using GraphPad Prism® software (GraphPad Software, Inc., San Diego, Calif.).

In vitro assays for functional GLP-1 receptor activation can be performed using known methods and cells and tissues. For example, exendin-4 stimulation of GLP-1 receptor bearing cells can induce an increase in adenylate cyclase activation, cAMP synthesis, membrane depolarization, rise in intracellular calcium and increase in glucose-induced insulin secretion. See e.g., Holz et al., 1995, J. Biol. Chem. 270(30):17749-57. Cell-based assays using the rMTC 6-23 (clone 6) cell line can be used to determine GLP-1 receptor agonist activity of a compound based on the cAMP generated. In one embodiment of the bioassay the GLP-1 receptor agonist activity of a compound is quantitatively determined by correlations to cAMP production in cell-based assays with 6-23 (clone 6) cells. The cell-based assay uses living 6-23 (clone 6) cells. The 6-23 (clone 6) cells are available from the American Type Culture Collection as ATCC® No. CRL-1607™ and the European Collection of Cell Cultures as ECACC No. 87042206. In another embodiment the cell-based assay is a homogeneous time-resolved fluorescence assay (HTRF®). HTRF® kits are commercially available from Cisbio International (Bedford, Mass.). Methods for using HTRF® kits are known in the art and the kits generally include instruction manuals, e.g., on how to prepare samples, standards, calibration curves, and conduct experiments. Homogeneous time-resolved fluorescence cell-based assays are described in U.S. Pat. No. 5,527,684, the disclosure of which is incorporated by reference herein, and Document Reference No. 62AM4PEB rev02 (August 2007) available from Cisbio HTRF® Product Center. See www.htrf.com/products/gper/camp/, the disclosure of which is incorporated by reference herein. In a preferred method the bioassay uses the rat thyroid carcinoma 6-23 (clone 6) cells in a cell-based assay using the HTRF® cAMP dynamic 2 1,000 assay kit, available from Cisbio as Catalog No. 62AM4PEB. The HTRF® standards and calibrations are prepared following the instructions in the kit. Assays may be performed with or without the presence of albumin.

In vivo assays for activity and duration of action and pharmacokinetics can be done using known methods. For example, duration can be performed using an oral glucose tolerance test (OGTT) in which the drug is administered to the subject at a desired time point before the glucose is administered orally (to measure drug duration of action; OGTT DOA) and glucose blood levels are measured (e.g. readily done in mice). Activity and duration can also be measured using an intravenous glucose tolerance test (IVGTT) in which the drug is administered to the subject at a desired time point before the glucose is administered IV (IVGTT DOA) and blood glucose levels are measured (e.g. can readily be done in rats). Preferred engineered compounds have a desired effect on blood glucose of at least 24 hours duration after a single dose of drug, preferably at least 3 days, at least 4 days, at least 5 days, at least 6 days, and at least 1 week after the single dose of drug is given.

For example, test polypeptide is injected subcutaneously at t=0 immediately following a baseline sample into NIH/Swiss female mice. Blood samples are taken at desired time periods such as t=2, 4, and 8 hours during day 1 and then daily through day 5 or through to day 7 or longer. Blood glucose is measured with a OneTouch® Ultra® (LifeScan, Inc., a Johnson & Johnson Company, Milpitas, Calif.). For a duration of activity (DOA) determination, such as for glucose control activity of a drug, an OGTT or IVGTT can be performed at the desired point after drug administration. Body weight can also be measured, as well as food intake, or other pharmacological or pharmacokinetic parameter. For example, female NIH/Swiss mice (8-20 weeks old) are group housed with a 12:12 hour light:dark cycle with lights on at 0600. Water and a standard pelleted mouse chow diet were available ad libitum, except as noted. The morning of the experiment, animals are divided into experimental groups and fasted starting at approximately 0630 hrs. In a typical study, n=2 cages with 3 mice/cage. At time=0 min, a blood glucose sample is taken and immediately followed by an intraperitoneal injection of vehicle or compound in an amount ranging from about 1 nmol/kg to 25 nmol/kg. Blood glucose can be measured at 30, 60, 120, 180, and 240 min and daily for a week or longer after the single dose. In a variation of the experiment, doses are provided daily or even weekly over a longer period such as 14 or 28 days. Percent pre-treatment is calculated by dividing the blood glucose at the measured time point, e.g. 60 minutes or 1 day, by the blood glucose at time=0 min. Significant treatment effects were identified by ANOVA (p<0.05). Where a significant difference exists, test means are compared to the control mean using Dunnett's test (Prism® v. 4.01, GraphPad Software Inc., San Diego, Calif.). Blood glucose can measured with a OneTouch® Ultra® (LifeScan, Inc., a Johnson & Johnson Company, Milpitas, Calif.). * p<0.05 vs. vehicle control; ANOVA, Dunnett's test. Other parameters can also be measured.

In Vivo Assay for Food Intake Inhibition:

The engineered polypeptides may be tested for their duration and extent of appetite suppression and for their duration and extent of effect on body weight loss in various known methods. For example, the polypeptides may be tested for appetite suppression in the mouse food intake assay and for their effect on body weight gain in diet-induced obesity (DIO) mice. An experimental protocol for such assays are described below.

For example, female NIH/Swiss mice (8-24 weeks old) are group housed with a 12:12 hour light:dark cycle with lights on at 0600. Water and a standard pelleted mouse chow diet are available ad libitum, except as noted. Animals are fasted starting at approximately 1500 hrs, 1 day prior to experiment. The morning of the experiment, animals are divided into experimental groups. In a typical study, n=4 cages with 3 mice/cage. At time=0 min, all animals are given an intraperitoneal injection of vehicle or test compound, typically in an amount ranging from about 2 nmol/kg to 75 nmol/kg, and immediately given a pre-weighed amount (10-15 g) of standard chow. Food is removed and weighed at various times, typically 30, 60, and 120 minutes or longer, such as daily, to determine the amount of food consumed (Morley, Flood et al., 1994, Am. J. Physiol. 267: R178-R184). Food intake is calculated by subtracting the weight of the food remaining at the e.g., 30 or 60 minute time point, from the weight of the food provided initially at time=0. Significant treatment effects are identified by ANOVA (p<0.05). Where a significant difference exists, test means are compared to the control mean using Dunnett's test (Prism® v. 2.01, GraphPad Software Inc., San Diego, Calif.). Body weight can also be measured.

Body Weight, Fat Redistribution, and Lean Body Mass Assays:

Assays for body weight and related effects can also be performed as follows. Diet-induced obesity (DIO) in the in the Sprague-Dawley rat is a valuable model for the study of obesity and regulation of energy homeostasis. These rats were developed from a line of (Crl:CD®(SD)BR) rats that are prone to become obese on a diet relatively high in fat and energy. See, for example, Levin, 1994, Am. J. Physiol. 267:R527-R535, Levin et al., 1997, Am. J. Physiol. 273:R725-R730. DIO male rats are obtained from Charles River Laboratories, Inc. (Wilmington, Mass.). The rats are housed individually in shoebox cages at 22° C. in a 12/12-hour light dark cycle. Rats are maintained ad-libitum on a moderately high fat diet (32% kcal from fat; Research Diets D1226B). The animals typically achieve a mean body weight of about 500 g. Levin DIO rats are habituated to caging environment for 7 days. During the 3 nights of habituation, animals receive a single intraperitoneal (IP) injection of vehicle. On test day, rats are administered a single IP injection of compound or vehicle (e.g. 10% DMSO) at the onset of the dark cycle. Food intake is measured by an automated food intake measuring system (BioDAQ, Research Diets) at 5 sec intervals throughout the course of the study. Body weight is recorded nightly.

Body composition can be measured prior to and after drug treatment using NMR (Echo Medical Systems, Houston, Tex.). For body composition measurements, rats are briefly placed (˜1 min) in a well-ventilated plexiglass tube that was then inserted into a specialized rodent NMR machine. This enabled the calculation of changes in actual grams of fat and dry lean tissue (e.g., grams of body fat after treatment−grams of body fat at baseline=change in grams of body fat) and changes in % body composition for fat and dry lean tissue (e.g., % body fat after treatment−% body fat at baseline=change in % body fat). All data are represented as mean±SEM. Analysis of variance (ANOVA) and post-hoc tests are used to test for group difference. A P-value <0.05 is considered significant. Statistical analysis and graphing are performed using PRISM® 4 for Windows (GraphPad Software, Inc., San Diego, Calif.). Graphs and results are typically presented as vehicle-corrected changes in percent body weight, body fat and changes in body protein

### VI. Pharmaceutical Compositions of Exendin ABD for Transmucosal Delivery

Provided are compositions comprising (a) an Exendin ABD, either of the ABD1 or ABD2 type, and (b) at least one permeation enhancer. The formulations are useful for transmucosal, non-invasive delivery of the Exendin ABD to a subject in need of therapeutic treatment. The formulation can be administered via any transmucosal surface, but particularly via an oral, sub-lingual, buccal, nasal, pulmonary, vaginal or rectal route. Preferably, via oral or sub-lingual delivery, more preferably the oral route (for delivery to and primary absorption by the small intestine).

As used herein, the term “transmucosal” means delivery to and absorption via mucosal tissue, encompassing the mucosal tissue of the mouth, small intestine, large intestine, nasal cavity, rectum or vagina. More specifically what is intended by the phrase is that the primary delivery route of the active ingredient Exendin ABD occurs through the mucosal tissue of the targeted mucosa. As used herein, the phrase “oral dosage form”, when used in the general sense, includes disintegrable,/dissolvable tablets, pellets, granules, microcapsules, powders, capsules, caplets, gels, creams, films, sprays, and the like, suitable for oral delivery.

The formulations provide non-invasive, transmucosal delivery of Exendin ABD compounds that have good duration of action, high potency and/or convenient dosing regimens including oral administration, and method of use thereof. The engineered polypeptides Exendin ABD incorporate an albumin binding domain in combination with a biologically active peptide having exendin-4 activity. Without wishing to be bound by any theory, it is believed that because the engineered polypeptides described herein can bind albumin with high affinity, the compounds can be sequestered (e.g., bound to albumin) while in the circulation leading to increased duration of action, due for example to decreased renal clearance and/or degradation. Surprisingly, the compounds are active while bound to circulating serum albumin. Because the compounds also comprise a sequence with exendin-4 activity, diseases amendable to such treatment include obesity and overweight, diabetes, dyslipidemia, hyperlipidemia, short bowel syndrome, Alzheimer's disease, fatty liver disease, NASH, Parkinson's disease, cardiovascular disease, and other disorders of the central nervous system, or combinations thereof

Accordingly, provided are compositions and improved compositions for non-invasive, transmucosal delivery comprising an Exendin ABD, as defined herein, and at least one transmucosal permeation enhancer. The permeation enhancer or combination of permeation enhancers provides formulations for non-invasive, transmucosal delivery, or improved transmucosal delivery. Further improvement can be achieved by the incorporation of additional agents as described herein. The permeation enhancer can enhance paracellular permeation, open cell tight junctions, enhance transcellular permeation, inhibit an intestinal protease, enhance solubility of a different permeation enhancer and/or be mucoadhesive. The composition can optionally, but preferably, further comprise a second permeation enhancer, wherein the second permeation enhancer enhances paracellular permeation, opens cell tight junctions, enhances transcellular permeation, inhibits an intestinal protease, enhances solubility of a different permeation enhancer in the composition and/or is a mucoadhesive. The composition can further optionally but preferably comprise a third permeation enhancer, wherein the third permeation enhancer enhances paracellular permeation, opens cell tight junctions, enhances transcellular permeation, inhibits an intestinal protease, enhances solubility of a different permeation enhancer in the composition and/or is a mucoadhesive. The composition can optionally further comprise (c) an inhibitor of an intestinal protease, a mucoadhesive, a surfactant, an oil, an emulsifier or a mixture thereof, to further improve bioavailability of the delivered Exendin ABD. The composition can optionally further comprise (d) a pH lowering agent to further improve bioavailability of the delivered Exendin ABD by decreasing activity of proteases at the site of delivery. The composition can optionally further comprise conventional formulation agents (e) including a bulking agent, a polypeptide stabilizing agent, or other excipient, or a mixture thereof

In a preferred formulation embodiment, the composition comprises an Exendin ABD formulated with a permeation enhancer that is a non-conjugated bile acid or salt and a permeation enhancer that is an aromatic alcohol. Preferably the non-conjugated bile acid or salt, in addition to enhancing permeation, enhances the solubility of the aromatic acid at the site of absorption, e.g. small intestine.

The permeation enhancer enhances transmucosal permeation, or more specifically enhances permeation of an Exendin ABD across a mucosal, cell-lined barrier, e.g. intestinal wall, nasal lining, oral cavity, tongue, allowing Exendin ABD absorption and subsequent circulation in the subject's blood, by increasing permeability of a mucosa to the active agent, or otherwise facilitates penetration of the drug through a mucosa. Preferably the permeation enhancer is on the GRAS list (“generally regarded as safe”). Preferably the permeation enhancer enhances permeation of the active by enhancing paracellular permeation, opening cell tight junctions, enhancing transcellular permeation, inhibiting a protease at the site of delivery and absorption, enhancing solubility of a different permeation enhancer in the composition and/or is a mucoadhesive. To be clear, a preferred permeation enhancer provides one or more of these functions. For example, sodium chenodeoxycholate enhances permeation and improves solubility of an aromatic alcohol permeation enhancer at the site of absorption. Preferably the composition further comprises a second permeation enhancer, wherein the second permeation enhancer enhances transmucosal permeation, or more specifically enhances permeation of an Exendin ABD across a mucosal, cell-lined barrier, e.g. intestinal wall, nasal lining, oral cavity, tongue, allowing Exendin ABD absorption and subsequent circulation in the subject's blood, by enhancing paracellular permeation, opening cell tight junctions, enhancing transcellular permeation, inhibiting a protease at the site of delivery and absorption, enhancing solubility of a different permeation enhancer in the composition and/or is a mucoadhesive. The composition can further comprise a third permeation enhancer, wherein the third permeation enhancer enhances transmucosal permeation, or more specifically enhances permeation of an Exendin ABD across a mucosal, cell-lined barrier, e.g. intestinal wall, nasal lining, oral cavity, tongue, allowing Exendin ABD absorption and subsequent circulation in the subject's blood, by enhancing paracellular permeation, opening cell tight junctions, enhancing transcellular permeation, inhibiting a protease at the site of delivery and absorption, enhancing solubility of a different permeation enhancer in the composition and/or is a mucoadhesive.

The composition can optionally further comprise (c) an inhibitor of an intestinal protease, a mucoadhesive, a surfactant, an oil, an emulsifier or a mixture thereof. The composition can optionally further comprise (d) a pH lowering agent that lowers the pH at the site of absorption to inhibit protease activity. The composition can further optionally comprise (e) one or more conventional additive used in the formulation of pharmaceutical products, preferably a bulking agent or a polypeptide stabilizing agent, or other typical excipient useful for polypeptide formulations, or a mixture thereof. Typically the agent (e) improves the manufacturing of the final formulation product, e.g. tablet, granules, solution or capsule, or improves stability of the Exendin ABD during formulation or during storage of the formulated product.

Preferably the composition comprises two permeation enhancers: a non-conjugated bile acid or salt, and an aromatic alcohol. The non-conjugated bile acid or salt enhances solubility of the other permeation enhancer aromatic alcohol at the site of absorption. See WO 2004/091667 A1, which is incorporated herein by reference for its disclosure of enhancer formulations, enhancers, and assays to screen effectiveness of enhancer and formulations.

It has been found that surprisingly such formulations provide superior transmucosal absorption of Exendin ABD, when compared other formulations. As demonstrated in the Examples, such formulations provide relatively rapid intestinal absorption of an Exendin ABD and uptake into a subject's blood, with a relatively flat pharmacokinetic profile, and achieving long duration of circulation of the Exendin ABD in the subject.

Preferably the aromatic alcohol permeation enhancer is propyl gallate, butyl hydroxy anisole (BHA), butylated hydroxy toluene (BHT), or an analog or derivative thereof. More preferably the aromatic alcohol permeation enhancer is propyl gallate. The aromatic alcohols, and particularly propyl gallate, are believed to act not only as a permeation enhancer but is believed to also inhibit proteases at the site of absorption. The aromatic alcohol analog or derivative of propyl gallate can be an ester of gallic acid, where the ester may be linear or branched chain C1-12 alkyl, C1-12 alkyloxy, C1-12 alkylthio or C2-12 alkenyl ester, and is optionally substituted with halogen or with linear or branched chain C1-12 alkyl, C1-12 alkyloxy, C1-12 alkylthio or C2-12 alkenyl esters. The aromatic alcohol analog or derivative of BHA can be a hydroxy anisole or hydroxy toluene where the methyl group or the methoxy group linked to the aromatic ring and/or the hydrogen ortho to the hydroxyl group are replaced by linear or branched chain C1-C12 alkyl, C1-12 alkyloxy, C1-22 alkylthio or C2-12 alkenyl, either unsubstituted or substituted in any position, especially by halogen atoms.

The non-conjugated bile acid or salt can be chenodeoxycholic acid, deoxycholic acid, ursodeoxycholic acid, glycochenodeoxycholic acid, glycodeoxycholic acid, or a pharmaceutically acceptable salt thereof, and is preferably chenodeoxycholic acid, deoxycholic acid, cholic acid or a salt thereof, and more preferably chenodeoxycholic acid or salt thereof. A preferred salt is a sodium salt. Preferred permeation enhancer is sodium chenodeoxycholate.

Accordingly, a preferred formulation in one in which (b) comprises propyl gallate and a non-conjugated bile acid or salt, most preferably where the bile acid is chenodeoxycholate, and more preferably is sodium chenodeoxycholate.

It is believed that peristaltic action in the intestine tends to encourage rapid dispersion of pharmaceutical formulations over a larger surface area, so that it is important that the permeation enhancers, e.g. bile salts, in the formulation dissolve rapidly and remain in solution for a long as possible at the local pH conditions, so that the local concentration of the permeation enhancer is maintained as high as possible to provide maximal permeation effect. Some non-conjugated bile acids, such as chenodeoxycholate or deoxycholate are often not soluble at pH values much below pH 7. While not to be bound by theory, it is believed the aromatic alcohol maintains the non-conjugated bile acid or salt in solution at pH values around or below 7, equivalent to pH values commonly encountered in the small intestine. Propyl gallate is also not very water soluble at the relatively high doses at which is used herein, and co-formulation with bile acid or salt, preferably sodium chenodeoxycholate, enhances the solubilization of propyl gallate in water and in simulated intestinal fluids.

Generally, the composition will contain a ratio by weight of the non-conjugated bile salt to the aromatic alcohol from 10:1 to 1:10 (w/w), from 3:1 to 1:3, from 2:1 to 1:2, from 2:1 to 1:1, and preferably 2:1 (w/w).

Generally, the weight ratio of non-conjugated bile salt and aromatic alcohol, combined weight, to the engineered polypeptide can be from 1:1 to 200:1 (w/w), more preferably from 3:1 to 100:1, and most preferably from 5:1 to 50:1. It can be at least 5:1 and at least 10:1.

The amount of permeation enhancer or enhancers and Exendin ABD in the composition of the invention is chosen so as to achieve, at the mucosal cell barrier, e.g. the intestinal cell barrier layer, i.e. intestinal wall, an effective concentration of each permeation enhancer so as to cause enhanced absorption of sufficient Exendin ABD to exert its normal beneficial effect (or achieve therapeutic levels after one or more doses, typically after no more than five doses). For example, the practitioner would select the amounts of the non-conjugated bile salt and propyl gallate and Exendin ABD based on the amount (for example, blood concentration level) of the Exendin ABD necessary for therapeutic effect. In a preferred embodiment, the weight ratio of the total weight of the non-conjugated bile salt and aromatic alcohol to the Exendin ABD in the composition is 1 from 1:1 to 200:1, more preferably from 3:1 to 100:1, and most preferably from 5:1 to 50:1.

In one embodiment the formulation comprises an amount and ratio of the two permeation enhancers as in formulation OF1, either liquid or dry form, as described in the Examples herein, and that is formulated with any of Exendin ABD herein. Exendin ABD concentration will generally vary from 0.5 to 2.0 milligrams for daily to weekly delivery. For delivery at less frequent schedule, the active concentration will increase accordingly.

The absolute amount of the Exendin ABD in any formulation herein would be selected based on the dosage of the substance required to exert its normal beneficial effect with respect to the dosage regimen used and the patient concerned. Determination of these dosage amounts falls within the mantle of the practitioner of the invention.

The compositions herein optionally further comprise (e) any conventional additive used in the formulation of pharmaceutical products including, for example, preferably a bulking agent, a polypeptide stabilizing agent, anti-oxidants, anti-microbials, suspending agents, fillers, diluents, absorbents, glidants, binders, anti-caking agents, lubricants, disintegrants, swelling agents, viscosity regulators, plasticizers, and flavoring agents. Suitable swelling agents include sodium starch glycolate, pregelatinized starch, microcrystalline cellulose, crosprovidone and magnesium aluminum silicate or mixtures thereof. Sodium starch glycolate and other polysaccharide-based swelling agents may be present in an amount of from 5 to 10% by weight. Crosprovidone may be present in an amount of from 5 to 30% by weight.

In the composition of the invention where the mixture is contained in a capsule or tablet, the formulation is preferably substantially anhydrous. In more preferred embodiments of the invention the entire composition is substantially anhydrous. Substantially anhydrous in the context of this invention means less than 5%, preferably less than 1% and more preferably less than 0.5% water by weight of the mixture.

In the composition of the invention, when a capsule is used for delivery, the mixture contained in the capsule may be a liquid, semi-solid (e.g. gel), which is either in the form of a solution or a microparticulate dispersion where the Exendin ABD is incorporated into the formulation either in the form of a solution or as a microparticulate dispersion. Alternatively, the composition may be in the form of a solid, e.g. tablet, film.

The compositions of the invention can be produced by preparing a substantially anhydrous mixture of the Exendin ABD (e.g. lyophilized, spray-dried, granulated) and the permeation enhancer(s) and then filling uncoated capsules with the mixture and then coating them with an appropriate polymer mixture to achieve the desired permeability properties. Depending on the nature of additional excipients employed, the pharmaceutical composition of the invention may be in liquid, solid, semi-solid or gel form.

The pharmaceutical composition of the invention is suitable for administration via any route giving access to different mucosal tissues such as buccal and sub-lingual mucosa, the nasal palate, the lungs, the rectum, the intestinal tract (including the large and small intestines) and the vagina. In the case of liquid, semi-solid or gel formulations, these may be either anhydrous or aqueous. Where the intended site of action of the composition of the invention is the intestine, it is desirable that the composition is enclosed within an enteric coating which can withstand the stomach, so that the components of the formulation remain together, undiluted, preferably in close association, until they reach the tissues of the small intestine or colon, preferably small intestine. Such formulations will suitably be anhydrous. Compositions in liquid form will suitably be administered as enteric-coated capsules, while solid formulations may be administered either within enteric-coated capsules, or in tablet form, preferably enteric-coated tablets.

The enteric coating is selected to withstand the stomach acidic pH and to become permeable at the desired location in the intestine. This is preferably determined by the pH conditions that occur along the length of the intestine. Where the site of action is the small intestine, it is preferred that the enteric coating becomes permeable and releases its contents at a pH of from 3 to 7, preferably from 5.5 to 7, more preferably from 5.5 to 6.5, and most preferably at pH 5.5.

Where the intended site of action is the colon, it is preferred that the enteric coating becomes permeable and releases its contents at a pH of 6.8 or above.

Suitable enteric coatings are well known in the art and include cellulose acetate, phthalate, shellac and polymethacrylates such as those selected from the L and S series of Eudragits (Evonik Industries), in particular Eudragits L 30 D-55, L 100-55, L 100, L 12.5, S 100, S 12.5, and FS 30 D, or mixtures thereof to achieve the pH required to target a particular section of the intestine. Preferably dissolution will occur at the duodenum, jejunem or ileum, preferably at the duodenum or jejunem.

Suitable plasticizers or wetting agents, such as triethyl citrate and polysorbate 80 may also be included in the coating mixture.

Selection of an appropriate coating for the capsule, which is preferably an HPMC or a gelatin capsule, can readily be made by the person skilled in the art based on their knowledge and the available literature supporting the enteric coatings, such as the Eudragit products. The Examples herein demonstrate one coating solution and method. When the intended site of action is the nasal mucosa, the formulation may be in the form of an aqueous solution or as a dry powder, which can be administered as a spray. When the intended site of action is the rectum, an appropriate method of administration is as an anhydrous liquid or solid enclosed within a capsular shell, or incorporated into the matrix of an erodible suppository. For vaginal application, the Exendin ABD formulation can also be in gel or foam form.

Methods applicable for preparing and for testing Exendin ABD formulation (solid, semi-solid or liquid) for transmucosal delivery, including those preferably comprising a non-conjugated bile acid or salt and an aromatic alcohol, are known in the art. For example see WO 2004/091667.

While these composition can be liquid or semi-solid, advantageous are solid-dose formulations, especially for oral or sub-lingual administration. Solid form can be provided as an enteric-coated capsule or tablet. Alternatively, these formulations, and any of the other formulations described herein, can be incorporated into a pharmaceutical formulation as a dispersion in a non-aqueous liquid.

Irrespective of its form, the composition can be administered and/or delivered to a specifictargeted mucosal site, e.g. intestine, nasal or oral cavity site, wherein the composition dissolves at or near the targeted mucosa site as it comes into contact with a body fluid, e.g. bile, chime, saliva or mucosa, at or near the site. In some embodiments, the composition dissolves within a period of from about 1 minute to about 30 minutes, generally no more than 60 minutes, upon reaching its site of action.

In another embodiment of the formulation, the formulation for transmucosal delivery comprises an aromatic alcohol absorption enhancer and a biguanide. The compounds act synergistically to enhance drug absorption. See published application number US20090041849A1 which is incorporated herein by reference for teaching such base formulations that can be used with the Exendin ABD described herein. Preferably the aromatic alcohol permeation enhancer is propyl gallate, butyl hydroxy anisole (BHA), butylated hydroxy toluene (BHT), or an analog or derivative thereof. More preferably the aromatic alcohol permeation enhancer is propyl gallate. The aromatic alcohols, and particularly propyl gallate, are believed to act not only as a permeation enhancer but is believed to also inhibit proteases at the site of absorption. The aromatic alcohol analog or derivative of propyl gallate can be an ester of gallic acid, where the ester may be linear or branched chain C1-12 alkyl, C1-12 alkyloxy, C1-12 alkylthio or C2-12 alkenyl ester, and is optionally substituted with halogen or with linear or branched chain C1-12 alkyl, C1-12 alkyloxy, C1-12 alkylthio or C2-12 alkenyl esters. The aromatic alcohol analog or derivative of BHA can be a hydroxy anisole or hydroxy toluene where the methyl group or the methoxy group linked to the aromatic ring and/or the hydrogen ortho to the hydroxyl group are replaced by linear or branched chain C1-C12 alkyl, C1-12 alkyloxy, C1-22 alkylthio or C2-12 alkenyl, either unsubstituted or substituted in any position, especially by halogen atoms.

The biguanides for use in the formulations for transmucosal delivery will suitably have the following formula

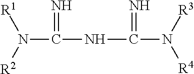

wherein R¹, R², R³ and R⁴ are each independently chosen from hydrogen, optionally substituted alkyl, optionally substituted phenyl, ethylene glycol, diethylene glycol, triethylene glycol and tetraethylene glycol, or one of R¹, R², R³ and R⁴ may be

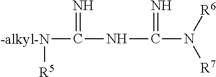

where R⁵, R⁶ and R⁷ are each independently chosen from hydrogen, optionally substituted alkyl, optionally substituted phenyl, ethylene glycol, diethylene glycol, triethylene glycol and tetraethylene glycol. Substituents for the alkyl and phenyl groups include halo, e.g. chloro, bromo, fluoro or iodo, hydroxy and amino. The alkyl groups preferably have from 1 to 6 carbons, and may be saturated or unsaturated, straight chain or branched. The biguanide may be included in the composition of the invention as a pharmaceutically acceptable salt. Preferred biguanides for use in the present invention include metformin, phenformin and chlorhexidine or pharmaceutically acceptable salts thereof. The pharmaceutically acceptable salts are suitably the chloride, bromide, iodide or salts of organic acids such as the acetate, propionate, mesylate (methyl sulphonate) or glucuronate. The biguanide may be present in the composition in an amount of at least 50% by weight, preferably from 60 to 95% and more preferably from 80 to 90%. The alcohol may be present in the composition in an amount of from 5 to 30% by weight, preferably from 10 to 20%. Accordingly, a preferred Exendin ABD transmucosal formulation is one in which (b) comprises propyl gallate and a biguanide, preferably where the biguanide is metformin, phenformin and chlorhexidine or pharmaceutically acceptable salts, and more preferably metformin.

In a further embodiment of the formulations containing a non-conjugated bile acid and an aromatic alcohol, to achieve even further bioavailability, the composition further comprises as third permeation enhancer a salt of a medium chain fatty acid which has a carbon chain length of the carboxylate moiety of from 6 to 20 carbon atoms. The salt of the medium chain fatty acid is solid at room temperature, which facilitates sold dose forms, but can be formulated in liquid dose. Preferably, the medium chain fatty acid carbon chain length is from 8 to 14 carbon atoms. Preferably the fatty acid salt is a salt of caprylate, caprate and laurate. Preferably the salt of the medium chain fatty acid is a sodium salt. More preferably the medium chain fatty acid salt is selected from the group consisting of sodium caprylate, sodium caprate and sodium laurate. The engineered polypeptide and the medium chain fatty acid can be present in a molar ratio of from 1:100000 to 10:1 (engineered polypeptide:enhancer) or 1:50000 or 1:20000 or 1:10000. See for example the compositions and product forms, e.g. tablet, as described in WO2009137078(A1) which is incorporated by reference.

In a further embodiment of the formulations containing a non-conjugated bile acid (or alternatively a biguanide) and an aromatic alcohol, to achieve even further bioavailability, the composition further comprises as third permeation enhancer that is an ester of a medium chain fatty acid which has a carbon chain length of the carboxylate moiety of from 6 to 20 carbon atoms. Preferably the fatty acid ester is of caprylate, caprate and laurate. The ester of the medium chain fatty acid is solid at room temperature to facilitate a solid formulation, however such an ester can still be formulated as a liquid form. The medium chain fatty acid carbon chain length is preferably from 8 to 14 carbon atoms.

In a further embodiment of the formulations containing a non-conjugated bile acid and an aromatic alcohol, to achieve even further bioavailability, the composition further comprises as third permeation enhancer that is an ether of a medium chain fatty acid which has a carbon chain length of the carboxylate moiety of from 6 to 20 carbon atoms. Preferably the fatty acid ether is an ether of caprylate, caprate and laurate. The ether of the medium chain fatty acid is solid at room temperature, but can be formulated at liquid dose if needed. Preferably the medium chain fatty acid carbon chain length is from 8 to 14 carbon atoms.

Accordingly the composition can comprise a non-conjugated bile acid or salt (or alternatively a biguanide), an aromatic alcohol and at least one permeation-enhancing medium chain fatty acid salt, ester or ether. A preferred aromatic alcohol is propyl gallate. A preferred bile salt is sodium chenodeoxycholate. A determination of improvement to absorption by the presence of the third permeation enhancer can be made as would be known in the art, for example, using cell-based or tissue-based in vitro permeation assays, with an Exendin ABD, or by direct testing of a formulation in vivo to determine improvement in bioavailability compared a formulation absent one or more of the medium chain fatty acid, salt, ester or ether. See for example the compositions and product forms, e.g. tablet, as described in WO2009137078(A1) which is incorporated by reference.

In a further embodiment of the formulations containing a non-conjugated bile acid (or alternatively a biguanide) and an aromatic alcohol, to achieve even further bioavailability, the composition further comprises as agent (c) or as a third permeation enhancer, at least one intestinal protease inhibitor. The at least one protease inhibitor preferably is a serine protease inhibitor. More preferably the protease inhibitor is a Bowman-Birk inhibitor. The protease inhibitor can be a trypsin inhibitor. Accordingly, the trypsin inhibitor can be selected from a lima bean trypsin inhibitor, aprotinin, soy bean trypsin inhibitor (SBTI), bovine bean trypsin inhibitor (BPTI) or ovomucoid, or any combination thereof. The protease inhibitor can be a suicide inhibitor, a transition state inhibitor, a protein protease inhibitor, a protease-inhibiting chelating agent, or any combination thereof. The protease inhibitor can be a metalloprotease inhibitor. The protease-inhibiting chelating agent preferably chelates zinc. The protease inhibitor can be a cysteine protease inhibitor. The protease inhibitor can be threonine protease inhibitor. The protease inhibitor can be an aspartic protease inhibitor. For oral delivery to the intestine, the protease inhibitor preferably inhibits chymotrypsin. For formulation delivered to the intestine, the protease inhibitor inhibits protease digestion of the engineered polypeptide in the small intestine of a subject. In one embodiment, the composition can comprise as agent (c) one or more of the protease inhibitors described herein. Accordingly, the composition can comprise a non-conjugated bile acid or salt (or alternatively a biguanide), an aromatic alcohol and at least one intestinal protease inhibitor of an intestinal protease that is able to digest the delivered Exendin ABD. A preferred aromatic alcohol is propyl gallate. A preferred bile salt is sodium chenodeoxycholate. Such a determination can be made as is known in the art, for example, using intestinal fluids in vitro with an Exendin ABD, or by direct testing of a formulation in vivo to determine improvement in bioavailability compared a formulation absent one or more of the protease-inhibitors.

In a further embodiment of the formulations containing a non-conjugated bile acid (or alternatively a biguanide) and an aromatic alcohol, to achieve even further bioavailability the composition further comprises as a third permeation enhancer a permeation-enhancing chelating agent. The chelating agent preferably opens tight junctions to facilitate permeation. The chelating agent preferably chelates calcium ions, magnesium ions or both. Even more preferably the chelating agent both opens tight junctions and inhibits an intestinal protease. One preferred chelating agent is ethylenediaminetetraacetic acid (EDTA) or a salt thereof. Preferably the chelating agent is a sodium salt of ethylenediaminetetraacetic acid. In another embodiment the chelating agent is ethylene glycol tetraacetic acid (EGTA) or a salt thereof. The chelating agent is preferably a sodium salt of ethylene glycol tetraacetic acid. Generally the formulation comprises at least 1% by weight of the chelating agent, more preferably at least about 2% by weight, even more preferably the formulation mixture comprises at least 10% by weight of the chelating agent. Accordingly, the composition can comprise a non-conjugated bile acid or salt (or alternatively a biguanide), an aromatic alcohol and at least one permeation-enhancing chelating agent. A preferred aromatic alcohol is propyl gallate. A preferred bile salt is sodium chenodeoxycholate. A determination of the improvement to Exendin ABD bioavailability provided by the chelating agent can be made as is known in the art, for example, for example, using cell-based or tissue-based in vitro permeation assays, or by direct testing of a formulation in vivo to determine improvement in bioavailability compared a formulation absent one or more of the permeation-enhancing chelating agents. In an even more preferred embodiment the chelating agent also provides stabilization of an Exendin ABD, such as protection against degradation during manufacturing or during storage.

In a further embodiment of the formulations containing a non-conjugated bile acid (or alternatively a biguanide) and an aromatic alcohol, to achieve even further bioavailability the composition further comprises as a third permeation enhancer a mucoadhesive permeation enhancer. The mucoadhesive permeation enhancer preferably opens tight junctions to facilitate permeation. The mucoadhesive permeation enhancer can be a mucoadhesive permeation enhancer chitosan or a cross-linked poly(acrylic acid). Preferably it is an N,N,N-methylated chitosan (TMC). Preferably the cross-linked poly(acrylic acid) is Carbomer 971. Preferably the formulation mixture comprises at least 1% by weight of the mucoadhesive permeation enhancer, more preferably at least 10% by weight of the mucoadhesive permeation enhancer. A preferred aromatic alcohol is propyl gallate. A preferred bile salt is sodium chenodeoxycholate. A determination of the improvement to Exendin ABD bioavailability provided by the mucoadhesive permeation enhancer can be made as is known in the art, for example, for example, using cell-based or tissue-based in vitro permeation assays, or by direct testing of a formulation in vivo to determine improvement in bioavailability compared a formulation absent one or more of the permeation-enhancing mucoadhesive agent.

In a further embodiment of the formulations containing a non-conjugated bile acid (or alternatively a biguanide) and an aromatic alcohol, to achieve even further bioavailability of the Exendin ABD the composition further comprises as a third permeation enhancer a cationic, anionic or nonionic surfactant, or mixture thereof. The surfactant permeation enhancer can be a polysorbate, polysorbate 80, hexadecyldimethylbenzylammonium chloride, N-hexadecylpyridinium bromide, dodecyltrimethylammonium bromide, hexadecyltrimethylammonium bromide, tetradecyl-β-D-maltoside, octylglucoside, 3-(N,N-dimethylpalmitylammonio)propanesulfonate (PPS); myristyldimethyl ammonio propane sulfonate; decyldimethyl ammonio propane sulfonate, or a mixture thereof. Preferably it is 3-(N,N-dimethylpalmitylammonio)propanesulfonate (PPS); myristyldimethyl ammonio propane sulfonate; decyldimethyl ammonio propane sulfonate, or mixture thereof. More preferably the third enhancer is the surfactant permeation enhancer is 3-(N,N-dimethylpalmitylammonio)propanesulfonate. Preferably the formulation mixture comprises at least 1% by weight of the third permeation enhancer, more preferably at least 10% by weight of the third permeation enhancer. A preferred aromatic alcohol is propyl gallate. A preferred bile salt is sodium chenodeoxycholate. A determination of the improvement to Exendin ABD bioavailability provided by the third permeation enhancer can be made as is known in the art, for example, for example, using cell-based or tissue-based in vitro permeation assays, or by direct testing of a formulation in vivo to determine improvement in bioavailability compared a formulation absent one or more of the third permeation-enhancing surfactants.

In a further embodiment of the formulations containing a non-conjugated bile acid (or alternatively a biguanide) and an aromatic alcohol, to achieve even further bioavailability of the Exendin ABD the composition further comprises as a third permeation enhancer sodium N-(8-[2-hydroxybenzoyl]amino) caprylate (SNAC); N-(5-chlorosalicyloyl)-8-aminocaprylic acid (5-CNAC); sodium N-[10-(2-hydroxy-benzoyl)amino]decanoate (SNAD), or mixture thereof. Preferably the formulation mixture comprises at least 1% by weight of the third permeation enhancer, more preferably at least 10% by weight of the third permeation enhancer. A preferred aromatic alcohol is propyl gallate. A preferred bile salt is sodium chenodeoxycholate. A determination of the improvement to Exendin ABD bioavailability provided by the third permeation enhancer can be made as is known in the art, for example, for example, using cell-based or tissue-based in vitro permeation assays, or by direct testing of a formulation in vivo to determine improvement in bioavailability compared a formulation absent one or more of the third permeation-enhancing agent.

In a further embodiment of the formulations containing a non-conjugated bile acid (or alternatively a biguanide) and an aromatic alcohol, to achieve even further bioavailability of the Exendin ABD the composition further comprises as agent a pharmaceutically acceptable pH-lowering agent; wherein the pH-lowering agent is present in said finished pharmaceutical product in a quantity which, if said product were added to 10 milliliters of 0.1M aqueous sodium bicarbonate solution, would be sufficient to lower the pH of said solution to no higher than 5.5. Preferably the formulation mixture comprises the pH-lowering compound present in a quantity which, if said product were added to 10 milliliters of 0.1M sodium bicarbonate solution, would be sufficient to lower the pH of said solution to no higher than 3.5. The pH-lowering agent acts to lower the pH at the site of absorption, presumably by inhibiting protease reactions, since as determined herein the Exendin ABD are more stable to pancreatin proteases at acidic pH. A preferred aromatic alcohol is propyl gallate. A preferred bile salt is sodium chenodeoxycholate. If the Exendin ABD is determined to be less stable than desired when in contact with the pH-lowering agent in a dry formulation, the composition, e.g., if in the form of a solid, can comprise a layer that separates the pH-lowering agent from the layer comprising the Exendin ABD, and further in order to prevent the pH-lowering agent from causing premature dissolution of a pH sensitive enteric coating, can comprise an outer surface that is substantially free of an acid-resistant protective vehicle. Preferably the pH-lowering agent has a solubility in water of at least 30 grams per 100 milliliters of water at room temperature. Preferably the composition is formulated in granules containing a pharmaceutical binder and, uniformly dispersed in said binder, is the pH-lowering agent and the permeation enhancers and Exendin ABD. The composition can comprise a lamination having a first layer comprising the pharmaceutically acceptable pH-lowering agent and a second layer comprising the engineered polypeptide, wherein the first and second layers are united with each other, but the pH-lowering agent and the polypeptide are substantially separated within the lamination such that less than about 0.1% of the polypeptide contacts the pH-lowering agent to prevent substantial mixing between the first layer material and the second layer material and thus to avoid interaction in the lamination between the pH-lowering agent and the Exendin ABD. The permeation enhancer can be in either layer or even a third layer. Alternatively the pH-lowering agent and the Exendin ABD can be formulated separately as granules. The pH-lowering agent is selected from the group consisting of citric acid, tartaric acid and an acid salt of an amino acid. The pH-lowering agent is preferably selected from the group consisting of dicarboxylic acids and tricarboxylic acids. Preferably the pH-lowering agent is present in an amount not less than 300 milligrams. See for example the formulations and product forms, e.g. tablet, described publication WO2008150426(A1) incorporated herein by reference. A determination of the improvement to Exendin ABD bioavailability provided by the pH-lowering agent can be made as is known in the art, for example, for example, using cell-based or tissue-based in vitro permeation assays, or by direct testing of a formulation in vivo to determine improvement in bioavailability compared a formulation absent one or more of the pH lowering agents.

In yet another embodiment of each of the formulations above, the non-conjugated bile acid (or alternatively a biguanide) and an aromatic alcohol, is either absent or is replaced by one or more of any other permeation enhancer, preferably a permeation enhancer disclosed herein. For example, an Exendin ABD formulation can be absent the non-conjugated bile acid (or alternatively a biguanide) and an aromatic alcohol, and contain comprise as a first permeation enhancer sodium N-(8-[2-hydroxybenzoyl]amino) caprylate (SNAC); N-(5-chlorosalicyloyl)-8-aminocaprylic acid (5-CNAC); sodium N-[10-(2-hydroxy-benzoyl)amino]decanoate (SNAD), or mixture thereof. Similarly, as an example, a formulation can comprise as a first permeation enhancer a salt, ester or ether of a medium chain fatty acid which has a carbon chain length of the carboxylate moiety of from 6 to 20 carbon atoms as described herein. Similarly, specifically contemplated are Exendin ABD formulations where any of the third permeation enhancers herein can be the first permeation enhancer. Each can then be combined with each other (e.g. medium chain fatty acid salt with a surfactant permeation enhancer or a mucoadhesive permeation enhancer) or can further comprise a protease inhibitor, a chelator, a pH lowering agent, as described herein.

A further example of one such embodiment is where a pH-lowering agent is present sufficient to inhibit an intestinal protease and the permeation enhancer is a surfactant. Suitable formulations and product forms, e.g. tablet, layered tablet or granules, can be found in WO2008150426(A1) which is incorporated herein by reference. A preferred surfactant permeation enhancer is selected from the group consisting of acylcarnitines, phospholipids, bile acids and sucrose esters. The surfactant permeation enhancer is selected from the group consisting of (a) an anionic agent that is a cholesterol derivative, (b) a mixture of a negative charge neutralizer and an anionic surface active agent, (c) non-ionic surface active agents, and (d) cationic surface active agents. Preferably the surfactant is lauroyl-L-carnitine. The acid can preferably be citric acid. The ratio of the two agents can be that as found in the formulation designated formulation OF2 described in the Examples. Accordingly, in such an embodiment of the Exendin ABD composition, the composition comprises a surfactant as a first permeation enhancer and a pharmaceutically acceptable pH-lowering agent; wherein the pH-lowering agent is present in said finished pharmaceutical product in a quantity which, if said product were added to 10 milliliters of 0.1M aqueous sodium bicarbonate solution, would be sufficient to lower the pH of said solution to no higher than 5.5. Preferably the formulation mixture comprises the pH-lowering compound present in a quantity which, if said product were added to 10 milliliters of 0.1M sodium bicarbonate solution, would be sufficient to lower the pH of said solution to no higher than 3.5. The pH-lowering agent acts to lower the pH at the site of absorption, presumably by inhibiting protease reactions, since as determined herein the Exendin ABD are more stable to pancreatin proteases at acidic pH. If the Exendin ABD is determined to be less stable than desired when in contact with the pH-lowering agent in a dry formulation, the composition, e.g., if in the form of a solid, can comprise a layer that separates the pH-lowering agent from the layer comprising the Exendin ABD, and further in order to prevent the pH-lowering agent from causing premature dissolution of a pH sensitive enteric coating, can comprise an outer surface that is substantially free of an acid-resistant protective vehicle. Preferably the pH-lowering agent has solubility in water of at least 30 grams per 100 milliliters of water at room temperature. Preferably the composition is formulated in granules containing a pharmaceutical binder and, uniformly dispersed in said binder, is the pH-lowering agent and the permeation enhancers and Exendin ABD. The composition can comprise a lamination having a first layer comprising the pharmaceutically acceptable pH-lowering agent and a second layer comprising the engineered polypeptide, wherein the first and second layers are united with each other, but the pH-lowering agent and the polypeptide are substantially separated within the lamination such that less than about 0.1% of the polypeptide contacts the pH-lowering agent to prevent substantial mixing between the first layer material and the second layer material and thus to avoid interaction in the lamination between the pH-lowering agent and the Exendin ABD. The permeation enhancer can be in either layer or even a third layer. Alternatively the pH-lowering agent and the Exendin ABD can be formulated separately as granules. The pH-lowering agent is selected from the group consisting of citric acid, tartaric acid and an acid salt of an amino acid. The pH-lowering agent is preferably selected from the group consisting of dicarboxylic acids and tricarboxylic acids. In a preferred formulation the agent is citric acid. Preferably the pH-lowering agent is present in an amount not less than 300 milligrams.

The compositions herein can comprise human serum albumin, which can bind to the Exendin ABD to provide manufacturing or storage stability and further protect the Exendin ABD from proteases at the site of absorption.

As described herein, the Exendin ABD composition can be in liquid, solid or semi-solid form that is suitable for transmucosal delivery. Preferably the form is a solid. The composition can be encapsulated in an enteric capsule or coated with an enteric coating that becomes permeable at a pH from 4 to 7. The enteric coating can become permeable at a pH from 5 to 7. Preferably the enteric coating becomes permeable at a pH from 5.5 to 7, and even more preferably at a pH from 5.5 to 6.5, and most preferably at pH 5.5.

As described herein the Exendin ABD composition can further comprising one or more other agents, a conventional excipient, selected from the group consisting of diluents, lubricants, disintegrants, plasticizers, anti-tack agents, opacifying agents, pigments, stabilizers, and flavorings. The at least one diluent or bulking agent is an inert filler chosen from microcrystalline cellulose, lactose, dibasic calcium phosphate and saccharides. The inert filler can be a lactose which is lactose monohydrate or lactose anhydrous. The inert filler can be at least one saccharide selected from the group consisting of mannitol, trehalose, starch, sorbitol, sucrose, and glucose. As described herein the conventional agent can be at least one lubricant selected from the group consisting of colloidal silicon dioxide, talc, magnesium stearate, calcium stearate, and stearic acid. As described herein the conventional agent can comprise at least one disintegrant selected from the group consisting of lightly crosslinked polyvinylpyrrolidone, corn starch, potato starch, maize starch and modified starches, croscarmellose sodium, crospovidone, and sodium starch glycolate.

The composition can be in the form of a microparticulate dispersion, an emulsion, an enteric coated tablet, or in a capsule as a solution, emulsion or multiparticulate form of microparticles, microgranules, granules or pellets. The tablet and multiparticulate form can be an instant release form. The multiparticulate can be in the form of a tablet. The composition can be and is preferably a compressible composition which is capable of being compressed into a solid pharmaceutical dosage form which is effective in delivering the engineered polypeptide and permeation enhancer to an intestine. The compressible form can be a compressible powder or compressible granules. The composition can be in the form of a flowable powder or granules suitable for loading into a capsule.

As described herein any of the Exendin compositions for transmucosal delivery can be used as a medicament.

The pharmaceutical composition as described herein is a pharmaceutical composition for mucosal, oral, nasal, sublingual, pulmonary, rectal, vaginal or buccal delivery. Preferably, the composition is a pharmaceutical composition for oral delivery or for sub-lingual delivery. Preferably the pharmaceutical composition for oral delivery comprises a tablet, granules, microparticles, emulsion or a capsule.

The Exendin ABD compositions for transmucosal delivery can be administered once daily, twice weekly, thrice weekly, once weekly, twice monthly, once monthly or even less often. Preferably administration is once daily or once weekly. Preferably the once daily administration provides a sub-therapeutic dose where the Exendin ABD is present in the circulating blood at a level that adds to the next dose, such that a therapeutic blood level can be achieved after 2, 3, 4, 5 and no more than six daily doses. Preferably the dosing regimen provides a sustained average concentration, more preferably a steady state plasma concentration that is therapeutic.

The Exendin ABD compositions for transmucosal delivery find use for treating a disease or disorder in a subject in need of such treatment. The disease or disorder can be diabetes, overweight, obesity, Alzheimer's disease, Parkinson's disease, fatty liver disease, dyslipidemia, coronary artery disease, stroke, short bowel syndrome (SBS), hyperlipidemia, nonalcoholic steatohepatitis (NASH), nonalcoholic fatty liver disease (NAFLD), hypoglycemia unawareness (HU), restrictive lung disease including sarcoidosis, or metabolic syndrome X. Preferably the disease or disorder is diabetes, overweight, obesity, short bowel syndrome (SBS) or Parkinson's disease. Even more preferably the disease or condition is diabetes, type I diabetes, type II diabetes, prediabetes, impaired glucose tolerance, gestational diabetes, NASH, HIV-treatment-induced diabetes, steroid-induced diabetes, or latent autoimmune diabetes in adults (LADA). And even more preferably the disease or condition is type II diabetes or obesity. Most preferably the disease or condition is type II diabetes. The compositions will be provided to a subject in need thereof in an amount of Exendin ABD effective to treat said disease or disorder. Since the Exendin ABD can reduce body weight, then in any of the diseases or conditions described herein the subject can be, and is preferably also overweight or obese. In one embodiment, the subject in need of such treatment is taking insulin, preferably a basal insulin, and more preferably the basal insulin is glargine, degludec or detemir.

The formulations provide the ability to non-invasively deliver the long lasting Exendin ABD at intervals of, for example, 8 hr, 12 hr, 1 day, 2 days, 3 days, 4 days, 5 days, 6 days, 1 week, 2 weeks, 3 weeks, 1 month or even longer. Preferred is once daily, once weekly and once monthly delivery. In a preferred embodiment, administration is once every 24 hours or once a day (i.e., “once daily”). In a more preferred embodiments, administration is once a week (i.e., “once weekly”). Preferably the engineered polypeptide is selected from the engineered polypeptides set forth in Tables 2, 3A, 3B, 3C and 3D herein. In some embodiments, the engineered polypeptide is selected from the engineered polypeptides set forth in Tables 2, 3A, 3B and 3X herein. In some embodiments, the engineered polypeptide is selected from the engineered polypeptides set forth in Table 2 and Table 3C herein. More preferably, an ABD1-containing Exendin ABD compound is Cmpd 15, Cmpd 21 or Cmpd 31, or has at least 95% amino acid sequence identity thereto, and an ABD2-containing Exendin ABD compound is Cmpd 2-5, 2-9 or 2-11, or has at least 95% amino acid sequence identity thereto.

The engineered polypeptides described herein can be administered alone or can be co-administered to a subject. Co-administration is meant to include simultaneous or sequential administration of the compounds individually or in combination (more than one compound). For example, it has been found that obesity can be beneficially treated with a combination therapy including leptin (e.g., metreleptin) and an amylin (e.g., pramlintide). See e.g., U.S. Published Appl. No. 2008/0207512. Accordingly, an engineered polypeptide described herein including an ABD and an exendin compound useful for treatment of e.g., obesity and overweight, can be administered alone to achieve such treatment or co-administered with either a leptin or leptin agonist, e.g. metreleptin, and/or an amylin or amylin agonist, e.g. pramlintide.

In some embodiments, the formulations and methods described herein further provide that the exendin, exendin analog or exendin analog agonist engineered polypeptide is co-administered with one or more anti-diabetic agents, such as anti-hyperglycemia agents, e.g. insulin (including regular, short acting, long-acting, and basal insulins), amylins, pramlintide, metformin and thiazolidinediones (including rosiglitazone and pioglitazone).

In some embodiments, the formulations and methods described herein further provide that the exendin, exendin analog or exendin analog agonist engineered polypeptide is co-administered with one or more cholesterol and/or triglyceride lowering agents. Exemplary agents include HMG CoA reductase inhibitors (e.g., atorvastatin, fluvastatin, lovastatin, pravastatin, rosuvastatin, simvastatin); bile ace sequestrants (e.g., colesevelam, cholestyramine, colestipol); fibrates (e.g., fenofibrate, clofibrate, gemfibrozil); ezetimibe, nicotinic acid, probucol, a lovastatin/niacin combination; an atorvastatin/amlodipine combination; and a simvastatin/ezetimibe combination.

The present disclosure provides the composition for use as a medicament, i.e. for use in therapy, since the exendin moiety is a therapeutically active compound, and surprisingly retains activity when fused to ABD.

Another aspect of the engineered polypeptides is that the ABD can provide an increase in the solubility in aqueous solution of a poor or low soluble exendin variant. This property can be imparted by the ABD itself or because of the ensuing complex of the engineered polypeptide bound to highly soluble albumin in vivo or in vitro, which association increases the solubility of the engineered polypeptide in aqueous solution. Thus, in an embodiment of this further aspect, there is provided a composition, including an exendin compound which per se has a solubility in water of no more than 1 mg/ml, or no more than 2 mg/ml or no more than 5 mg/ml, covalently coupled to an albumin binding domain as a fusion protein or conjugate as described herein, wherein the compound and the albumin binding polypeptide, fusion protein or conjugate are covalently coupled and the solubility of the engineered polypeptide is greater than that of the unfused (or not conjugated) native exendin compound.

The composition has an ability to associate with albumin in vivo or in vitro. In certain cases, it may be of benefit to form a complex of the composition with albumin outside of a living organism, i.e. to add exogenous albumin to the composition. Such a composition may be lyophilized, providing a formulation that is suitable for storage at ambient temperature. Thus, the present disclosure also provides a composition as defined above which further includes albumin, such as human serum albumin, and which may optionally be in dry form.

Co-administration can be achieved by separately administering the exendin, exendin agonist, or exendin analog agonist engineered polypeptide with the second agent, or by administering a single pharmaceutical formulation including the exendin, exendin agonist, or exendin analog agonist engineered polypeptide and the second agent. Appropriate dosage regimens for the second agents are generally known in the art.

The preparations can also be co-administered, when desired, with other active substances (e.g. to reduce metabolic degradation) as known in the art or other therapeutically active agents. An exendin engineered polypeptide described herein can be administered with other active anti-diabetes or anti-obesity agents, such as leptin or leptin agonists and amylin or amylin agonist compounds, e.g. the amylins, including davalintide and their analogs.

Amylins.

Amylin is a peptide hormone synthesized by pancreatic β-cells that is co-secreted with insulin in response to nutrient intake. The sequence of amylin is highly preserved across mammalian species, with structural similarities to calcitonin gene-related peptide (CGRP), the calcitonins, the intermedins, and adrenomedullin, as known in the art. The glucoregulatory actions of amylin complement those of insulin by regulating the rate of glucose appearance in the circulation via suppression of nutrient-stimulated glucagon secretion and slowing gastric emptying. In insulin-treated patients with diabetes, pramlintide, a synthetic and equipotent analogue of human amylin, reduces postprandial glucose excursions by suppressing inappropriately elevated postprandial glucagon secretion and slowing gastric emptying. The sequences of rat amylin, human amylin and pramlintide follow:

(SEQ ID NO: 6);

KCNTATCATQRLANFLVRSSNNLGPVLPPTNVGSNTY

(SEQ ID NO: 7)

KCNTATCATQRLANFLVHSSNNFGAILSSTNVGSNTY;

(SEQ ID NO: 8)

KCNTATCATQRLANFLVHSSNNFGPILPPTNVGSNTY.

Davalintide.

Davalintide, also known as “AC-2307” is a potent amylin agonist useful in the treatment of a variety of disease indications. See WO 2006/083254 and WO 2007/114838, each of which is incorporated by reference herein in its entirety and for all purposes. Davalintide is a chimeric peptide, having an N-terminal loop region of amylin or calcitonin and analogs thereof, an alpha-helical region of at least a portion of an alpha-helical region of calcitonin or analogs thereof or an alpha-helical region having a portion of an amylin alpha-helical region and a calcitonin alpha-helical region or analog thereof, and a C-terminal tail region of amylin or calcitonin. The sequences of human calcitonin, salmon calcitonin and davalintide follow:

| | |
|---|---|
| CGNLSTCMLGTYTQDFNKFHTFPQTAIGVGAP; | (SEQ ID NO: 9) |
| CSNLSTCVLGKLSQELHKLQTYPRTNTGSGTP; | (SEQ ID NO: 10) |
| KCNTATCVLGRLSQELHRLQTYPRTNTGSNTY. | (SEQ ID NO: 11) |

Without wishing to be bound by any theory, it is believed that amylins and davalintide, and fragment and analogs thereof, can require C-terminal amidation to elicit a full biological response. It is understood that amylin compounds such as those described herein which include amylins and/or davalintide, and fragment and analogs thereof, can be amidated at the C-terminal

“Amylin agonist compounds” include native amylin peptides, amylin analog peptides, and other compounds (e.g., small molecules) that have amylin agonist activity. The “amylin agonist compounds” can be derived from natural sources, can be synthetic, or can be derived from recombinant DNA techniques. Amylin agonist compounds have amylin agonist receptor binding activity and may include amino acids (e.g., natural, unnatural, or a combination thereof), peptide mimetics, chemical moieties, and the like. The skilled artisan will recognize amylin agonist compounds using amylin receptor binding assays or by measuring amylin agonist activity in soleus muscle assays. In one embodiment, amylin agonist compounds will have an IC₅₀ of about 200 nM or less, about 100 nM or less, or about 50 nM or less, in an amylin receptor binding assay, such as that described herein, in U.S. Pat. No. 5,686,411, and US Publication No. 2008/0176804, the disclosures of which are incorporated by reference herein in their entireties and for all purposes. In one embodiment, amylin agonist compounds will have an EC₅₀ of about 20 nM or less, about nM 15 or less, about nM 10 or less, or about nM 5 or less in a soleus muscle assay, such as that described herein and in U.S. Pat. No. 5,686,411. In one embodiment, the amylin agonist compound has at least 90% or 100% sequence identity to ^{25,28,29}Pro-human-amylin. In one embodiment, the amylin agonist compound is a peptide chimera of amylin (e.g., human amylin, rat amylin, and the like) and calcitonin (e.g., human calcitonin, salmon calcitonin, and the like). Suitable and exemplary amylin agonist compounds are also described in US Publication No. 2008/0274952, the disclosure of which is incorporated by reference herein in its entirety and for all purposes.

When co-administered with another active agent, the compounds can be administered simultaneously or sequentially, together or separately formulated. Since the engineered compounds herein are inherently long-acting, they are suitable for once daily, once weekly or longer administration. Accordingly, the other agent may be administered either in one or multiple doses, e.g. once daily, twice daily, three times daily, once weekly, as needed, during the period of dosing for the exendin engineered polypeptide, e.g. once weekly.

Single and multiple-use formulations of other agents such as amylin compounds have been reported. For example, pramlintide has been formulated for and successfully administered for once, twice and three times daily administration for treating diabetes and for treating obesity.

A stabilizer may be included in the formulations but is not necessarily needed. If included, however, a stabilizer useful in the practice of the present invention is a carbohydrate or a polyhydric alcohol. A suitable stabilizer useful in the practice of the present invention is approximately 1.0 to 10% (w/v) of a carbohydrate or polyhydric alcohol. The polyhydric alcohols and carbohydrates share the same feature in their backbones, i.e., —CHOH—CHOH—, which is responsible for stabilizing the proteins. The polyhydric alcohols include such compounds as sorbitol, mannitol, glycerol, and polyethylene glycols (PEGs). These compounds are straight-chain molecules. The carbohydrates, such as mannose, ribose, sucrose, fructose, trehalose, maltose, inositol, and lactose, on the other hand, are cyclic molecules that may contain a keto or aldehyde group. These two classes of compounds have been demonstrated to be effective in stabilizing protein against denaturation caused by elevated temperature and by freeze-thaw or freeze-drying processes. Suitable carbohydrates include: galactose, arabinose, lactose or any other carbohydrate which does not have an adverse affect on a diabetic patient, i.e., the carbohydrate is not metabolized to form unacceptably large concentrations of glucose in the blood. Such carbohydrates are well known in the art as suitable for diabetics. Sucrose and fructose are suitable for use with the compound in non-diabetic applications (e.g. treating obesity).

In certain embodiments, if a stabilizer is included, the compound is stabilized with a polyhydric alcohol such as sorbitol, mannitol, inositol, glycerol, xylitol, and polypropylene/ethylene glycol copolymer, as well as various polyethylene glycols (PEG) of molecular weight 200, 400, 1450, 3350, 4000, 6000, 8000 and even higher). Mannitol is the preferred polyhydric alcohol in some embodiments. Another useful feature of the lyophilized formulations of the present invention is the maintenance of the tonicity of the lyophilized formulations described herein with the same formulation component that serves to maintain their stability. In some embodiments, mannitol is the preferred polyhydric alcohol used for this purpose.

Antimicrobial agents should be evaluated to ensure compatibility with all other components of the formula, and their activity should be evaluated in the total formula to ensure that a particular agent that is effective in one formulation is not ineffective in another. It is not uncommon to find that a particular antimicrobial agent will be effective in one formulation but not effective in another formulation.

A preservative is, in the common pharmaceutical sense, a substance that prevents or inhibits microbial growth and may be added to pharmaceutical formulations for this purpose to avoid consequent spoilage of the formulation by microorganisms. While the amount of the preservative is not great, it may nevertheless affect the overall stability of the peptide.

While the preservative for use in the pharmaceutical compositions can range from 0.005 to 1.0% (w/v), in some embodiments range for each preservative, alone or in combination with others, is: benzyl alcohol (0.1-1.0%), or m-cresol (0.1-0.6%), or phenol (0.1-0.8%) or combination of methyl (0.05-0.25%) and ethyl or propyl or butyl (0.005%-0.03%) parabens. The parabens are lower alkyl esters of para-hydroxybenzoic acid. A detailed description of each preservative is set forth in Remington's Pharmaceutical Sciences (Id.) In some embodiments the permeation enhancer, which will be present at more than 1% by weight/weight or by weight/volume, can also act as a preservative. For example, propyl gallate when used at concentrations to enhance permeation of the Exendin ABD, may also act as a preservative.

Water of suitable quality for formulations can be prepared either by distillation or by reverse osmosis.

It is possible that other ingredients may be present in the pharmaceutical formulations. Such additional ingredients may include, e.g., wetting agents, emulsifiers, oils, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Additionally, polymer solutions, or mixtures with polymers provide the opportunity for controlled release of the peptide. Such additional ingredients, of course, should not adversely affect the overall stability of the pharmaceutical formulation of the present invention.

Each of the components of the pharmaceutical formulation described above is known in the art and is described in Pharmaceutical Dosage Forms: Parenteral Medications, Vol. 1, 2nd ed., Avis et al. Ed., Mercel Dekker, New York, N.Y. 1992, which is incorporated by reference in its entirety herein.

The formulations of the engineered polypeptides described herein are administered to a mucosa surface of a subject. These routes include, but are not limited to, oral, nasal, sublingual, pulmonary, vaginal and buccal routes, which may include administration of the peptide in liquid, semi-solid or solid form. Preferred route is oral, for delivery to the intestine, and sub-lingual. Administration via these routes can require substantially more compound to obtain the desired biological effects due to decreased bioavailability compared to parenteral delivery.

The compounds may be provided in dosage unit form containing an amount of the engineered polypeptide that will be effective in one or multiple doses.

As will be recognized by those in the field, an effective amount of the engineered polypeptide will vary with many factors including the age and weight of the subject, the subject's physical condition, the condition to be treated, and other factors known in the art. An effective amount of the engineered polypeptides will also vary with the particular combination administered. As described herein, administration of the engineered polypeptides in combination may allow for a reduced amount of any of the administered engineered polypeptides to be an effective amount.

Administration for transmucosal delivery can include transcellular, paracellular or receptor-mediated routes. Without wishing to be bound by any theory, the engineered polypeptides containing an exendin as described herein are orally available, in part because of their relatively small size and relative stability to gut enzymes. It has been reported that tight junctions between intestinal cells opened by absorption/permeation enhancers are less than 20 nm wide. See e.g., Chao et al., 1998, J. Drug Targeting, 6:37-43. Accordingly, a sufficiently small (for example, less than 10 kD or 15 kD) engineered polypeptide as described herein can transit the gut wall and bind albumin in the portal system, thereby gaining access to the circulation. Transmucosal delivery, preferably oral or sub-lingual, even more preferably oral delivery, of the engineered polypeptides may be twice daily, once daily, once every other day, once every three days, once weekly, once in two weeks, one in three weeks, or even once a month. In a preferred embodiment the transmucosal delivery system, e.g. tablet, capsule, film, preferably via an oral delivery to the intestine or sub-lingual, will have a relatively rapid uptake profile, e.g. from 1 to 4 hours, preferably 30 minutes to 60 minutes, after reaching the target site, in which case the inherently long-duration of action of the engineered polypeptide provides the extended duration of action desired, such as for once daily or once weekly administration. The duration of action can be selected, for example, by choice of ABD and its affinity for albumin. While not wishing to be bound by theory, it is believed that higher affinity to albumin will yield longer circulation times providing longer duration of action.

Delivery routes and formulations can be tested using known in vitro and in vivo methods. For example, a mouse can be orally gavaged with a solution containing an engineered polypeptide formulated with or without a permeation/absorption enhancer and/or protease inhibitor in order to test orally availability and effect of any added excipient. A formulation can be administered to an animal jejunum or duodenum as known in the art, and exemplified herein, to test a formulation. Tablets or capsules can be administered to a dog, pig, or primate, e.g. monkey, for further screening. Either or both pharmacodynamic (therapeutic effects) and pharmacokinetic (drug properties) can be measured over time, such as drug plasma levels, acute or chronic glucose and/or HbA1c lowering, insulin plasma levels, food intake inhibition, weight loss, and/or lipid levels, using methods known in the art.

A. Effective Dosages

Pharmaceutical compositions provided herein include compositions wherein the active ingredient is contained in a therapeutically effective amount, i.e., in an amount effective to achieve its intended purpose. The actual amount effective for a particular application will depend, inter alia, on the condition being treated. For example, when administered in methods to treat diabetes, such compositions will contain an amount of active ingredient effective to achieve the desired result (e.g. decreasing fasting blood glucose in a subject). When administered in methods to treat obesity, such compositions will contain an amount of active ingredient effective to achieve the desired result (e.g. decrease the body mass).

The dosage and frequency (single or multiple doses) of compound administered can vary depending upon a variety of factors, including route of administration; size, age, sex, health, body weight, body mass index, and diet of the recipient; nature and extent of symptoms of the disease being treated (e.g., the disease responsive to compounds described herein; fasting blood glucose); presence of other diseases or other health-related problems; kind of concurrent treatment; and complications from any disease or treatment regimen. Other therapeutic regimens or agents can be used in conjunction with the methods and compounds of the invention.

Therapeutically effective amounts for use in humans may be determined from animal models. For example, a dose for humans can be formulated to achieve a concentration that has been found to be effective in animals. The dosage in humans can be adjusted by monitoring one or more physiological parameters, including but not limited to blood sugar and body mass, and adjusting the dosage upwards or downwards, as described above and known in the art.

Dosages may be varied depending upon the requirements of the patient and the compound being employed. The dose administered to a patient, in the context of the present invention, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached. In one embodiment of the invention, the dosage range is 0.001% to 10% w/v or w/w. In another embodiment, the dosage range is 0.1% to 5% w/v w/w.

However, typical doses may contain from a lower limit of about 50 ug, 100 ug to 150 ug per day to an upper limit of about to 100 ug, to 300 ug, to 500 ug, to 1000 ug, to 1200 ug, to 1500 ug, to 2000 ug, to 3000 ug to 4000 ug to even 5000 ug of the engineered polypeptide per dose, as will be generally applicable for a once daily to once weekly delivery. For delivery at less frequent schedule, e.g once every two weeks, once a month, the active concentration will increase accordingly. Accordingly, an upper limit of about 4000 ug, 5000 ug, to 10000 ug to even 20000 ug per dose. In view of the extended half-life of the engineered polypeptides herein, sub-therapeutic doses can be administered at a dosing regimen in which the next dose is provided at a time point at which the polypeptide drug is still present in the subject's blood, such that the therapeutic level is achieved over two or more doses. Preferably, the regimen provides an average sustained plasma concentration that achieves a therapeutic level of the engineered polypeptide. Preferably the regimen achieves a steady state plasma concentration that achieves a therapeutic level of the engineered polypeptide. The doses may be delivered in discrete unit doses at the desired interval, e.g. daily or weekly.

Dosage amounts and intervals can be adjusted individually to provide levels of the administered compound effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

The surprising dose-sparing property of the engineered polypeptides of the present invention, along with their surprisingly long plasma half-life and duration of pharmacological action, provides for a superior pharmaceutical agent. Also surprising in the case of the exendin-containing engineered polypeptides are their oral availability. The superior properties including dose-sparing, allow for lower dosing, thus less or less severe side-effects and improved cost of goods, and/or more cost-effective and simpler formulations for once daily or once weekly administration not currently achieved by the parent compounds alone.

B. Toxicity

The ratio between toxicity and therapeutic effect for a particular compound is its therapeutic index and can be expressed as the ratio between LD₅₀ (the amount of compound lethal in 50% of the population) and ED₅₀ (the amount of compound effective in 50% of the population). Compounds that exhibit high therapeutic indices are preferred. Therapeutic index data obtained from cell culture assays and/or animal studies can be used in formulating a range of dosages for use in humans. The dosage of such compounds preferably lies within a range of plasma concentrations that include the ED₅₀ with little or no toxicity. The dosage may vary within this range depending upon the dosage form employed and the route of administration utilized. See, e.g. Fingl et al., In: THE PHARMACOLOGICAL BASIS OF THERAPEUTICS, Ch. 1, p. 1, 1975. The exact formulation, route of administration, and dosage can be chosen by the individual physician in view of the patient's condition and the particular method in which the compound is used.

Additional Exemplary Embodiments: The Examples provided below exemplify and illustrate the disclosure and should not be viewed as limiting.

### VII. Examples

Peptides useful in the examples following include: HaPGTFTSDLSKQMEEE AVRLFIEWLKNGGP SSGAPPP STGGGGSASLAEAKVLANRELDKYGVSDFYKRLINKAK TVEGVEALKLHILAALP; HAEGTFTSDVSSYLEGQAAKEFIAWLVKLAEAKVLAN RELDKYGVSDFYKRLINKAKTVEGVEALKLHILAALP (SEQ ID NO:164); HGEGTFTSDLSKQMEEEAVRLFIEWLKLAEAKVLANRELDKYGVSDFYKRLINKAKTV EGVEALKLHILAALP (SEQ ID NO:165); HGEGTFTSDLSKQMEEEAVRLFIEW LKNGGPSSGAPPPSGGSLKNAKEDAIAELKKAGITSDFYFNAVNKAKTVEEVNALKNEI LKALP (Cmpd 22) (SEQ ID NO:168); H(Aib)QGTFTSDYSKYLDEQAAKEFIAWLMN TYGVSDFYKRLINKAKTVEGVEALKLHILAALP (SEQ ID NO:171); HSQGTFTSDYSKYLDEQAAKEFIAWLMNTYGVSDFYKRLINKAKTVEGVEALKLHILA ALP (SEQ ID NO:172); HSQGTFTSDYSKYLDEQAAKEFIAWLMNTGGGSYGVSD FYKRLINKAKTVEGVEALKLHILAALP (SEQ ID NO:173); HaPGTFTSDLSKQMEEE AVRLFIEWLKNGGP SSGAPPP STGGGGSASLAEAKVLANRELDKYGVSDFYKRLINKAK TVEGVEALKLHILAALP (Cmpd 14), and [[Lys27#]HGEGTFTSDLSKQMEEEA VRLFIEWLKNGGPSSGAPPPS][LAEAKVLANRELDKYGVSDFYKRLINKAKTVEGVEAL KLHILAALP-GGG-#] (Cmpd 30). As customary in the art, a lower case single-letter amino acid abbreviation (e.g., “a”) indicates a D-amino acid (e.g., D-Ala). In the nomenclature of side chain linked peptide compounds, square brackets (“[ ]”) indicate separate fragments and crosshatch (“#”) indicates linking positions.

### Example 1

### Purification of Exendin Analog-ABD Engineered Polypeptide

Method.

Exemplary Cmpd 15 (SEQ ID NO:163) was initially produced having an N-terminal extension which incorporates a His₆ (SEQ ID NO:49) “tag” as known in the art, with sequence:

(SEQ ID NO: 50)
MAHHHHHHVGTGSNENLYFQHGEGTFTSDLSKQLEEEAVRLFIEWLKQGG
PSKEIISTGGGGSASLAEAKVLANRELDKYGVSDFYKRLINKAKTVEGVE
ALKLHILAALP.

Preparation of Cell Extract.

In order to prepare the cell extract, cell pellets from 50 mL of cell cultures were completely resuspended in 60 mL of lysis buffer (50 mM TrisHCl, 150 mM NaCl, pH 8.0). Resuspended cells were run through a microfluidizer (Microfluidics, MA) at 100 PSI three times. Cell extracts were centrifuged for 30 min at 16,000×g to remove debris. EGTA (150 mM stock) was added to cell extract to a final concentration of 3 mM.

Ni-NTA Chromatography.

Ten mL of 50% suspension of Ni-NTA superflow was packed to a 15 mL empty column. The column was washed with 10 mL of water, 50 mL of lysis buffer, and 20 mL of lysis buffer with 3 mM EGTA (50 mM TrisHCl, 150 mM NaCl, pH8.0, 3 mM EGTA). Cell extract was carefully added on the top of Ni-NTA column, and the flow-through was collected. The column was washed with 30 mL of lysis buffer with EGTA (50 mM TrisHCl, 150 mM NaCl, pH8.0, 3 mM EGTA). Ten mL of elution buffer (25 mM TrisHCl, 50 mM NaCl, 250 mM imidazole, pH8.0) was added to the top of column, and the elution fractions (2 mL/fraction) were collected. SDS-PAGE was run to check the flow through and each fraction. Fractions containing the His-tagged compound were pooled.

TEV Protease Digestion.

His₆-tagged compound was diluted three fold with 25 mM TrisHCl, 50 mM NaCl, pH8.0. β-mercaptoethanol (0.1%) and 2% of Turbo TEV protease (2 mg/mL, 10,000 units/mg, Accelagen), were added, and the result was mixed and incubated at RT for 2 hours and at 4° C. over night.

Removal of Cleaved his-Tag and Turbo TEV with Ni-NTA.

Six mL of 50% suspension of Ni-NTA superflow was packed to a 15 mL empty column. The column was washed with 20 mL of water and 20 mL of 50 mM TrisHCl, 100 mM NaCl, 45 mM imidazole, pH8.0. The TEV digest reaction was diluted 2-fold with 50 mM TrisHCl, 150 mM NaCl, pH8.0. Diluted digest reaction was carefully added to the top of Ni-NTA column, and the flow-through was collected. Ten mL of 50 mM TrisHCl, 100 mM NaCl, 45 mM imidazole, pH8.0, was added to the column to elute any unbound protein. The flow-throughs were collected and combined.

First Size Exclusion Chromatography (SEC).

The Ni-NTA flow-through was filtered with 0.2 um filter. Superdex 75 HiLoad 26/60 column was pre-equilibrated with 390 mL of PBS. Filtered flow-through was injected to the HiLoad 26/60 column with a sample pump. Protein was eluted with 1.5 CV of PBS, and the monomer peak was pooled.

Second Size Exclusion Chromatograph.

The first SEC pool was filtered with 0.2 um filter. A Superdex 75 HiLoad 26/60 column was pre-equilibrated with 390 mL of PBS. Filtered flow-through was injected to the column HiLoad 26/60 with a sample pump. Protein was eluted with 1.5 CV of PBS, and the monomer peak was pooled.

Third Size Exclusion Chromatography.

The second SEC pool was filtered with 0.2 um filter. A Superdex 75 HiLoad 26/60 column was pre-equilibrated with 390 mL of PBS. Filtered flow-through was injected to the column HiLoad 26/60 with a sample pump. Protein was eluted with 1.5 CV of PBS, and the monomer peak was pooled.

Removal of Residual Endotoxin with EndoTrap Red.

The third SEC pool still contained ˜20 EU/mg of endotoxin, which was removed by the use of EndoTrap Red. Briefly, 0.5 mL of gel slurry was activated by adding 1 mL of Regeneration Buffer to the slurry and mix by gently shaking the tube for approximately 5 seconds. The supernatant was centrifuged and aspirated. This step was repeated two additional times. One mL of Equilibration Buffer was added, and mixing was conducted by gently shaking the tube for approximately 5 seconds. The supernatant was centrifuged and aspirated. This step was repeated two additional times. Protein sample (5.5 mL) was added to the resin and incubated for 90 minutes at RT, with gentle rocking or rotating of the tube while incubating. The result was centrifuged at 1200×g for 5 minutes, and the supernatant was transferred to a clean tube.

Results.

The final purified protein migrated on SDS-PAGE gel as approximately a 6 kD protein under the conditions employed. The LC-MS showed a correct molecular weight of 9827 dalton. The protein yield was 3.3 mg from 50 mL of cell culture.

### Example 2

### Activities of Exendin-ABD Engineered Polypeptides

Exendin-ABD engineered polypeptides of the invention retained sufficient exendin activity in an in vitro cell activation assay. Additionally, the engineered polypeptides provided dramatically improved duration of action for blood glucose lowering and body weight loss, as when compared to exendin-4, when administered as a single dose to a mammal Surprisingly, duration of action can be extended to at least 1 day, even at least 4 days, and even at least 7 days, or longer, in a rodent model, which translates to at least one week duration of action in a human subject, thus suitable for twice daily, once daily, three times weekly, twice weekly or even once weekly administration.

Functional activity of the compounds disclosed herein can be determined using a cell line expressing GLP-1 receptor. See e.g., United States Patent Application Publication US20110097751A1, incorporated by reference for the assay method. In this example, functional activity was determined using cells that endogenously express GLP-1R, and cAMP induction is detected as a measure of exendin activity. An HTRF assay kit was used (Cisbio International (Bedford, Mass.). The bioassay used the rat thyroid carcinoma 6-23 (clone 6) cells in the cell-based assay using the HTRF® cAMP dynamic 2 1,000 assay kit, available from Cisbio as Catalog No. 62AM4PEB. The HTRF® standards and calibrations are prepared following the instructions in the kit. Accumulation of cAMP is measured following 30 minutes of compound treatment using the HTRF (CisBio) cell-based cAMP assay kit in 384-well format. Efficacy of peptides is determined relative to cell treatment with 10 uM forskolin (a constitutive activator of adenylate cyclase), and potency (EC₅₀) of peptides is determined by the analysis of a concentration-response curve using non-linear regression analysis fitted to a 4-parameter model. The results of the GLP-1 receptor functional activity (cAMP induction) for potency (EC₅₀) are provided in the following Table 5, where values normalized to an exendin-4 standard. The ABD domain did not bind nor activate the GLP-1 receptor.

TABLE 5
GLP-1R Functional Activity

| | GLP-1R |
|---|---|
| | Functional |
| | activity (EC₅₀) in |
| Description | nM |
| Exendin-4 | 0.004 |
| [Leu¹⁴,Gln²⁸]Exendin-4(1-32)-fGLP-1)33-37) | 0.016 |
| (SEQ ID NO: 4) | |
| Exendin-4 (1-28) amide | 0.011 |
| Cmpd 5 (SEQ ID NO: 40) | 0.982 |
| Cmpd 6 | 0.0325 |
| Cmpd 15 | 0.091 |
| Cmpd 8 | 0.048 |
| Cmpd 10 | 0.146 |
| Cmpd 21 (SEQ ID NO: 99) | 0.131 |
| Cmpd 31 | 0.62 |
| Cmpd 32 | 2.043 |
| Cmpd 33 | 0.77 |

### Example 3

### OGTT DOA Activity

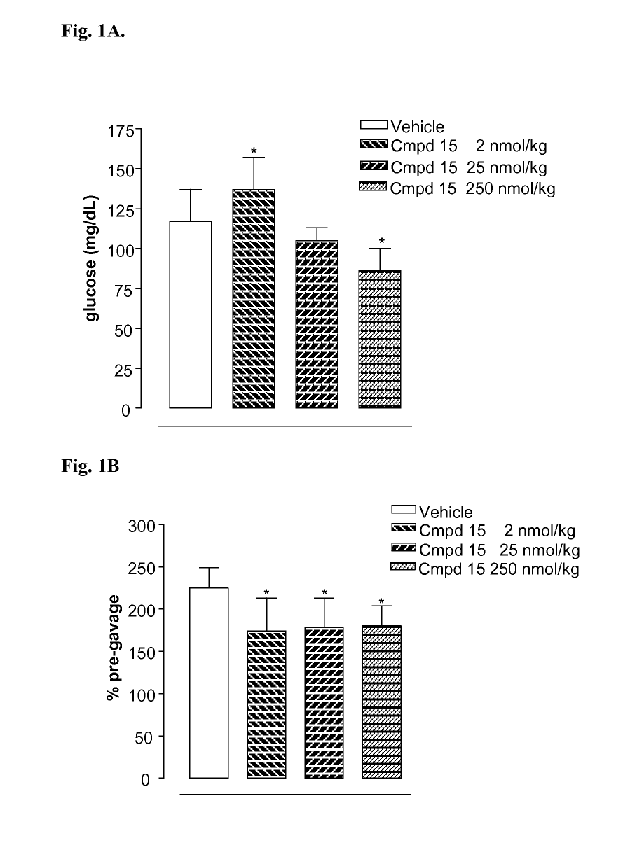

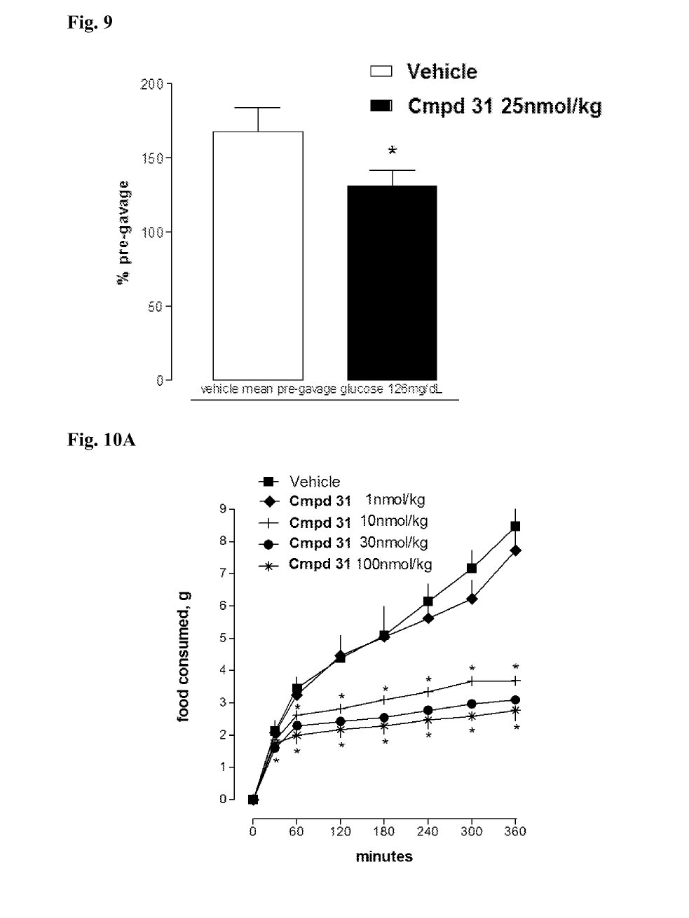

The effects on blood glucose prior to glucose gavage (1.5 k/kg dextrose) and at 30 minutes post-glucose gavage were investigated 1 day post dose of peptide compound with varying amounts of Cmpd 15, with results shown in FIGS. 1A-1B. Cmpd 31 at 25 nmol/kg also demonstrated activity at 24 hours post dosing, as shown in FIG. 9. Drug was administered to 4-hr fasted NIH/Swiss mice at the doses indicated in the figures. Bars represent mean±sd. Peptide was injected IP at t=−1 day. Glucose gavage (1.5 g/kg) given at t=0 to 4-hour fasted NIH/Swiss female mice. Blood glucose was measured with a OneTouch® Ultra® (LifeScan, Inc., a Johnson & Johnson Company, Milpitas, Calif.) * p<0.05 vs. vehicle control; ANOVA, Dunnett's test. This OGTT DOA indicates drug activity is present at least 24 hours after drug was administered. Exendin-4 (unconjugated) was ineffective in this assay when dosed at t-24 hours (1 day prior to the glucose assay), and even at higher doses.

### Example 4

### OGTT DOA Activity

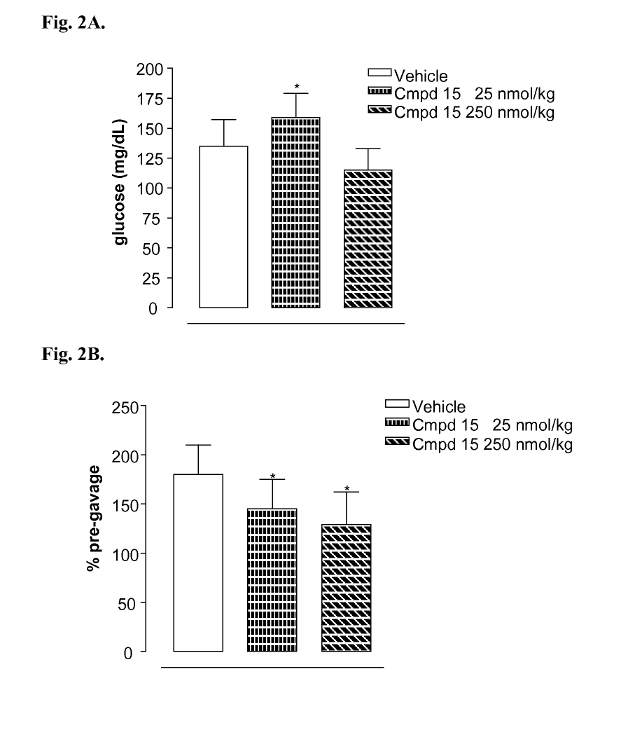

The effects on blood glucose prior to gavage (1.5 k/kg dextrose) and at 30 min were investigated 2 day post dose with varying amounts of Cmpd 15, with results shown in FIGS. 2A-2B. Drug was administered to 4-hr fasted NIH/Swiss mice at the doses indicated in the figures. This OGTT DOA indicates drug activity is present at least 48 hours after drug was administered.

### Example 5

### OGTT DOA Activity

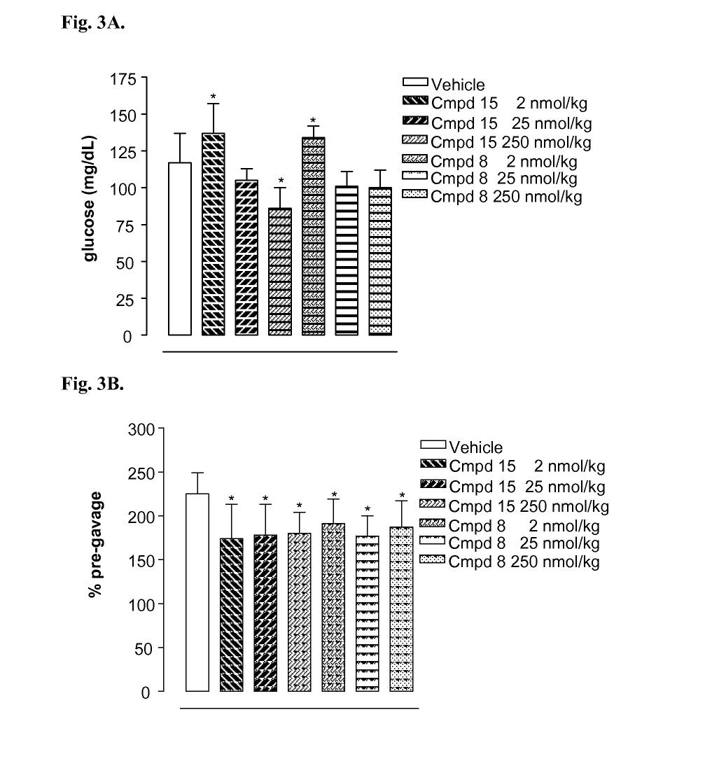

A comparison of the effects of Cmpds 15 and 8 on blood glucose was conducted, with results depicts in FIGS. 3A-3B. Drug was administered to 4-hr fasted NIH/Swiss mice at the doses indicated in the figures. This OGTT DOA indicates drug activity is present at least 24 hours after drug was administered.

### Example 6

### Effect of Cmpd 15 on HSD Fed Anesthetized Rats

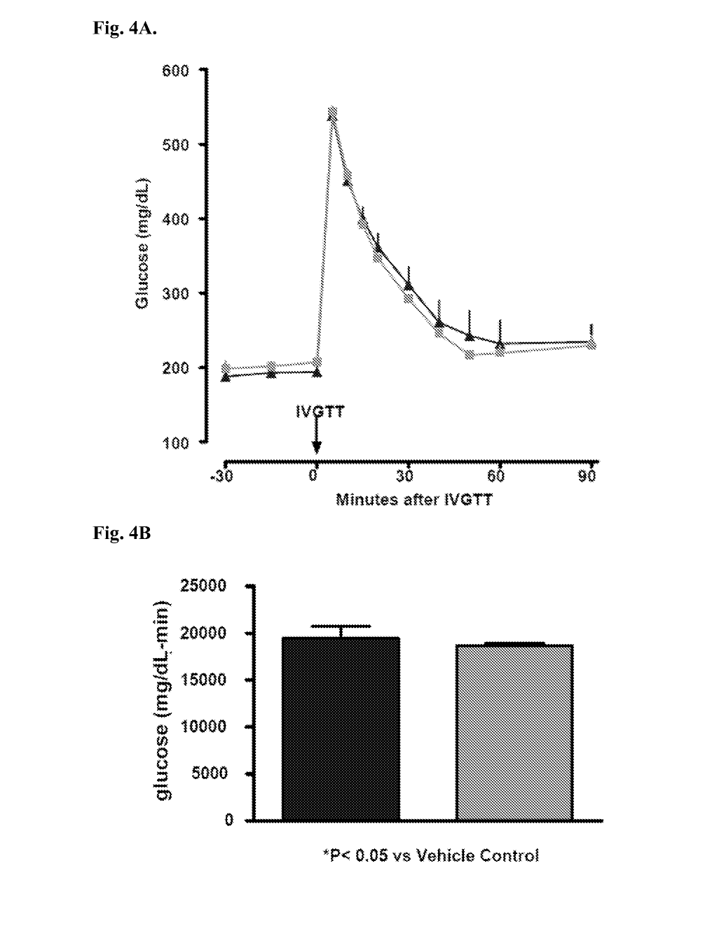

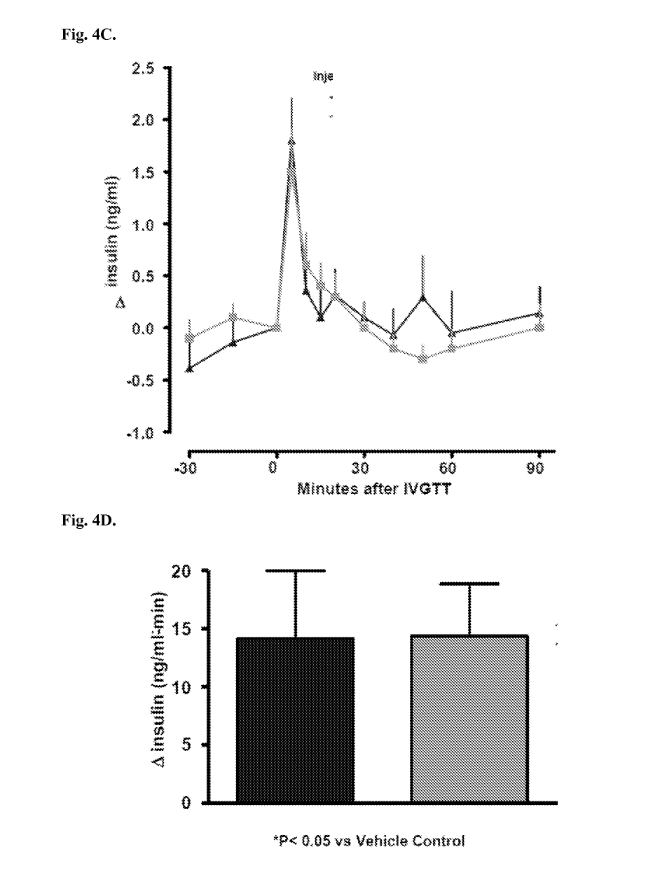

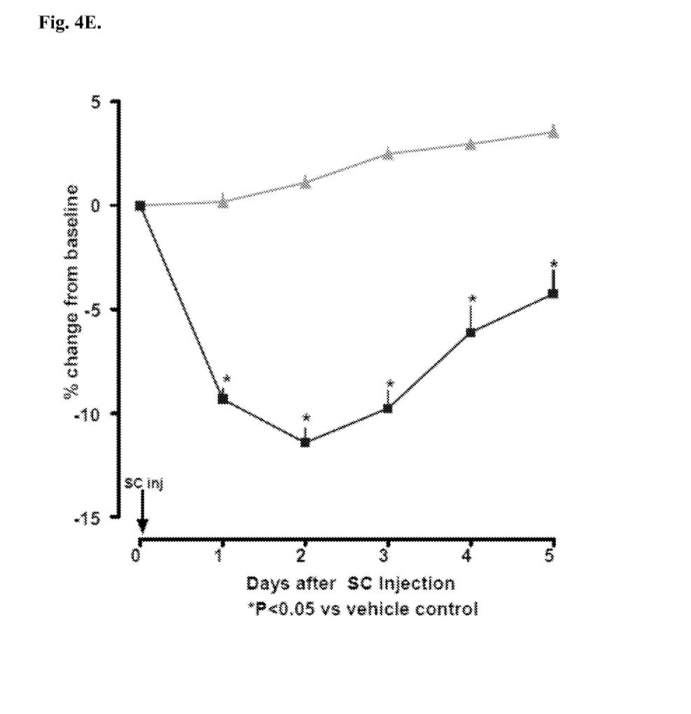

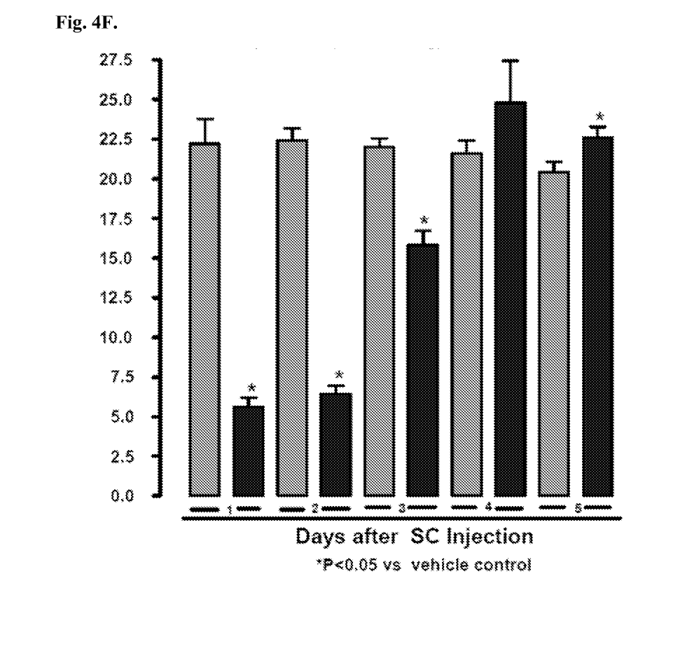

The effects of treatment with Cmpd 15 (240 nmol/kg) were investigated in Sprague Dawley fed anesthetized rats 5 days post dose. The time course of plasma glucose after IVGTT is depicted in FIG. 4A. Integrated (AUC₀₋₆₀) glucose levels are depicted in the histogram of FIG. 4B. The time course of the change in insulin levels in the test subjects was depicted in FIG. 4C. The integrated insulin levels (AUC₀₋₃₀) are depicted in FIG. 4D. The time course of body weight change (% change from baseline) is depicted for the test subjects in FIG. 4E. A histogram depiction of daily food intake for the test subjects is provided in FIG. 4F. This IVGTT DOA indicates drug activity is present at least 5 days hours after drug was administered, particularly for effects on body weight and daily food intake.

### Example 7

### Effect of Cmpd 15 in Ob/Ob Mice

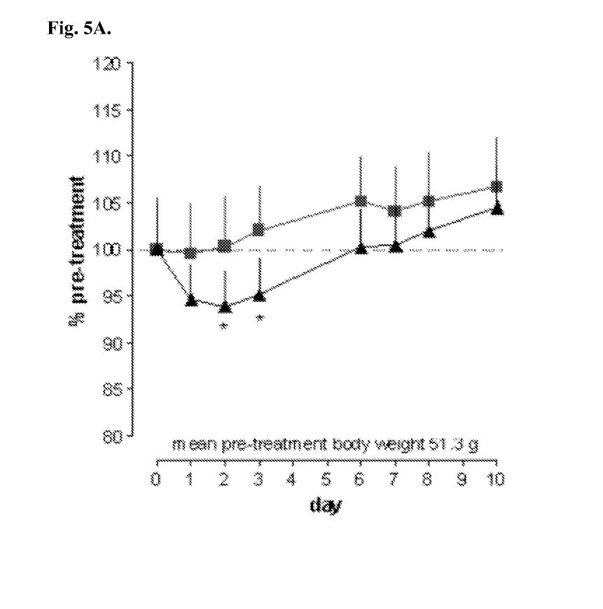

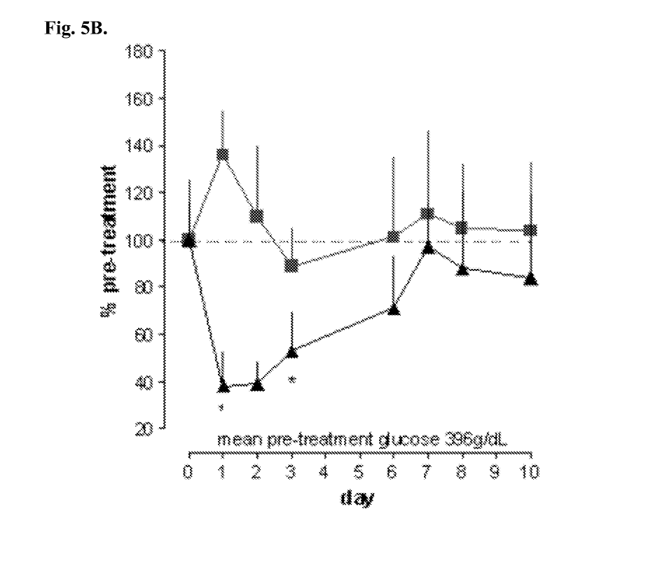

The time course of the effect of Cmpd 15 on body weight, glucose and HbA_{1c} in ob/ob mice was investigated post dose. As depicted in FIG. 5A, significant body weight loss attends treatment with 250 nmol/kg Cmpd 15. Changes in glucose (% pre-treatment) and in HbA1c (% pre-treatment) are depicted in FIGS. 5B-5C. Points represent mean±s.d. (standard deviation). Cmpd 15 was injected sc on day=0 immediately following baseline sample collection in non-fasted male ob/ob mice. Unless indicated otherwise, blood glucose measures described herein employed a OneTouch® Ultra® device (LifeScan, Inc. Miliptas, Calif.). Cmpd 21 also demonstrated body weight loss and reduction of HbA1c.

### Example 8

### Activity of Cmpd 15 in Zucker Diabetic Fatty (ZDF) Rats

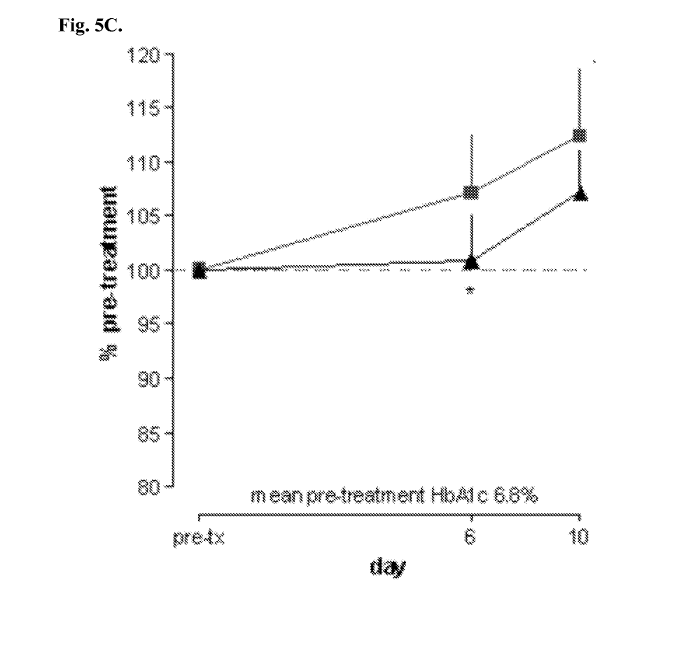

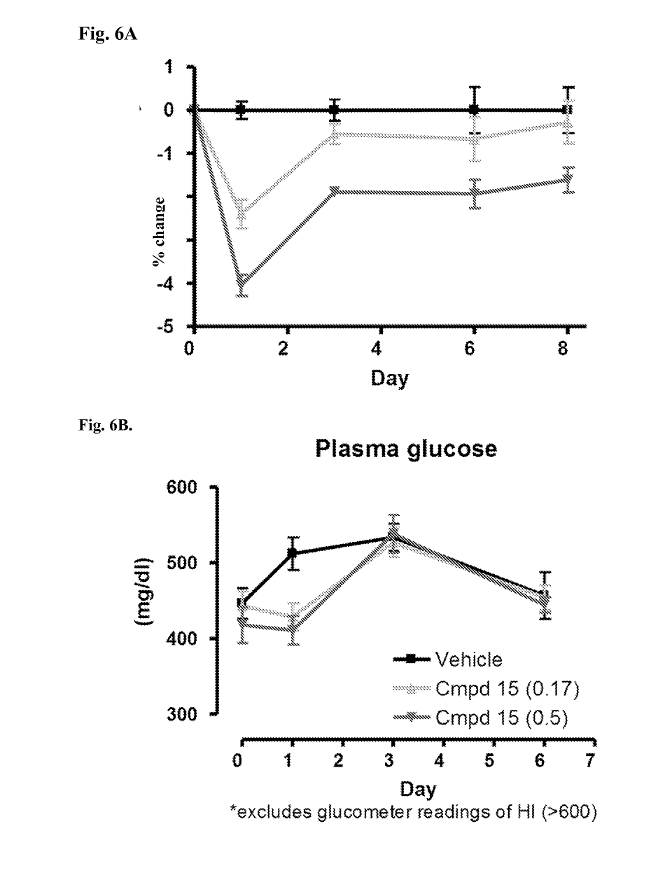

To assess the combined body weight and glucose lowering efficacy of exemplary compounds described herein, the dose dependent effects of Cmpd 15 in ˜14 week old male ZDF rats was investigated. Baseline glucose was 426 mg/dL, and baseline body weight was 431 g. Group size n=8. FIG. 6A depicts the time course of the change in body weight (% vehicle corrected) after treatment. FIG. 6B depicts the time course of plasma glucose.

### Example 9

### Activity of Cmpds 15, 8 and 10 on OGTT DOA (Duration of Action)

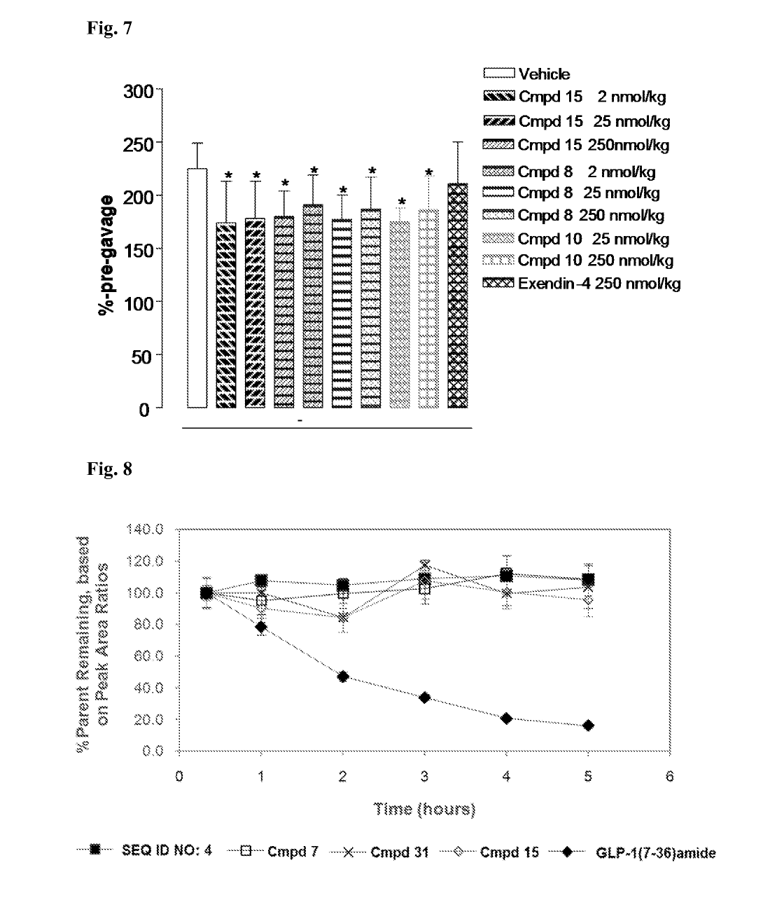

The effects of Cmpds 15, 8 and 10 on the change in blood glucose at 30 min (% pre-gavage) was investigated, as depicted in FIG. 7. In the figure, bars represent mean±s.d. Test compound was injected IP at t=−1 day. Glucose gavage (1.5 g/kg) given at t=0 to 4 hr fasted NIH/Swiss female mice. Blood glucose was measured as described herein. This OGTT DOA indicates drug activity is present at least 24 hours after drug was administered.

### Example 10

### Activity of Cmpds on OGTT DOA (Duration of Action) at 24 Hours

The effects of compounds disclosed herein on the change in blood glucose at 30 min (% pre-gavage) were investigated as described above. Test compound was injected IP at t=−1 day at 25 nmol/kg. Glucose gavage (1.5 g/kg) given at t=0 to 4 hr fasted NIH/Swiss female mice. Blood glucose was measured as described herein. This OGTT DOA indicates drug activity is present at least 24 hours after drug was administered. Results are presented in the following Table 6. Cmpd 30 (Lysine 27-linked) and Cmpd 32 gave no glucose lowering, indicating a lack of presence at 24 hours under these conditions. Exendin-4 (unconjugated) was ineffective in this assay when dosed at t-24 hours, and even at higher doses. Cmpd 14 with proline at position 3 was essentially inactive in the in vitro functional assay and inactive (and perhaps weight promoting) in the glucose lowering OGTT assay (data not shown). Cmpd 22 with an albumin binding sequence the PAB protein from P. magnus had little if any weight lowering (3%) in the above assay. Cmpd 19 and Cmpd 20 with truncated ABDs still maintained in vitro activity, but with reduced duration, having 6% and 8% glucose lowering in the OGTT DOA assays, respectively.

TABLE 6
Glucose Lowering in OGTT at 24 Hours Post Dose

| | % Glucose Lowering |
|---|---|
| Description | Compared to Vehicle |
| Cmpd 5 (SEQ ID NO: 40) | −28 |
| Cmpd 6 | −18 |
| Cmpd 15 | −21 |
| Cmpd 8 | −21 |
| Cmpd 10 | −22 |
| Cmpd 21 (SEQ ID NO: 99) | −23 |
| Cmpd 23 | −23 |
| Cmpd 24 | −17 |
| Cmpd 31 | −22 |
| Cmpd 33 | −19 |

### Example 11

### Serum Albumin Binding

Characterization of the binding of engineered polypeptide compounds to albumin can be performed by any number of methods, including that of Biacore described herein. In this example binding measurements were conducted with a BioRad ProteOn XPR36 system (Bio-Rad Laboratories, Hercules Calif., USA; ProteOn XPR36 Protein Interaction Array System catalog number #176-0100), using a GLC sensor chip at 25 degrees C. For amine coupling the GLC chip was activated for 5 minutes using a 1:1 mixture of sulfo-NHS/EDC diluted 30-fold from the initial stock in water as shown below. Each albumin sample was diluted to 25 ug/ml in 10 mM Na acetate pH 5.0 and injected for 5 minutes over separate sensor surfaces. Each surface was then blocked with 1 M ethanolamine pH 8.5. Each albumin was coupled at a density of 2000-5000 in resonance units. The binding of an engineered polypeptide was tested using 5 nM as the highest concentration in a three-fold dilution series. The running buffer contained 10 mM HEPES pH 7.4, 150 mM NaCl, 3 mM EDTA and 0.005% tween-20. All samples were tested using a 3-fold dilution series. Each concentration series was tested in duplicate. The dissociation phase for the highest concentration was monitored for 3 hours.

The relative K_{D} measured for the engineered polypeptides are presented in Table 7 below. The results show that the albumin binding polypeptides associate with serum albumins with high affinity. The number in parentheses represents the standard deviation in the last significant digit. As seen from the following table the exendin polypeptides fused to albumin binding domains of SEQ ID NO:35 retain extremely high affinity for serum albumin from various species, especially human serum albumin, even compared to the unconjugated ABD peptide itself

TABLE 7

| | | | | | Monkey | | | | | |
|---|---|---|---|---|---|---|---|---|---|---|
| Cmpd | Human SA | | Dog SA | | SA | | Mouse SA | | Rat SA | |
| SEQ ID NO: 35 | 16(4) | pM | 201(2) | pM | 123(1) | pM | 1.24(1) | nM | 18(5) | pM |
| Cmpd 15 | 68 | pM | 513 | pM | 91 | pM | 1.25 | nM | 200 | pM |
| Cmpd 21 | 85 | pM | 397 | pM | 78 | pM | 1.33 | nM | 16 | pM |
| (SEQ ID NO: 99) | | | | | | | | | | |

### Example 12

### Activity in the Presence of Serum Albumin

Characterization of the in vitro activity of the engineered polypeptide compounds in the presence of serum albumin was demonstrated. Assays can be run in the presence and absence of an albumin, particularly human serum albumin. The data above was determined in the presence of about 0.1% bovine serum albumin (BSA). The following table presents functional activity of receptor activation (cAMP induction) assay described above, but in the presence of serum albumin from various species. As can be seen, surprisingly, even when compounds are bound to serum albumin, such as to human serum albumin, despite the presence of the large serum albumin, with its potential for steric hindrance and even a change in the apparent Stoke's radius of the compounds resulting from albumin binding, the engineered polypeptide retains GLP-1 receptor agonist activity. Given the picomolar affinity of ABD and the engineered polypeptides to some species of serum albumin, e.g. human serum albumin, the engineered polypeptide is believed to be effectively fully bound to albumin present in the assay (and thus also in vivo in circulating blood). Because of the extremely high affinity of compound binding to albumin (as above) and the presence of high concentration of serum albumin in the blood, it is expected that the compounds will exist essentially in the bound state in vivo yet surprisingly provide sufficient exendin functions (as demonstrated herein).

TABLE 8

| | 0.1% | | | |
|---|---|---|---|---|
| | Bovine | 1% Bovine | 1% Human | 1% Rat |
| Cmpd | Albumin | Albumin | Albumin | Albumin |
| GLP-1(7-36) amide | 0.0306 | 0.0058 | 0.0112 | 0.0179 |
| Cmpd 15 | 0.7854 | 0.2204 | 0.185 | 0.2473 |
| Cmpd 21 (SEQ ID | 1.1013 | 0.2234 | 0.2022 | 0.2164 |
| NO: 99) | | | | |
| Cmpd 31 | 1.1408 | 0.2313 | 0.2139 | 0.2358 |
| GLP-1(7-36) amide | 0.0256 | 0.0224 | 0.0165 | 0.0153 |
| normal assay | | | | |
| conditions | | | | |

### Example 13

### Compounds are Stable to Human Plasma and Human Plasma Enzymes

Compounds were examined for stability to human plasma and human cell membrane proteases. Stability of representative peptides in human plasma was performed as follows. 10 μg/ml of compound in human plasma was prepared at sufficient volume to remove 100 μL samples every 10 minutes for the time period (5 hours), starting at the zero time point. Following the addition of compound to the human plasma, the sample is mixed gently and a 100 μL sample of the mixture was transferred to a microcentrifuge tube to represent the zero time point. The remainder of the sample was placed in an incubator at 37 degrees C., mixing at 600 RPM for sixty minutes. At 10 minute intervals, a 100 μl sample of the mixture was removed and transferred to a separate microcentrifuge tubes. Following the transfer of the 100 μL sample at the zero time point and each 10 minute interval, each collected sample was extracted by slow addition of 100 μl cold 0.2% formic acid:acetonitrile, while mixing. After addition of the acetonitrile solution, the sample was vortex mixed at high speed for 15 seconds. The extracted samples were stored at −20° C. for at least 20 minutes and then centrifuged at 11,000×g for 10 minutes at 5 degrees C. The supernatant of each sample was transferred to a new microcentrifuge tube, centrifuged again, and finally transferred for LC/MS analysis. Sample analysis was done on an Agilent HLPC (LC/MS 1200) using gradient 5-95% acetonitile in water containing 0.1% trifluoroacetic acid. Table 9 present results normalized to a standard (100%). FIG. 8 presents a time profile of percent of compound remaining in Human Plasma over the 5 hour time course.

TABLE 9

| | Percent |
|---|---|
| | Stable in |
| Cmpd | Human Plasma |
| GLP-1(7-37) amide (SEQ ID NO: 5) | 41.7 |
| HGEGTFTSDLSKQLEEEAVRLFIEWLKQGGPSKEIIS | 100 |
| (SEQ ID NO: 4) | |
| Cmpd 15 | 96.0 |
| Cmpd 21 (SEQ ID NO: 99) | 89.5 |
| Cmpd 31 | 93.7 |

Relative stability of representative peptides in a human kidney brush border membrane (KBBM) assay was performed as follows. Human kidney brush border membrane protein extracts are rich in various peptidases. Protein extract preparation (hKBBMP), 5 microL (approximately 7 micrograms/mL of protein) was diluted with 625 microL of HEPES buffer (25 mM, pH 7.4) in a polypropylene micro centrifuge tube with an O-ring seal to avoid solvent evaporation. In a separate vial, peptide stock solution (300 microM in 50% acetonitrile in water) was prepared and 70 microL of this solution was added to the above hKBBMP solution. The solution was gently mixed by manual shaking so that the final peptide concentration is 30 microM. Then 100 microL of this solution was aliquoted into six different tubes and into one tube 200 microL of enzyme stop solution (50% acetonitrile in water with 0.1% TFA) was added. This tube was used for the measurement of the initial peptide concentration at time t=0 minute while all other 5 tubes were incubated at 37 degrees C. using a water bath. At intervals of 1, 2, 3, 4 and 5 hour, each tube was taken out and quenched with 200 microL of stop solution. Finally, all six tubes were centrifuged at 1800×g for 10 min to remove any precipitated proteins. The supernatant (10 microL) was transferred into an HPLC auto sampler, and by using selected ion count method AUC was measured. Each sample was run in triplicates and average AUC was calculated for data analysis. Sample analysis was done on Agilent HPLC with mass detector with an acetonitrile with 0.1% TFA gradient. Percentage of parent peptide remaining from time t=0 to 5 hours of enzymatic digestion was plotted using GraphPad Prism® 5 software. The data was reported as relative peptide stability versus positive control for each peptide. As noted samples were run as n=6, and CV was within 20%. Results from the hKBBM stability assay are presented in Table 10.

TABLE 10

| | Percent |
|---|---|
| Cmpd | Stable |
| GLP-1(7-37) amide (SEQ ID NO: 5) | 16 |
| HGEGTFTSDLSKQLEEEAVRLFIEWLKQGGPSKEIIS | 100 |
| (SEQ ID NO: 4) | |
| Cmpd 21 (SEQ ID NO: 99) | 103 |

### Example 14

### Lack of Vacuolization

With some drugs, such as some pegylated proteins, undesirable vacuoles can form in cytoplasm of epithelial cells lining the proximal convoluted tubules, which is an undesirable toxicity measure. The engineered albumin binding compounds of the present application do not form kidney vacuoles. C57BL6 female mice (n=2 cages, 3 mice/cage) were weighed daily 3 hours prior to lights out Immediately after weighing, on days 0-6 mice were injected subcutaneously with test compound. Mice were sacrificed on day 7 and kidneys submitted for histopathology. Severity score for cytoplasmic vacuolation of renal cortical tubular epithelial cells was as follows: score 1=minimal (8-15%); 2=mild (16-35%); 3=moderate (36-60%); 4=marked (>60%). A positive control compound known to cause vacuole formation was scored as 3. The ABD polypeptide itself scored 0. Cmpd 15 scored 0.

### Example 15

### Effect on Inhibiting Food Intake in Normal Mice

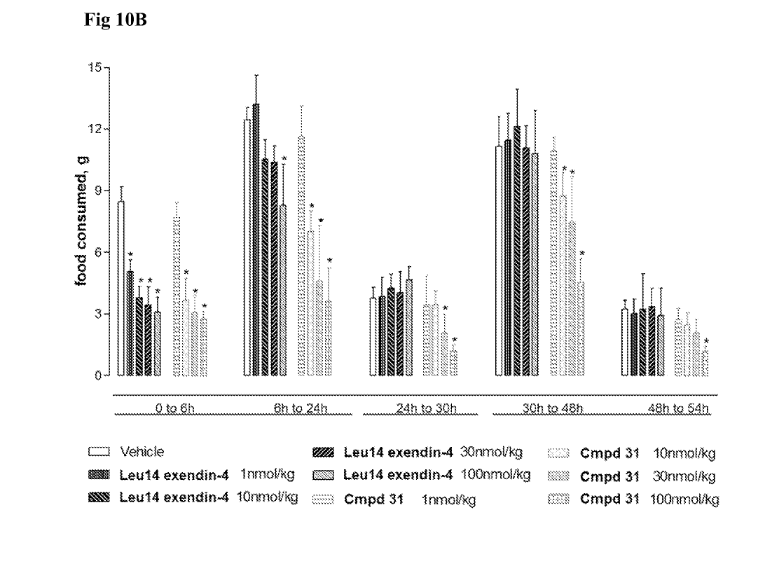

The time course of the effect of test compounds on inhibition of food intake of normal mice was determined. As depicted in FIG. 10A, dose-dependent, significant body weight loss attends treatment with Cmpd 31 over 6 hours. FIG. 10B demonstrates a dose-dependent, sustained inhibition of food intake after a single dose of compound, for at least 54 hours in normal mice. Effect of exendin analog is gone within 24 hours. Cmpd 31 still significantly inhibits food intake even at 3 days at the highest dose. Points represent mean±sd of n=4 cages (3 mice/cage). Peptide was injected IP at t=0. Food was introduced immediately after injection and amount consumed measured at t=30, 60, 120, 180, 240, 300, 360 min, 24 h, 30 h, 48 h, and 54 h. *p<0.05 vs. vehicle control; ANOVA, Dunnett's test. ED50's were ˜10 nmol/kg for Cmpd 31 and 2 nmol/kg for [Leu14] exendin-4.

### Example 16

### Effect of an Exendin-Albumin Binding Domain Polypeptide in Diabetic Ob/Ob Mice

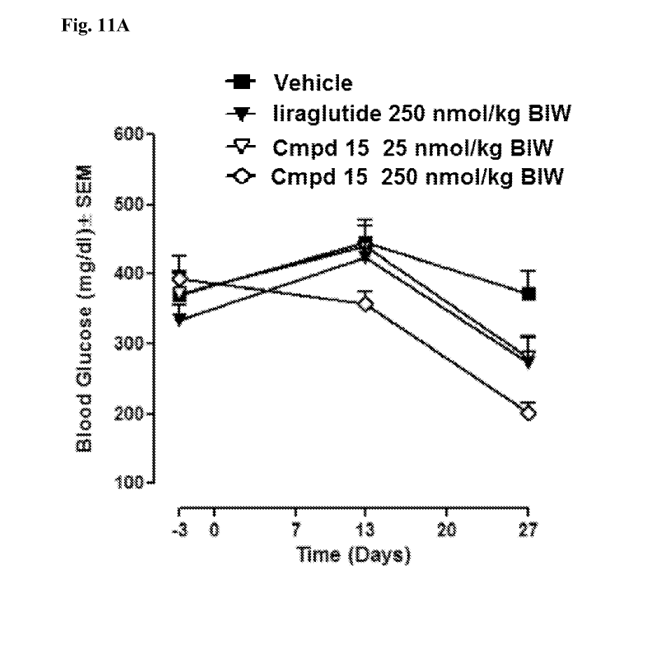

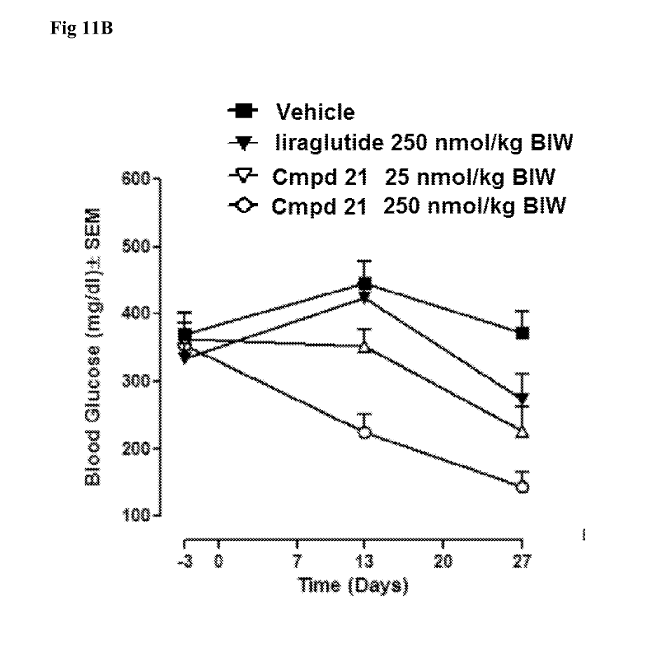

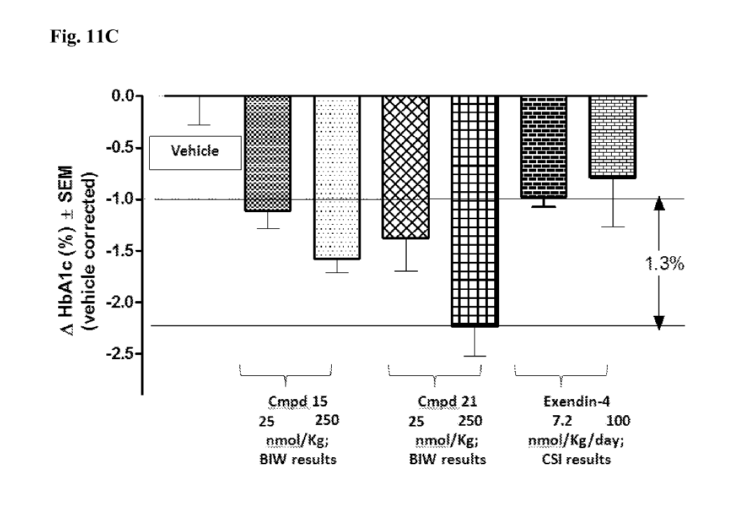

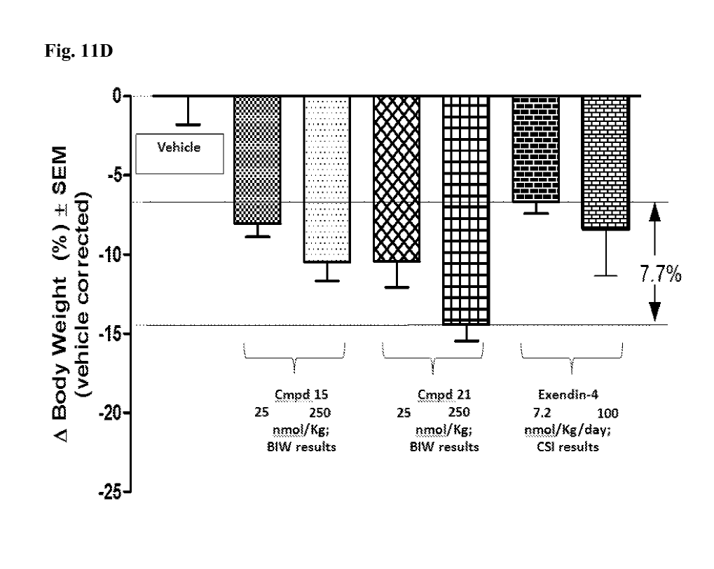

To demonstrate the effect of chronic exposure of an exendin-albumin binding domain engineered polypeptide described herein on glucose lowering, HbA1c lowering, and body weight reduction, diabetic ob/ob/mice were treated with Cmpd 15 and Cmpd 21. The time course of the effect of the test compound on body weight, glucose lowering and HbA_{1c} lowering in ob/ob mice was investigated post dose, with values at 4 weeks presented in FIGS. 11A, 11B, 11C and 11D. FIG. 11A (Cmpd 15) and 11B (Cmpd 21) depict changes in blood glucose compared to liraglutide, all given twice weekly (BIW). and FIG. 11C depicts lowering of HbA1c (% change from baseline) for Cmpd 15 and Cmpd 21 given twice weekly (BIW), compared to exendin-4 given by continuous subcutaneous infusion (CSI). FIG. 11D depicts reduction in body weight (% change from baseline) for Cmpd 15 and Cmpd 21 given twice weekly (BIW), compared to exendin-4 given by continuous subcutaneous infusion (CSI). Surprisingly, as seen from FIGS. 11A and 11B, each compound is superior to liraglutide at equimoloar dosing for glucose lowering upon chronic exposure. Further, at equimolar dosing to liraglutide, Cmpd 15 and Cmpd 21 were each more effective than liraglutide [N-epsilon-(gamma-Glu(N-alpha-hexadecanoyl))-Lys26,Arg34]-GLP-1-(7-37)-acid, a long-acting albumin binding GLP-1 derivative, in HbA1c lowering and body weight loss (data not shown). As depicted in FIG. 11C significant HbA1c lowering attends treatment and in FIG. 11D significant body weight loss attends treatment, with 25 and 250 nmol/kg of each compound provided intraperitoneally (IP) twice each week for 28 days. Points represent mean±s.d. (standard deviation). Each test compound was injected IP on day=0 immediately following baseline sample collection in non-fasted male ob/ob mice. The effects observed for the 25 nmol/kg biw (twice weekly) dose was greater than that observed for exendin-4 given at ˜7.2 nmol/kg/d by continuous infusion (CSI), a dose known to provide a maximal efficacy for exendin-4. Thus at a comparable equimolar dose, Cmpd 15 and Cmpd 21 exceeded the glycemic and body weight loss effects of the maximally efficacious dose of exendin-4. At 250 nmol/kg, Cmpd 15 was significantly greater than the maximally efficacious dose of exendin-4. Thus at a comparable dose, the exendin-4-GGS-ABD00239 compound (Cmpd 21) matched the glycemic and body weight loss effects of the maximally efficacious dose of exendin-4. At 250 nmol/kg, Cmpd 21 was twice as effective as the maximally efficacious dose of exendin-4. Further, surprisingly, at equimolar dosing the Cmpd 21 was more effective than liraglutide [N-epsilon-(gamma-Glu(N-alpha-hexadecanoyl))-Lys26,Arg34]-GLP-1-(7-37)-acid, an albumin binding GLP-1 derivative, for blood glucose lowering, HbA1c lowering and body weight loss (data not shown). Unless indicated otherwise, blood glucose measures described herein employed a OneTouch® Ultra® device (LifeScan, Inc. Miliptas, Calif.).

Surprisingly, despite the reduced in vitro potency compared to unconjugated exendin-4 as observed above, the acute (within 6 hours) in vivo activity of an exendin fused to an albumin binding polypeptide disclosed herein is similar to that of unconjugated exendin with regard to maximum efficacy and only slightly less (several fold) with regard to potency (ED50 for example), such as when measured by reduction of food intake in mice (data not shown). Even more surprisingly, the effect of chronic exposure demonstrates that an exendin fused to the albumin binding polypeptides disclosed herein is as potent or even has greater potency as exendin-4 (continuously infused) but is able to provide a greater maximal effect. Furthermore, in light of the very high affinity for mouse or rat albumin and low off rates, all of the engineered compounds are effectively bound to albumin in the in vivo assays (as well as in the in vitro assays). Thus the engineered polypeptides retained GLP-1R functional activity even when bound to albumin. This is surprising in part because albumin compounds, e.g. liraglutide, have been reported as significantly active only when dissociated from albumin. And others have reported a need to remove proteolytically an exendin from an albumin binding peptide to which it was conjugated in order to obtain exendin function. Accordingly, the in vivo activities as shown herein are even more impressive. This example also provides an example of activities of the engineered polypeptides using the new improved ABDs disclosed herein, e.g. PEP7986, having reduced immunogenecity.

### Example 17

### Long Duration and Action of the Engineered Polypeptides In Vivo

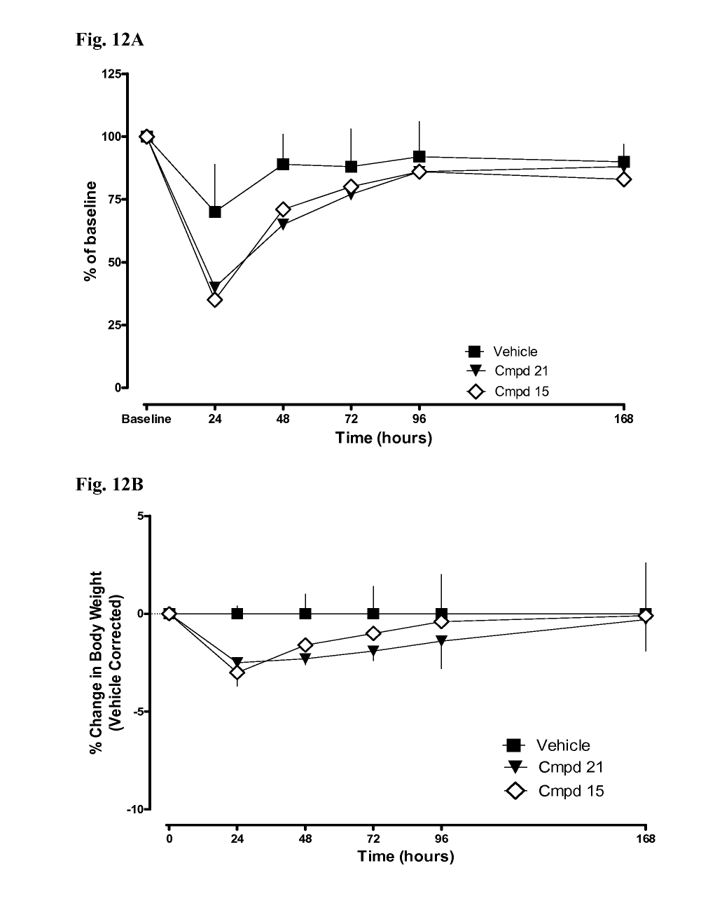

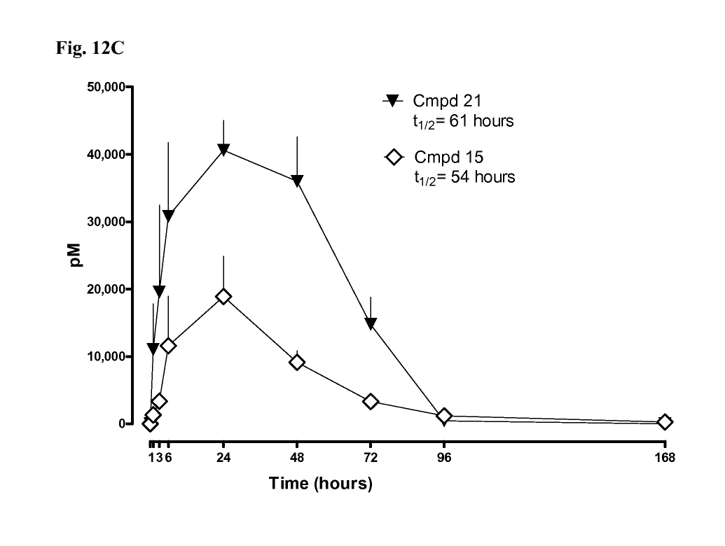

To further demonstrate the long half-life and long duration of activity of the engineered polypeptides described herein, the pharmacokinetic (PK) and pharmacodynamic (PD) properties were determined using rats. Pharmacokinetic profile and biological activity of exemplary engineered polypeptides Cmpd 15 and Cmpd 21 subcutaneously dosed in normal Harlan Sprague-Dawley (HSD) rats is presented. The recombinant engineered compounds Cmpd 21 and Cmpd 15 were injected subcutaneously at t=0 at 25 nmol/kg into normal HSD rats. Blood was collected via tail bleed at t=1 hour, 3 hours, 6 hours, 24 hours, 48 hours, 72 hours, 96 hours and 168 hours from fed HSD male rats. Food and body weights were measured daily. FIG. 12A depicts effect of Cmpd 15 and Cmpd 21 to reduce food intake. FIG. 12B depicts effect of Cmpd 15 and Cmpd 21 to reduce body weight. FIG. 12C depicts a PK profile of Cmpd 15 and Cmpd 21 after a single dose. Points represent mean±sd.

Exposure of at least up to seven (7) days was observed for both exemplary engineered polypeptides. Cmpd 15 has an apparent half-life of 54 hours and Cmpd 21 has an apparent half-life of 61 hours, in rats by this subcutaneous delivery. By allometric scaling and in view of the strong affinity of the engineered polypeptides for human albumin, physical and biological activity duration at least as long and even longer is expected in human subjects. Accordingly, the compounds have use for at least twice daily (e.g. morning and night), at least daily, twice weekly, and even once weekly administration, especially in human subjects.

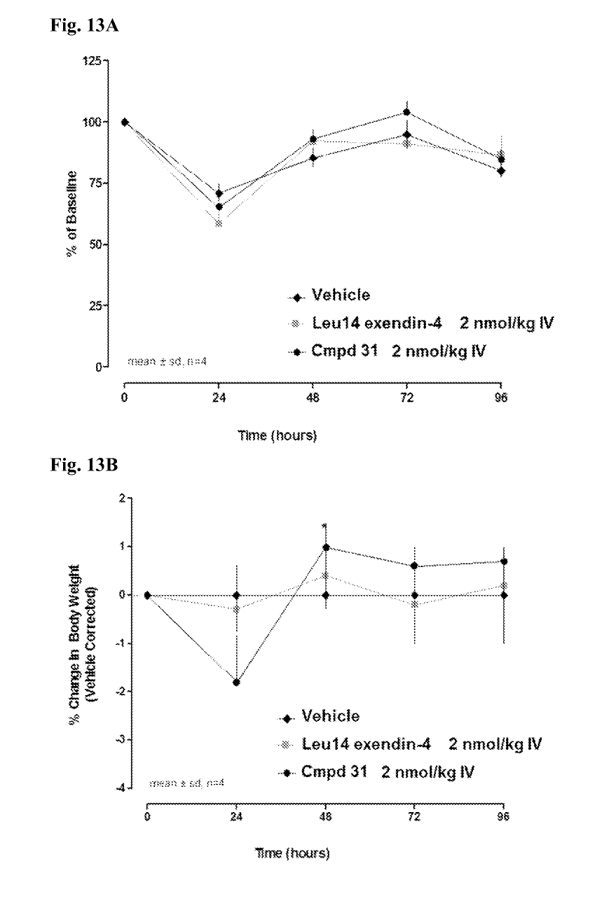

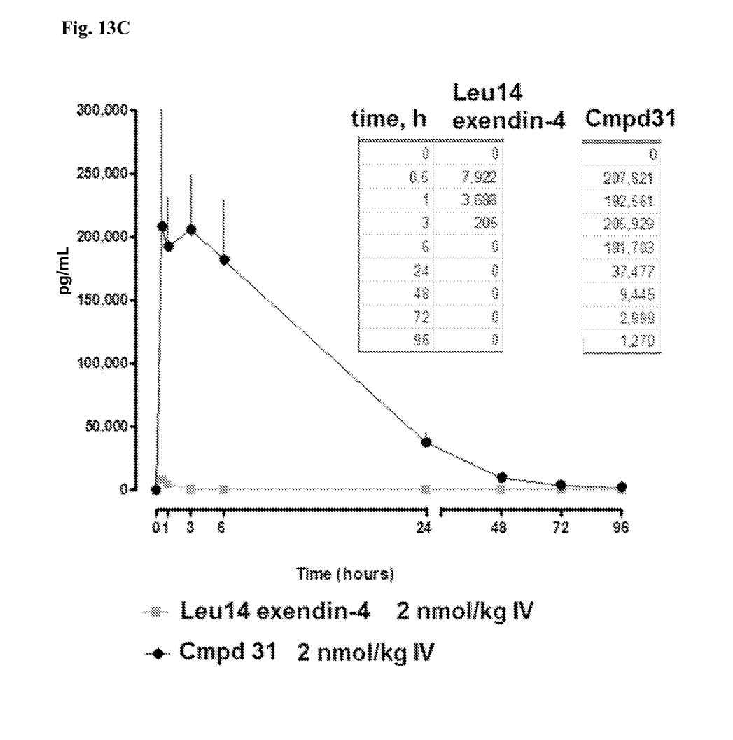

Pharmacokinetic profile and biological activity of an exemplary engineered polypeptide intravenously dosed in normal Harlan Sprague-Dawley (HSD) rats is presented. The recombinant engineered compound Cmpd 31 was injected intravenously at t=0 at 2 nmol/kg into normal HSD rats. Blood was collected via tail bleed at t=1 hour, 3 hours, 6 hours, 24 hours, 48 hours, 72 hours, 96 hours and 168 hours from fed HSD male rats. Food and body weights were measured daily. FIG. 13A depicts effect of Cmpd 31 to reduce food intake. FIG. 13B depicts effect of Cmpd 31 to reduce body weight. FIG. 13C depicts a PK profile of Cmpd 31 after a single IV dose. Half-life is estimated at about at least 14 hours, Points represent mean±sd.

Exposure of up to seven (7) days was observed for this exemplary engineered polypeptide, even at these relatively low doses. By allometric scaling and in view of the strong affinity of the engineered polypeptides for human albumin, physical and biological activity duration at least as long and even longer is expected in human subjects. Accordingly, the compounds have use for at least twice daily (e.g. morning and night), at least daily, twice weekly, and even once weekly administration, especially in human subjects.

### Example 18

### Oral Delivery of Engineered Polypeptides Achieves Systemic Distribution

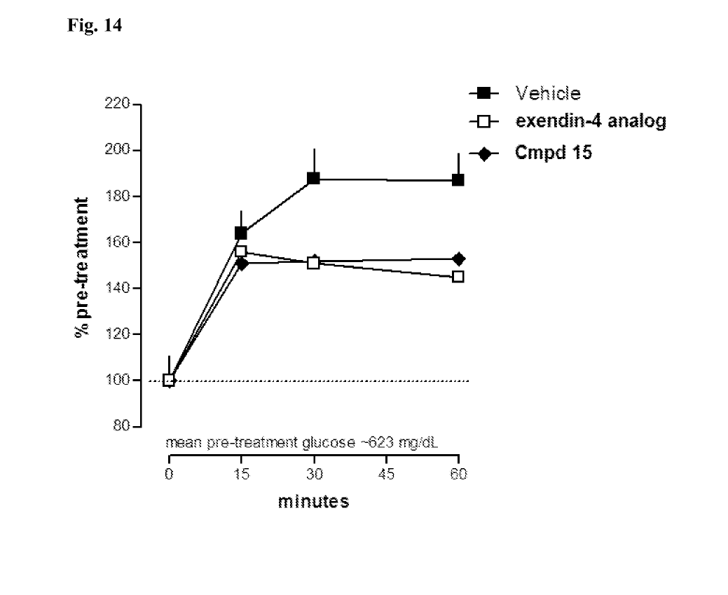

Oral delivery with intestinal uptake was investigated using a representative engineered compound. Diabetic db/db mice were dosed orally (peroral via gavage) with 240 nmol/kg of the following compounds, an exendin analog [Leu14,Gln28]Exendin-4-(1-32)-fGLP-1-(33-37) acid and Cmpd 15. The data demonstrate that the engineered peptides are orally bioavailable, even in a formulation PBS/propylene glycol (50:50) absent other specific excipients that might enhance delivery and uptake. Compared to the exendin analog, Cmpd 15 (both at 1 mg/kg dose) at more than twice the molecular weight of the exendin analog is also orally bioavailable in the same formulation. The results indicate that both compounds were active when dosed orally, and equally efficacious under the conditions tested to 120 minutes. The results are presented in FIG. 14. Points represent mean+/−sd. Peptides were dosed peroral by gavage at t=0 immediately following the taking of a baseline sample. Mice were 2-hour fasted db/db mice. Accordingly, the compounds presented herein have use for at least twice daily (e.g. morning and night), at least daily, thrice weekly, twice weekly, and even once weekly oral administration, especially in human subjects.

Examples 19-23 are provided to illustrate, amongst other things, the superior properties of the improved ABDs described herein with reduced immunogenicity properties, the ABD2, compared to ABD1 not modified for reduced immunogenicity.

### Example 19

### Cloning, Expression, Purification and Characterization of Albumin Binding Polypeptides

In this example, eight different albumin binding polypeptides, PEP07913 (SEQ ID NO:453), PEP07912 (SEQ ID NO:456), PEP07914 (SEQ ID NO:458), PEP07968 (i.e. DOTA conjugated to PEP07911, SEQ ID NO:459), PEP06923 (SEQ ID NO:454), PEP07271 (SEQ ID NO:455), PEP07554 (SEQ ID NO:456) and PEP07844 (SEQ ID NO:461), the amino acid sequences of which are set out in Table 1C, were cloned, purified and characterized.

### Material and Methods.

### Cloning of Albumin Binding Polypeptide Variants.

Mutations in G148-GA3 were generated using site directed mutagenesis with the appropriate oligonucleotides to obtain the desired albumin binding polypeptide variants. The gene fragments were amplified by PCR with primers adding specific endonuclease sites as well as an N-terminal MGSS sequence preceding the albumin binding polypeptide variants. The fragments were cleaved with NdeI and NotI, purified and ligated to a cloning vector, the plasmid pAY02556 (containing an origin of replication from pBR322, a kanamycin resistance gene and a T7 promoter for expression of the gene of interest), restricted with the same enzymes. Ligations were transformed to electrocompetent E. coli TOP 10 cells. The transformed cells were spread on TBAB plates (30 g/l tryptose blood agar base) supplemented with 50 μg/ml of kanamycin, followed by incubation at 37° C. overnight. The colonies were screened using PCR and sequencing of amplified fragments was performed using the biotinylated oligonucleotide and a BigDye® Terminator v3.1 Cycle Sequencing Kit (Applied Biosystems), used in accordance with the manufacturer's protocol. The sequencing reactions were purified by binding to magnetic streptavidin coated beads using a Magnatrix 8000 (NorDiag AB), and analyzed on ABI PRISM® 3100 Genetic Analyzer (PE Applied Biosystems). All albumin binding polypeptide variants were subcloned as monomers and the constructs encoded by the expression vectors were MGSS-[PP###], where PP### corresponds to the 46 amino acid residues constituting the sequence of the albumin binding polypeptide.

In addition, the gene fragments of G148-GA3, PP007 (SEQ ID NO:307), PP013 (SEQ ID NO:313) and PP037 (SEQ ID NO:337) were amplified by PCR with primers adding specific endonuclease sites as well as a hexahistidin sequence, a TEV protease site and a glycine residue before the 46 amino acid residues constituting the sequence of the albumin binding polypeptide. The polypeptides PEP07913 (SEQ ID NO:453), PEP07912 (SEQ ID NO:457), PEP07914 (SEQ ID NO:458) and PEP07968 (SEQ ID NO:459) correspond to the albumin binding polypeptides G148-GA3, PP007 (SEQ ID NO:307), PP013 (SEQ ID NO:313) and PP037 (SEQ ID NO:337) with glycine residues added. The fragments were cleaved with XbaI and NotI, purified and ligated to a cloning vector, the plasmid pAY02512 (containing an origin of replication from pBR322, a kanamycin resistance gene and a T7 promoter for expression of the gene of interest. The cloning site is preceded by a sequence encoding a peptide containing a hexahistidine tag followed by a cleavage site for the TEV protease), restricted with the same enzymes. Ligation, transformation and sequence verification were performed as described above. The four albumin binding polypeptide variants G148-GA3, PP007, PP013 and PP037 were subcloned as monomers and the constructs encoded by the expression vectors were MGSSHHHHHHLQSSGVDLGTENLYFQG-[PP###], where MGSSHHHHHHLQSSGVDLGTENLYFQG is SEQ ID NO:740, and “PP###” represents a ABD sequence described herein.

### Protein Expression.

The albumin binding polypeptide variants were expressed in E. coli BL21 (DE3) either with an N-terminal MGSS-extension or with an N-terminal His6-tag followed by a TEV-protease recognition site and a glycine residue. A colony of each albumin binding polypeptide variant was used to inoculate 4 ml TSB+YE medium supplemented with kanamycin to a concentration of 50 μg/ml. The cultures were grown at 37° C. for approximately 5 hours. 3 ml from each of the cultures was used to inoculate 800 ml TSB+YE supplemented with kanamycin to a concentration of 50 μg/ml in parallel bio reactors (Greta system, Belach Bioteknik AB). The cultivations were performed at 37° C., with aeration at 800 ml/minute and an agitation profile to keep dissolved oxygen levels above 30%, to an OD600 of 2, which was followed by addition of IPTG to a final concentration of 0.5 mM. Cultivation was continued for five hours after which the cultivation was cooled to 10° C., aeration was stopped and agitation lowered to 300 rpm. Cell pellets were harvested by centrifugation (15600×g, 4° C., 20 minutes) and stored at −20° C. until purification.

Purification of Albumin Binding Polypeptide Variants with a His6-Tag and a TEV-Protease Site.

Frozen cell pellets harboring soluble hexahistidine-tagged polypeptides PEP07913 (SEQ ID NO:453), PEP07912 (SEQ ID NO:456), PEP07914 (SEQ ID NO:458) and PEP07968 (SEQ ID NO:459) were suspended in 35 ml binding buffer (20 mM sodium phosphate, 0.5 M NaCl, 20 mM imidazole, pH 7.4) with an addition of 1000 U Benzonase® (1.01654.001, Merck) and disrupted by ultrasonication. For each of the polypeptides, the ultrasonicated suspension was clarified by centrifugation (1 h, 37000×g, 4° C.) and the supernatant was loaded onto a His GraviTrap™ column (11-0033-99, GE Healthcare). The column was washed with 10 ml washing buffer (20 mM sodium phosphate, 0.5 M NaCl, 60 mM imidazole, pH 7.4), before eluting the polypeptide with 3 ml elution buffer (20 mM sodium phosphate, 0.5 M NaCl, 0.5 M imidazole, pH 7.4). The buffer was exchanged to a cleavage buffer (50 mM Tris-HCl, 150 mM NaCl, pH 8) using PD-10 desalting column (17-0851-01, GE Healthcare). The amount of polypeptide product was determined by measuring the absorbance at 280 nm before adding DTT to a final concentration of 5 mM. His6-tagged TEV protease was added to the cleavage buffer at a 1:10 mass ratio relative to the polypeptide product. The cleavage was performed over night under slow mixing at 4° C. Imidazole was added to the cleavage mix, to a concentration of 20 mM, before loading the mix onto a His GraviTrap™ column (11-0033-99, GE Healthcare) for removing cleaved His6-tags, His6-tagged TEV protease and His6-tagged uncleaved product.

For each variant, the flow-through, containing the albumin binding polypeptide variant, was further purified by reversed phase chromatography (RPC), as follows. The flow-through fraction was loaded on 1 ml Resource 15 RPC column (GE Healthcare), previously equilibrated with RPC A Buffer (0.1% TFA in water). After column wash with 10 column volumes (CV) RPC A Buffer, bound polypeptides were eluted with a linear gradient of 0-50% RPC B Buffer (0.1% TFA in acetonitrile) during 10 CV. The flow rate was 2 ml/min and the absorbance at 280 nm was monitored. Fractions containing albumin binding polypeptide variant were identified by SDS-PAGE analysis and pooled.

The RPC-purified albumin binding polypeptide variants were further purified by gel filtration on 120 ml Superdex 75 (GE Healthcare) packed in an XK16 column (GE Healthcare). The running buffer was 1×PBS, and the flow rate 2 ml/min. Fractions containing pure albumin binding polypeptide variant were pooled and concentrated to approximately 1.3 mg/ml. Finally, the concentrate was purified from trace amounts of remaining endotoxins by using 1 ml columns of AffinityPak Detoxi-Gel Endotoxin removing gel (Pierce, prod#20344), according to the manufacture's recommendations.

The albumin binding polypeptide variant PEP07911 was conjugated with Mal-DOTA before the RPC-purification step, as follows. The buffer of the flow-through fraction from the IMAC-FT purification step was exchanged to 0.2 M NaAc, pH 5.5, using a disposable PD-10 desalting column (GE Healthcare). Maleimido-mono-amide-DOTA (Macrocyclics, cat. no. B-272) was added at 5-fold molar excess and incubated for 60 minutes at 30° C. under continuous shaking. The resulting polypeptide was denoted PEP07968.

Purification of Albumin Binding Polypeptide-Variants without His6-Tag.

Frozen cell pellets harboring soluble albumin binding polypeptide variants PEP06923 (SEQ ID NO:454), PEP07271 (SEQ ID NO:455), PEP07554 (SEQ ID NO:456) and PEP07844 (SEQ ID NO:461) were suspended in 20 mM Tris-HCl, pH 8 and disrupted by ultrasonication. For each of the polypeptide variants, the ultrasonicated suspension was clarified by centrifugation (30 min, 32000×g, 4° C.) and the supernatant was loaded onto a HSA-Sepharose column (GE Healthcare). After washing with TST-buffer (25 mM Tris-HCl, 1 mM EDTA, 200 mM NaCl, 0.05% Tween 20, pH 8.0), followed by 5 mM NH4Ac, pH 5.5, bound albumin binding polypeptide variant was eluted with 0.5 M HAc, pH 3.2.

The albumin binding polypeptide variants were further purified by reversed phase chromatography (RPC), as follows. For each of the variants, the eluate from the HSA-affinity purification step was loaded on 1 ml Resource 15 RPC column (GE Healthcare), previously equilibrated with RPC A Buffer (0.1% TFA in water). After column wash with 10 CV RPC A Buffer, bound polypeptides were eluted with a linear gradient of 0-50% RPC B Buffer (0.1% TFA in acetonitrile) during 10 CV. The flow rate was 2 ml/min and the absorbance at 280 nm was monitored. Fractions containing pure albumin binding polypeptide variants were identified by SDS-PAGE analysis and pooled. Finally, the buffer was exchanged to 1×PBS (2.68 mM KCl, 137 mM NaCl, 1.47 mM KH2PO4, 8.1 mM Na2HPO4, pH 7.4) using a disposable PD-10 desalting column (GE Healthcare).

### Characterization of Purified Albumin Binding Polypeptide-Variants.

The concentration was assessed by measuring the absorbance at 280 nm using a NanoDrop® ND-1000 Spectrophotometer. The proteins were further analyzed with SDS-PAGE and LC-MS.

For the SDS-PAGE analysis, approximately 10 μg of each albumin binding polypeptide variant was mixed with NuPAGE LDS Sample Buffer (Invitrogen), incubated at 70° C. for 15 min and loaded onto NuPAGE 4-12% Bis-Tris Gels (Invitrogen). The gels were run with NuPAGE MES SDS Running Buffer (Invitrogen) in an XCell II SureLock Electrophoresis Cell (Novex) employing the Sharp Prestained Standard (Invitrogen) as molecular weight marker and using PhastGel BlueR (GE Healthcare) for staining.

To verify the identity of the albumin binding polypeptide variants, LC/MS analyses were performed using an Agilent 1100 LC/MSD system, equipped with API-ESI and a single quadruple mass analyzer. Approximately 10 μg of each of the purified albumin binding polypeptide variants was loaded on a Zorbax 300SB-C8 Narrow-Bore column (2.1×150 mm, 3.5 μm, Agilent Technologies) at a flow-rate of 0.5 ml/min. Polypeptides were eluted using a linear gradient of 10-70% solution B for 15 min at 0.5 ml/min. The separation was performed at 30° C. The ion signal and the absorbance at 280 and 220 nm were monitored. The molecular weights of the purified albumin binding polypeptide variants were confirmed by MS.

### Results

The expression levels of the albumin binding polypeptide variants were 10-30 mg product/g cell pellet, as estimated from SDS-PAGE analysis.

For all variants, the purity, as determined by SDS-PAGE analysis, exceeded 95% and the LC/MS analysis verified the correct molecular weights. After purification, between 1 and 8 mg of pure polypeptide was obtained for each of the eight albumin binding polypeptide variants.

### Example 20

### Affinity Determination for Albumin Binding Polypeptides

In this example, PEP06923 (SEQ ID NO:454), PEP07271 (SEQ ID NO:455), PEP07844 (SEQ ID NO:461), PEP07912 (SEQ ID NO:457), PEP07913 (SEQ ID NO:453), PEP07914 (SEQ ID NO:458) and PEP07968, (i.e. DOTA-conjugated to PEP07911, SEQ ID NO:459), synthesized or expressed and purified in Example 1 were characterized for affinity to human serum albumin (HSA) using a Biacore instrument. PEP07913 corresponds to the amino acid sequence of G148-GA3 with addition of a N-terminal glycine residue, whereas PEP07271 (SEQ ID NO:455), PEP07844 (SEQ ID NO:461), PEP07912 (SEQ ID NO:457), PEP07914 (SEQ ID NO:458) and PEP07968 correspond to the albumin binding polypeptides of PP001 (SEQ ID NO:301), PP043 (SEQ ID NO:343), PP007 (SEQ ID NO:307), PP013 (SEQ ID NO:313) and PP037 (SEQ ID NO:337) with different N-terminal amino acid additions.

### Material and Methods.

Biosensor analysis on a Biacore2000 instrument (GE Healthcare) was performed with HSA (Albucult®, Novozyme), immobilized by amine coupling onto the carboxylated dextran layer of the surfaces of CM-5 chips (research grade; Biacore) according to the manufacturer's recommendations. Surface 1 of the chip was activated and deactivated and used as a reference cell (blank surface) during injections, whereas surface 2 comprised HSA immobilized to 731 resonance units (RU) and surface 4 comprised HSA immobilized to 955 RU. The purified albumin binding polypeptide variants were diluted in running buffer HBS-EP (GE Healthcare) to 2.5 nM, 10 nM and 40 nM, and injected at a constant flow-rate of 50 μl/min for 5 minutes, followed by injection of HBS-EP for 60 minutes. The surfaces were regenerated with one injection of 25 μl HCl, 10 mM. The affinity measurements were performed in two sets; in the first set HBS-EP, PEP06923, PEP07271, PEP07912, PEP07913, PEP07914 and PEP07968 were injected (chip surface 2), and in the second set HBS-EP, PEP06923, PEP07844, PEP07912 and PEP07914 were injected (chip surface 4). PEP06923 was injected twice in each run as a control. The results were analyzed with a BiaEvaluation software (GE Healthcare). Curves of the blank surface were subtracted from the curves of the ligand surfaces.

### Results.

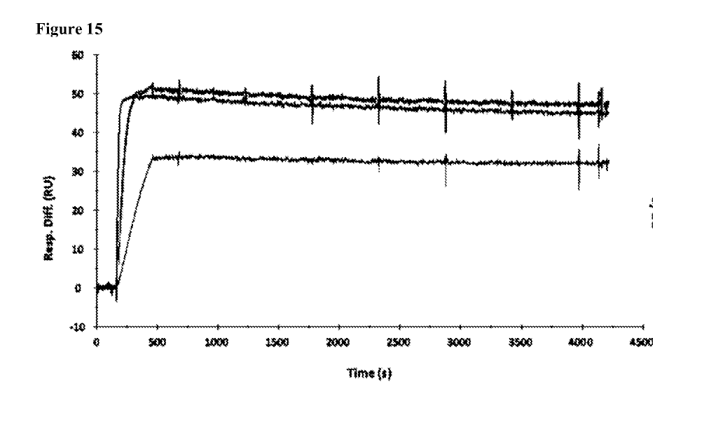

The Biacore 2000 instrument has a technical limitation, hindering measurements of very high affinity. Hence, the purpose of the Biacore study was not to determine the exact kinetic parameters of the albumin binding polypeptide variants' affinity for HSA. However, the results provide a quantitative estimation of the relative affinities of these polypeptides for albumin. After subtraction of reference surface and buffer injection, curves were fitted to a 1:1 (Langmuir) binding model using BIAevaluation software with correction for mass transfer and with RUmax set as a local parameter. Curves are shown in FIG. 15. The relative K_{D}, kₐ (kₒₙ) and k_{d} (k_{off}) values were estimated and are presented in the Tables below.

TABLE 11
Kinetic parameters (kₐ , k_{d} and K_{D}) of albumin binding polypeptides to
HSA, 1st set

| | kₐ (Ms⁻¹) | k_{d} (s⁻¹) | K_{D} (M) |
|---|---|---|---|
| PEP07913 | 5.7 × 10⁵ | 9.3 × 10⁻⁴ | 1.6 × 10⁻⁹ |
| PEP06923 (1) | 2.9 × 10⁷ | 2.9 × 10⁻⁵ | 9.9 × 10⁻¹³ |
| PEP06923 (2) | 2.6 × 10⁷ | 2.8 × 10⁻⁵ | 1.1 × 10⁻¹² |
| PEP07271 | 3.9 × 10⁶ | 2.9 × 10⁻⁵ | 7.5 × 10⁻¹² |
| PEP07912 | 4.6 × 10⁶ | 2.8 × 10⁻⁵ | 6.2 × 10⁻¹² |
| PEP07914 | 3.5 × 10⁶ | 2.5 × 10⁻⁵ | 7.2 × 10⁻¹² |
| PEP07968 | 3.0 × 10⁶ | 2.7 × 10⁻⁵ | 9.0 × 10⁻¹² |

TABLE 12
Kinetic parameters (kₐ , k_{d} and K_{D}) of albumin binding polypeptides to
HSA, 2nd set

| | kₐ (Ms⁻¹) | k_{d} (s⁻¹) | K_{D} (M) |
|---|---|---|---|
| PEP06923 (1) | 2.0 × 10⁷ | 2.6 × 10⁻⁵ | 1.3 × 10⁻¹² |
| PEP06923 (2) | 2.1 × 10⁷ | 2.5 × 10⁻⁵ | 1.2 × 10⁻¹² |
| PEP07912 | 5.4 × 10⁶ | 2.8 × 10⁻⁵ | 5.2 × 10⁻¹² |
| PEP07914 | 3.8 × 10⁶ | 2.6 × 10⁻⁵ | 6.9 × 10⁻¹² |
| PEP07844 | 5.4 × 10⁶ | 2.3 × 10⁻⁵ | 4.4 × 10⁻¹² |

As shown in Table 11 and 12, PEP07271 (SEQ ID NO:455), PEP07844 (SEQ ID NO:461), PEP07912 (SEQ ID NO:457), PEP07914 (SEQ ID NO:458) and PEP07968 (PEP07911 conjugated with DOTA, SEQ ID NO:459) all seem to have approximately the same affinity for HSA, widely exceeding the affinity of the parent G148-GA3 (PEP07913; SEQ ID NO:453). The HSA affinity of these polypeptides is slightly lower compared to PEP06923 (SEQ ID NO:454), despite similar off-rate.

### Example 21

### Determination of Melting Temperature (Tm) for Albumin Binding Polypeptides

In this example, the albumin binding polypeptide variants PEP07913 (SEQ ID NO:453), PEP06923 (SEQ ID NO:454), PEP07271 (SEQ ID NO:455), PEP07554 (SEQ ID NO:456), PEP07912 (SEQ ID NO:457), PEP07914 (SEQ ID NO:458), PEP07968 (PEP07911 conjugated with DOTA, SEQ ID NO:459) and PEP07844 (SEQ ID NO:461), expressed and purified as described in Example 1, and the albumin polypeptide variant PEP07975 (i.e. DOTA-conjugated PEP07834, SEQ ID NO:460, via Cys14 with maleimido-mono-amide-DOTA (Macrocyclics, Cat. No. B-272), were analyzed by CD analysis. PEP07913 corresponds to the sequence of G148-GA3 having an N-terminal glycine residue, PEP06923 is an engineered high affinity derivative previously described by Jonsson et al, supra, whereas PEP07271, PEP07554, PEP07912, PEP07914, PEP07968, PEP07844 and PEP07975 are examples of the 46 amino acid residues albumin binding polypeptides of PP001 (SEQ ID NO:301), PP007 (SEQ ID NO:307), PP013 (SEQ ID NO:313), PP037 (SEQ ID NO:337) and PP043 (SEQ ID NO:343) having different N-terminal amino acid additions according to the present disclosure.

### Material and Methods.

Purified albumin binding polypeptide variants were diluted in 1×PBS, to final concentrations between 0.4 and 0.5 mg/ml. Circular dichroism (CD) analysis was performed on a Jasco J-810 spectropolarimeter in a cell with an optical path-length of 1 mm. In the variable temperature measurements, the absorbance was measured at 221 nm from 20° to 90° C., with a temperature slope of 5° C./min.

### Results.

The melting temperatures (Tm) of the different albumin binding polypeptide variants were calculated by determining the midpoint of the transition in the CD vs. temperature plot. The results are summarized in Table 13 below.

TABLE 13
Determined Tm values of tested albumin binding polypeptide variants

| Variant | SEQ ID NO: # | N-terminal sequence³ | Tm (° C.) |
|---|---|---|---|
| PEP07913 | SEQ ID NO: 153 | GL | 61 |
| PEP06923 | SEQ ID NO: 154 | GSSL | 57 |
| PEP07271 | SEQ ID NO: 155 | GSSL | 65 |
| PEP07554 | SEQ ID NO: 156 | GSSL | 58 |
| PEP07912 | SEQ ID NO: 157 | GL | 53 |
| PEP07914 | SEQ ID NO: 158 | GL | 59 |
| PEP07968 | SEQ ID NO: 159¹ | GL | 53 |
| PEP07975 | SEQ ID NO: 160^{1,2} | AL | 50 |
| PEP07844 | SEQ ID NO: 161 | GSSL | 65 |

¹The peptide is conjugated with maleimide-DOTA at the cysteine
²The peptide is amidated at the C-terminus
³Leucine (underlined) is the residue in position 1 of the 46 amino acid sequence of the albumin binding polypeptide as defined in the first aspect of the present disclosure

The polypeptide PEP07968 is identical to PEP07912, except for the former having a cysteine residue in position 14, which is conjugated with maleimide DOTA, and the latter a serine residue. Thus, the DOTA modification should not affect the melting temperature. Also PEP07975 is maleiamide-conjugated with DOTA using Cys14, and is identical to PEP07968 except for the C-terminal amide (resulting from the peptide synthesis) and for having an N-terminal alanine instead of a glycine. Furthermore, comparing PEP07912 and PEP07554 reveals that an N-terminal serine gives a higher melting temperature than a glycine in the same position (5° C. difference in Tm). Thus, all albumin binding polypeptide variants according to the present disclosure show Tm above 55° C., except PEP07912 and DOTA-conjugated variants. Taking into consideration the importance of the N-terminal portion, all the tested albumin binding polypeptides are superior to the derivative of Jonsson et al, e.g. PEP06923.

### Example 22

### Serum Response Analysis

The percentage of human serum containing IgG, capable of binding to a set of albumin binding polypeptides as disclosed herein was analyzed by ELISA. In total, 149 serum samples corresponding to 127 individuals were screened.

### Material and Methods.

ELISA plates (96-well, half area plates (Costar, cat. No. 3690)) were coated with 50 μl/well of Albucult® (Novozyme) diluted to 8 μg/ml in coating buffer (Sigma, cat. No. 3041). The plates were coated over night for three days at 4° C. On the day of analysis, the plates were washed twice with tap water and blocked for 2 hours with 100 μl of phosphate buffered saline (PBS) containing 0.05% casein (PBSC). The plates were emptied and 50 μl/well of the albumin binding polypeptides PEP07913 (SEQ ID NO:453), PEP06923 (SEQ ID NO:454), PEP07271 (SEQ ID NO:455), PEP07912 (SEQ ID NO:457), PEP07554 (SEQ ID NO:456), PEP07914 (SEQ ID NO:458), PEP07968 (DOTA conjugated PEP07911, SEQ ID NO:459) and PEP07844 (SEQ ID NO:461), diluted to 2 μg/ml in PBSC were added according to a pre-made plate layout. After incubation for two hours at room temperature (RT), the plates were washed in PBSC four times using an automated ELISA washer. The 149 serum samples from 129 individuals were diluted 50 times in PBSC by adding 24 μl serum to 1174 μl PBSC. 50 μl of the diluted sera was added per well according to the pre-made plate layout. Each serum sample was tested as a singlet. Positive and negative controls were included on each plate and for each albumin binding polypeptide. Albumin binding antibodies (50 μl, 0.5 μl/ml immunoglobulin solution prepared in house from sera from primates immunized with PEP06923) was added as a positive control and 50 μl PBSC was used as a negative control. The plates were incubated for one hour at RT and subsequently washed four times in PBSC using an automated ELISA washer. The bound IgG was detected with 50 μl/well of anti-human IgG (Southern Biotech, cat no 2040-05) diluted 10 000 times in PBSC. After washing four times in PBSC using an automated ELISA washer, 50 μl/well of substrate was added (Pierce cat. No. 34021). The reaction was stopped after 10-15 minutes by the addition of 50 μl H2SO4 to each well, prior to measuring the absorbance using a multi-well plate reader (Victor3, Perkin Elmer).

### Results.

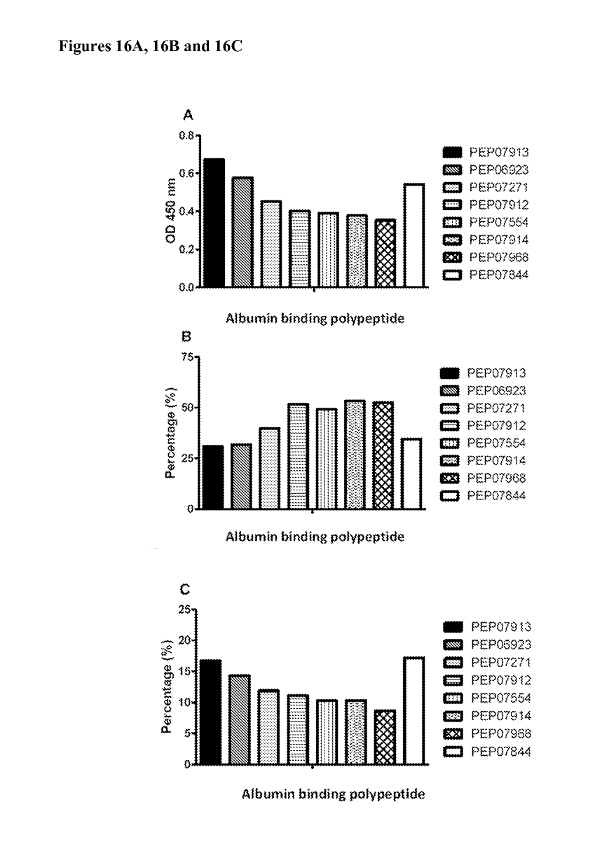

Of the 149 sera screened for IgG binding to the albumin binding polypeptides, 23 were negative for all eight polypeptides (OD-value<0.1), i.e. showed no IgG bound to the polypeptides. The analysis was performed with the 126 sera that were positive for one or more albumin binding polypeptides. The average absorbance was calculated (FIG. 16A) and the percentage of sera with OD-values values either <0.15 (FIG. 16B) or >1.0 (FIG. 16C). The highest average OD-value and the highest percentage of serum with IgG binding were obtained with PEP07913 (SEQ ID NO:453), PEP06923 (SEQ ID NO:454) and PEP07844 (SEQ ID NO:461), whereas least reactivity was found against PEP07968 (DOTA conjugated PEP07911, SEQ ID NO:459), PEP07914 (SEQ ID NO:458) and PEP07954 (SEQ ID NO:456).

Thus, the most reactive albumin binding polypeptides were the parental G148-GA3 (PEP07913, SEQ ID NO:453) and the previously affinity improved derivative (PEP06923, SEQ ID NO:454), having helix 1 retained from G148-GA3. The third of the more reactive polypeptides (PEP07844, SEQ ID NO:461) contains the original lysine in position 14 in helix 1. This residue is intended for conjugation, and will therefore not be exposed when used as such. The identical albumin binding polypeptide variant, except for having an alanine in position 14 (PEP07554, SEQ ID NO:456), is one of the least reactive.

### Example 23

### Immunogenicity Testing of Albumin Binding Polypeptides

PEP07913 (SEQ ID NO:453), PEP07912 (SEQ ID NO:457), PEP07914 (SEQ ID NO:458), and PEP07968 (i.e. DOTA conjugated PEP07911, SEQ ID NO:459) were screened for their ability to induce T cell proliferation in peripheral blood mononuclear cells (PBMC) from 52 human Caucasian individuals (obtained from CRI-Labo Medische Analyse, Gent, Belgium). PEP07913 corresponds to the sequence of G148-GA3 having an N-terminal glycine residue, whereas PEP07912, PEP07914 and PEP07968, are examples of the 46 amino acid residues albumin binding polypeptides of PP007 (SEQ ID NO:307), PP013 (SEQ ID NO:313) and PP037 (SEQ ID NO:337) having different N-terminal amino acid additions according to the present disclosure.

### Materials and Methods.

PBMCs, prepared according to standard cell biological methods, were added to a tissue culture (TC) treated 96-well round bottom plate (Falcon) in an amount of 300 000 PBMCs/well. The cells were stimulated by addition of 100 μl/well of albumin binding polypeptides PEP07913, PEP07912, PEP07914 and PEP07968 in AIMV medium (Invitrogen) additionally containing 900 μg/ml (3-fold molar excess) of recombinant human albumin (Albucult®, Novozyme). This corresponded to a final concentration of albumin binding polypeptide of 30 μg/ml. The stimulation was done in eight-replicates, i.e. the same albumin binding polypeptide was added to eight wells in identical amounts and under the same conditions. In positive control wells, the cells were stimulated with either 30 μg/ml Keyhole Limpet Hemocyanin (KLH, Calbiochem) or 30 μg/ml tetanus toxoid (TT, Statens Serum Institut). In negative control wells, only AIMV medium with or without 900 μg/ml of albumin were added.

Cell proliferation was assessed after seven days of culturing using Alexa Fluor 488 Click-iT EdU flow cytometry assay kit (Invitrogen). 1 μM/well of EdU incorporation marker was added on day six. On day seven, cells were washed, dissociated from the plate, washed again and stained for 30 minutes with anti-CD3-PerCP reagent (Becton Dickinson) and anti-CD4-Alexa647 reagent (Becton Dickinson). Following staining, the cells were washed, fixed (BD cellfix, BD biosciences), permeabilized (using saponin) and stained for EdU by addition of Click-iT reagent according to the manufacturer's protocol (Invitrogen). After completed staining, cells were washed again and analyzed using flow cytometry (FACSCantoII, BD Biosciences). To assess the number of proliferating cells, a fixed number of fluospheres (Invitrogen) was added to each well before analysis. All staining procedures and washes were performed directly in the 96-well plate.

The raw FACSCantoII data were gated hierarchically on CD3+ CD4+ T cells and the number of gated cells as well as their fluorescence intensity of EdU-Alexa Flour 488 incorporation marker were recorded. The mean values of the number of proliferating cells/eight-plicate of protein treated wells were compared to the positive and negative controls and the resulting ratios, described as stimulation indices (SI), were calculated. Based on the SI and the variation between replicates, threshold SI-values were set to 2.0 and 0.5 for stimulation and inhibition, respectively.

### Results.

The albumin binding polypeptides PEP07913, PEP07912, PEP07914 and PEP07968 were assessed for their immunogenic potential in the presence of 3-fold excess of recombinant human albumin in a target human population using an in vitro PBMC proliferation assay. Compared to the albumin control, PEP07913 induced CD3+CD4+ T cells proliferation in 6 of 52 donors, PEP07912 in 5 of 52 donors and PEP07914 and PEP07968 in 1 of 52 donors (FIG. 17A).

The mean stimulation index (SI) for all 52 donors was not significantly different for PEP07914 and PEP07968 compared to the negative control containing recombinant human albumin (p=0.79 and 0.48 respectively, FIG. 17B). The SI for PEP07913 was significantly higher (p=0.002) whereas the SI for PEP07912 was higher but not significant (p=0.03, FIG. 17B).

As compared to buffer only, the number of responding individuals was 10 for PEP07912, 7 for PEP07912, 2 for PEP07914, 1 for PEP07968, 2 for recombinant human albumin, and 49 and 51 for the two positive controls TT and KLH, respectively (FIG. 17C). The albumin binding polypeptides were ranked according to their immunogenicity in the following order: PEP07913>PEP07912>PEP07914>PEP07968. Both PEP07914 and PEP07968 were defined as non-immunogenic. The above results thus demonstrate that the immunogenic potential of the albumin binding polypeptides of the present disclosure is low, as compared to the positive controls.

### Example 24

### A Purification of an Exendin Analog-ABD Type Engineered Polypeptide

Method. Engineered polypeptides have been produced having an N-terminal extension which incorporates a His₆ (SEQ ID NO:49) “tag” as known in the art, for example as in sequence: MAHHHHHHVGTGSNENLYFQHGEGTFTSDLSKQLEEEAVRLFIEWLKQGGPSKEIISTG GGGSASLAEAKVLANRELDKYGVSDFYKRLINKAKTVEGVEALKLHILAALP (SEQ ID NO:50) which contains ABD00239 (SEQ ID NO:35) rather than an ABD with improved immunogenicity and yields Cmpd 15, provides an example of recombinant synthesis applicable to the engineered polypeptides of the present invention starting from, for example, a similar expression peptide, e.g. MAHHHHHHVGTGSNENLYFQHGEGTFTSDLSKQLEEEAVRLFIEWLKQGGPSKEIISTG GGGSASGSLAEAKEAANAELDSYGVSDFYKRLIDKAKTVEGVEALKDAILAALP (SEQ ID NO:734), which yields mature engineered polypeptide HGEGTFTSDLSKQLEEEAVRLFIEWLKQGGP SKEIISTGGGGSASGSLAEAKEAANAELD SYGVSDFYKRLIDKAKTVEGVEALKDAILAALP (Cmpd 2-5) (SEQ ID NO:620).

Preparation of Cell Extract.

In order to prepare the cell extract, cell pellets from 50 mL of cell cultures were completely resuspended in 60 mL of lysis buffer (50 mM TrisHCl, 150 mM NaCl, pH 8.0). Resuspended cells were run through a microfluidizer (Microfluidics, MA) at 100 PSI three times. Cell extracts were centrifuged for 30 min at 16,000×g to remove debris. EGTA (150 mM stock) was added to cell extract to a final concentration of 3 mM.

Ni-NTA Chromatography.

Ten mL of 50% suspension of Ni-NTA superflow was packed to a 15 mL empty column. The column was washed with 10 mL of water, 50 mL of lysis buffer, and 20 mL of lysis buffer with 3 mM EGTA (50 mM TrisHCl, 150 mM NaCl, pH8.0, 3 mM EGTA). Cell extract was carefully added on the top of Ni-NTA column, and the flow-through was collected. The column was washed with 30 mL of lysis buffer with EGTA (50 mM TrisHCl, 150 mM NaCl, pH8.0, 3 mM EGTA). Ten mL of elution buffer (25 mM TrisHCl, 50 mM NaCl, 250 mM imidazole, pH8.0) was added to the top of column, and the elution fractions (2 mL/fraction) were collected. SDS-PAGE was run to check the flow through and each fraction. Fractions containing the His-tagged compound were pooled.

TEV Protease Digestion.

His₆-tagged compound was diluted three fold with 25 mM TrisHCl, 50 mM NaCl, pH8.0. β-mercaptoethanol (0.1%) and 2% of Turbo TEV protease (2 mg/mL, 10,000 units/mg, Accelagen), were added, and the result was mixed and incubated at RT for 2 hours and at 4° C. over night.

Removal of Cleaved his-Tag and Turbo TEV with Ni-NTA.

Six mL of 50% suspension of Ni-NTA superflow was packed to a 15 mL empty column. The column was washed with 20 mL of water and 20 mL of 50 mM TrisHCl, 100 mM NaCl, 45 mM imidazole, pH8.0. The TEV digest reaction was diluted 2-fold with 50 mM TrisHCl, 150 mM NaCl, pH8.0. Diluted digest reaction was carefully added to the top of Ni-NTA column, and the flow-through was collected. Ten mL of 50 mM TrisHCl, 100 mM NaCl, 45 mM imidazole, pH8.0, was added to the column to elute any unbound protein. The flow-throughs were collected and combined.

First Size Exclusion Chromatography (SEC).

The Ni-NTA flow-through was filtered with 0.2 um filter. Superdex 75 HiLoad 26/60 column was pre-equilibrated with 390 mL of PBS. Filtered flow-through was injected to the HiLoad 26/60 column with a sample pump. Protein was eluted with 1.5 CV of PBS, and the monomer peak was pooled.

Second Size Exclusion Chromatograph.

The first SEC pool was filtered with 0.2 um filter. A Superdex 75 HiLoad 26/60 column was pre-equilibrated with 390 mL of PBS. Filtered flow-through was injected to the column HiLoad 26/60 with a sample pump. Protein was eluted with 1.5 CV of PBS, and the monomer peak was pooled.

Third Size Exclusion Chromatography.

The second SEC pool was filtered with 0.2 um filter. A Superdex 75 HiLoad 26/60 column was pre-equilibrated with 390 mL of PBS. Filtered flow-through was injected to the column HiLoad 26/60 with a sample pump. Protein was eluted with 1.5 CV of PBS, and the monomer peak was pooled.

Removal of Residual Endotoxin with EndoTrap Red.

The third SEC pool still contained ˜20 EU/mg of endotoxin, which was removed by the use of EndoTrap Red. Briefly, 0.5 mL of gel slurry was activated by adding 1 mL of Regeneration Buffer to the slurry and mix by gently shaking the tube for approximately 5 seconds. The supernatant was centrifuged and aspirated. This step was repeated two additional times. One mL of Equilibration Buffer was added, and mixing was conducted by gently shaking the tube for approximately 5 seconds. The supernatant was centrifuged and aspirated. This step was repeated two additional times. Protein sample (5.5 mL) was added to the resin and incubated for 90 minutes at RT, with gentle rocking or rotating of the tube while incubating. The result was centrifuged at 1200×g for 5 minutes, and the supernatant was transferred to a clean tube.

Results.

The final purified protein migrated on SDS-PAGE gel as approximately a 6 kD protein under the conditions employed. The LC-MS showed a correct molecular weight of 9827 dalton. The protein yield was 3.3 mg from 50 mL of cell culture.

### Example 25

### Activities of Exendin-ABD Engineered Polypeptides

Exendin-ABD engineered polypeptides of the invention retained sufficient exendin activity in an in vitro cell activation assay. Additionally, the engineered polypeptides provided dramatically improved duration of action for blood glucose lowering and body weight loss, as when compared to exendin-4, when administered as a single dose to a mammal Surprisingly, duration of action can be extended to at least 1 day, even at least 4 days, and even at least 7 days, or longer, in a rodent model, which can translate to at least one week duration of action in a human subject, thus suitable for twice daily, once daily, three times weekly, twice weekly or even once weekly administration.

### Example 26

### Albumin Binding

Characterization of the binding of engineered polypeptide compounds to albumin can be performed by any number of methods, including Biacore described herein. In this example binding measurements were conducted with a BioRad ProteOn XPR36 system (Bio-Rad Laboratories, Hercules Calif., USA; ProteOn XPR36 Protein Interaction Array System catalog number #176-0100), using a GLC sensor chip at 25 degrees C. For amine coupling the GLC chip was activated for 5 minutes using a 1:1 mixture of sulfo-NHS/EDC diluted 30-fold from the initial stock in water as shown below. Each albumin sample was diluted to 25 ug/ml in 10 mM Na acetate pH 5.0 and injected for 5 minutes over separate sensor surfaces. Each surface was then blocked with 1 M ethanolamine pH 8.5. Each albumin was coupled at a density of 2000-5000 in resonance units. The binding of an engineered polypeptide was tested using 5 nM as the highest concentration in a three-fold dilution series. The running buffer contained 10 mM HEPES pH 7.4, 150 mM NaCl, 3 mM EDTA and 0.005% tween-20. All samples were tested using a 3-fold dilution series. Each concentration series was tested in duplicate. The dissociation phase for the highest concentration was monitored for 3 hours.

The relative K_{D} measured for the engineered polypeptides are presented in Table 14 below. The results show that the albumin binding polypeptides associate with albumins with high affinity. The number in parentheses represents the standard deviation in the last significant digit. As seen from the following table the exendin polypeptides fused to albumin binding domains of ABD00239 (to create polypeptides Cmpd 15 or Cmpd 21) or an ABD with improved immunogenecity (to create engineered polypeptides such as Cmpd 2-5) retain extremely high affinity for serum albumin from various species, especially human serum albumin, even compared to the unconjugated ABD PEP07986 itself. The engineered polypeptides retain high affinity even compared to the identical exendin analog-linker construct fused to prior ABD00239 in place of the new PEP7986 (compare Cmpd 15 to Cmpd 2-5).

TABLE 14

| | | | Monkey | | |
|---|---|---|---|---|---|
| Cmpd | Human SA | Dog SA | SA | Mouse SA | Rat SA |
| ABD00239 | 16.1(4) pM | 201(2) pM | 123(1) pM | 1.24(1) nM | 18.3(5) pM |
| PEP07986 | 9.5(2) pM | 126(2) pM | 84.0(8) pM | 160(2) pM | 5.7(2) pM |
| Cmpd 21 | 84.7(9) pM | 397(2) pM | 77.5(6) pM | 1.332(6) nM | 16(1) pM |
| (SEQ ID | | | | | |
| NO: 99) | | | | | |
| Cmpd 15 | 68(1) pM | 513(3) pM | 90.9(9) pM | 1.253(8) nM | 200(200) pM |
| (SEQ ID | | | | | |
| NO: 163) | | | | | |
| Cmpd 2-5 | 160(1) pM | 606(4) pM | 140(1) pM | 300.2(5) pM | 12.5(2) pM |

### Example 27

### Activity of Compounds in a GLP-1 Receptor Functional Assay

Functional activity of the compounds can be determined using a cell line expressing GLP-1 receptor. See for example United States Patent Application Publication US20110097751A1, incorporated by reference for the assay method. In this example, functional activity was determined using cells that endogenously express GLP-1R, and cAMP induction is detected as a measure of exendin activity. An HTRF assay kit was used (Cisbio International (Bedford, Mass.). The bioassay used the rat thyroid carcinoma 6-23 (clone 6) cells in the cell-based assay using the HTRF® cAMP dynamic 2 1,000 assay kit, available from Cisbio as Catalog No. 62AM4PEB. The HTRF® standards and calibrations are prepared following the instructions in the kit. Accumulation of cAMP is measured following 30 minutes of compound treatment using the HTRF (CisBio) cell-based cAMP assay kit in 384-well format. Efficacy of peptides is determined relative to cell treatment with 10 uM forskolin (a constitutive activator of adenylate cyclase), and potency (EC50) of peptides is determined by the analysis of a concentration-response curve using non-linear regression analysis fitted to a 4-parameter model. The results of the GLP-1 receptor functional activity (cAMP induction) for potency (EC50) are provided in the following Table 15.

TABLE 15
GLP-1R Functional Activity

| | GLP-1R |
|---|---|
| | Functional |
| | activity (EC₅₀) in |
| Description | nM |
| Exendin-4 | 0.004 |
| [Leu¹⁴,Gln²⁸]Exendin-4(1-32)-fGLP-1)33-37) | 0.016 |
| (SEQ ID N0: 4) | |
| Exendin-4 (1-28) amide | 0.011 |
| Cmpd 21 (SEQ ID NO: 99) | 0.131 |
| Cmpd 2-5 | 0.486 |
| Cmpd 2-6 | 0.560 |
| Cmpd 2-7 | 0.904 |
| Cmpd 2-8 | 0.612 |
| Cmpd 2-9 | 3.21 |
| Cmpd 2-10 | 0.575 |
| Cmpd 2-11 | 1.28 |

Characterization of the in vitro activity of the engineered polypeptide compounds in the presence of serum albumin was demonstrated. Assays can be run in the presence and absence of an albumin, particularly human serum albumin. The data above was determined in the presence of about 0.1% bovine serum albumin (BSA). The following Table 16 presents functional activity of receptor activation (cAMP induction) assay described above, but in the presence of serum albumin from various species and at increased amounts. As can be seen, surprisingly, even when Cmpd 2-11 is bound to serum albumin, such as to human serum albumin, despite the presence of the ABD and the large serum albumin with its potential for steric hindrance and change in the apparent Stoke's radius of the compounds resulting from albumin binding, the engineered polypeptide retains GLP-1 receptor agonist activity. Given the picomolar affinity of ABD and the engineered polypeptides to some species of serum albumin, e.g. human serum albumin, the engineered polypeptide is believed to be effectively fully bound to albumin present in the assay (and thus also fully bound in vivo in circulating blood). Because of the extremely high affinity of compound binding to albumin (as above) and the presence of a high concentration of serum albumin in the blood, it is expected that the compounds will exist essentially in the bound state in vivo yet surprisingly provide sufficient exendin functions (as demonstrated herein).

TABLE 16
GLP-1R Functional Activity in Varying Amounts of Serum Albumin

| | GLP-1R Activation: | | | |
|---|---|---|---|---|
| | cAMP Induction EC50 (nM) | | | |
| | 0.1% | | | |
| | Bovine | 1% Bovine | 1% Human | 1% Rat |
| Cmpd | Albumin | Albumin | Albumin | Albumin |
| GLP-1(7-36) amide | 0.0306 | 0.0058 | 0.0112 | 0.0179 |
| Cmpd 15 | 0.7854 | 0.2204 | 0.185 | 0.2473 |
| Cmpd 21 | 1.1013 | 0.2234 | 0.2022 | 0.2164 |
| (SEQ ID NO: 99) | | | | |
| Cmpd 2-11 | 0.8719 | 0.1774 | 0.2079 | 0.1854 |

### Example 28

### OGTT DOA (Oral Glucose Tolerance Test Duration of Action) In Vivo Activity

The effect of lowering blood glucose prior to oral gavage (1.5 k/kg dextrose) and at 30 min after gavage were investigated 1 day post dose with indicated compounds, dosed at 25 nmol/Kg, with results shown in the following table. Drug was administered to 4-hr fasted NIH/Swiss mice. At 24 hours post drug administration, an OGTT was performed to assess duration of compound activity. Blood glucose was measured with a OneTouch® Ultra® (LifeScan, Inc., a Johnson & Johnson Company, Milpitas, Calif.). * p<0.05 vs. vehicle control; ANOVA, Dunnett's test. Glucose lowering as percent lowering compared to vehicle is given (negative value indicates lowering of glucose) at 30 minutes post gavage. This OGTT DOA indicates drug activity is present at least 24 hours (for the period of time prior to the OGTT) after drug was administered. Results are presented in Table 17. Exendin-4 (unconjugated to ABD), Leu14 exendin-4 (unconjugated to ABD) and unconjugated ABD2 PEP07986 are inactive in this assay when dosed at t-24 hours, even when provided at even higher doses. When the unconjugated exendin compounds are given at 30 nmol/kg immediately before an OGTT, both exendin-4 and Leu14 exendin-4 provide a −41% change in glucose from basal. Cmpd 2-12, having a C-termnally truncated exendin-4(1-28) fused to an ABD via a relatively short linker (HGEGTFTSDLSKQLEEEAVRLFIEWLKNTGGGGSASGSLAEAKEAANAELDSYGVSDF YKRLIDKAKTVEGVEALKDAILAALP; SEQ ID NO:741) was active in vitro against the GLP-1 receptor, but did not display 24 h duration in this duration assay. In this case, increasing the linker length or the length of the exenind moiety resulted in a desirable 24 h or longer duration in this assay. This duration of action assay as well as the other in vivo assays presented herein demonstrate that the improved engineered polypeptides are extremely stable to human plasma and human cell membrane proteases.

TABLE 17
OGTT DOA

| | Percent glucose lowering compared |
|---|---|
| Description | to vehicle |
| Cmpd 2-5 | −19, −22 |
| Cmpd 2-6 | −22 |
| Cmpd 2-7 | −16 |
| Cmpd 2-8 | −18, −17 |
| Cmpd 2-9 | −12 |
| Cmpd 2-10 | −12 |
| Cmpd 2-11 | −23 |

### Example 29

### Long Duration and Action of the Engineered Polypeptides In Vivo

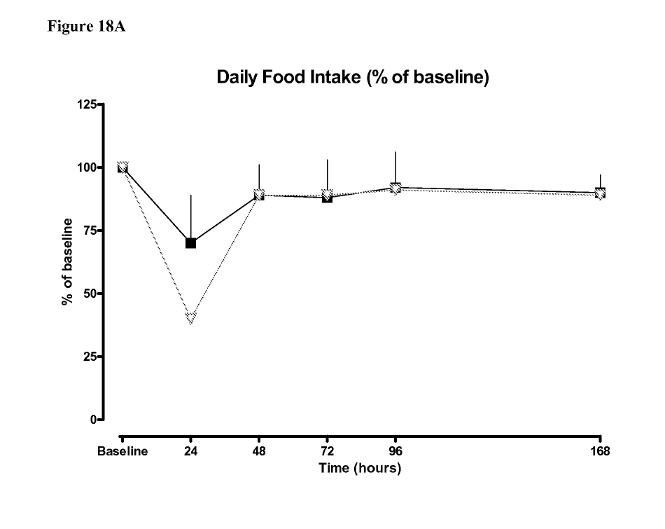

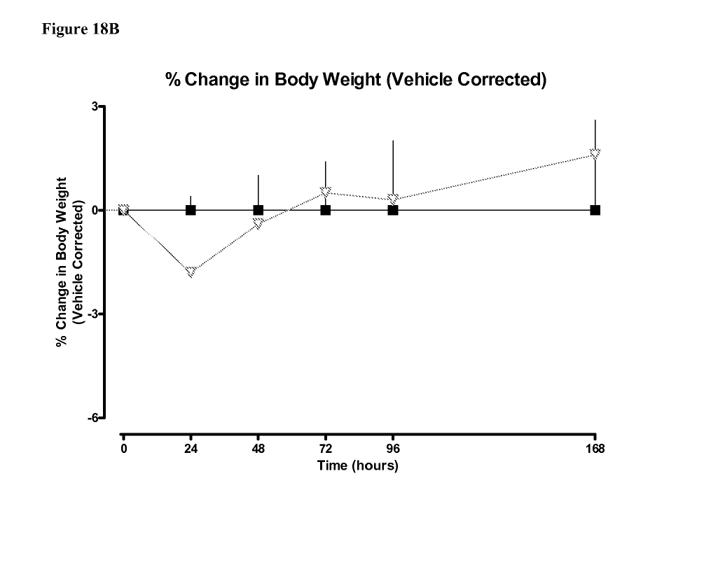

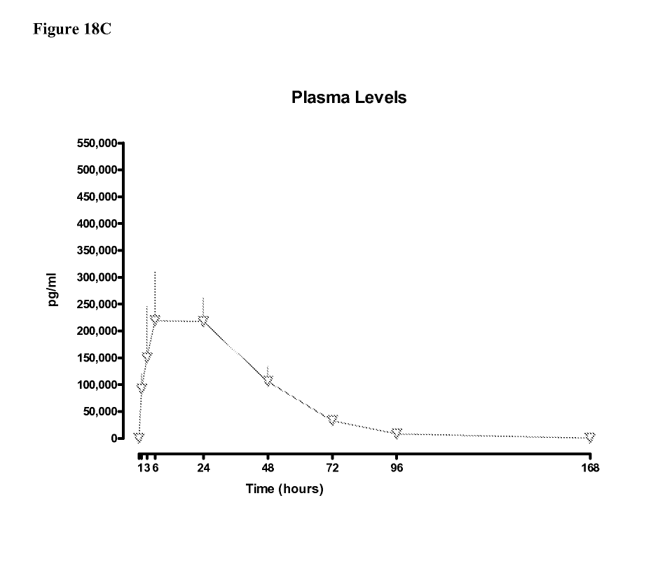

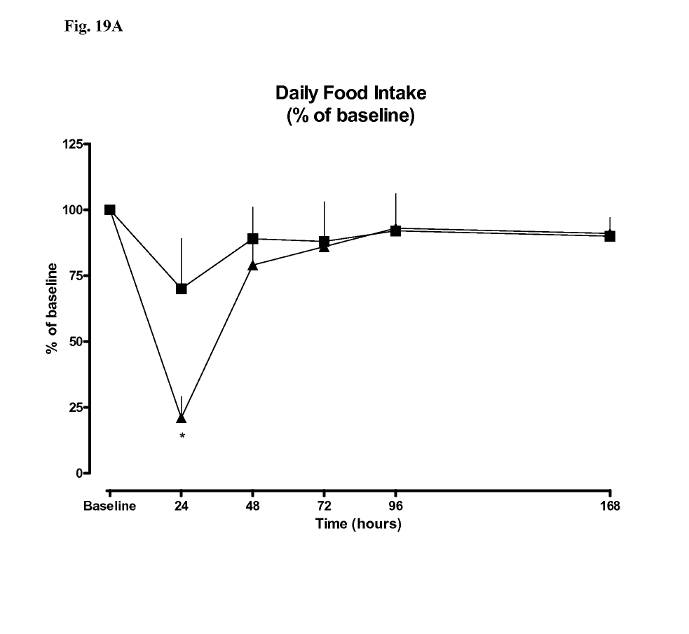

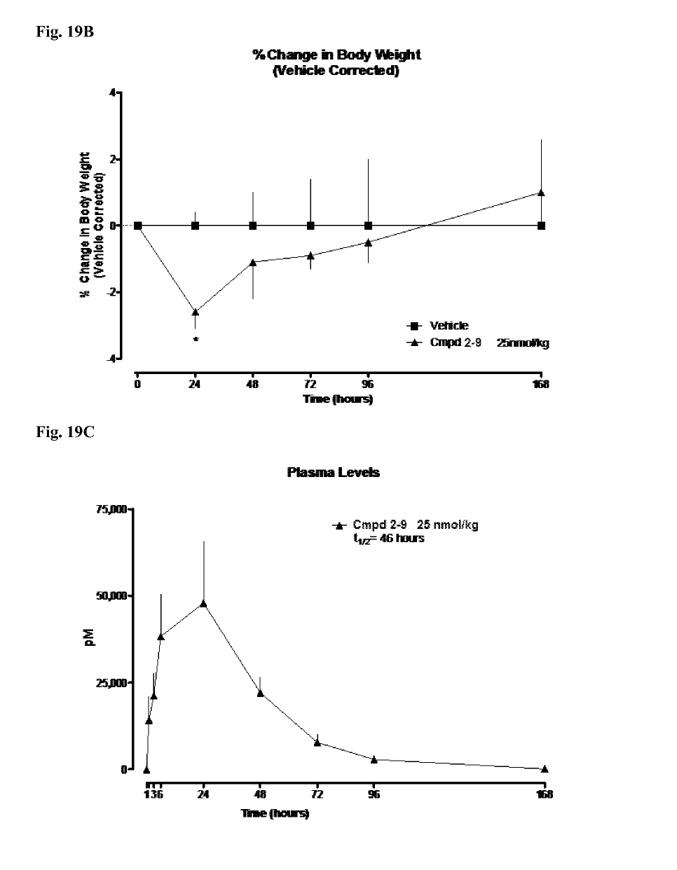

To further demonstrate the long half-life and long duration of activity of the engineered polypeptides described herein, the pharmacokinetic (PK) and pharmacodynamic (PD) properties were determined using rats. Pharmacokinetic profile and biological activity of an exemplary engineered polypeptide subcutaneously dosed in normal Harlan Sprague-Dawley (HSD) rats is presented. The recombinant engineered compounds Cmpd 2-11 (having Leu14 exendin-4 fused at its C-terminus via a glycine linker to PEP07986) and Cmpd 2-9 were injected subcutaneously at t=0 at 25 nmol/kg into normal HSD rats. Blood was collected via tail bleed at t=1 hour, 3 hours, 6 hours, 24 hours, 48 hours, 72 hours, 96 hours and 168 hours from fed HSD male rats. Food and body weights were measured daily. FIG. 18A (Cmpd 2-11) and 19A (Cmpd 2-9) depict effect of compound to reduce food intake. FIG. 18B (Cmpd 2-11) and 19B (Cmpd 2-9) depict effect of compound to reduce body weight. FIG. 18C (Cmpd 2-11) and 19C (Cmpd 2-9) depict a PK profile of the compound after a single dose. Points represent mean±sd. In the figures, vehicle is solid square, Cmpd 2-11 is open inverted triangle and Cmpd 2-9 is closed triangle. The plasma maximal concentration in FIG. 18C is equivalent to about 25,000 picomolar.

Exposure of up to seven (7) days was observed for these exemplary engineered polypeptides Cmpd 2-11, with a half-life of forty-two (42) hours, and Cmpd 2-9 with a half-life of forty-six (46) hours, in rats by this route of administration. By allometric scaling and in view of the strong affinity of the engineered polypeptides for human albumin, physical and biological activity duration at least as long and even longer is expected in human subjects. Accordingly, the compounds have use for at least daily, twice weekly, and even weekly administration, especially in human subjects.

### Example 30

### Long Duration and Action and Absolute Plasma Half-Life of the Engineered Polypeptides In Vivo

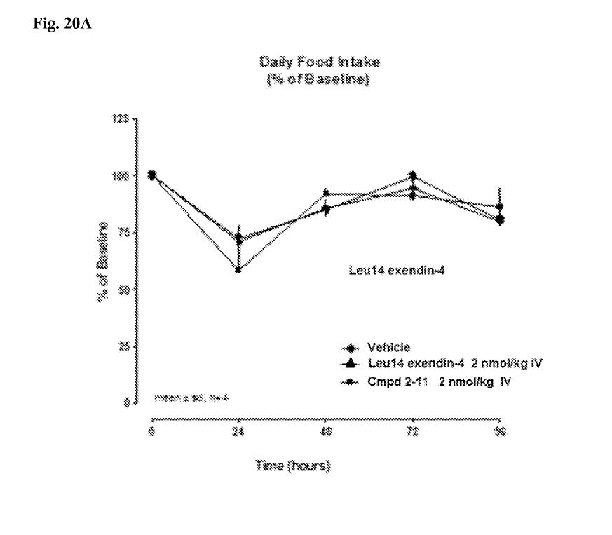

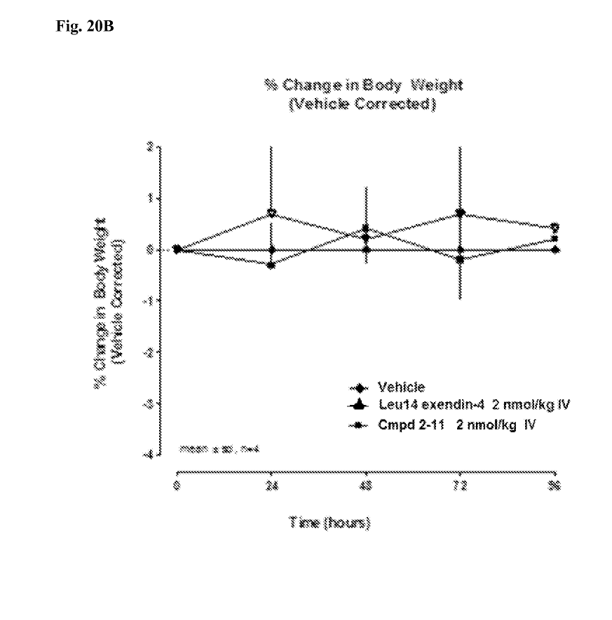

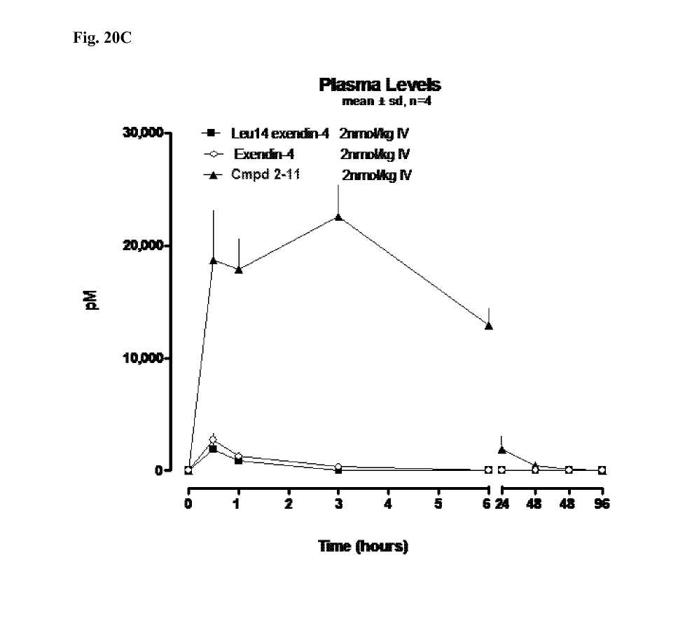

Pharmacokinetic profile and biological activity of an exemplary engineered polypeptide intravenously dosed in normal Harlan Sprague-Dawley (HSD) rats is presented, from which an absolute plasma half-life can be calculated. The recombinant engineered compound Cmpd 2-11 was injected intravenously at t=0 at 2 nmol/kg into normal HSD rats. Unconjugated exendin-4 and exendin-4 analog Leu14 exendin-4 were injected at 2 nmol/kg intravenously. Blood was collected via tail bleed at t=1 hour, 3 hours, 6 hours, 24 hours, 48 hours, 72 hours, 96 hours and 168 hours from fed HSD male rats. Food and body weights were measured daily. FIG. 20A depicts effect of Cmpd 2-11 to reduce food intake. FIG. 20B depicts effect of Cmpd 2-11 to reduce body weight. FIG. 20C depicts a PK profile of Cmpd 2-11 after a single IV dose. As expected the half-life of the unconjugated exendins is less than 15 minutes. An absolute half-life for the exemplary engineered polypeptide Cmpd 2-11 is estimated at about at least 12.3 hours. Points represent mean±sd.

Exposure of up to four days was observed for this exemplary engineered polypeptide, even at these relatively low doses, by this route of administration. By allometric scaling and in view of the strong affinity of the engineered polypeptides for human albumin, physical and biological activity duration at least as long and even longer is expected in human subjects. Accordingly, the compounds have use for at least twice daily (e.g. morning and night), at least daily, twice weekly, and even once weekly administration, especially in human subjects.

### Example 31

### Sub-Chronic Dosing Provides Superimpositioning and Exendin-Like Pharmacology

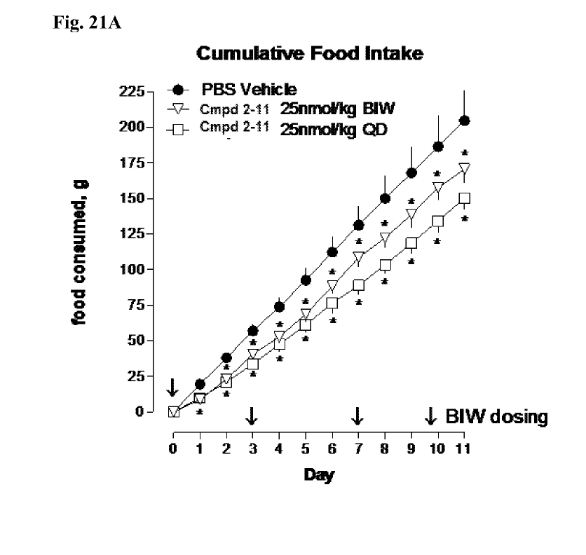

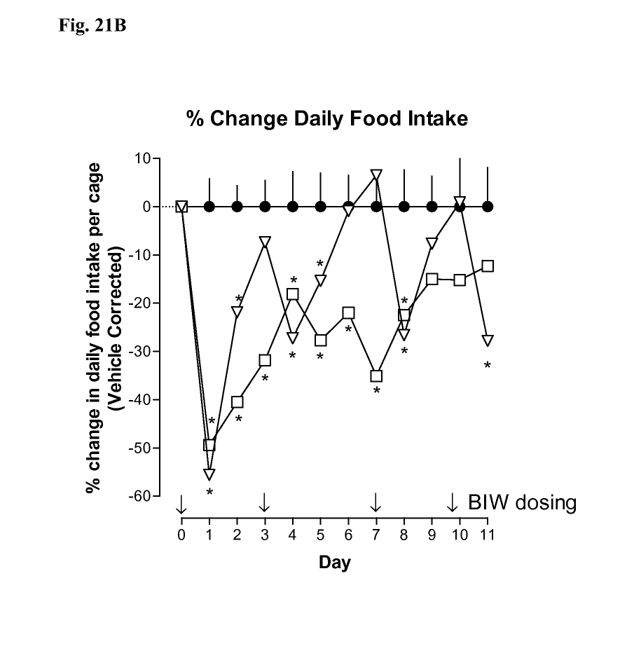

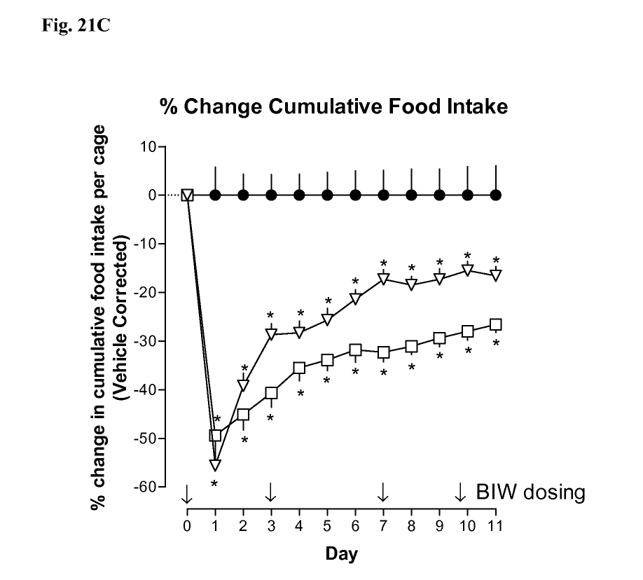

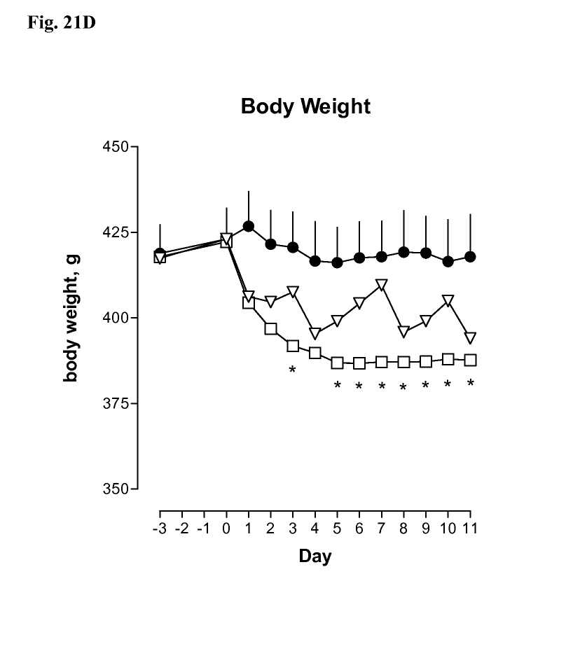

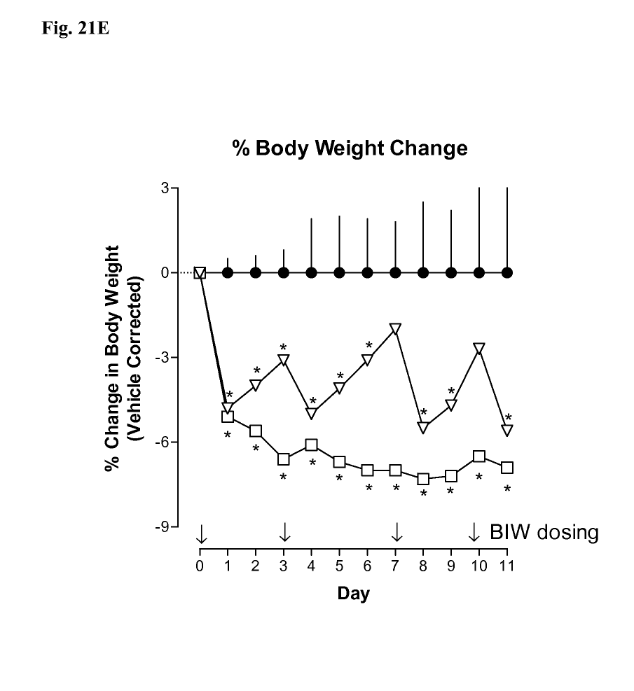

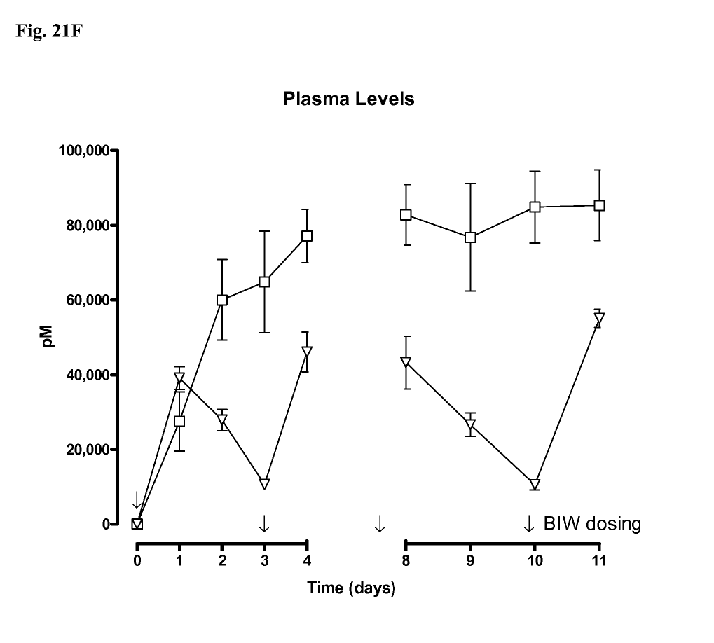

To demonstrate the effect of sub-chronic exposure of an exemplary engineered polypeptide in vivo, Cmpd 2-11 was administered subcutaneously twice weekly or daily for 14 days. Food intake inhibition, body weight decrease and plasma levels of Cmpd 2-11 were determined daily. Normal lean HSD rats were treated subcutaneously with 25 nmol/kg Cmpd 2-11 over 14 days as indicated in FIGS. 21A-21F, either twice weekly (BIW; open inverted trangles) as indicated by the down arrows or daily (QD; open square). Both BIW and QD administration inhibited daily food intake compared to vehicle (closed circle), as shown by cumulative food intake FIG. 21A, percent change in daily food intake FIG. 21B and percent change in cumulative food intake FIG. 21C. Both BIW and QD administration resulted in body weight loss as indicated by the reduction in total body weight FIG. 21D and greater percent negative change in body weight FIG. 21E compared to vehicle. FIG. 21F depicts a PK profile of Cmpd 2-11 given BIW or QD. Points represent mean±s.d. (standard deviation) with 4 to 6 animals per point. The test compound was injected subcutaneously on day=0 immediately following baseline sample collection.

As can be seen both modes of administration provide a superimpositioning effect leading to higher plasma levels of compound upon each subsequent dose, until a steady state is obtained. With as little as 11 days of treatment, the efficacy observed for the BIW (twice weekly) dose began to approximate that observed for Leu14 exendin-4 given at ˜7.2 nmol/kg/d by continuous infusion (CSI)—about 7% lower vehicle-corrected body weight at steady state level. The QD dosing provides a smoother profile, however, when translated to larger animals and those having longer inherent albumin plasma-half-lifes, smoother plasma levels approximating the QD pattern observed in the rats, are expected for BIW, thrice weekly and even weekly administration of this and any engineered polypeptide described herein. The QD dose with this engineered polypeptide achieved or surpassed efficacy of the unconjugated infused exendin or exendin analog during the short treatment period, which is therefore expected to be the case for each of the engineered polypeptides described herein.

### Example 32

### Lack of Vacuolization

With some drugs, such as some pegylated proteins, undesirable vacuoles can form in cytoplasm of epithelial cells lining the proximal convoluted tubules, which is an undesirable toxicity measure. The engineered albumin binding compounds of the present application do not form kidney vacuoles. C57BL6 female mice (n=2 cages, 3 mice/cage) were weighed daily 3 hours prior to lights out Immediately after weighing, on days 0-6 mice were injected subcutaneously with test compound. Mice were sacrificed on day 7 and kidneys submitted for histopathology. Severity score for cytoplasmic vacuolation of renal cortical tubular epithelial cells was as follows: score 1=minimal (8-15%); 2=mild (16-35%); 3=moderate (36-60%); 4=marked (>60%). A positive control compound known to cause vacuole formation was scored as 3. The ABD polypeptide PEP07986 itself scored 0. The engineered polypeptide Cmpd 2-5 scored 0.

### Example 33

### Composition and Preparation of OF1 Formulations Liquid Form

Preparation of liquid form formulations containing Exendin ABD compounds and comparator compounds formulated with propyl gallate and sodium chenodeoxycholate, designated formulation OF1, are described. Comparator Cmpd X, provided for comparison only, is an exendin containing polypeptide comprising a peptide sequence with affinity for albumin where the peptide sequence has no significant homology or structural motif in common with the ABD sequences of the engineered Exendin ABD compounds disclosed herein. The OF1 compositions in liquid form are shown in the following table.

| Ingredient | Function | Conc. (% w/w) |
|---|---|---|
| Test Compound | Active ingredient | Varied |
| Sodium | Absorption enhancer; | 20 |
| chenodeoxycholate | Solubilization enhancer for | |
| | aromatic alcohol | |
| Propyl gallate | Absorption enhancer; | 10 |
| | Solubilization enhancer for | |
| | non-conjugated bile salt; | |
| | Enzyme inhibitor | |
| MilliQ water | Solvent | QS to 100 |

A 10 gram scale preparation was performed as follows. Two grams (2.0 g) of sodium chenodeoxycholate and 1.0 g of propyl gallate were weighed separately. To a 20 mL scintillation glass vial containing a mini stirring bar, 6.8 g water was added and gently warmed before adding the 2.0 g of sodium chenodeoxycholate powder. The vial was sealed and the solution stirred for about 5 to 10 minutes with heating until dissolution was complete. The solution was removed from the heat, the 1.0 g of propyl gallate was added to the warm solution and stirred for about 5 to 10 minutes until dissolution was complete. The solution was allowed to cool to room temperature and pH was measured and adjusted as needed to pH to 7.4 using 30 mg/ml NaOH solution. The total weight was 9.8 to 10.0 g. The desired amount of dry test compound was added to the vial with gentle stirring at room temperature. All formulations, including placebo, were prepared fresh on the day of dosing described in the Examples herein and were used within 6 hours of preparation. In the Examples herein the liquid form of the OF1 formulations were dosed intra-duodenum (ID) or intra jejunum (IJ) via cannulation to rats.

### Example 34

### Composition and Preparation of OF1 Formulations Solid Form

Preparation of solid form formulations containing Exendin ABD compounds and comparator compounds formulated with propyl gallate and sodium chenodeoxycholate, designated formulation OF1, in enteric coated capsules, are described. The OF1 compositions in solid form are shown in the following table.

| | | Amount |
|---|---|---|
| Ingredient | Function | (mg/capsule) |
| Test Compound | Active ingredient | Varied |
| Sodium | Solubilization enhancer for | 57 |
| chenodeoxycholate | aromatic alcohol; Absorption | |
| | enhancer | |
| Propyl gallate | Absorption enhancer; Enzyme | 29 |
| | inhibitor | |
| Sucrose | Bulking agent | 22.6 |
| Gelatin capsule, | Encapsulation device | n/a |
| size 3 | | |

Enteric-coated capsules of Exendin ABD compounds or comparator compounds in solid OF1 formulations were prepared as follows. Lyophilized powder of test compounds, sodium chenodeoxycholate, propyl gallate, and sucrose were mixed by gentle shaking until homogenous and filled into size 3 gelatin capsules. The filled capsules were subsequently coated, as is known in the art, with a suspension consisting of Eudragit L100-55, triethyl citrate (TEC), and talc in 90 ml water:isopropanol:alcohol (34.29:51.42:4.29 v/v/v) as described in the following table.

| Ingredient | Function | Dry Powder (%) |
|---|---|---|
| Eudragit L100- | Enteric coating polymer | 62.5 |
| 55 | | |
| Triethyl citrate | Plasticizer | 6.25 |
| (TEC) | | |
| Talc | Anti-tacking agent | 31.25 |

The enteric coating polymer was selected to withstand stomach acid but dissolve at pH 5.5, to release OF1 and test compound in the small intestine. As described in the Examples herein, the capsules were dosed to male beagle dogs or cynomolgus monkeys for pharmacokinetic (PK) studies.

Stability of Exendin ABD compounds OF1 solid form formulations was determined during storage of capsules at 5° C. at 0 and 4 weeks. The capsules were stored in a sealed 20 mL scintillation vial and placed in 2-8° C. for the period of the study. Content and purity of each test compound were determined by RP-HPLC using a Vydac C18 column, 0.1% TFA in mixtures of aqueous and acetonitrile as mobile phases, with UV detection at 214 nm, as described in the following table.

| Parameter | Value | | |
|---|---|---|---|
| Mobile Phase 1 (MP1) | 0.1% TFA in 98.75/1.25 water/ACN v/v | | |
| Mobile Phase 2 (MP2) | 0.1% TFA in 20/80 water/ACN v/v | | |
| Column | Vydac C18, 4.6 × 250 mm, 5 μm particle | | |
| | size, 300 Angstrom pore size, part number | | |
| | 218TP54 | | |
| | Time (min) | MP1 (%) | MP2 (%) |
| Gradient conditions | 0 | 65 | 35 |
| | 2 | 65 | 35 |
| | 22 | 30 | 70 |
| | 32 | 0 | 100 |
| | 35 | 0 | 100 |
| | 35.5 | 65 | 35 |
| | 45 | 65 | 35 |

| | |
|---|---|
| Flow rate | 1.0 mL/min |
| Column temperature | 30 ± 5° C. |
| Sample temperature | 5 ± 3° C. |
| Injection volume | 40 μL |
| Detection, UV | 214 nm |
| Absorbance | |
| Run time | 45 min |
| Progeny Compounds | 14-16 minutes |
| Retention time | |

Results of the stability study indicated that neither the amount nor the purity of Cmpd 103 or Cmpd X in OF1 changed significantly between the zero and 4 week time-points.

### Example 35

### Composition and Preparation of OF2 Formulations Liquid Form

Preparation of liquid form formulations containing Exendin ABD compounds and comparator compounds formulated with lauroyl-L-carnitine and citric acid, designated formulation OF2, are described. The OF2 compositions in liquid form are shown in the following table.

| Ingredient | Function | Unit Formula |
|---|---|---|
| Test Compound | Active ingredient | Varied |
| Citric acid | pH modifier; Protease | 0.5 g |
| | inhibitor | |
| Lauroyl-L- | Absorption enhancer | 0.05 g |
| carnitine (C12) | | |
| Water | Solvent | Q.S. to 1 gram |

A 10 gram scale preparation was performed as follows. One-half gram (0.5 g) of lauroyl-L-carnitine and 5.0 g of citric acid were weighed separately. To a 20 mL scintillation glass vial containing a mini stirring bar, 4.3 g water was added. The citric acid was added to the water and stirred until dissolution. The lauroyl-L-carnitine was then added and dissolved by stirring (or sonication if necessary). After all solids were dissolved, the solution was q.s. to a total weight of 10 g with water. The desired amount of dry test compound was added to the vial with gentle stirring at room temperature. All formulations, including placebo, were prepared, stored at room temperature and used as described in the Examples within 3 days of preparation. In the Examples herein the liquid form of the OF2 formulations were dosed intra-duodenum (ID) or intra jejunum (IJ) via cannulation to rats.

### Example 36

### Composition and Preparation of OF3 Formulations Liquid Form

Preparation of liquid form formulations containing Exendin ABD compounds and comparator compounds formulated with sodium caprate, designated formulation OF3, are described. The OF3 compositions in liquid form are shown in the following table.

| Ingredient | Function | Unit Formula (g) |
|---|---|---|
| Test Compound | Active ingredient | Varied |
| Sodium caprate | Absorption enhancer | 0.1 g |
| WFI | Solvent | Q.S. to 1 gram |

A 10 gram scale preparation was performed as follows. One gram (1.0 g) of sodium was added to 8.8 g water in a 20 mL scintillation glass vial containing a mini stirring bar, and then q.s. to 10 gram. The solution was stirred until dissolution was complete. The desired amount of dry test compound was added to the vial with gentle stirring at room temperature until all solid was dissolved. The OF3 formulations were prepared, stored at room temperature and used within 3 days in the Examples described herein. Formulations were dosed intra-duodenum (ID) or intra jejunum (IJ) via cannulation to rats.

### Example 37

### Composition and Preparation of OF3 Formulations Solid Form

Preparation of solid form formulations containing Exendin ABD compounds and comparator compounds formulated with sodium caprate, designated formulation OF3, in enteric coated capsules, are described. The OF3 compositions in solid form are shown in the following table.

| Ingredient | Function | Amount (mg/capsule) |
|---|---|---|
| Test Compound | Active ingredient | Varied |
| Sodium caprate | Absorption enhancer | 100 |
| Sucrose | Bulking agent | 10 |
| Gelatin capsule, | Encapsulation | n/a |
| size 3 | | |

Enteric-coated capsules of Exendin ABD compounds or comparator compounds in solid OF1 formulations were prepared as follows. Lyophilized powder of test compounds, sodium caprate powder, and sucrose were mixed by gentle shaking until homogenous and filled into size 3 gelatin capsules. The filled capsules were subsequently coated, as is known in the art, with a suspension consisting of Eudragit L100-55, triethyl citrate (TEC), and talc, in 90 ml water:isopropanol:alcohol (34.29:51.42:4.29 v/v/v) as described in the following table.

| Ingredient | Function | Dry Powder (%) |
|---|---|---|
| Eudragit L100-55 | Enteric coating polymer | 62.5 |
| Triethyl citrate | Plasticizer | 6.25 |
| (TEC) | | |
| Talc | Anti-tacking agent | 31.25 |

The enteric coating polymer was selected to withstand stomach acid but dissolve at pH 5.5, to release OF3 permeation enhancer and test compound in the small intestine.

As described in the Examples herein, the capsules were dosed to male beagle dogs or cynomolgus monkeys for pharmacokinetic (PK) studies.

Stability of Exendin ABD compounds in OF3 solid form formulations was determined during storage of capsules at 5° C. at 0 and 4 weeks. The capsules were stored in a sealed 20 mL scintillation vial and placed at 2-8° C. for the period of the study. Content and purity of each test compound were determined by RP-HPLC using a Vydac C18 column, 0.1% TFA in mixtures of aqueous and acetonitrile as mobile phases, with UV detection at 214 nm, as described in the above Example. Neither the amount nor the purity of Cmpd 103 or Cmpd X in OF3 changed significantly between the zero and 4 week time-points.

### Example 38

### Composition and Preparation of OF4 Formulations Liquid Form

Preparation of liquid form formulations containing Exendin ABD compounds and comparator compounds formulated with tetradecyl-beta-d-maltoside (TDM), designated formulation OF4, are described. The OF4 compositions in liquid form are shown in the following table.

| Ingredient | Function | Unit Formula |
|---|---|---|
| Test Compound | Active ingredient | Varied |
| Tetradecyl-β-d- | Absorption enhancer | 2.5 mg |
| maltoside (TDM) | | |
| WFI | Solvent | Q.S. to 1 gram |

A 10 gram scale preparation was performed as follows. Twenty five mg (25 mg) of TDM weighed and added to 8.8 g water in a 20 mL scintillation glass vial containing a mini stirring bar, and then q.s. to 10 gram. The solution was stirred until dissolution was complete. The desired amount of dry test compound was added to the solution with gentle stirring at room temperature until all solid was dissolved. The OF4 formulations were prepared, stored at room temperature and used within 3 days in the Examples described herein. Formulations were dosed intra-duodenum (ID) or intra jejunum (IJ) via cannulation to rats.

### Example 39

### Comparison of Formulations for Transmucosal Permeation

The capability of different formulations of Cmpd 103, Cmpd 202 and Cmpd X for transmucosal permeation were compared by determination of blood plasma levels of each compound after delivery by intra-duodenal administration. This route demonstrates transmucosal permeation capability of each formulation and compound and is applicable to other routes of transmucosal delivery, e.g. nasal, oral, buccal, sub-lingual, etc. Intra-duodenal administration demonstrates capability after delivery via an oral route, such as by formulations of the of engineered Exendin ABD compounds designed to release drug and excipients in the small intestine, e.g. enteric coated tablet or capsule. Investigated were formulations of test compounds in PBS (Phosphate buffered Saline), OF1, OF2, OF3 and OF4 formulations described herein. Briefly, OF1 contains sodium chenodeoxycholate (20% w/w) and propyl gallate (10% w/w); OF2 contains 17% citric acid (w/w) and 1.7% carnitine (w/w); OF3 contains sodium caprate at the concentration indicated herein; OF4 contains 0.25% tetradecyl-β-d-maltoside (TDM).

Overnight-fasted male Harlan-Sprague Dawley rats were anesthetized and underwent midline laparotomy and saphenous artery and vein cannulations. Injections, intravenous (IV) or Intra-duodenal (ID), started at t=0, one hour after post-surgical stabilization. Blood pressure was monitored and blood collected at time points 0, 5, 15, 30, 45, 60 and 120 minutes post delivery of compound.

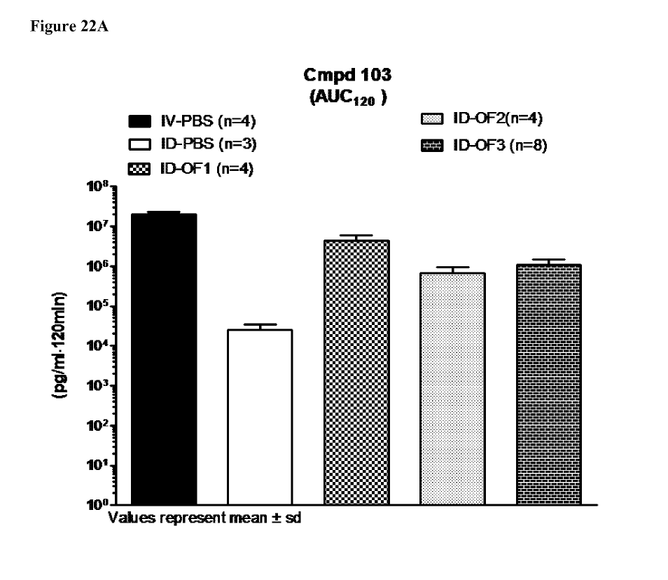

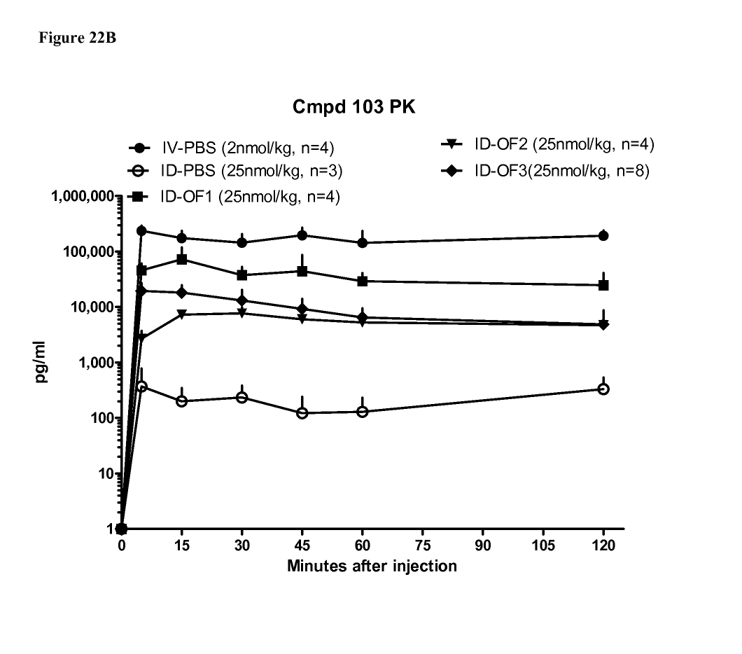

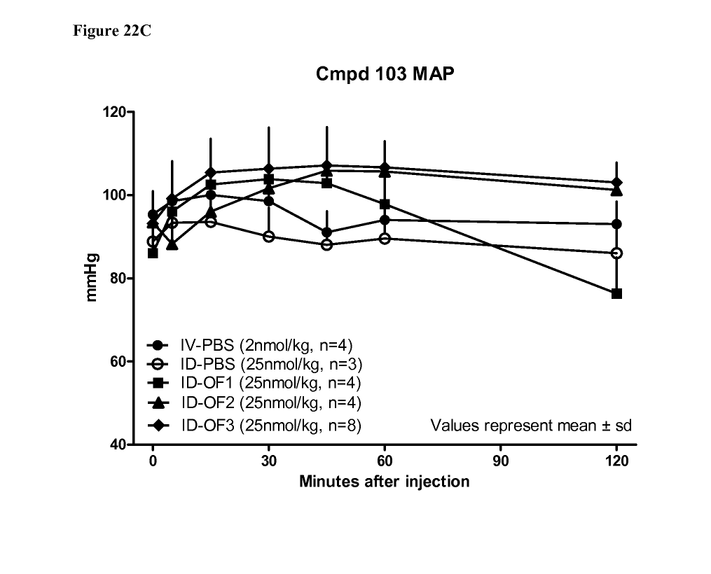

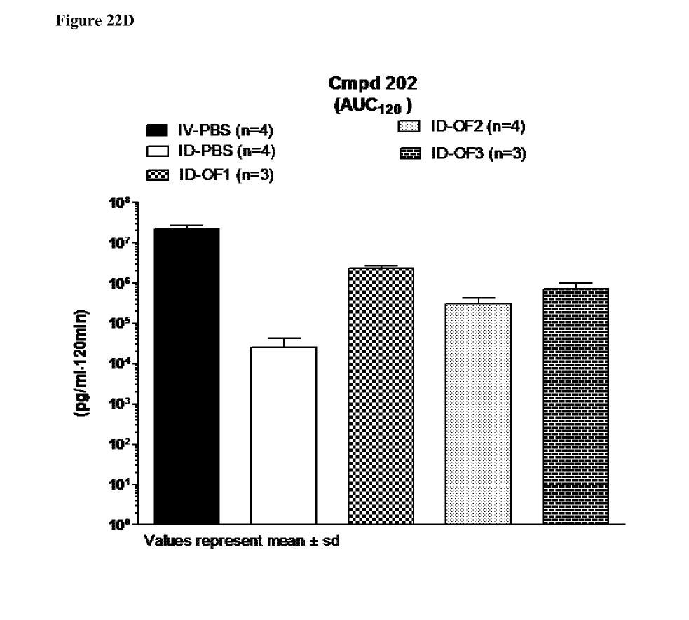

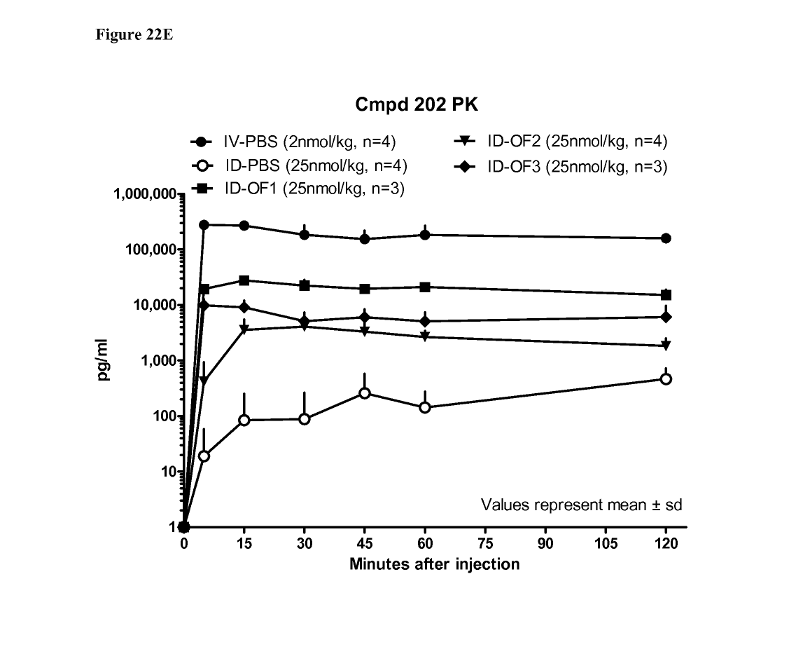

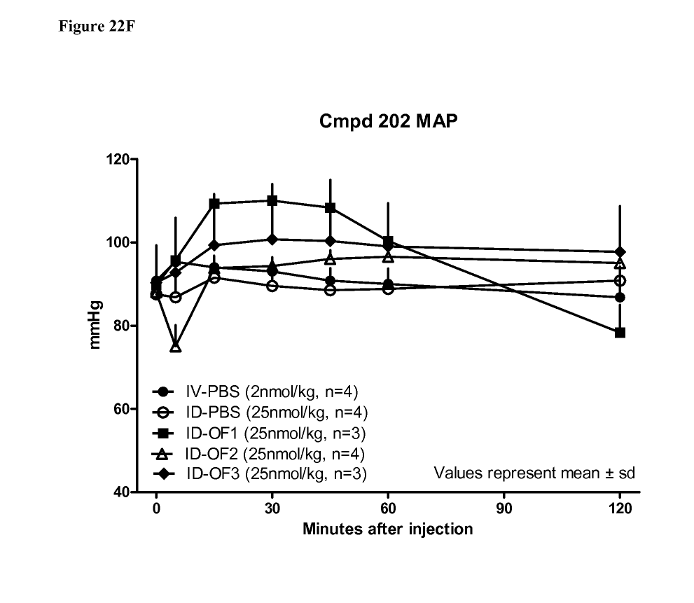

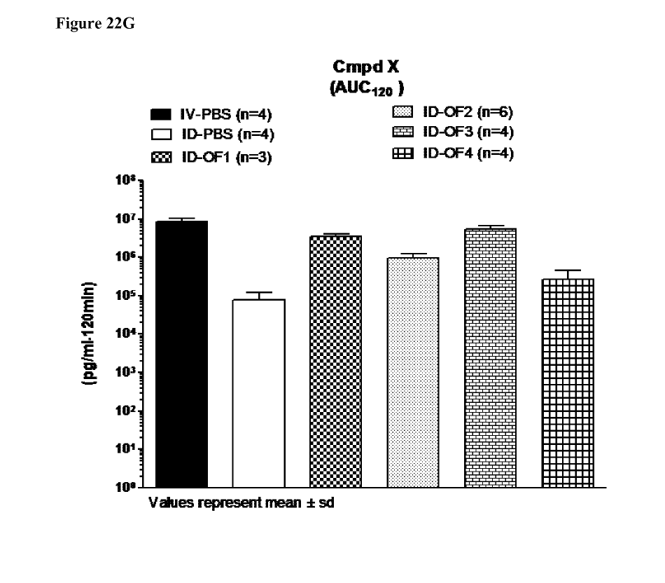

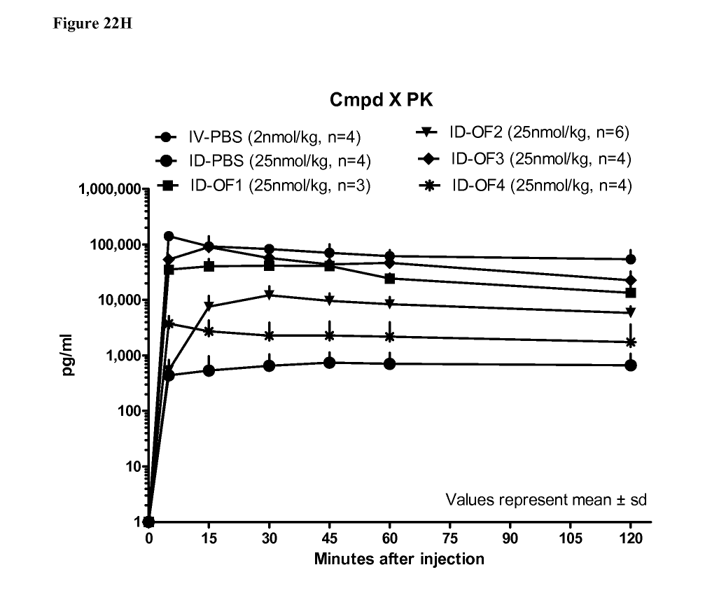

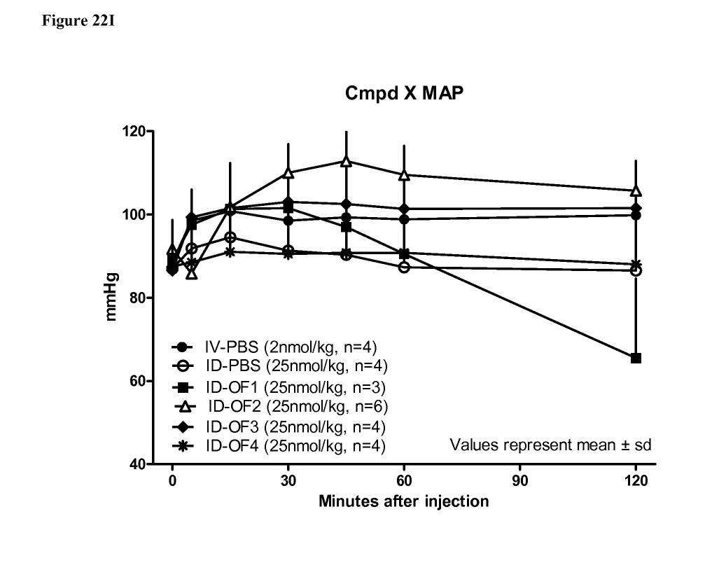

The results are presented in FIGS. 22A-22I for Exendin ABD compounds Cmpd 103 (Cmpd 31) and Cmpd 202 (Cmpd 2-11) and of comparator compound Cmpd X. Results for intravenous administration in PBS (IV-PBS) are also shown. FIG. 22A depicts blood plasma exposure as AUC at 120 minutes after delivery of the formulations described herein for Cmpd 103. FIG. 22B presents a pharmacokinetic profile over 120 minutes for the formulations of Cmpd 103. FIG. 22C presents profiles of blood pressure after delivery of the formulations of Cmpd 103. FIG. 22D depicts blood plasma exposure as AUC at 120 minutes after delivery of the formulations described herein for Cmpd 202. FIG. 22E presents a pharmacokinetic profile over 120 minutes for the formulations of Cmpd 202. FIG. 22F presents blood pressure profiles after delivery of the formulations of Cmpd 202. FIG. 22G depicts blood plasma exposure as AUC at 120 minutes after delivery of the formulations described herein for Cmpd X. FIG. 22H presents a pharmacokinetic profile over 120 minutes for the formulations of Cmpd X. FIG. 22I presents profiles of blood pressure after delivery of the formulations of Cmpd X.

AUC from 0-2 hours and absolute bioavailability (compared to IV) were determined and are presented in the following tables.

| | | | AUC | |
|---|---|---|---|---|
| | | | 0-2 hours | |
| Cmpd | Dose | Dose | normalized | Bioavailability |
| 103 | ug/kg | nmol/kg | (×1,000,000) | (percent) |
| IV-PBS | 18.9 | 2 | 20.170 | 100 |
| ID-PBS | 236.6 | 25 | 0.002 | 0.01% |
| ID-ORF1 | 236.6 | 25 | 0.345 | 1.71 |
| ID-ORF2 | 236.6 | 25 | 0.052 | 0.26 |
| ID-ORF3 | 236.6 | 25 | 0.088 | 0.44 |

| | | | AUC | |
|---|---|---|---|---|
| | | | 0-2 hours | |
| Cmpd | Dose | Dose | normalized | Bioavailability |
| 202 | ug/kg | nmol/kg | (×1,000,000) | (percent) |
| IV-PBS | 18.5 | 2 | 22.032 | 100 |
| ID-PBS | 231.6 | 25 | 0.002 | 0.01 |
| ID-ORF1 | 231.6 | 25 | 0.189 | 0.86 |
| ID-ORF2 | 231.6 | 25 | 0.025 | 0.11 |
| ID-ORF3 | 231.6 | 25 | 0.058 | 0.26 |

| | | | AUC | |
|---|---|---|---|---|
| | | | 0-2 hours | |
| | Dose | Dose | normalized | Bioavailability |
| Cmpd X | ug/kg | nmol/kg | (×1,000,000) | (percent) |
| IV-PBS | 11.4 | 2 | 8.456 | 100 |
| ID-PBS | 142.1 | 25 | 0.006 | 0.07 |
| ID-ORF1 | 142.1 | 25 | 0.277 | 3.27 |
| ID-ORF2 | 142.1 | 25 | 0.076 | 0.90 |
| ID-ORF3 | 142.1 | 25 | 0.437 | 5.17 |
| ID-ORF4 | 142.1 | 25 | 0.021 | 0.25 |

OF1 provided an unexpectedly superior profile, including amount absorbed and bioavailability, for the Exendin ABDs, whereas OF3 provided a superior profile for Cmpd X. OF4 was not effective for the ABD Exendin compounds.

### Example 40

### Transmucosal Permeation Via Jejunum of Rat

Transmucosal permeation was further investigated with OF1 by delivery to the jejunum. This route demonstrates transmucosal permeation capability of the formulation and compound and is applicable to other routes of transmucosal delivery, e.g. nasal, oral, buccal, sub-lingual, etc. Intra jejunal administration demonstrates capability after delivery via an oral route, such as by formulations of the of engineered Exendin ABD compounds designed to release drug and excipients at the jejunum in the small intestine, e.g. enteric coated tablet or capsule.

Male Sprague Dawley rats with jejunal catheters were purchased from Taconic (USA). For each compound, an intra-jejunal (IJ) bolus dose equivalent to 1 mg/kg exendin-4 was administered to fed conscious animals at t=0, and blood was collected from the tail vein at time points t=0, 0.5, 2, 6, 24 and 48 hours. Since each test compound has a molecular weight greater than exendin-4, a greater amount of each test compound was administered in order to provide a molar equivalent to 1 mg/kg exendin-4. IJ instillation in conscious fed male Sprague Dawley rats simulates release from an enteric coated capsule or tablet of OF1, which is a mixture of a permeation enhancer (propyl gallate), which may also have enzyme inhibitor activity, and a propyl gallate solubility enhancer (sodium chenodeoxycholate), which may also have permeation enhancer activity, as described herein. N=6 rats, mean±sd. A compound compound-specific ELISA assay was used to detect Cmpd 102, Cmpd 201, Cmpd 103 and Cmpd 202; LCMS/MS was used to detect Cmpd 101 and liraglutide. Plasma levels were expressed as picomolar at the time indicated.

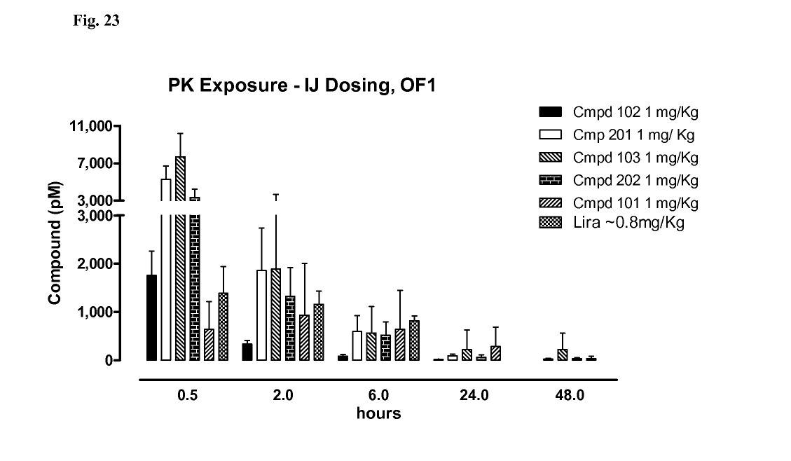

Results are presented in FIG. 23. FIG. 23 depicts blood plasma exposure at the indicated time point of each exemplary engineered polypeptide. Cmpd 101 is also referred to as Cmpd 15. Cmpd 102 is also referred to as Cmpd 21. Cmpd 103 is also referred to as Cmpd 31. Cmpd 201 is also referred to as Cmpd 2-9. Cmpd 202 is also referred to as Cmpd 2-11. As can be seen, greater than twenty-four (24) exposure is observed for the engineered constructs in OF1 via after intestinal uptake. Compatibility of each Exendin ABD compound with both excipients of OF1 was observed. Superiority over a similar molar amount of comparator albumin-binding compound liraglutide was observed. This was even more surprising since liraglutide was determined to be relatively resistant to pancreatic enzymes as determined herein.

### Example 41

### Transmucosal Permeation Via Oral Delivery

Transmucosal permeation of an exemplary Exendin ABD compound was further investigated with OF1 by oral delivery to a beagle dog. This route demonstrates capability of Exendin ABD systemic uptake after oral administration route with formulations suitable for permeation across the intestinal wall, such as by formulations of the engineered Exendin ABD compounds designed to release drug and excipients at the small intestine, e.g. enteric coated tablet or capsule.

Plasma levels of an exemplary engineered Exendin ABD compound, Cmpd 103 (also referred to as Cmpd 15), after a single oral administration of Cmpd 103 formulated in OF1 encapsulated in an enteric coated capsule to beagle dogs, were investigated. A dose equivalent to 0.1 mg/kg exendin-4 was administered in one capsule. The capsule contained 2.26 mg. Compound was measured via a compound-specific ELISA. n=1.

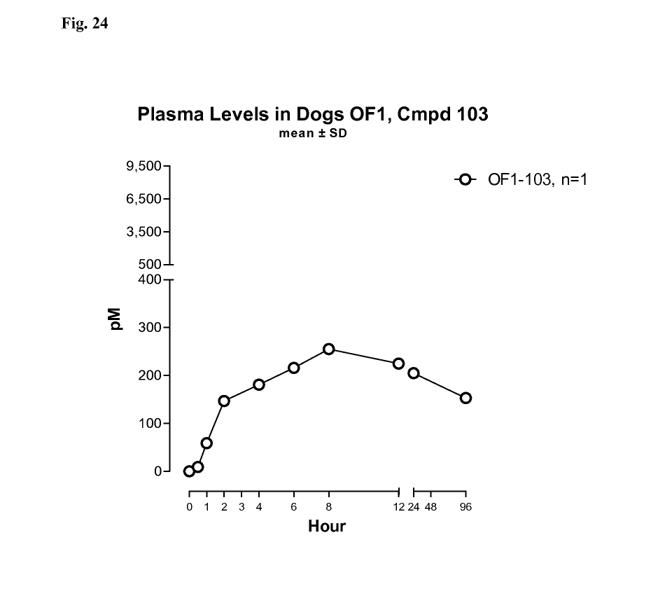

Results are presented in FIG. 24. FIG. 24 present a pharmacokinetic profile in blood plasma of Cmpd 103 (Cmpd 15) formulated in OF1 after the single oral administration in solid form in an enteric coated capsule to beagle dogs. Greater than five (5) days of exposure was observed. Relatively rapid uptake was observed. After about one hour, therapeutic levels were achieved and were sustained over the time course. This study further demonstrates the oral bioavailability and uptake of the engineered compounds and their long duration after intestinal uptake. The greater than five (5) days of exposure observed in the dog is predictive of once weekly, even twice monthly, or certainly more often, e.g. once every 24 hours, transmucosal administration to primates, especially humans. A sustained therapeutic level is achieved with a relatively low Cmax that enables patient tolerability and increased compliance and acceptance.

In contrast, exendin-4 delivered in the same formulation provided exposure over no more than about 4-6 hours, not suitable for once daily dosing to achieve 18-24 hour exposure, and a relatively high Cmax compared to the desired therapeutic target concentration, which would not be conducive to patient tolerability. Similarly, like exendin-4, Cmpd X achieved a relatively high Cmax shortly after delivery but which rapidly decreased after within about 8 hours, which PK profile could compromise patient tolerability.

### Example 42

### Transmucosal Permeation Via Jejunum of a Primate

Transmucosal permeation of an exemplary Exendin ABD compound was further investigated with OF1 by oral delivery to a primate. This route demonstrates capability of Exendin ABD systemic uptake after oral administration route with formulations suitable for permeation across the intestinal wall, such as by formulations of the engineered Exendin ABD compounds designed to release drug and excipients at the small intestine, e.g. enteric coated tablet or capsule.

Plasma levels of an exemplary engineered Exendin ABD compound, Cmpd 103 (also referred to as Cmpd 15), after a single oral administration of Cmpd 103 formulated in OF1 encapsulated in an enteric coated capsule to cynomolgus monkey, were investigated. A dose equivalent to 0.1 mg/kg exendin-4 was administered in one capsule. The capsule contained 2.26 mg. Compound was measured via a compound-specific ELISA. n=1. Male cynomolgus monkeys, n=3 per treatment, were fasted overnight, and dosed on Day=1, t=0. A half banana was given following the 2 hr sample collection. One-half daily food ration was given ˜4 hours post-dose, and the remaining daily food rations were fed ˜6 hours post-dose. Also provided is the profile of Cmpd 103 intravenously administered at a bolus dose equivalent to 10 μg/kg exendin-4. Each peroral dose contained 2.1 mg Cmpd 103 per capsule. Cmpd X was delivered at 1.3 mg per capsule. These doses were equivalent to 0.170 mg/kg exendin-4. Compounds measured by a compound-specific ELISA.

The enteric coating protects the formulation from the low stomach and gastric acid and keeps the formulation intact. The enteric coating dissolves at a desired pH at the desired location in the intestine, in this case at near neutral pH. Permeability enhancer(s) and solubility enhancer are then released with the drug polypeptide, and permeation across the mucosal barrier and intestinal cell wall barrier is facilitated.

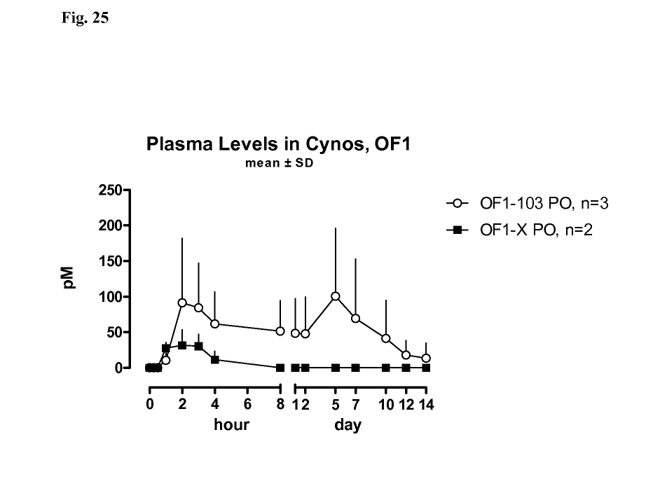

FIG. 25 presents pharmacokinetic profiles in blood plasma of exemplary engineered polypeptide Cmpd 103 (Cmpd 15) and Cmpd X formulated in OF1 after the single oral administration to a cynomolgus monkey. Cmpd X is provided for comparison only. Greater than fourteen (14) days of exposure was observed for the exemplary Exendin ABD compound Cmpd 103, whereas Cmpd X provided less than one day of exposure. Relatively rapid uptake was observed for Cmpd 103, demonstrating that a sustained release formulation is not required. After about one hour, therapeutic levels were achieved and were sustained over the time course. This study further demonstrates the oral bioavailability and uptake of the engineered compounds and their long duration after intestinal uptake. The greater than fourteen (14) days of exposure observed in the monkey is predictive of once weekly, even twice monthly or longer, and certainly more often, e.g. once every 24 hours, transmucosal administration to primates, especially humans. The turnover of albumin in the cynomolgus monkey is about 11-13 days, and about 20 days in humans. The Exendin ABD compounds appear to circulate at least as long if not longer than the serum albumin of the recipient species. A flatter, more even, patient-tolerable PK profile was observed, which provides continuous sustained exposure at therapeutic levels, which when combined with a Cmax that is relatively low compared to the average sustained plasma concentration, will enhance patient tolerability, compliance, and acceptance enabling increased therapy effectiveness, particularly for human patients.

### Example 43

### Stability in a Pancreatin Enzyme Mixture

Relative stability of Exendin ABD compounds and comparator compounds in a pancreatin enzyme mixture was investigated. Pancreatin is a mixture of several intestinal digestive enzymes produced by the exocrine cells of the pancreas. It is composed of amylase, lipase and proteases elastase, trypsin and chymotrypsin.

A six (6) microliter aliquot of Pancreatin (Sigma-Aldrich, USA Cat. No. P7545-25 g) stock solution (1 mg/ml) was added to 994 microliters of 50 mM phosphate buffer pH 6.8 in a 1.5 mL polypropylene microcentrifuge tube with an O-ring seal to prevent evaporation. A twenty (20) microliter aliquot of a stock solution of the test compound (300 micromolar in 50% acetonitrile) was added to 80 microliter of 50 mM phosphate buffer pH 6.8 in a 1.5 mL polypropylene microcentrifuge tube with an O-ring seal to prevent evaporation. Then 100 uL of the diluted, buffered pancreatin mixture was added to the 100 uL of test compound solution and the mixture was mixed by brief manual shaking. Next, 25 ul of the reaction mixture was aliquoted into each of six polypropylene microcentrifuge tubes. One tube contained 50 μL of Stop solution (0.2N Hydrochloric acid) to provide a sample to determine the initial concentration of peptide (t=0). All six reaction tubes were then incubated at 37° C. with mixing at 500 RPM in a Vortemp 56 incubator. The digestion reaction was stopped at each desired time-point by adding 50 uL Stop solution. Time-points were 0, 10, 20, 30, 60 and 120 minutes. Each quenched tube was then placed back into the incubator until the last time-point was stopped, at which time each was centrifuged at 10000×g for 3 minutes at room temperature. Each supernatant was removed and 50 uL supernatant was transferred to an autosampler vial for analysis. Sample analysis was performed with an Agilent HLPC (LC/MS 1200) using a gradient of 5-95% acetonitrile in water containing 0.1% trifluoroacetic acid over 11 minutes. The most intense ion of each intact test compound was monitored to determine the rate of degradation and to quantify the amount of intact compound remaining in the reaction solution. Results for the 30 and 60 minute time-points are expressed as percent of initial peptide in the following table, and show that the Exendin ABD compounds are susceptible to pancreatic enzymes.

| | Percent Remaining at | Percent Remaining |
|---|---|---|
| Compound | 30 minutes | at 60 minutes |
| GLP-1 (7-36) (Human) | 39 | 24 |
| 14Leu exendin-4 | 77 | 60 |
| Cmpd 31 | | |
| Cmpd 2-11 | 75 | 52 |
| PEP07986 | 64 | 48 |
| Liraglutide | 100 | 100 |

The above study was repeated with the reaction mixture buffered to pH 4 and pH 5. Lowering the pH to pH 5 decreased the extent of degradation by about one-half to one-fourth, with essentially no degradation at pH 4.0, which is apparently a consequence of the pH dependence of the proteases in the Pancreatin mixture. These studies indicate that formulation agents that inhibit intestinal proteases, e.g. protease inhibitor, pH-lowering agent, can further increase bioavailability of the Exendin ABD compounds when included in the formulations as described herein.

1. A pharmaceutical composition comprising (a) an engineered polypeptide comprising an Albumin Binding Domain polypeptide (ABD) sequence, and a first peptide hormone domain (HD1) sequence selected from an exendin sequence, an exendin analog sequence, an exendin active fragment sequence or an exendin analog active fragment sequence, and (b) a mucosal permeation enhancer, with the proviso that the composition does not comprise a Phosphate Buffered Saline comprising propylene glycol (50:50 v/v).

2. The composition according to claim 1, where the engineered polypeptide further comprises a first linker (L1) covalently linking said ABD sequence and said HD1 sequence.

3. The composition according to claim 1, wherein said engineered polypeptide comprises said ABD sequence as a C-terminal moiety and said HD1 sequence as an N-terminal moiety.

4-5. (canceled)

6. The composition according to claim 1, wherein said HD1 sequence is said exendin sequence or said exendin analog sequence.

7-13. (canceled)

14. The composition according to claim 1, wherein said exendin analog sequence comprises from 1 to 5 amino acid modifications relative to exendin-4 sequence, said modifications independently selected from any one or combination of an insertion, deletion, addition and substitution.

15. The composition according to claim 1, wherein said ABD sequence comprises an Albumin Binding Motif (ABM) sequence, an ABD1 sequence or an ABD2 sequence.

16-33. (canceled)

34. The composition according to claim 1, wherein said ABD sequence comprises an amino acid sequence selected from the amino acid sequence comprising:
formula (iii)
(SEQ ID NO: 594)
LA X3 AK X6 X7 AN X10 ELD X14 YGVSDF YKRLIDKAKT V
EGVEALKDA ILAALP
wherein independently of each other
X3 is selected from E, S, Q and C;
X6 is selected from E, S and C;
X7 is selected from A and S;
X10 is selected from A, S and R;
X14 is selected from A, S, C and K;
the leucine at position 45 is present or absent; and
the proline at position 46 is present or absent;
formula (iv) an amino acid sequence which has at least 95% identity to the sequence defined in (iii),
with the proviso that X7 is not L, E or D;
or alternatively,
with the proviso that the amino acid sequence is not defined by the following sequence:
(SEQ ID NO: 593)
LAEAK Xa Xb A Xc Xd EL Xe KY GVSD X5 YK X8 X9 I
X11 X12 A X14 TVEGV X20 AL X23 X24 X25 ILAALP
wherein independently of each other,
Xa is selected from V and E;
Xb is selected from L, E and D;
Xc is selected from N, L and I;
Xd is selected from R and K;
Xe is selected from D and K;
X5 is selected from Y and F;
X8 is selected from N, R and S;
X9 is selected from V, I, L, M, F and Y;
X11 is selected from N, S, E and D;
X12 is selected from R, K and N;
X14 is selected from K and R;
X20 is selected from D, N, Q, E, H, S, R and K;
X23 is selected from K, I and T;
X24 is selected from A, S, T, G, H, L and D; and
X25 is selected from H, E and D.

35. The composition according to claim 15, wherein said ABD sequence comprises an amino acid sequence comprising:
formula (iii)
(SEQ ID NO: 594)
LA X3 AK X6 X7 AN X10 ELD X14 YGVSDF YKRLIDKAKT
VEGVEALKDA ILAALP
wherein independently of each other
X3 is selected from E, S, Q and C;
X6 is selected from E, S and C;
X7 is selected from A and S;
X10 is selected from A, S and R;
X14 is selected from A, S, C and K;
the leucine at position 45 is present or absent; and
the proline at position 46 is present or absent.

36. The composition according to claim 15, wherein said ABD sequence comprises an amino acid sequence comprising formula (iv) or an amino acid sequence which has at least 95% identity to the sequence defined in (iii), with the proviso that X7 is not L, E or D; or alternatively, with the proviso that the amino acid sequence is not defined by the following sequence:
(SEQ ID NO: 593)
LAEAK Xa Xb A Xc Xd EL Xe KY GVSD X5 YK X8 X9 I
X11 X12 A X14 TVEGV X20 AL X23 X24 X25 ILAALP
wherein independently of each other, Xa is selected from V and E; Xb is selected from L, E and D; Xc is selected from N, L and I; Xd is selected from R and K; Xe is selected from D and K; and X5 is selected from Y and F; X8 is selected from N, R and S; X9 is selected from V, I, L, M, F and Y; X11 is selected from N, S, E and D; X12 is selected from R, K and N; X14 is selected from K and R; X20 is selected from D, N, Q, E, H, S, R and K; X23 is selected from K, I and T; X24 is selected from A, S, T, G, H, L and D; and X25 is selected from H, E and D.

37-67. (canceled)

68. The composition according to claim 1, wherein the ABD sequence is selected from SEQ ID NOs:301-463 or SEQ ID NOs:500-733.

69-102. (canceled)

103. The composition of claim 1, wherein the engineered polypeptide comprises (SEQ ID NO:40), (SEQ ID NO:41), (SEQ ID NO:42), (SEQ ID NO:43), (SEQ ID NO:51), (SEQ ID NO:163), (SEQ ID NO:99), (SEQ ID NO:169), (SEQ ID NO:170), (SEQ ID NO:95), (SEQ ID NO:97), (SEQ ID NO:96), (SEQ ID NO:55), (SEQ ID NO:53), (SEQ ID NO:62), (SEQ ID NO:67), (SEQ ID NO:166), (SEQ ID NO:167), (SEQ ID NO:51), (SEQ ID NO:52), (SEQ ID NO:53), (SEQ ID NO:54), (SEQ ID NO:55), (SEQ ID NO:56), (SEQ ID NO:57), (SEQ ID NO:58), (SEQ ID NO:59), (SEQ ID NO:60), (SEQ ID NO:61), (SEQ ID NO:62), (SEQ ID NO:63), (SEQ ID NO:64), (SEQ ID NO:65), (SEQ ID NO:66), (SEQ ID NO:67), (SEQ ID NO:68), (SEQ ID NO:70), (SEQ ID NO:71), (SEQ ID NO:72), (SEQ ID NO:73), (SEQ ID NO:74), (SEQ ID NO:75), (SEQ ID NO:76), (SEQ ID NO:77), (SEQ ID NO:78), (SEQ ID NO:79), (SEQ ID NO:80), (SEQ ID NO:81), (SEQ ID NO:82), (SEQ ID NO:83), (SEQ ID NO:84), (SEQ ID NO:85), (SEQ ID NO:86), (SEQ ID NO:87), (SEQ ID NO:88), (SEQ ID NO:89), (SEQ ID NO:90), (SEQ ID NO:91), (SEQ ID NO:92), (SEQ ID NO:93), (SEQ ID NO:94), (SEQ ID NO:95), (SEQ ID NO:96), (SEQ ID NO:97), (SEQ ID NO:98), (SEQ ID NO:99), (SEQ ID NO:100) (SEQ ID NO:101), (SEQ ID NO:102), (SEQ ID NO:103), (SEQ ID NO:104), (SEQ ID NO:105), (SEQ ID NO:106), (SEQ ID NO:107), (SEQ ID NO:108) or (SEQ ID NO:109).

104-107. (canceled)

108. The composition of claim 1, wherein the engineered polypeptide is selected from the group consisting of:
(SEQ ID NO: 620)
HGEGTFTSDLSKQLEEEAVRLFIEWLKQGGPSKERSTGGGGSASGSLAEA
KEAANAELDSYGVSDFYKRLIDKAKTVEGVEALKDAILAALP;
(SEQ ID NO: 621)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAALP;
(SEQ ID NO: 622)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAAL;
(SEQ ID NO: 623)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAALP;
(SEQ ID NO: 624)
HGEGTFTSDLSKQMEEEAVRLFIEWLKNGGPSSGAPPPSGGSLAEAKEAA
NAELDSYGVSDFYKRLIDKAKTVEGVEALKDAILAALP;
(SEQ ID NO: 625)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAALP;
(SEQ ID NO: 626)
HGEGTFTSDLSKQLEEEAVRLFIEWLKNGGPSSGAPPPSGGSLAEAKEAA
NAELDSYGVSDFYKRLIDKAKTVEGVEALKDAILAALP;
(SEQ ID NO: 627)
HGEGTFTSDLSKQLEEEAVRLFIEWLKQGGPSKERSTGGGGSASGSLAEA
KEAANAELDSYGVSDFYKRLIDKAKTVEGVEALKDAILAAL;
(SEQ ID NO: 628)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAAL;
(SEQ ID NO: 629)
HGEGTFTSDLSKQLEEEAVRLFIEWLKNGGPSSGAPPPSTGGGGSASGSL
AEAKEAANAELDSYGVSDFYKRLIDKAKTVEGVEALKDAILAA;
(SEQ ID NO: 630)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAAL;
(SEQ ID NO: 631)
HGEGTFTSDLSKQMEEEAVRLFIEWLKNGGPSSGAPPPSGGSLAEAKEAA
NAELDSYGVSDFYKRLIDKAKTVEGVEALKDAILAAL;
(SEQ ID NO: 632)
HGEGTFTSDLSKQLEEEAVRLFIEWLKNGGPSSGAPPPSGGSLAEAKEAA
NAELDSYGVSDFYKRLIDKAKTVEGVEALKDAILAAL;
(SEQ ID NO: 633)
HGEGTFTSDLSKQLEEEAVRLFIEWLKQGGPSKERSTGGGGSASGSLAEA
KEAANAELDSYGVSDFYKRLIDKAKTVEGVEALKDAILAA;
(SEQ ID NO: 634)
HGEGTFTSDLSKQLEEEAVRLFIEWLKNGGPSSGAPPKSTGGGGSASGSL
AEAKEAANAELDSYGVSDFYKRLIDKAKTVEGVEALKDAILAA;
(SEQ ID NO: 635)
HGEGTFTSDLSKQMEEEAVRLFIEWLKNGGPSSGAPPPSTGGGGSASGSL
AEAKEAANAELDSYGVSDFYKRLIDKAKTVEGVEALKDAILAA;
(SEQ ID NO: 636)
HGEGTFTSDLSKQMEEEAVRLFIEWLKNGGPSSGAPPPSGGSLAEAKEAA
NAELDSYGVSDFYKRLIDKAKTVEGVEALKDAILAA;
and
(SEQ ID NO: 637)
HGEGTFTSDLSKQLEEEAVRLFIEWLKNGGPSSGAPPPSGGSLAEAKEAA
NAELDSYGVSDFYKRLIDKAKTVEGVEALKDAILAA.

109-110. (canceled)

111. The composition according to claim 1, wherein the engineered polypeptide has at least 95% sequence identity with Cmpd 2-5, Cmpd 2-9 or Cmpd 2-11.

112-122. (canceled)

123. The composition according to claim 1, wherein said engineered polypeptide has a plasma half-life of at least 40 hours.

124-129. (canceled)

130. The composition according to claim 1, wherein the permeation enhancer enhances paracellular permeation, opens cell tight junctions, enhances transcellular permeation, inhibits an intestinal protease, enhances solubility of a different permeation enhancer and/or is a mucoadhesive.

131-167. (canceled)

168. The composition according to claim 130, further comprising a permeation enhancer that is a salt of a medium chain fatty acid which has a carbon chain length of the carboxylate moiety of from 6 to 20 carbon atoms.

169-204. (canceled)

205. The composition according to claim 1, wherein the permeation enhancer is a salt, ester or ether of a medium chain fatty acid which has a carbon chain length of the carboxylate moiety of from 6 to 20 carbon atoms.

206-220. (canceled)

221. The composition according to claim 1, wherein the permeation enhancer is a cationic, anionic or nonionic surfactant, or mixture thereof.

222-280. (canceled)

281. A pharmaceutical composition according to claim 1, for use in treating a disease or disorder in a subject in need of such treatment.

282-284. (canceled)

285. A method for treating a disease or disorder in a subject, comprising administering a composition according to claim 1 to a subject in need thereof in an amount effective to treat said disease or disorder.

286-294. (canceled)
